(19) United States
(12) Patent Application Publication
(10) Pub. No.: US 2024/0101681 A1
(43) Pub. Date: Mar. 28, 2024

(54) METHODS OF USE OF ANTI-SORTILIN ANTIBODIES

(71) Applicant: Alector LLC, South San Francisco, CA (US)

(72) Inventors: Arnon ROSENTHAL, Woodside, CA (US); Felix Leejia YEH, Belmont, CA (US); Michael F. WARD, San Francisco, CA (US); Robert PAUL, San Francisco, CA (US); Hua LONG, San Carlos, CA (US); Yijie LIAO, Pleasanton, CA (US)

(73) Assignee: Alector LLC, South San Francisco, CA (US)

(21) Appl. No.: 18/039,945

(22) PCT Filed: Dec. 1, 2021
(86) PCT No.: PCT/US2021/072682

§ 371 Date: Jun. 1, 2023

Related U.S. Application Data
(60) Provisional application No. 63/120,670, filed on Dec. 2, 2020.
(60) Provisional application No. 63/271,658, filed on Oct. 25, 2021.

(51) Int. Cl. C07K 16/28; A61K 39/395; A61K 45/06

(52) U.S. Cl. CPC C07K 16/286; A61K 39/3955; A61K 45/06; A61K 2039/505

(57) ABSTRACT

The present disclosure is generally directed to the use of antibodies that specifically bind one or more epitopes within a sortilin protein, e.g., human sortilin or mammalian sortilin, for treating and/or delaying progression of a disease, disorder, or injury in an individual in need thereof.

### CROSS-REFERENCE TO RELATED APPLICATIONS

This application claims the benefit of U.S. Provisional Application No. 63/120,670, filed Dec. 2, 2020 and U.S. Provisional Application No. 63/271,658, filed Oct. 25, 2021, the contents of which are hereby incorporated by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 735022003640SEQLIST.TXT, date recorded: Nov. 29, 2021, size: 55,902 bytes).

### FIELD

The present disclosure relates to therapeutic uses of anti-sortilin antibodies.

### BACKGROUND

Sortilin is a Type I transmembrane protein that acts both as a receptor of several ligands, and in the sorting of select cargo from the trans-Golgi network (TGN) to late endosomes and lysosomes for degradation. Sortilin binds the secreted protein progranulin (PGRN) and targets it for lysosomal degradation, thus negatively regulating extracellular levels of progranulin (Hu, F et al. (2010) Neuron 68, 654-667). In line with this, deficiency of sortilin significantly increases plasma progranulin levels both in mouse models in vivo and human cells in vitro (Carrasquillo, M. M et al., (2010) Am J Hum Genet 87, 890-897; Lee, W. C et al., (2014) Hum Mol Genet 23, 1467-1478). Moroever, a polymorphism in sortilin was shown to be strongly associated with progranulin serum levels in humans (Carrasquillo M M e al., (2010), Am J Hum Genet. 10; 87(6):890-7).

Progranulin (PGRN), encoded by the GRN gene, is a secreted, growth factor-like, trophic, and anti-inflammatory protein, which also plays a role as an adipokine involved in diet-induced obesity and insulin resistance (Nguyen D A et al., (2013). Trends in Endocrinology and Metabolism, 24, 597-606).

Progranulin deficiency accounts for roughly 25% of all heritable forms of frontotemporal dementia (FTD), an early-onset neurodegenerative disease. Patients with heterozygous loss-of-function mutations in progranulin have ˜50% reduced extracellular levels of the protein and they will invariably develop FTD, making progranulin a causal gene for the disease (Baker, M et al., (2006) Nature 442, 916-919; Carecchio M et al., (2011) J Alzheimers Dis 27, 781-790; Cruts, M et al., (2008) Trends Genet 24, 186-194; Galimberti, D et al., (2010) J Alzheimers Dis 19, 171-177).

Progranulin has also been associated with Parkinson's disease. The GRN Parkinson's disease risk allele is associated with reduced progranulin levels in plasma, cerebrospinal fluid, and brain. In addition, homozygous loss of function of progranulin causes neuronal ceroid lipofuscinosis, and heterozygous loss of function of progranulin causes frontotemporal dementia (FTD), sometimes with Parkinsonism presentation (Smith et al., Am J Hum Genet (2012) 90(6):1102-1107; Boeve et al., Brain (2006) 129(Pt 11):3103-3114; and Wauters et al., Neurobiol Aging (2018) 67:84-94). Furthermore, reduced plasma progranulin levels are associated with increased severity of Parkinson's disease (Yao et al., Neurosci Lett (2020) 725:134873). Progranulin deficiency also worsens outcomes in rodent Parkinson's disease models (Martens et al., J Clin Invest (2012) 122(11):3955-3959), while delivery of the progranulin gene improves those outcomes (Van Kampen et al., PLoS One (2014) 7; 9(5):e97032).

In addition, progranulin mutant alleles have been identified in Alzheimer's disease patients (Seelaar, H et al., (2011). Journal of neurology, neurosurgery, and psychiatry 82, 476-486). Importantly, progranulin acts protectively in several disease models, with increased progranulin levels accelerating behavioral recovery from ischemia (Tao, J et al., (2012) Brain Res 1436, 130-136; Egashira, Y. et al., (2013) J Neuroinflammation 10, 105), attenuating pathology in a model of amyotrophic lateral sclerosis (Laird, A. S et al., (2010). PLoS One 5, e13368.) and arthritis (Tang, W et al., (2011). Science 332, 478-484), and preventing memory deficits in an Alzheimer's disease model (Minami, S. S et al., (2014). Nat Med 20, 1157-1164).

Through its various interactions with proteins such as progranulin, sortilin and its multiple ligands have been shown to be involved in various diseases, disorders, and conditions, such as frontotemporal dementia (FTD), amyotrophic lateral sclerosis (ALS), amyotrophic lateral sclerosis-frontotemporal dementia phenotypes, Alzheimer's disease, Parkinson's disease, depression, neuropsychiatric disorders, vascular dementia, seizures, retinal dystrophy, age related macular degeneration, glaucoma, traumatic brain injury, aging, wound healing, stroke, arthritis, and atherosclerotic vascular diseases.

Novel therapeutic antibodies targeting sortilin are one solution to treating diseases associated with sortilin or progranulin activity. However, administration of monoclonal antibodies may present a challenge for such therapeutic use. For example, therapeutic antibodies generally have limited oral bioavailability, so they are typically administered intravenously, subcutaneously, or intramuscularly (Ovacik, M and Lin, L, (2018) Clin Transl Sci 11, 540-552). Of available administration options, subcutaneous administration is the most convenient because it can be done at home and often by the patient, while intravenous administration delivers higher systemic exposures. Because delivery to the cerebrospinal fluid (CSF) requires high systemic doses, intravenous administration is usually employed because subcutaneous administration may not deliver sufficiently high doses. However, intravenous administration may be challenging for patients with neurodegenerative diseases, such as Alzheimer's disease, Parkinson's disease or FTD, in which treatment may be administered over the course of many years and patient compliance may otherwise be difficult.

Accordingly, there is a need for therapeutic antibodies that specifically bind sortilin proteins in order to treat one or more diseases, disorders, and conditions associated with sortilin activity. Furthermore, there are additional needs for identifying methods of treating patients with the correct dose and of administering that dose in ways that ease patient compliance.

All references cited herein, including patents, patent applications and publications, are hereby incorporated by reference in their entirety.

### SUMMARY

In one aspect, provided herein is a method of treating or delaying the progression of a disease, disorder, or injury in an individual, comprising administering to the individual an anti-sortilin antibody at a dose of at least about 6 mg/kg, wherein the antibody comprises a light chain variable domain and a heavy chain variable domain, wherein the heavy chain variable domain comprises an HVR-H1 comprising the amino acid sequence of SEQ ID NO:6, an HVR-H2 comprising the amino acid sequence of SEQ ID NO: 7, and an HVR-H3 comprising the amino acid sequence of SEQ ID NO:8, and the light chain variable domain comprises an HVR-L1 comprising the amino acid sequence of SEQ ID NO:9, an HVR-L2 comprising the amino acid sequence of SEQ ID NO:10, and an HVR-L3 comprising the amino acid sequence of SEQ ID NO:11.

In another aspect, provided herein is a method of increasing progranulin levels in an individual having a disease, disorder, or injury, comprising administering to the individual an anti-sortilin antibody at a dose of at least about 6 mg/kg, wherein the antibody comprises a light chain variable domain and a heavy chain variable domain, wherein the heavy chain variable domain comprises an HVR-H1 comprising the amino acid sequence of SEQ ID NO:6, an HVR-H2 comprising the amino acid sequence of SEQ ID NO: 7, and an HVR-H3 comprising the amino acid sequence of SEQ ID NO:8, and the light chain variable domain comprises an HVR-L1 comprising the amino acid sequence of SEQ ID NO:9, an HVR-L2 comprising the amino acid sequence of SEQ ID NO:10, and an HVR-L3 comprising the amino acid sequence of SEQ ID NO: 11. In some embodiments, the increase in progranulin levels comprises an increase in the level of progranulin in the cerebrospinal fluid of the individual, the plasma of the individual, or both.

In another aspect, provided herein is a method of treating or delaying the progression of a disease, disorder, or injury in an individual, comprising administering to the individual an anti-sortilin antibody by subcutaneous injection at a dose of between about 150 mg and about 600 mg, wherein the antibody comprises a light chain variable domain and a heavy chain variable domain, wherein the heavy chain variable domain comprises an HVR-H1 comprising the amino acid sequence of SEQ ID NO:6, an HVR-H2 comprising the amino acid sequence of SEQ ID NO: 7, and an HVR-H3 comprising the amino acid sequence of SEQ ID NO:8, and the light chain variable domain comprises an HVR-L1 comprising the amino acid sequence of SEQ ID NO:9, an HVR-L2 comprising the amino acid sequence of SEQ ID NO:10, and an HVR-L3 comprising the amino acid sequence of SEQ ID NO:11.

In another aspect, provided herein is a method of increasing progranulin levels in an individual having a disease, disorder, or injury, comprising administering to the individual an anti-sortilin antibody by subcutaneous injection at a dose of between about 150 mg and about 600 mg, wherein the antibody comprises a light chain variable domain and a heavy chain variable domain, wherein the heavy chain variable domain comprises an HVR-H1 comprising the amino acid sequence of SEQ ID NO:6, an HVR-H2 comprising the amino acid sequence of SEQ ID NO: 7, and an HVR-H3 comprising the amino acid sequence of SEQ ID NO:8, and the light chain variable domain comprises an HVR-L1 comprising the amino acid sequence of SEQ ID NO:9, an HVR-L2 comprising the amino acid sequence of SEQ ID NO:10, and an HVR-L3 comprising the amino acid sequence of SEQ ID NO:11.

In some embodiments, which may be combined with any of the preceding embodiments, the method comprises administering the anti-sortilin antibody by intravenous infusion or by subcutaneous injection. In some embodiments, the method comprises administering the anti-sortilin antibody by intravenous infusion at a dose of at least about 6 mg/kg, at least about 15 mg/kg, at least about 30 mg/kg, or at least about 60 mg/kg. In some embodiments, the method comprises administering the anti-sortilin antibody by intravenous infusion at a dose of between about 6 mg/kg and about 30 mg/kg. In some embodiments, the method comprises administering the anti-sortilin antibody by intravenous infusion at a dose of about 6 mg/kg, about 15 mg/kg, about 30 mg/kg, or about 60 mg/kg.

In some embodiments, the method comprises: (a) administering the anti-sortilin antibody by intravenous infusion at an initial dose of about 60 mg/kg, followed by one or more lower doses of between about 6 mg/kg and about 59 mg/kg, or between about 6 mg/kg and about 30 mg/kg, or between about 6 mg/kg and about 15 mg/kg, of the anti-sortilin antibody; (b) administering the anti-sortilin antibody by intravenous infusion at an initial dose of about 30 mg/kg, followed by one or more lower doses of between about 6 mg/kg and about 29 mg/kg, or between about 6 mg/kg and about 15 mg/kg, of the anti-sortilin antibody; (c) administering the anti-sortilin antibody by intravenous infusion at an initial dose of about 15 mg/kg, followed by one or more lower doses of between about 6 mg/kg and about 14 mg/kg of the anti-sortilin antibody; (d) administering the anti-sortilin antibody by intravenous infusion at an initial dose of about 6 mg/kg, followed by one or more higher doses of between about 7 mg/kg and about 30 mg/kg, between about 15 mg/kg and about 30 mg/kg, or between about 30 mg/kg and about 60 mg/kg, of the anti-sortilin antibody; (e) administering the anti-sortilin antibody by intravenous infusion at an initial dose of about 15 mg/kg, followed by one or more higher doses of between about 16 mg/kg and about 30 mg/kg, or between about 30 mg/kg and about 60 mg/kg, of the anti-sortilin antibody; or (f) administering the anti-sortilin antibody by intravenous infusion at an initial dose of about 30 mg/kg, followed by one or more higher doses of between about 31 mg/kg and about 60 mg/kg of the anti-sortilin antibody

In some embodiments, the method comprises administering the anti-sortilin antibody about once every four weeks or less frequently. In some embodiments, the method comprises administering the anti-sortilin antibody about once every four weeks, about once every five weeks, about once every six weeks, about once every seven weeks, about once every eight weeks, about once every nine weeks, or about once every ten weeks. In some embodiments, the method comprises administering the anti-sortilin antibody by intravenous infusion at a dose of about 15 mg/kg about once every four weeks, about once every six weeks, or about once every eight weeks. In some embodiments, the method comprises administering the anti-sortilin antibody by intravenous infusion at a dose of about 15 mg/kg about once every six weeks or about once every eight weeks. In some embodiments, the method comprises administering the anti-sortilin antibody by intravenous infusion at a dose of about 30 mg/kg about once every four weeks, about once every six weeks, or about once every eight weeks. In some embodiments, the method comprises administering the anti-sortilin antibody by intravenous infusion at a dose of about 30 mg/kg about once every six weeks or about once every eight weeks. In some embodiments, the method comprises administering the anti-sortilin antibody by intravenous infusion at a dose of about 60 mg/kg about once every four weeks, about once every six weeks or about once every eight weeks. In some embodiments, the method comprises administering the anti-sortilin antibody by intravenous infusion at a dose of about 60 mg/kg about once every six weeks or about once every eight weeks. In some embodiments, the method comprises administering the anti-sortilin antibody by subcutaneous injection at a dose of at least about 270 mg. In some embodiments, the method comprises administering the anti-sortilin antibody by subcutaneous injection at a dose of between about 150 mg and about 600 mg. In some embodiments, the method comprises administering the anti-sortilin antibody by subcutaneous injection at a dose of any of about 150 mg, about 270 mg, about 300 mg, or about 600 mg. In some embodiments, the method comprises administering the anti-sortilin antibody by subcutaneous injection at a dose of about 150 mg. In some embodiments, the method comprises administering the anti-sortilin antibody by subcutaneous injection at a dose of about 270 mg. In some embodiments, the method comprises administering the anti-sortilin antibody by subcutaneous injection at a dose of about 300 mg. In some embodiments, the method comprises administering the anti-sortilin antibody by subcutaneous injection at a dose of about 600 mg. In some embodiments, the method comprises administering the anti-sortilin antibody by subcutaneous injection any of about every two weeks, about every four weeks, about every six weeks, or about every eight weeks. In some embodiments, the method comprises administering the anti-sortilin antibody by subcutaneous injection about once every four weeks. In some embodiments, which may be combined with any of the preceding embodiments, the anti-sortilin antibody comprises a heavy chain variable domain comprising the amino acid sequence of SEQ ID NO: 20 and a light chain variable domain comprising the amino acid sequence of SEQ ID NO: 21. In some embodiments, the anti-sortilin antibody has a human IgG1 isotype. In some embodiments, the anti-sortilin antibody comprises an Fc region comprising amino acid substitutions L234A, L235A, and P331S, wherein the numbering of the residue position is according to EU numbering. In some embodiments, the anti-sortilin antibody comprises a heavy chain constant region comprising the amino acid sequence of SEQ ID NO: 25 or 26. In some embodiments, the anti-sortilin antibody comprises a light chain comprising the amino acid sequence of SEQ ID NO: 30, and a heavy chain comprising the amino acid sequence of SEQ ID NO: 31 or 32. In some embodiments, the anti-sortilin antibody comprises a light chain comprising the amino acid sequence of SEQ ID NO: 30, and a heavy chain comprising the amino acid sequence of SEQ ID NO: 32. In some embodiments, the anti-sortilin antibody comprises a light chain comprising the amino acid sequence of SEQ ID NO: 30, and a heavy chain comprising the amino acid sequence of SEQ ID NO: 31.

In some embodiments, which may be combined with any of the preceding embodiments, the method further comprises measuring the level of progranulin in a sample of blood or cerebrospinal fluid obtained from the individual before and after the individual has received one or more doses of the anti-sortilin antibody.

In some embodiments, which may be combined with any of the preceding embodiments, the method further comprises measuring the level of sortilin in white blood cells in a sample of blood obtained from the individual before and after the individual has received one or more doses of the anti-sortilin antibody.

In some embodiments, which may be combined with any of the preceding embodiments, the method further comprises measuring the level of neurofilament light chain (NF-L), Tau, one or more biomarkers of neuroinflammation, one or more inflammatory biomarkers, one or more biomarkers of complement function, and/or one or more biomarkers of microglial activity in a sample of blood or cerebrospinal fluid obtained from the individual before and after the individual has received one or more doses of the anti-sortilin antibody. In some embodiments, (a) the one or more biomarkers of neuroinflammation are selected from the group consisting of IL-6, SPP1, IFI2712A, CHIT1, YKL-40, GFAP, YWHAE, CSF1, AIF1, LY86, CD86, and TOP2A; (b) the one or more inflammatory biomarkers are selected from the group consisting of osteopontin (SPP1), YWHAE (14-3-3 protein epsilon), allograft inflammatory factor 1 (AIF1), colony stimulating factor 1 (CSF1), chitinase 1 (CHIT1), lymphocyte antigen 86 (LY86), and CD86; (c) the one or more biomarkers of complement function are selected from the group consisting of C1qb and C1qc; and/or (d) the one or more biomarkers of microglial activity are selected from the group consisting of YKL-40, GFAP and interleukin-6.

In some embodiments, which may be combined with any of the preceding embodiments, administration to the individual of one dose of 6 mg/kg or 15 mg/kg of the anti-sortilin antibody by intravenous infusion results in an increase in the level of progranulin in the plasma of the individual of at least about 1.4-fold, at least about 1.8-fold, at least about 2-fold, at least about 2.2-fold, at least about 2.4-fold, at least about 2.6-fold, at least about 2.8-fold, or at least about 3-fold, compared to the level of progranulin in the plasma of the individual prior to administration of the anti-sortilin antibody. In some embodiments, the increase in the level of progranulin in the plasma of the individual is present within about 1 day after administration of the anti-sortilin antibody. In some embodiments, the dose of the anti-sortilin antibody is 6 mg/kg, and the increase in the level of progranulin in the plasma of the individual is present at about 1 day, at about 2 days, at about 5 days, at about 7 days, at about 12 days, at about 17 days, at about 24 days, at about 29 days, or at about 42 days after administration of the anti-sortilin antibody. In some embodiments, the dose of the anti-sortilin antibody is 15 mg/kg, and the increase in the level of progranulin in the plasma of the individual is present at about 1 day, at about 2 days, at about 5 days, at about 7 days, at about 12 days, at about 17 days, at about 24 days, at about 29 days, at about 42 days, or at about 56 days after administration of the anti-sortilin antibody.

In some embodiments, which may be combined with any of the preceding embodiments, administration to the individual of one dose of 6 mg/kg, 15 mg/kg, 30 mg/kg or 60 mg/kg of the anti-sortilin antibody by intravenous infusion results in an increase in the level of progranulin in the plasma of the individual of at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300%, compared to the level of progranulin in the plasma of the individual prior to administration of the anti-sortilin antibody. In some embodiments, the dose of the anti-sortilin antibody is 6 mg/kg, and the increase in the level of progranulin in the plasma of the individual is present at about 1 day, at about 7 days, at about 14 days, at about 21 days, or at about 28 days after administration of the anti-sortilin antibody. In some embodiments, the dose of the anti-sortilin antibody is 15 mg/kg, and the increase in the level of progranulin in the plasma of the individual is present at about 1 day, at about 7 days, at about 14 days, at about 21 days, at about 28 days, at about 35 days, or at about 42 days after administration of the anti-sortilin antibody. In some embodiments, the dose of the anti-sortilin antibody is 30 mg/kg, and the increase in the level of progranulin in the plasma of the individual is present at about 1 day, at about 7 days, at about 14 days, at about 21 days, at about 28 days, at about 35 days, at about 42 days, at about 49 days, or at about 56 days after administration of the anti-sortilin antibody. In some embodiments, the dose of the anti-sortilin antibody is 60 mg/kg, and the increase in the level of progranulin in the plasma of the individual is present at about 1 day, at about 7 days, at about 14 days, at about 21 days, at about 28 days, at about 35 days, at about 42 days, at about 49 days, at about 56 days, at about 63 days, at about 70 days, at about 77 days, or at about 84 days after administration of the anti-sortilin antibody.

In some embodiments, which may be combined with any of the preceding embodiments, administration to the individual of one dose of about 150 mg of the anti-sortilin antibody by subcutaneous injection results in an increase in the level of progranulin in the plasma of the individual of at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, or at least about 200%, compared to the level of progranulin in the plasma of the individual prior to administration of the anti-sortilin antibody. In some embodiments, which may be combined with any of the preceding embodiments, administration to the individual of one dose of about 300 mg of the anti-sortilin antibody by subcutaneous injection results in an increase in the level of progranulin in the plasma of the individual of at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, or at least about 200%, compared to the level of progranulin in the plasma of the individual prior to administration of the anti-sortilin antibody. In some embodiments, which may be combined with any of the preceding embodiments, administration to the individual of one dose of about 600 mg of the anti-sortilin antibody by subcutaneous injection results in an increase in the level of progranulin in the plasma of the individual of at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, or at least about 200%, compared to the level of progranulin in the plasma of the individual prior to administration of the anti-sortilin antibody. In some embodiments, the increase in the level of progranulin in the plasma of the individual is present at about 1 day, at about 7 days, at about 14 days, at about 21 days, or at about 28 days after administration of the anti-sortilin antibody.

In some embodiments, which may be combined with any of the preceding embodiments, administration to the individual of one dose of 6 mg/kg of the anti-sortilin antibody by intravenous infusion results in an increase in the level of progranulin in the cerebrospinal fluid of the individual of at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, or at least about 20%, compared to the level of progranulin in the cerebrospinal fluid of the individual prior to administration of the anti-sortilin antibody. In some embodiments, the increase in the level of progranulin in the cerebrospinal fluid of the individual is present at about 1 day, at about 2 days, at about 5 days, at about 7 days, at about 12 days, at about 14 days, at about 17 days, at about 21 days, at about 24 days, at about 28 days, at about 29 days, at about 35 days, at about 42 days, at about 49 days, or at about 56 days after administration of the anti-sortilin antibody.

In some embodiments, which may be combined with any of the preceding embodiments, administration to the individual of one dose of 15 mg/kg of the anti-sortilin antibody by intravenous infusion results in an increase in the level of progranulin in the cerebrospinal fluid of the individual of at least about 5%, at least about 10%, at least about 20%, at least about 30%, at least about 40%, at least about 50%, at least about 60%, at least about 70%, or at least about 80%, compared to the level of progranulin in the cerebrospinal fluid of the individual prior to administration of the anti-sortilin antibody. In some embodiments, the increase in the level of progranulin in the cerebrospinal fluid of the individual is present at about 1 day, at about 2 days, at about 5 days, at about 7 days, at about 12 days, at about 14 days, at about 17 days, at about 21 days, at about 24 days, at about 28 days, at about 29 days, at about 35 days, at about 42 days, at about 49 days, or at about 56 days after administration of the anti-sortilin antibody.

In some embodiments, which may be combined with any of the preceding embodiments, administration to the individual of one dose of 30 mg/kg of the anti-sortilin antibody by intravenous infusion results in an increase in the level of progranulin in the cerebrospinal fluid of the individual of at least about 5%, at least about 10%, at least about 20%, at least about 30%, at least about 40%, at least about 50%, at least about 60%, or at least about 70%, compared to the level of progranulin in the cerebrospinal fluid of the individual prior to administration of the anti-sortilin antibody. In some embodiments, the increase in the level of progranulin in the cerebrospinal fluid of the individual is present at about 1 day, at about 2 days, at about 5 days, at about 7 days, at about 12 days, at about 14 days, at about 17 days, at about 21 days, at about 24 days, at about 28 days, at about 29 days, at about 35 days, at about 42 days, at about 49 days, or at about 56 days after administration of the anti-sortilin antibody.

In some embodiments, which may be combined with any of the preceding embodiments, administration to the individual of one dose of 60 mg/kg of the anti-sortilin antibody by intravenous infusion results in an increase in the level of progranulin in the cerebrospinal fluid of the individual of at least about 5%, at least about 10%, at least about 20%, at least about 30%, at least about 40%, at least about 50%, at least about 60%, or at least about 70%, compared to the level of progranulin in the cerebrospinal fluid of the individual prior to administration of the anti-sortilin antibody. In some embodiments, the increase in the level of progranulin in the cerebrospinal fluid of the individual is present at about 1 day, at about 7 days, at about 14 days, at about 21 days, at about 28 days, at about 35 days, at about 42 days, at about 49 days, at about 56 days, at about 63 days, at about 70 days, at about 77 days, or at about 84 days after administration of the anti-sortilin antibody.

In some embodiments, which may be combined with any of the preceding embodiments, administration to the individual of one dose of about 150 mg of the anti-sortilin antibody by subcutaneous injection results in an increase in the level of progranulin in the cerebrospinal fluid of the individual of at least about 5%, at least about 10%, at least about 20%, at least about 30%, at least about 40%, at least about 50%, at least about 60%, or at least about 70%, compared to the level of progranulin in the cerebrospinal fluid of the individual prior to administration of the anti-sortilin antibody. In some embodiments, which may be combined with any of the preceding embodiments, administration to the individual of one dose of about 300 mg of the anti-sortilin antibody by subcutaneous injection results in an increase in the level of progranulin in the cerebrospinal fluid of the individual of at least about 5%, at least about 10%, at least about 20%, at least about 30%, at least about 40%, at least about 50%, at least about 60%, or at least about 70%, compared to the level of progranulin in the cerebrospinal fluid of the individual prior to administration of the anti-sortilin antibody. In some embodiments, which may be combined with any of the preceding embodiments, administration to the individual of one dose of about 600 mg of the anti-sortilin antibody by subcutaneous injection results in an increase in the level of progranulin in the cerebrospinal fluid of the individual of at least about 5%, at least about 10%, at least about 20%, at least about 30%, at least about 40%, at least about 50%, at least about 60%, or at least about 70%, compared to the level of progranulin in the cerebrospinal fluid of the individual prior to administration of the anti-sortilin antibody. In some embodiments, the increase in the level of progranulin in the cerebrospinal fluid of the individual is present at about 1 day, at about 7 days, at about 14 days, or at about 21 days after administration of the anti-sortilin antibody.

In some embodiments, which may be combined with any of the preceding embodiments, the disease, disorder, or injury is selected from frontotemporal dementia, progressive supranuclear palsy, Alzheimer's disease, vascular dementia, seizures, retinal dystrophy, amyotrophic lateral sclerosis (ALS), traumatic brain injury, a spinal cord injury, dementia, stroke, Parkinson's disease, acute disseminated encephalomyelitis, retinal degeneration, age related macular degeneration, glaucoma, multiple sclerosis, septic shock, bacterial infection, arthritis, or osteoarthritis. In some embodiments, the individual is at risk for the disease, disorder, or injury. In some embodiments, the disease, disorder, or injury is Parkinson's disease. In some embodiments, the individual is at risk for Parkinson's disease. In some embodiments, the disease, disorder, or injury is sporadic Parkinson's disease. In some embodiments, the individual has at least one pathogenic mutation in the GBA1 gene. In some embodiments, the individual is homozygous or heterozygous for the at least one pathogenic mutation in the GBA1 gene. In some embodiments, the at least one pathogenic mutation in the GBA1 gene is selected from the group consisting of c.1226A>G, c.1448T>C, IVS2+1G>A, RecNciI, 84insGG, and any combination thereof; or wherein the at least one pathogenic mutation in the GBA1 gene is a mutation in the GBA1 gene resulting in an amino acid substitution in a GBA1 gene product selected from the group consisting of N370S, L444P, R120W, H255Q, D409H, E326K, T369M, R496H, and any combination thereof. In some embodiments, the Parkinson's disease is classified as being between Stage I and Stage III, based on the Hoehn and Yahr criteria. In some embodiments, the individual is taking one or more treatments for Parkinson's disease prior to administration of the anti-sortilin antibody. In some embodiments, the individual continues taking the one or more treatments for Parkinson's disease after the start of treatment with the anti-sortilin antibody. In some embodiments, the method comprises administering the anti-sortilin antibody in combination with one or more treatments for Parkinson's disease. In some embodiments, the one or more treatments for Parkinson's disease are selected from a glutamate antagonist, an anticholinergic, a dopamine agonist, Levodopa (L-DOPA and decarboxylase [DDC] inhibitor), a monoamine oxidase B (MAO-B) inhibitor, a catechol-O-methyltransferase (COMT) inhibitor, a beta blocker, a selective serotonin uptake inhibitor (SSRI), a tricyclic antidepressant (TCA), or indomethacin. In some embodiments, the method further comprises measuring the levels of progranulin, GCase, and/or alpha-synuclein in a sample of plasma or cerebrospinal fluid obtained from the individual before and after the individual has received one or more doses of the anti-sortilin antibody. In some embodiments, the method further comprises measuring the level of one or more biomarkers of lysosomal function in a sample of plasma or cerebrospinal fluid obtained from the individual before and after the individual has received one or more doses of the anti-sortilin antibody. In some embodiments, the one or more biomarkers of lysosomal function are selected from GCase protein, GCase activity, lyso-Gb1, one or more cathepsins, LAMP1, N-acetyl-D-glucosamine kinase (NAGK), and glucosylceramide; optionally, wherein the one or more cathepsins are selected from cathepsin B (CTSB) and cathepsin D (CTSD). In some embodiments, the method further comprises assessing cognitive function of the individual before and after the individual has received one or more doses of the anti-sortilin antibody. In some embodiments, cognitive function is assessed using the Montreal Cognitive Assessment (MoCA). In some embodiments, the method further comprises assessing motor function of the individual before and after the individual has received one or more doses of the anti-sortilin antibody. In some embodiments, motor function is assessed using the Movement Disorder Society (MDS)-sponsored revision of the Unified Parkinson's Disease Rating Scale (UPDRS) Part III (MDS-UPDRS Part III), or the UPDRS total scale. In some embodiments, the method comprises administering the anti-sortilin antibody to the individual for at least about 6 months or at least about 24 weeks.

In some embodiments, the disease, disorder, or injury is frontotemporal dementia (FTD). In some embodiments, the individual: (a) is heterozygous for one or more mutations in the GRN gene, optionally wherein the one or more mutations are loss-of-function mutations; (b) is heterozygous for a C9orf72 hexanucleotide repeat expansion; (c) has symptoms of FTD, does not have symptoms of FTD, or has pre-symptomatic FTD; and/or (d) has FTD-GRN, or FTD caused by one or more mutations in the GRN gene. In some embodiments, (a) the individual has pre-symptomatic FTD and: (i) elevated levels of one or more biomarkers selected from the group consisting of Nfl, SPP1, YWHAE, AIF1, CSF1, CHIT1, and LY86, and/or (ii) reduced levels of one or more biomarkers selected from the group consisting of NAGK and CTSB; or (b) the individual does not have symptoms of FTD and: (i) is heterozygous for one or more mutations in the GRN gene, and/or (ii) has reduced PGRN levels or function

In some embodiments, the disease, disorder, or injury is Alzheimer's disease.

In some embodiments, the disease, disorder, or injury is ALS.

In some embodiments, which may be combined with any of the preceding embodiments, the method further comprises measuring the levels of progranulin, GCase, and/or alpha-synuclein in a sample of plasma or cerebrospinal fluid obtained from the individual before and after the individual has received one or more doses of the anti-sortilin antibody. In some embodiments, which may be combined with any of the preceding embodiments, the method further comprises measuring the level of one or more biomarkers of lysosomal function in a sample of plasma or cerebrospinal fluid obtained from the individual before and after the individual has received one or more doses of the anti-sortilin antibody. In some embodiments, the one or more biomarkers of lysosomal function are selected from GCase protein, GCase activity, lyso-Gb1, one or more cathepsins, LAMP1, N-acetyl-D-glucosamine kinase (NAGK), and glucosylceramide; optionally, wherein the one or more cathepsins are selected from cathepsin B (CTSB) and cathepsin D (CTSD). In some embodiments, which may be combined with any of the preceding embodiments, the method further comprises assessing cognitive function of the individual before and after the individual has received one or more doses of the anti-sortilin antibody. In some embodiments, which may be combined with any of the preceding embodiments, the individual is a human.

In another aspect, provided herein is a method of monitoring treatment of an individual being administered an anti-sortilin antibody, comprising measuring the level of one or more biomarkers, wherein the one or more biomarkers are selected from progranulin, GCase, neurofilament light chain (NF-L), Tau, one or more markers of neuroinflammation, one or more inflammatory biomarkers, one or more biomarkers of complement function, one or more biomarkers of microglial activity, or alpha-synuclein, wherein the level of the one or more biomarkers is measured in a sample of plasma or cerebrospinal fluid obtained from the individual before and after the individual has received one or more doses of an anti-sortilin antibody.

In another aspect, provided herein is a method of monitoring treatment of an individual being administered an anti-sortilin antibody, comprising measuring the level of one or more biomarkers of lysosomal function in a sample of plasma or cerebrospinal fluid obtained from the individual before and after the individual has received one or more doses of an anti-sortilin antibody.

In some embodiments, which may be combined with any of the preceding embodiments, the one or more biomarkers of lysosomal function are selected from GCase protein, GCase activity, lyso-Gb1, one or more cathepsins, LAMP1, N-acetyl-D-glucosamine kinase (NAGK), and glucosylceramide; optionally, wherein the one or more cathepsins are selected from cathepsin B (CTSB) and cathepsin D (CTSD). In some embodiments, the method further comprises assessing the activity of the anti-sortilin antibody in the individual based on the level of the one or more biomarkers in the sample.

In some embodiments, which may be combined with any of the preceding embodiments, (a) the one or more markers of neuroinflammation are selected from IL-6, SPP1, IFI2712A, CHIT1, YKL-40, GFAP, YWHAE, CSF1, AIF1, LY86, CD86, and TOP2A; (b) the one or more inflammatory biomarkers are selected from osteopontin (SPP1), YWHAE (14-3-3 protein epsilon), allograft inflammatory factor 1 (AIF1), colony stimulating factor 1 (CSF1), chitinase 1 (CHIT1), lymphocyte antigen 86 (LY86), and CD86; (c) the one or more biomarkers of complement function are selected from C1qb and C1qc; and/or (d) the one or more biomarkers of microglial activity are selected from YKL-40, GFAP and interleukin-6.

In another aspect, provided herein is an antibody that binds to a sortilin protein, wherein the antibody comprises a heavy chain variable domain comprising the amino acid sequence of SEQ ID NO: 20 and a light chain variable domain comprising the amino acid sequence of SEQ ID NO: 21, and wherein the antibody has a human IgG1 isotype and comprises an Fc region comprising amino acid substitutions L234A, L235A, and P331S, wherein the numbering of the residue position is according to EU numbering.

In another aspect, provided herein is an antibody that binds to a sortilin protein, wherein the antibody comprises a light chain comprising the amino acid sequence of SEQ ID NO: 30, and a heavy chain comprising the amino acid sequence of SEQ ID NO: 31 or 32.

In another aspect, provided herein is an anti-sortilin antibody at a dose of at least about 6 mg/kg for use in a method of treating or delaying the progression of a disease, disorder, or injury in an individual, wherein the antibody comprises a light chain variable domain and a heavy chain variable domain, wherein the heavy chain variable domain comprises an HVR-H1 comprising the amino acid sequence of SEQ ID NO:6, an HVR-H2 comprising the amino acid sequence of SEQ ID NO: 7, and an HVR-H3 comprising the amino acid sequence of SEQ ID NO:8, and the light chain variable domain comprises an HVR-L1 comprising the amino acid sequence of SEQ ID NO:9, an HVR-L2 comprising the amino acid sequence of SEQ ID NO:10, and an HVR-L3 comprising the amino acid sequence of SEQ ID NO:11.

In another aspect, provided herein is an anti-sortilin antibody at a dose of at least about 6 mg/kg for use in a method for increasing progranulin levels in an individual having a disease, disorder, or injury, wherein the antibody comprises a light chain variable domain and a heavy chain variable domain, wherein the heavy chain variable domain comprises an HVR-H1 comprising the amino acid sequence of SEQ ID NO:6, an HVR-H2 comprising the amino acid sequence of SEQ ID NO: 7, and an HVR-H3 comprising the amino acid sequence of SEQ ID NO:8, and the light chain variable domain comprises an HVR-L1 comprising the amino acid sequence of SEQ ID NO:9, an HVR-L2 comprising the amino acid sequence of SEQ ID NO:10, and an HVR-L3 comprising the amino acid sequence of SEQ ID NO:11.

In another aspect, provided herein is an antibody that binds to a sortilin protein for use in a method of treating or delaying the progression of a disease, disorder, or injury in an individual, wherein the antibody comprises a heavy chain variable domain comprising the amino acid sequence of SEQ ID NO: 20 and a light chain variable domain comprising the amino acid sequence of SEQ ID NO: 21, and wherein the antibody has a human IgG1 isotype and comprises an Fc region comprising amino acid substitutions L234A, L235A, and P331S, wherein the numbering of the residue position is according to EU numbering.

In another aspect, provided herein is an antibody that binds to a sortilin protein for use in a method for increasing progranulin levels in an individual having a disease, disorder, or injury, wherein the antibody comprises a heavy chain variable domain comprising the amino acid sequence of SEQ ID NO: 20 and a light chain variable domain comprising the amino acid sequence of SEQ ID NO: 21, and wherein the antibody has a human IgG1 isotype and comprises an Fc region comprising amino acid substitutions L234A, L235A, and P331S, wherein the numbering of the residue position is according to EU numbering.

In another aspect, provided herein is an antibody that binds to a sortilin protein for use in a method of treating or delaying the progression of a disease, disorder, or injury in an individual, wherein the antibody comprises a light chain comprising the amino acid sequence of SEQ ID NO: 30, and a heavy chain comprising the amino acid sequence of SEQ ID NO: 31 or 32.

In another aspect, provided herein is an antibody that binds to a sortilin protein for use in a method for increasing progranulin levels in an individual having a disease, disorder, or injury, wherein the antibody comprises a light chain comprising the amino acid sequence of SEQ ID NO: 30, and a heavy chain comprising the amino acid sequence of SEQ ID NO: 31 or 32.

In another aspect, provided herein is a use of an anti-sortilin antibody at a dose of at least about 6 mg/kg in the manufacture of a medicament for treating or delaying the progression of a disease, disorder, or injury in an individual, wherein the antibody comprises a light chain variable domain and a heavy chain variable domain, wherein the heavy chain variable domain comprises an HVR-H1 comprising the amino acid sequence of SEQ ID NO:6, an HVR-H2 comprising the amino acid sequence of SEQ ID NO: 7, and an HVR-H3 comprising the amino acid sequence of SEQ ID NO:8, and the light chain variable domain comprises an HVR-L1 comprising the amino acid sequence of SEQ ID NO:9, an HVR-L2 comprising the amino acid sequence of SEQ ID NO:10, and an HVR-L3 comprising the amino acid sequence of SEQ ID NO:11.

In another aspect, provided herein is a use of an anti-sortilin antibody at a dose of at least about 6 mg/kg in the manufacture of a medicament for increasing progranulin levels in an individual having a disease, disorder, or injury, wherein the antibody comprises a light chain variable domain and a heavy chain variable domain, wherein the heavy chain variable domain comprises an HVR-H1 comprising the amino acid sequence of SEQ ID NO:6, an HVR-H2 comprising the amino acid sequence of SEQ ID NO: 7, and an HVR-H3 comprising the amino acid sequence of SEQ ID NO:8, and the light chain variable domain comprises an HVR-L1 comprising the amino acid sequence of SEQ ID NO:9, an HVR-L2 comprising the amino acid sequence of SEQ ID NO: 10, and an HVR-L3 comprising the amino acid sequence of SEQ ID NO: 11.

In another aspect, provided herein is a use of an antibody that binds to a sortilin protein in the manufacture of a medicament for treating or delaying the progression of a disease, disorder, or injury in an individual, wherein the antibody comprises a heavy chain variable domain comprising the amino acid sequence of SEQ ID NO: 20 and a light chain variable domain comprising the amino acid sequence of SEQ ID NO: 21, and wherein the antibody has a human IgG1 isotype and comprises an Fc region comprising amino acid substitutions L234A, L235A, and P331S, wherein the numbering of the residue position is according to EU numbering.

In another aspect, provided herein is a use of an antibody that binds to a sortilin protein in the manufacture of a medicament for increasing progranulin levels in an individual having a disease, disorder, or injury, wherein the antibody comprises a heavy chain variable domain comprising the amino acid sequence of SEQ ID NO: 20 and a light chain variable domain comprising the amino acid sequence of SEQ ID NO: 21, and wherein the antibody has a human IgG1 isotype and comprises an Fc region comprising amino acid substitutions L234A, L235A, and P331S, wherein the numbering of the residue position is according to EU numbering.

In another aspect, provided herein is a use of an antibody that binds to a sortilin protein in the manufacture of a medicament for treating or delaying the progression of a disease, disorder, or injury in an individual, wherein the antibody comprises a light chain comprising the amino acid sequence of SEQ ID NO: 30, and a heavy chain comprising the amino acid sequence of SEQ ID NO: 31 or 32.

In another aspect, provided herein is a use of an antibody that binds to a sortilin protein in the manufacture of a medicament for increasing progranulin levels in an individual having a disease, disorder, or injury, wherein the antibody comprises a light chain comprising the amino acid sequence of SEQ ID NO: 30, and a heavy chain comprising the amino acid sequence of SEQ ID NO: 31 or 32.

### BRIEF DESCRIPTION OF THE DRAWINGS

FIG. 1 is a diagram of the design of the study described in Example 1. CSF=cerebrospinal fluid; D=Study Day; IV=intravenous; SC=subcutaneous. The study days on which CSF samples are obtained during the study are indicated

FIGS. 2A-2C show the levels of progranulin in the plasma of subjects in Cohort 1 of the study described in Examples 1-2. FIG. 2A provides the levels of progranulin in plasma at the indicated times (Predose, EOI, 4HR, 8HR, 12HR) on Day 1 of the study for each of subjects 1-8 in Cohort 1 administered anti-sortilin antibody ALX at a dose of 6 mg/kg. The y-axes are labeled at 100, 150, and 200 ng/ml. FIG. 2B provides the levels of progranulin in plasma at the indicated times (Predose, EOI, 4HR, 8HR, 12HR) on Day 1 of the study for each of subjects 1-3 in Cohort 1 administered placebo. The y-axes are labeled at 100, 150, and 200 ng/ml. FIG. 2C shows the average levels of progranulin in plasma for subjects in Cohort 1 administered placebo or antibody ALX on the indicated days of the study.

FIGS. 3A-3C show the levels of progranulin in the plasma of subjects in Cohort 2 of the study described in Examples 1-2. FIG. 3A provides the levels of progranulin in plasma at the indicated time points on Day 1 of the study for each of subjects 1-6 in Cohort 2 administered anti-sortilin antibody ALX at a dose of 15 mg/kg. FIG. 3B provides the levels of progranulin in plasma at the indicated time points on Day 1 of the study for each of subjects 1-2 in Cohort 2 administered placebo. FIG. 3C shows the average levels of progranulin in plasma for subjects in Cohort 2 administered placebo or antibody ALX on the indicated days of the study.

FIG. 4 shows the average levels of progranulin in plasma on the indicated days of the study for subjects in Cohorts 1 and 2 administered anti-sortilin antibody ALX or placebo.

FIGS. 5A-5D provide the levels of progranulin in the plasma of subjects in Cohorts 1 and 2 of the study described in Examples 1-2. FIG. 5A shows the level of progranulin in plasma for each subject administered placebo in Cohorts 1 and 2 of the study on the indicated days after administration of placebo. Each line represents one subject. FIG. 5B shows the levels of progranulin in plasma for each subject administered antibody ALX (6 mg/kg) in Cohort 1 on the indicated days after administration of the antibody. Each line represents one subject. FIG. 5C shows the levels of progranulin in plasma for each subject administered antibody ALX (15 mg/kg) in Cohort 2 on the indicated days after administration of the antibody. Each line represents one subject. FIG. 5D shows the average levels of progranulin in plasma for subjects administered antibody ALX or placebo in Cohorts 1 and 2 on the indicated days after administration of antibody or placebo. All subjects administered placebo in Cohorts 1 and 2 were pooled (“pooled placebo”). SD=single dose; IV=intravenous.

FIG. 6 shows the change in the levels of progranulin in the CSF of subjects in Cohorts 1 and 2 of the study described in Examples 1-2. The levels of progranulin in CSF are shown as the mean percentage change on the indicated days of the study compared to baseline progranulin levels in CSF. The numbers of subjects included in the analysis of progranulin CSF levels for each cohort on each study day are shown under the graph.

FIGS. 7A-7B provide the mean concentration (ng/ml) of anti-sortilin antibody ALX in the serum of cynomolgus monkeys administered the antibody intravenously once per week for four weeks at doses of 20 mg/kg, 60 mg/kg, and 200 mg/kg, as described in Example 4. FIG. 7A shows the mean concentration of anti-sortilin antibody ALX in serum at the indicated times (hours) after each dose of antibody administered on Day 1, Day 8, and Day 15 of the study. FIG. 7B shows the mean concentration of anti-sortilin antibody ALX in serum at the indicated times (hours) after each dose of antibody administered on Day 22 and Day 29 of the study.

FIGS. 8A-8C show the levels of sortilin expression in white blood cells (WBCs), serum progranulin, and CSF progranulin in cynomolgus monkeys administered antibody ALX intravenously once per week for four weeks at doses of 0 mg/kg (control), 20 mg/kg, 60 mg/kg, and 200 mg/kg, as described in Example 4. FIG. 8A shows the level of sortilin (SORT1) expression in white blood cells as the percent of baseline levels at the indicated times after administration of the first dose of antibody ALX. FIG. 8B shows the level of progranulin in serum as the percent of baseline levels at the indicated times after administration of the first dose of antibody ALX. FIG. 8C shows the level of progranulin in CSF as the percent of baseline levels at the indicated times after administration of the first dose of antibody ALX. In FIGS. 8A-8C, the arrows indicate the times of administration of a dose of antibody ALX.

FIG. 9 is a diagram of participant disposition of the enrolled population at various time points during the study. IV=intravenous; SD=single dose; SC=subcutaneous. ^{a}Withdrawal by the subject (n=2); ^{b}Withdrawal by the subject; ^{c}Physician decision; ^{d}Physician decision (n=1) and withdrawal by the subject (n=3); ^{e}Adverse event.

FIGS. 10A-10B show the mean concentration (ng/ml) of anti-sortilin antibody ALX in the serum and CSF of subjects in Cohorts administered with a single intravenous dose of antibody ALX at doses of 6 mg/kg (Cohort 1), 15 mg/kg (Cohort 2), 30 mg/kg (Cohort 3), 60 mg/kg (Cohort 4), or administered with a single subcutaneous dose of ALX antibody at 600 mg (SD SC Cohort), as described in Example 5. FIG. 10A shows the serum concentration of ALX at the indicated times (days) after administration of a single intravenous or subcutaneous dose of ALX. FIG. 10B shows the mean CSF concentration of ALX at the indicated times (days) after administration of a single intravenous or subcutaneous dose of ALX.

FIGS. 11A-11B show the mean percentage change from baseline in plasma and CSF concentrations of progranulin administered with a single intravenous dose of antibody ALX at doses of 6 mg/kg (Cohort 1), 15 mg/kg (Cohort 2), 30 mg/kg (Cohort 3), 60 mg/kg (Cohort 4), or administered with a single subcutaneous dose of ALX antibody at 600 mg (SD SC Cohort), as described in Example 5. FIG. 11A shows the mean percentage change from baseline in plasma concentrations of progranulin at the indicated times (days) after administration of a single dose of antibody ALX. FIG. 11B shows the mean percentage change from baseline in CSF concentration of progranulin at the indicated times (days) after administration of a single dose of antibody ALX.

FIG. 12 shows a diagram of the design of a single dose subcutaneous cohort. Subjects are administered a single subcutaneous dose of 150 mg antibody ALX. The single dose is administered on Day 1, as indicated by the arrow. The study days on which CSF samples are obtained are indicated with a square. LP=lumbar puncture; BL=baseline; SC=subcutaneous; EoS=end of study.

FIG. 13 shows a diagram of the design of a multi-dose subcutaneous cohort. Seven doses of 300 mg of antibody ALX are administered to subjects (n=10) on Day 1, Day 15, Day 29, Day 43, Day 57, Day 71, and Day 85, as indicated by arrows. The study days on which CSF samples are obtained are indicated with a square. LP=lumbar puncture; BL=baseline; SC=subcutaneous; HV=healthy volunteer; EoS=end of study.

FIG. 14 shows a diagram of the design of a multi-dose intravenous cohort. Four doses of 30 mg/kg of antibody ALX or placebo are administered in a ratio of 8:2 (antibody ALX:placebo) to subjects (n=10) on Day 1, Day 29, Day 57, and Day 85, as indicated by arrows. The study days on which CSF samples are obtained are indicated with a square. LP=lumbar puncture; BL=baseline; IV=intravenous; HV=healthy volunteer; EoS=end of study.

FIG. 15 shows a diagram of the design of the study described in Example 1. CSF=cerebrospinal fluid; DLAE=dose-limiting adverse event; IV=intravenous; PD=pharmacodynamics; PK=pharmacokinetics; SC=subcutaneous; SD=single dose. ^{a}Post-dose CSF sampling occurred at 2 time points on days 25, 43, or 57; ^{b}Post-dose CSF sampling occurred at 2 time points on days 25, 43, 57, or 85; ^{c}Post-dose CSF sampling occurred at 2 time points on days 43, 57, or 85; ^{d}Post-dose CSF sampling occurred at 2 time points on days 25, 43, 57, or 85; ^{e}Participants in the 6-mg/kg, 15-mg/kg, and 30-mg/kg dose groups were followed for 12 weeks; ^{f}Participants in the 60-mg/kg dose group were followed for 16 weeks; ^{g}Final study visit for participants in the 6-mg/kg, 15-mg/kg, and 30-mg/kg dose groups; ^{h}Final study visit for participants in the 60-mg/kg dose group; ⁱPost-dose CSF sampling occurred at 2 time points on days 8, 13, 18, 25, or 43.

### DETAILED DESCRIPTION

### Definitions

As used herein, the term “preventing” includes providing prophylaxis with respect to occurrence or recurrence of a particular disease, disorder, or condition in an individual. An individual may be predisposed to or susceptible to a particular disease, disorder, or condition, or at risk of developing such a disease, disorder, or condition, but has not yet been diagnosed with the disease, disorder, or condition.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

As used herein, the term “treatment” or “treating” refers to clinical intervention designed to alter the natural course of the individual being treated during the course of clinical pathology. Desirable effects of treatment include decreasing the rate of progression, delaying the progression, ameliorating or palliating the pathological state, and remission or improved prognosis of a particular disease, disorder, or condition. An individual is successfully “treated,” for example, if one or more symptoms associated with a particular disease, disorder, or condition are mitigated or eliminated.

An “effective amount” refers to at least an amount effective, at dosages and for periods of time necessary, to achieve the desired therapeutic or prophylactic result. An effective amount can be provided in one or more administrations. An effective amount herein may vary according to factors such as the disease state, age, sex, and weight of the individual, and the ability of the treatment to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, administration “in conjunction” or “in combination” with another compound or composition includes simultaneous administration and/or administration at different times. Administration in conjunction or in combination also encompasses administration as a co-formulation or administration as separate compositions, including at different dosing frequencies or intervals, and using the same route of administration or different routes of administration.

An “individual” for purposes of treatment, prevention, or reduction of risk refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. In some embodiments, the individual is human.

The terms “sortilin,” “sortilin protein,” and “sortilin polypeptide” are used interchangeably herein to refer to any native sortilin from any mammalian source, including primates (e.g., humans and cynomolgus monkeys) and rodents (e.g., mice and rats), unless otherwise indicated. In some embodiments, the term encompasses both wild-type sequences and naturally occurring variant sequences, e.g., splice variants or allelic variants. In some embodiments, the term encompasses “full-length,” unprocessed sortilin as well as any form of sortilin that results from processing in the cell. In some embodiments, the sortilin is human sortilin. In some embodiments, the amino acid sequence of an exemplary human sortilin is SEQ ID NO: 1.

The terms “anti-sortilin antibody,” an “antibody that binds to sortilin,” and “antibody that specifically binds sortilin” refer to an antibody that is capable of binding sortilin with sufficient affinity such that the antibody is useful as a diagnostic and/or therapeutic agent in targeting sortilin. In one embodiment, the extent of binding of an anti-sortilin antibody to an unrelated, non-sortilin polypeptide is less than about 10% of the binding of the antibody to sortilin as measured, e.g., by a radioimmunoassay (RIA), a bio-layer interferometry assay (e.g., using a ForteBio system), or a surface plasmon resonance (SPR) assay, see, e.g., Hearty et al., Methods Mol Biol (2012) 907:411-42; Vishal and Rafique, Analytical Biochemistry (2017) 536, pp. 16-31; Estep et al., MAbs (2013) 5(2):270-8; and Friguet et al., Analytical Biochemistry (1993) 210(2): 344-350. In certain embodiments, an antibody that binds to sortilin has a dissociation constant (KD) of <1 μM, <100 nM, <10 nM, <1 nM, <0.1 nM, <0.01 nM, or <0.001 nM (e.g., 10⁻⁸ M or less, e.g. from 10⁻⁸ M to 10⁻¹³ M, e.g., from 10⁻⁹ M to 10⁻¹³ M). In certain embodiments, an anti-sortilin antibody binds to an epitope of sortilin that is conserved among sortilin from different species.

The term “immunoglobulin” (Ig) is used interchangeably with “antibody” herein. The term “antibody” herein is used in the broadest sense and specially covers monoclonal antibodies, polyclonal antibodies, multispecific antibodies (e.g., bispecific antibodies) including those formed from at least two intact antibodies, and antibody fragments so long as they exhibit the desired biological activity.

“Native antibodies” are usually heterotetrameric glycoproteins of about 150,000 Daltons, composed of two identical Light (“L”) chains and two identical heavy (“H”) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, CT, 1994, page 71 and Chapter 6.

The light chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”), and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three-dimensional configurations of different classes of immunoglobulins are well known and described generally in, for example, Abbas et al., Cellular and Molecular Immunology, 4^{th} ed. (W.B. Saunders Co., 2000).

The “variable region” or “variable domain” of an antibody, such as an anti-sortilin antibody of the present disclosure, refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

The term “variable” refers to the fact that certain segments of the variable domains differ extensively in sequence among antibodies, such as anti-sortilin antibodies of the present disclosure. The variable domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent-cellular toxicity.

An “isolated” antibody, such as an anti-sortilin antibody of the present disclosure, is one that has been identified, separated and/or recovered from a component of its production environment (e.g., naturally or recombinantly). Preferably, the isolated polypeptide is free of association with all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant T cells since at least one component of the antibody's natural environment will not be present. Ordinarily, however, an isolated polypeptide or antibody will be prepared by at least one purification step.

The term “monoclonal antibody” as used herein refers to an antibody, such as a monoclonal anti-sortilin antibody of the present disclosure, obtained from a population of substantially homogeneous antibodies, i.e., the individual antibodies comprising the population are identical except for possible naturally occurring mutations and/or post-translation modifications (e.g., isomerizations, amidations, etc.) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, but not limited to one or more of the following methods, immunization methods of animals including, but not limited to rats, mice, rabbits, guinea pigs, hamsters and/or chickens with one or more of DNA(s), virus-like particles, polypeptide(s), and/or cell(s), the hybridoma methods, B-cell cloning methods, recombinant DNA methods, and technologies for producing human or human-like antibodies in animals that have parts or all of the human immunoglobulin loci or genes encoding human immunoglobulin sequences.

The terms “full-length antibody,” “intact antibody” or “whole antibody” are used interchangeably to refer to an antibody, such as an anti-sortilin antibody of the present disclosure, in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10):1057-1062 (1995)); single-chain antibody molecules and multispecific antibodies formed from antibody fragments.

Papain digestion of antibodies, such as anti-sortilin antibodies of the present disclosure, produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Functional fragments” of antibodies, such as anti-sortilin antibodies of the present disclosure, comprise a portion of an intact antibody, generally including the antigen binding or variable region of the intact antibody or the F region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the variable domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains.

As used herein, a “chimeric antibody” refers to an antibody (immunoglobulin), such as a chimeric anti-sortilin antibody of the present disclosure, in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity. Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies, such as humanized forms of anti-sortilin antibodies of the present disclosure, are chimeric antibodies comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one that possesses an amino-acid sequence corresponding to that of an antibody, such as an anti-sortilin antibody of the present disclosure, produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries and yeast-based platform technologies. Human antibodies can be prepared by administering the antigen to a transgenic animal that has been modified to produce such antibodies in response to antigenic challenge, but whose endogenous loci have been disabled, e.g., immunized xenomice as well as generated via a human B-cell hybridoma technology.

The term “hypervariable region,” “HVR,” or “HV,” when used herein refers to the regions of an antibody-variable domain, such as that of an anti-sortilin antibody of the present disclosure, that are hypervariable in sequence and/or form structurally defined loops. Generally, antibodies comprise six HVRs; three in the V_{H} (H1, H2, H3), and three in the V_{L} (L1, L2, L3). In native antibodies, H3 and L3 display the most diversity of the six HVRs, and H3 in particular is believed to play a unique role in conferring fine specificity to antibodies. Naturally occurring camelid antibodies consisting of a heavy chain only are functional and stable in the absence of light chain.

A number of HVR delineations are in use and are encompassed herein. In some embodiments, the HVRs may be Kabat complementarity-determining regions (CDRs) based on sequence variability and are the most commonly used (Kabat et al., supra). In some embodiments, the HVRs may be Chothia CDRs. Chothia refers instead to the location of the structural loops (Chothia and Lesk J. Mol. Biol. 196:901-917 (1987)). In some embodiments, the HVRs may be AbM HVRs. The AbM HVRs represent a compromise between the Kabat CDRs and Chothia structural loops, and are used by Oxford Molecular's AbM antibody-modeling software. In some embodiments, the HVRs may be “contact” HVRs. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat numbering) |
| H1 | H31-H35 | H26-H35 | H26-H32 | H30-H35 (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

An “acceptor human framework” as used herein is a framework comprising the amino acid sequence of a V_{L} or V_{H} framework derived from a human immunoglobulin framework or a human consensus framework. An acceptor human framework “derived from” a human immunoglobulin framework or a human consensus framework may comprise the same amino acid sequence thereof, or it may comprise pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, preferable those changes occur at only three, two, or one of positions 71H, 73H and 78H; for instance, the amino acid residues at those positions may by 71A, 73T and/or 78A. In one embodiment, the VL acceptor human framework is identical in sequence to the V_{L} human immunoglobulin framework sequence or human consensus framework sequence.

A “human consensus framework” is a framework that represents the most commonly occurring amino acid residues in a selection of human immunoglobulin V_{L} or V₁ framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, MD (1991). Examples include for the V_{L}, the subgroup may be subgroup kappa I, kappa II, kappa III or kappa IV as in Kabat et al., supra. Additionally, for the V_{H}, the subgroup may be subgroup I, subgroup II, or subgroup III as in Kabat et al., supra.

An “amino acid modification” at a specified position, e.g., of an anti-sortilin antibody of the present disclosure, refers to the substitution or deletion of the specified residue, or the insertion of at least one amino acid residue adjacent to the specified residue. Insertion “adjacent” to a specified residue means insertion within one to two residues thereof. The insertion may be N-terminal or C-terminal to the specified residue. The preferred amino acid modification herein is a substitution.

An “affinity-matured” antibody, such as an anti-sortilin antibody of the present disclosure, is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As use herein, the term “specifically recognizes” or “specifically binds” refers to measurable and reproducible interactions such as attraction or binding between a target and an antibody, such as an anti-sortilin antibody of the present disclosure that is determinative of the presence of the target in the presence of a heterogeneous population of molecules including biological molecules. For example, an antibody, such as an anti-sortilin antibody of the present disclosure, that specifically or preferentially binds to a target or an epitope is an antibody that binds this target or epitope with greater affinity, avidity, more readily, and/or with greater duration than it binds to other targets or other epitopes of the target. It is also understood by reading this definition that, for example, an antibody (or a moiety) that specifically or preferentially binds to a first target may or may not specifically or preferentially bind to a second target. As such, “specific binding” or “preferential binding” does not necessarily require (although it can include) exclusive binding. An antibody that specifically binds to a target may have an association constant of at least about 10³ M⁻¹ or 10⁴ M⁻¹, sometimes about 10⁵M⁻¹ or 10⁶M⁻¹, in other instances about 10⁶M⁻¹ or 10⁷ M⁻¹, about 10⁸M⁻⁴ to 10⁹M⁻¹, or about 10¹⁰ M⁻¹ to 10¹¹ M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

As used herein, an “interaction” between a sortilin protein and a second protein encompasses, without limitation, protein-protein interaction, a physical interaction, a chemical interaction, binding, covalent binding, and ionic binding. As used herein, an antibody “inhibits interaction” between two proteins when the antibody disrupts, reduces, or completely eliminates an interaction between the two proteins. An antibody of the present disclosure, or fragment thereof, “inhibits interaction” between two proteins when the antibody or fragment thereof binds to one of the two proteins.

Antibody “effector functions” refer to those biological activities attributable to the Fc region (a native sequence Fc region or amino acid sequence variant Fc region) of an antibody, and vary with the antibody isotype.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. Other FcRs, including those to be identified in the future, are encompassed by the term “FcR” herein. FcRs can also increase the serum half-life of antibodies. As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full length of the sequences being compared.

An “isolated” cell is a molecule or a cell that is identified and separated from at least one contaminant cell with which it is ordinarily associated in the environment in which it was produced. In some embodiments, the isolated cell is free of association with all components associated with the production environment. The isolated cell is in a form other than in the form or setting in which it is found in nature. Isolated cells are distinguished from cells existing naturally in tissues, organs, or individuals. In some embodiments, the isolated cell is a host cell of the present disclosure.

An “isolated” nucleic acid molecule encoding an antibody, such as an anti-sortilin antibody of the present disclosure, is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of the present disclosure.

“Carriers” as used herein include pharmaceutically acceptable carriers, excipients, or stabilizers that are nontoxic to the cell or mammal being exposed thereto at the dosages and concentrations employed.

The term “about” as used herein refers to the usual error range for the respective value readily known to the skilled person in this technical field. Reference to “about” a value or parameter herein includes (and describes) embodiments that are directed to that value or parameter per se.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that aspect and embodiments of the present disclosure described herein include “comprising,” “consisting,” and “consisting essentially of” aspects and embodiments.

### Overview

The present disclosure relates to methods of treating and/or delaying the progression of a disease, disorder, or injury in an individual by administering an anti-sortilin antibody to the individual. Non-limiting examples of diseases, disorders, or injuries that may be treated or delayed include frontotemporal dementia, progressive supranuclear palsy, Alzheimer's disease, vascular dementia, seizures, retinal dystrophy, amyotrophic lateral sclerosis (ALS), traumatic brain injury, a spinal cord injury, dementia, stroke, Parkinson's disease, acute disseminated encephalomyelitis, retinal degeneration, age related macular degeneration, glaucoma, multiple sclerosis, septic shock, bacterial infection, arthritis, and osteoarthritis. In some embodiments, the disease or disorder that may be treated or delayed according to the methods provided herein is a neurodegenerative disease, such as Alzheimer's disease, Parkinson's disease, or frontotemporal dementia. In some embodiments, the disease or disorder that may be treated or delayed according to the methods provided herein is Alzheimer's disease. In some embodiments, the disease or disorder that may be treated or delayed according to the methods provided herein is Parkinson's disease. In some embodiments, the disease or disorder that may be treated or delayed according to the methods provided herein is frontotemporal dementia. In some embodiments, the disease or disorder that may be treated or delayed according to the methods provided herein is ALS. In any of the above embodiments, the individual is at risk of the disease or disorder. As described below, the methods of the present disclosure meet the need in the art for identifying methods of treating patients with a suitable dose of an anti-Sortilin antibody of the disclosure, and/or of administering that dose in ways that encourage patient compliance.

Patients with neurodegenerative diseases such as Alzheimer's disease, Parkinson's disease, ALS, and frontotemporal dementia are typically affected by the diseases for long periods of time, and thus require regular treatment over the course of many years. As intravenous administration of therapeutics cannot be done at home, patients must be transported to infusion centers, which may become a burden on both the patient and caregiver. In addition, the memory loss, mood swings, aggression, and other behavioral symptoms that may be associated with these diseases can make patient compliance difficult. Thus, infrequent intravenous administration of treatments for patients with neurodegenerative diseases such as Alzheimer's disease, Parkinson's disease, ALS, and frontotemporal dementia are advantageous for patients suffering from such diseases. Furthermore, subcutaneous administration, e.g., by subcutaneous injection, is a convenient mode of administration of treatments for patients with neurodegenerative diseases such as Alzheimer's disease, Parkinson's disease, ALS, and frontotemporal dementia because it can be done at home, e.g., by a caregiver or by the patient themselves.

Advantageously, intravenous administration to humans of a single dose of an anti-sortilin antibody of the present disclosure according to the methods provided herein results in pharmacodynamic (PD) effects, e.g., increases in progranulin levels in plasma and cerebrospinal fluid, that persist over an extended period of time (e.g., up to about 57 days or longer after a single dose of anti-sortilin antibody). See, e.g., Examples 1-2.

Furthermore, advantageously, administration of multiple does of an anti-Sortilin antibody of the disclosure to cynomolgus monkeys results in a decrease in sortilin expression in white blood cells, along with increases in progranulin levels in serum and cerebrospinal fluid that persist for several weeks after the last dose of anti-sortilin antibody (e.g., up to about 6 weeks or longer), indicating a long-lasting pharmacodynamic effect of the anti-Sortilin antibody of the disclosure (see, e.g., Example 4).

Thus, the methods provided herein permit relatively infrequent administration of an anti-sortilin antibody of the disclosure by intravenous infusion (see, e.g., Examples 1-3) or by subcutaneous administration (see, e.g., Example 1), which are particularly beneficial for patients with neurodegenerative diseases, such as Alzheimer's disease, Parkinson's disease, ALS, and frontotemporal dementia.

Accordingly, in some embodiments, the present disclosure relates to methods of treating and/or delaying the progression of a disease, disorder or injury in an individual by administering to the individual an anti-sortilin antibody of the disclosure at a dose of at least about 6 mg/kg. In some embodiments, administration of the antibody is by intravenous infusion or by subcutaneous injection. In some embodiments, the methods provided herein comprise administering the anti-sortilin antibody about once every four weeks or less frequently. In some embodiments, the disease, disorder or injury is Alzheimer's disease. In some embodiments, the disease, disorder or injury is Parkinson's disease. In some embodiments, the disease, disorder or injury is ALS. In some embodiments, the disease, disorder or injury is frontotemporal dementia.

All references cited herein, including patents, patent applications and publications, are hereby incorporated by reference in their entirety.

### Therapeutic Uses

The present disclosure provides methods of treating and/or delaying the progression of a disease, disorder, or injury in an individual, comprising administering to the individual an anti-sortilin antibody. As disclosed herein, anti-sortilin antibodies of the present disclosure may be used for treating and/or delaying progression of frontotemporal dementia, progressive supranuclear palsy, Alzheimer's disease, vascular dementia, seizures, retinal dystrophy, amyotrophic lateral sclerosis, traumatic brain injury, a spinal cord injury, dementia, stroke, Parkinson's disease, acute disseminated encephalomyelitis, retinal degeneration, age related macular degeneration, glaucoma, multiple sclerosis, septic shock, bacterial infection, arthritis, or osteoarthritis. In some embodiments, the disease or disorder is frontotemporal dementia. In some embodiments, the disease or disorder is Alzheimer's disease. In some embodiments, the disease or disorder is Parkinson's disease.

### Parkinson's Disease

Parkinson's disease, which may be referred to as idiopathic or primary parkinsonism, hypokinetic rigid syndrome (HRS), or paralysis agitans, is a neurodegenerative brain disorder that affects motor system control. The progressive death of dopamine-producing cells in the brain leads to the major symptoms of Parkinson's. Most often, Parkinson's disease is diagnosed in people over 50 years of age. Symptoms of Parkinson's disease include, without limitation, tremors (e.g., of the hands, arms, legs, jaw, and face), muscle rigidity in the limbs and trunk, slowness of movement (bradykinesia), postural instability, difficulty walking, neuropsychiatric problems, changes in speech or behavior, depression, anxiety, pain, psychosis, dementia, hallucinations, and sleep problems.

Parkinson's disease is idiopathic (having no known cause) in most people. Most cases of Parkinson's disease occur in people with no apparent history of the disorder in their family and are thought to arise from complex interactions between environmental and genetic factors. Such cases of Parkinson's disease are referred to as “sporadic” Parkinson's disease.

In addition, about 15% of Parkinson's disease patients have a known family history of Parkinson's disease, with certain familial cases being associated with mutations in genes such as PARK2, LRRK2, PARK7, PINK1, PRKN, or SNCA.

Mutations in the GBA1 and/or the UCHL1 genes have also been associated with Parkinson's disease and are thought to modify the risk of developing the disease. For example, about 5% of Parkinson's disease patients carry a mutation in the GBA1 gene, which encodes the lysosomal enzyme beta-glucocerebrosidase (GBA). It has been found that heterozygous or homozygous GBA1 mutations increase the risk of Parkinson's disease by between about 20-fold and about 30-fold. Furthermore, GBA1 mutations in Parkinson's disease have been associated with an earlier age of onset and a more rapid cognitive and motor decline. See, e.g., Stoker et al., Pathological Mechanisms and Clinical Aspects of GBA1 Mutation-Associated Parkinson's Disease, Parkinson's Disease: Pathogenesis and Clinical Aspects, Codon Publications; 2018, Chapter 3; medlineplus[dot]gov/genetics/condition/parkinson-disease/#resources; and www[dot]ninds[dot]nih[dot]gov/Disorders/All-Disorders/Parkinsons-Disease-Challenges-Progress-and-Promise.

Progranulin (PGRN), encoded by the GRN gene, has also been associated with Parkinson's disease. The GRN Parkinson's disease risk allele is associated with reduced progranulin levels in plasma, cerebrospinal fluid, and brain, while homozygous loss of function of progranulin causes neuronal ceroid lipofuscinosis and heterozygous loss of function of progranulin causes frontotemporal dementia (FTD), sometimes with Parkinsonism presentation (Smith et al., Am J Hum Genet (2012) 90(6):1102-1107; Boeve et al., Brain (2006) 129(Pt 11):3103-3114; and Wauters et al., Neurobiol Aging (2018) 67:84-94). Furthermore, reduced plasma progranulin levels are associated with increased severity of Parkinson's disease (Yao et al., Neurosci Lett (2020) 725:134873). Progranulin deficiency also worsens outcomes in rodent Parkinson's disease models (Martens et al., J Clin Invest (2012) 122(11):3955-3959), while delivery of the progranulin gene improves those outcomes (Van Kampen et al., PLoS One (2014) 7; 9(5):e97032).

Progranulin is the ˜88-kDa precursor protein for granulins. It is a highly conserved, multifunctional secreted glycoprotein that forms a unique “beads-on-a-string” structure. Its activities include regulating central nervous system (CNS) neuroinflammation, influencing cell signaling pathways by controlling excitotoxicity, oxidative stress, synaptogenesis, inflammation, and amyloid production (Hsiung et al., GeneReviews. University of Washington, Seattle), acting as an autocrine neuronal growth factor, promoting neurite outgrowth in the brain (Gass et al., Mol Neurodegener (2012) 10(7):33), and promoting and enhancing the survival of motor and cortical neurons (De Muynck et al., Neurobiol Aging (2013) 34(11):2541-2547).

Recent genome-wide association studies (GWAS) have revealed that genetic determinants of Parkinson's disease risk are enriched for lysosomal genes, such as GBA, GPNMB, GALC, CTSB, and GRN (Nalls et al., Lancet Neurol (2019) 18(2):1091-1102). Furthermore, it is known that after receptor-mediated uptake, progranulin is transported to the lysosome, and that its complete absence in humans causes an early onset lysosomal storage disorder (Paushter et al., Acta Neuropathol (2018) 136(1):1-17). These observations suggest that progranulin plays a role in lysosomal function. Emerging evidence also suggests that progranulin interacts with lysosomal enzymes such as cathepsin D and glucocerebrosidase (GCase). In addition, it is thought that progranulin, together with its binding partner prosaposin (PSAP), regulates the activity of these enzymes (Arrant et al., Acta Neuropathologica Comm (2019) 7(1):218; Valdez et al., Hum Mol Genet (2020) 29(5):716-726; Zhou et al., PLoS One (2019) 14(7):e0212382; Valdez et al., Hum Mol Genet (2017) 26(24):4861-4872; Zhou et al., J Cell Biol (2015) 210(6):991-1002; and Zhou et al., Mol Neurodegener (2017) 23; 12(1):62).

Accordingly, increasing progranulin levels, e.g., in the cerebrospinal fluid and/or plasma of an individual having or at risk for having Parkinson's disease, may treat and/or delay progression of Parkinson's disease, e.g., by increasing GCase activity, reducing inflammation, and/or improving lysosomal function.

In some embodiments, administering an anti-sortilin antibody of the present disclosure may treat and/or delay the progression of Parkinson's disease in an individual. In some embodiments, administering an anti-sortilin antibody of the present disclosure to an individual having or at risk for having Parkinson's disease may increase levels of progranulin in the individual, e.g., in plasma and/or cerebrospinal fluid; and/or decrease levels of sortilin; and/or inhibit the interaction between sortilin and one or more proteins; and/or inhibit the interaction between sortilin and progranulin, thereby treating and/or delaying progression of Parkinson's disease.

In some embodiments, the disease or disorder to be treated according to the methods provided herein is Parkinson's disease, such as sporadic Parkinson's disease or Parkinson's disease in an individual having at least one pathogenic mutation in the GBA1 gene or in a GBA1 gene product. In some embodiments, the Parkinson's disease to be treated according to the methods provided herein is sporadic Parkinson's disease. In some embodiments, the Parkinson's disease to be treated according to the methods provided herein is Parkinson's disease in an individual having at least one pathogenic mutation in the GBA1 gene or in a GBA1 gene product. In some embodiments, the individual is homozygous or heterozygous for one or more mutations in the GBA1 gene or in a GBA1 gene product. Pathogenic mutations in the GBA1 gene or GBA1 gene product are known in the art. See, e.g., Avenali et al., Front Aging Neurosci, 2020; O'Reagan et al., J. Parkinsons Dis, 7:411-422 (2017); Zhang et al., Front. Mol. Neurosci, 11:43 (2018); and Zhang et al., Parkisons Dis, 3136415 (2018). Exemplary pathogenic mutations in the GBA1 gene or GBA1 gene product include, without limitation, N370S (c.1226A>G), L444P (c.1448T>C), R120W, IVS2+1G>A, H255Q, D409H, RecNciI, E326K, T369M, R496H, and 84insGG. The presence of a mutation in the GBA1 gene or a GBA1 gene product may be assessed using any method known in the art, such as sequencing, polymerase chain reaction, hybridization methods such as fluorescence in situ hybridization, mass spectrometry, and immunoblotting.

In some embodiments, an individual treated according to the methods provided herein has Parkinson's disease that is classified as between Stage I and Stage III, based on the Hoehn and Yahr criteria, e.g., as described by Hoehn and Yahr, Neurology (1967) 17:427-42 and Goetz et al., Mov Disord (2004) 19:1020-8.

In some embodiments, an individual treated according to the methods provided herein is taking one or more treatments for Parkinson's disease prior to administration of an anti-sortilin antibody of the disclosure, such as a glutamate antagonist, an anticholinergic, a dopamine agonist, Levodopa (L-DOPA and decarboxylase [DDC] inhibitor), a monoamine oxidase B (MAO-B) inhibitor, a catechol-O-methyltransferase (COMT) inhibitor, a beta blocker, a selective serotonin uptake inhibitor (SSRI), a tricyclic antidepressant (TCA), or indomethacin. In some embodiments, the individual continues taking the one or more treatments for Parkinson's disease after the start of treatment with an anti-sortilin antibody of the disclosure. In some embodiments, the methods provided herein comprise administering an anti-sortilin antibody of the disclosure in combination with one or more treatments for Parkinson's disease, such as a glutamate antagonist, an anticholinergic, a dopamine agonist, Levodopa (L-DOPA and decarboxylase [DDC] inhibitor), a monoamine oxidase B (MAO-B) inhibitor, a catechol-O-methyltransferase (COMT) inhibitor, a beta blocker, a selective serotonin uptake inhibitor (SSRI), a tricyclic antidepressant (TCA), and indomethacin.

Treatment or delay of Parkinson's disease may be assessed using any method known in the art.

In some embodiments, treatment or delay of Parkinson's disease in an individual treated according to the methods provided herein is assessed based on the cognitive function of the individual before and after the individual has received one or more doses of the anti-sortilin antibody. Cognitive function may be assessed using any method known in the art, such as the Montreal Cognitive Assessment (MoCA). See, e.g., Nareddine et al., J American Geriatrics Soc (2005) 53(4):695-9. In some embodiments, treatment or delay of Parkinson's disease is assessed based on one or more clinical assessments for Parkinson's disease. Examples of clinical assessments for Parkinson's disease that may be used to assess treatment or delay of Parkinson's disease in an individual treated according the methods provided herein include, without limitation, the Movement Disorder Society (MDS)-sponsored revision of the Unified Parkinson's Disease Rating Scale (UPDRS) Part III (MDS-UPDRS Part III), or the UPDRS total scale. See, e.g., Goetz et al., (2008) Movement Disorders 23(15):2129-2170.

In some embodiments, treatment or delay of Parkinson's disease in an individual treated according to the methods provided herein is assessed based on the levels of one or more biomarkers of Parkinson's disease, such as progranulin, GCase protein or activity, and/or alpha-synuclein protein. In some embodiments, the levels of the one or more biomarkers of Parkinson's disease are assessed in a sample of plasma or cerebrospinal fluid obtained from the individual before and after the individual has received one or more doses of the anti-sortilin antibody. Non-limiting examples of methods that may be used to measure the levels of the one or more biomarkers in a sample obtained from the individual include SOMASCAN assay (see, e.g., Candia et al. (2017) Sci Rep 7, 14248), Western blots, mass spectrometry, flow cytometry, and enzyme-linked immunosorbent assay (ELISA).

In some embodiments, treatment or delay of Parkinson's disease in an individual treated according to the methods provided herein is assessed based on the levels of one or more biomarkers of lysosomal function. In some embodiments, the levels of the one or more biomarkers of lysosomal function are assessed in a sample of plasma or cerebrospinal fluid obtained from the individual before and after the individual has received one or more doses of the anti-sortilin antibody. In some embodiments, the one or more biomarkers of lysosomal function are GCase protein, GCase activity, glucosylsphingosine (lyso-Gb1), and/or glucosylceramide. Non-limiting examples of methods that may be used to measure the levels of the one or more biomarkers in a sample obtained from the individual include SOMASCAN assay (see, e.g., Candia et al. (2017) Sci Rep 7, 14248), Western blots, mass spectrometry, flow cytometry, and enzyme-linked immunosorbent assay (ELISA). In some embodiments, the levels of lyso-Gb1 or glucosylceramide may be measured using any method known in the art, such as mass spectrometry, e.g., high-pressure liquid chromatography-tandem mass spectrometry.

In some embodiments, treatment with an anti-sortilin antibody of the disclosure according to the methods provided herein results in an increase in the level of progranulin protein in the plasma or cerebrospinal fluid of the individual of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to the level of progranulin protein in the plasma or cerebrospinal fluid of the individual before administration of the anti-sortilin antibody. In some embodiments, treatment with an anti-sortilin antibody of the disclosure according to the methods provided herein results in an increase in the level of progranulin protein in the plasma or cerebrospinal fluid of the individual of at least about 100%, at least about 125%, at least about 150%, at least about 175%, at least about 200%, at least about 225%, at least about 250%, at least about 275%, at least about 300%, or more compared to the level of progranulin protein in the plasma or cerebrospinal fluid of the individual before administration of the anti-sortilin antibody.

In some embodiments, treatment with an anti-sortilin antibody of the disclosure according to the methods provided herein results in an increase in the level of GCase protein in the plasma or cerebrospinal fluid of the individual of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to the level of GCase protein in the plasma or cerebrospinal fluid of the individual before administration of the anti-sortilin antibody. In some embodiments, treatment with an anti-sortilin antibody of the disclosure according to the methods provided herein results in an increase in the level of GCase protein in the plasma or cerebrospinal fluid of the individual of at least about 100%, at least about 125%, at least about 150%, at least about 175%, at least about 200%, at least about 225%, at least about 250%, at least about 275%, at least about 300%, or more compared to the level of GCase protein in the plasma or cerebrospinal fluid of the individual before administration of the anti-sortilin antibody.

In some embodiments, treatment with an anti-sortilin antibody of the disclosure according to the methods provided herein results in an increase in the level of GCase activity in the plasma or cerebrospinal fluid of the individual of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, compared to the level of GCase activity in the plasma or cerebrospinal fluid of the individual before administration of the anti-sortilin antibody. In some embodiments, treatment with an anti-sortilin antibody of the disclosure according to the methods provided herein results in an increase in the level of GCase activity in the plasma or cerebrospinal fluid of the individual of at least about 100%, at least about 125%, at least about 150%, at least about 175%, at least about 200%, at least about 225%, at least about 250%, at least about 275%, at least about 300%, or more compared to the level of GCase activity in the plasma or cerebrospinal fluid of the individual before administration of the anti-sortilin antibody.

In some embodiments, treatment with an anti-sortilin antibody of the disclosure according to the methods provided herein results in a decrease in the level of alpha-synuclein protein in the plasma or cerebrospinal fluid of the individual of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or about 100%, compared to the level of alpha-synuclein protein in the plasma or cerebrospinal fluid of the individual before administration of the anti-sortilin antibody.

In some embodiments, treatment with an anti-sortilin antibody of the disclosure according to the methods provided herein results in a decrease in the level of glucosylsphingosine (lyso-Gb1) in the plasma or cerebrospinal fluid of the individual of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or about 100%, compared to the level of lyso-Gb1 in the plasma or cerebrospinal fluid of the individual before administration of the anti-sortilin antibody.

In some embodiments, treatment with an anti-sortilin antibody of the disclosure according to the methods provided herein results in a decrease in the level of glucosylceramide in the plasma or cerebrospinal fluid of the individual of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or about 100%, compared to the level of glucosylceramide in the plasma or cerebrospinal fluid of the individual before administration of the anti-sortilin antibody.

In some embodiments, treatment or delay of Parkinson's disease in an individual treated according to the methods provided herein is assessed based on the levels of one or more proteins in the cerebrospinal fluid proteome. In some embodiments, the levels of one or more proteins in the cerebrospinal fluid proteome are assessed in a sample of cerebrospinal fluid obtained from the individual before and after the individual has received one or more doses of the anti-sortilin antibody. Non-limiting examples of methods that may be used to measure the levels of the one or more proteins in the cerebrospinal fluid proteome in a sample obtained from the individual include SOMASCAN assay (see, e.g., Candia et al. (2017) Sci Rep 7, 14248), mass spectrometry and Western blots.

### Frontotemporal Dementia

Frontotemporal dementia (FTD) is a condition resulting from the progressive deterioration of the frontal lobe of the brain. Over time, the degeneration may advance to the temporal lobe. Second only to Alzheimer's disease (AD) in prevalence, FTD accounts for 20% of pre-senile dementia cases. The clinical features of FTD include memory deficits, behavioral abnormalities, personality changes, and language impairments (Cruts, M. & Van Broeckhoven, C., Trends Genet. 24:186-194 (2008); Neary, D., et al., Neurology 51:1546-1554 (1998); Ratnavalli, E., Brayne, C., Dawson, K. & Hodges, J. R., Neurology 58:1615-1621 (2002)).

A substantial portion of FTD cases are inherited in an autosomal dominant fashion, but even in one family, symptoms can span a spectrum, from FTD with behavioral disturbances, to Primary Progressive Aphasia, to Cortico-Basal Ganglionic Degeneration. FTD, like most neurodegenerative diseases, can be characterized by the pathological presence of specific protein aggregates in the diseased brain. Historically, the first descriptions of FTD recognized the presence of intraneuronal accumulations of hyperphosphorylated Tau protein in neurofibrillary tangles or Pick bodies. A causal role for the microtubule associated protein Tau was supported by the identification of mutations in the gene encoding the Tau protein in several families (Hutton, M., et al., Nature 393:702-705 (1998)). However, the majority of FTD brains show no accumulation of hyperphosphorylated Tau, but do exhibit immunoreactivity to ubiquitin (Ub) and TAR DNA binding protein (TDP43) (Neumann, M., et al., Arch. Neurol. 64:1388-1394 (2007)). A majority of those FTD cases with Ub inclusions (FTD-U) were shown to carry mutations in the progranulin gene.

Progranulin mutations result in haploinsufficiency and are known to be present in nearly 50% of familial FTD cases, making progranulin mutation a major genetic contributor to FTD. Without wishing to be bound by theory, it is believed that the loss-of-function heterozygous character of progranulin mutations indicates that in healthy individuals, progranulin expression plays a dose-dependent, critical role in protecting healthy individuals from the development of FTD. Accordingly, increasing levels of progranulin by inhibiting the interaction between sortilin and progranulin, can treat and/or delay the progression of FTD.

In some embodiments, administering an anti-sortilin antibody of the present disclosure may treat and/or delay the progression of FTD in an individual. In some embodiments, administering an anti-sortilin antibody of the present disclosure to an individual having or at risk for having FTD may increase levels of progranulin in the individual, e.g., in plasma and/or cerebrospinal fluid; and/or decrease levels of sortilin; and/or inhibit the interaction between sortilin and one or more proteins; and/or inhibit the interaction between sortilin and progranulin, thereby treating and/or delaying progression of FTD.

In some embodiments, treatment and/or delay of FTD progression may be determined by a change from baseline in neurocognitive and/or functional tests or assessments (i.e., clinical outcome assessments). Non-limiting examples of neurocognitive and functional tests that may be used to evaluate the treatment and/or delay of FTD progression include the Frontotemporal Dementia Clinical Rating Scale (FCRS), the Frontotemporal Dementia Rating Scale (FRS), the Clinical Global Impression-Improvement (CGI-I) assessment, the Neuropsychiatric Inventory (NPI) assessment, the Color Trails Test (CTT) Part 2, the Repeatable Battery for the Assessment of Neuropsychological Status (RBANS), the Delis-Kaplan Executive Function System Color-Word Interference Test, the Interpersonal Reactivity Index, the Winterlight Lab Speech Assessment (WLA), and the Summerlight Lab Speech Assessment (SLA). In some embodiments, treatment and/or delay of FTD progression may be determined by a change from baseline in one neurocognitive and/or functional test or assessment. In some embodiments, treatment and/or delay of FTD progression may be determined by a change from baseline in more than one neurocognitive and/or functional tests or assessments (e.g., 2, 3, 4, 5, 6, 7, 8, 9 or more neurocognitive and/or functional tests or assessments).

In some embodiments, treatment and/or delay of FTD progression is determined by a change from baseline in global and/or regional brain volumes, volume of white matter hyperintensities, brain perfusion, fractional anisotropy, mean diffusivity, axial diffusivity, and radial diffusivity, and/or functional brain activity. In certain embodiments, brain perfusion is measured by arterial spin labeling MRI. In certain embodiments, radial diffusivity is measured by diffusion tensor imaging. In certain embodiments, functional brain activity is measured by functional MRI.

In some embodiments, treatment and/or delay of FTD progression is determined by a change from baseline in markers of neurodegeneration in whole blood, plasma, and CSF. Markers of neurodegeneration may include, without limitation, neurofilament light chain (Nfl), Tau, and/or pTau. Neurofilament light chain may be measured by methods including, without limitation, assays from Quanterix and/or Roche Diagnostics. In some embodiments, treatment and/or delay of FTD progression is determined by a change from baseline in markers of lysosomal function. Markers of lysosomal function may be, without limitation, Cathepsins, such as Cathepsin B (CTSB), NAGK (N-acetyl-D-glucosamine kinase), GCase protein, GCase activity, glucosylsphingosine (lyso-Gb1), and/or glucosylceramide. In some embodiments, treatment and/or delay of FTD progression is determined by a change from baseline in the levels of inflammatory markers, such as Osteopontin (SPP1), YWHAE (14-3-3 protein epsilon), allograft inflammatory factor 1 (AIF1), colony stimulating factor 1 (CSF1), chitinase 1 (CHIT1), lymphocyte antigen 86 (LY86), and CD86. Certain biomarkers of lysosomal function may be overexpressed when PGRN is deficient. For example, cathepsin D (CTSD) and Lamp1 are overexpressed in PGRN-deficient mice (GRN knockout mice). See, Huang et al. (2020) Acta Neuropath Comm 8:163; Götzl et al., (2014) Acta Neuropathol 127(6):845-60; and Lui et al., (2016) Cell 165:921-935. Restoration of PGRN function may therefore decrease expression of biomarkers of lysosomal function that are increased when PGRN is deficient. Certain biomarkers of complement function show overexpression when PGRN is deficient. For example, C1qb and C1qc (subunits that make up the complement protein C1q) show increased levels in PGRN-deficient (GRN knockout) mice. See, Huang et al. (2020) Acta Neuropath Comm 8:163; Götzl et al., (2014) Acta Neuropathol 127(6):845-60; and Lui et al., (2016) Cell 165:921-935. Restoration of PGRN function may therefore decrease expression of biomarkers of complement function that are increased when PGRN is deficient. In some embodiments, treatment and/or delay of FTD progression is determined by a change from baseline in markers of microglial activity. Markers of microglial activity may be, without limitation, YKL-40 and/or Interleukin-6. In some embodiments, treatment and/or delay of FTD progression is determined by a change from baseline of messenger ribonucleic acid (mRNA) expression in peripheral cells. In some embodiments, treatment and/or delay of FTD progression is determined by a change from baseline in analytes relevant to FTD disease biology and/or response to anti-sortilin antibody. In some embodiments, the levels of one or more proteins, such as any of the markers described above, may be measured in a sample obtained from an individual, such as a sample of whole blood, plasma, and/or CSF. Non-limiting examples of methods that may be used to measure the levels of one or more proteins in a sample obtained from an individual include SOMASCAN assay (see, e.g., Candia et al. (2017) Sci Rep 7, 14248), Western blots, mass spectrometry, flow cytometry, and enzyme-linked immunosorbent assay (ELISA).

In some embodiments, treatment and/or delay of FTD progression is determined by a change from baseline in neuroinflammation and/or microglial activation. Neuroinflammation and/or microglial activation may be measured by any known method in the art. In certain embodiments, neuroinflammation and/or microglial activation may be measured using Translocator Protein-Positron Emission (TSPO-PET) imaging.

In some embodiments, an individual treated according to the methods provided herein is heterozygous for a mutation in GRN (the Granulin gene). In some embodiments, the mutation in GRN is a loss-of-function mutation. In some embodiments, the individual is heterozygous for a C9orf72 hexanucleotide repeat expansion. In some embodiments, an individual treated according to the methods provided herein shows symptoms of FTD. In some embodiments, an individual treated according to the methods provided herein does not show symptoms of FTD. In some such embodiments, the individual is pre-symptomatic. In some such cases, the individual has been identified as being heterozygous for a mutation in the GRN gene or as having reduced PGRN levels or function, but does not show symptoms of FTD. In some such cases, the subject has FTD-GRN, or FTD caused by mutation in the GRN gene. In some embodiments of a pre-symptomatic individual, the individual may show elevated levels of one or more biomarkers relative to normal levels, such as elevated levels of Nfl, SPP1, YWHAE, AIF1, CSF1, CHIT1, or LY86 and/or may show reduced levels of one or more biomarkers relative to normal levels, such as reduced levels of NAGK or CTSB.

### Alzheimer's Disease

Alzheimer's disease (AD) is the most common form of dementia. There is no cure for the disease, which worsens as it progresses, and eventually leads to death. Most often, AD is diagnosed in people over 65 years of age. However, the less-prevalent early-onset Alzheimer's can occur much earlier.

Common symptoms of Alzheimer's disease include, behavioral symptoms, such as difficulty in remembering recent events, cognitive symptoms, confusion, irritability and aggression, mood swings, trouble with language, and long-term memory loss. As the disease progresses, bodily functions are lost, ultimately leading to death. Alzheimer's disease develops for an unknown and variable amount of time before becoming fully apparent, and it can progress undiagnosed for years.

It has been shown that sortilin binds to amyloid precursor protein (APP) and the APP processing enzyme BACEL. Without wishing to be bound by theory, it is believed that these interactions are involved in Alzheimer's disease. Accordingly, and without wishing to be bound by theory, it is believed that anti-sortilin antibodies of the present disclosure can be utilized to inhibit such interactions and prevent, reduce the risk of, or treat Alzheimer's disease in individuals in need thereof.

In some embodiments, administering an anti-sortilin antibody of the present disclosure may treat and/or delay the progression of Alzheimer's disease in an individual. In some embodiments, administering an anti-sortilin antibody of the present disclosure to an individual having or at risk for having Alzheimer's disease may increase levels of progranulin in the individual, e.g., in plasma and/or cerebrospinal fluid; and/or decrease levels of sortilin; and/or inhibit the interaction between sortilin and one or more proteins; and/or inhibit the interaction between sortilin and progranulin, thereby treating and/or delaying progression of Alzheimer's disease. In some embodiments, and without wishing to be bound by theory, it is believed that anti-sortilin antibodies of the present disclosure that inhibit the interaction between sortilin and neurotrophins of the present disclosure (e.g., pro-neurotrophins, pro-neurotrophin-3, pro-neurotrophin-4/5, pro-NGF, pro-BDNF, neurotrophin-3, neurotrophin-4/5, NGF, BDNF, etc.), p75, amyloid precursor protein (APP), and/or A beta peptide, or that inhibit one or more activities of sortilin can be utilized to treat and/or delay the progression of Alzheimer's disease in individuals in need thereof.

### Vascular Dementia

Vascular dementia (VaD) is a subtly progressive worsening of memory and other cognitive functions that is believed to be due to cerebrovascular disease (vascular disease within the brain). Cerebrovascular disease is the progressive change in the blood vessels (vasculature) in the brain (cerebrum). The most common vascular change associated with age is the accumulation of cholesterol and other substances in the blood vessel walls. This results in the thickening and hardening of the walls, as well as narrowing of the vessels, which can result in a reduction or even a complete stopping of blood flow to brain regions supplied by the affected artery. Vascular dementia patients often present with similar symptoms to Alzheimer's disease (AD) patients. However, the related changes in the brain are not due to AD pathology but to chronic reduced blood flow in the brain, eventually resulting in dementia. VaD is considered one of the most common types of dementia in older adults. Symptoms of VaD include difficulties with memory, difficulty with organization and solving complex problems, slowed thinking, distraction or “absent mindedness,” difficulty retrieving words from memory, changes in mood or behavior such as depression, irritability, or apathy, and hallucinations or delusions.

Without wishing to be bound by theory, it is believed that one or more activities of sortilin, or one or more interactions between sortilin and progranulin, neurotrophins of the present disclosure (e.g., pro-neurotrophins, pro-neurotrophin-3, pro-neurotrophin-4/5, pro-NGF, pro-BDNF, neurotrophin-3, neurotrophin-4/5, NGF, BDNF, etc.), neurotensin, lipoprotein lipase, apolipoprotein AV, and/or receptor-associated protein are involved in vascular dementia. Accordingly, and without wishing to be bound by theory, it is believed that anti-sortilin antibodies of the present disclosure that inhibit the interaction between sortilin and neurotrophins of the present disclosure (e.g., pro-neurotrophins, pro-neurotrophin-3, pro-neurotrophin-4/5, pro-NGF, pro-BDNF, neurotrophin-3, neurotrophin-4/5, NGF, BDNF, etc.), neurotensin, p75, sortilin propeptide (Sort-pro), amyloid precursor protein (APP), A beta peptide, lipoprotein lipase (LpL), apolipoprotein AV (APOA5), apolipoprotein E (APOE), and/or receptor associated protein (RAP); or that inhibit one or more activities of sortilin can be utilized to prevent, reduce the risk of, or treat vascular dementia in individuals in need thereof.

In some embodiments, administering an anti-sortilin antibody of the present disclosure may treat and/or delay the progression of vascular dementia in an individual. In some embodiments, administering an anti-sortilin antibody of the present disclosure to an individual having or at risk for having vascular dementia may increase levels of progranulin in the individual, e.g., in plasma and/or cerebrospinal fluid; and/or decrease levels of sortilin; and/or inhibit the interaction between sortilin and one or more proteins; and/or inhibit the interaction between sortilin and progranulin, thereby treating and/or delaying progression of vascular dementia.

### Dementia

Dementia is a non-specific syndrome (i.e., a set of signs and symptoms) that presents as a serious loss of global cognitive ability in a previously unimpaired person, beyond what might be expected from normal aging. Dementia may be static, e.g., as the result of a unique global brain injury. Alternatively, dementia may be progressive, resulting in long-term decline due to damage or disease in the body. While dementia is much more common in the geriatric population, it can also occur before the age of 65. Cognitive areas affected by dementia include, without limitation, memory, attention span, language, and problem solving. Generally, symptoms must be present for at least six months before an individual is diagnosed with dementia. Exemplary forms of dementia include, without limitation, frontotemporal dementia, Alzheimer's disease, vascular dementia, semantic dementia, and dementia with Lewy bodies.

Without wishing to be bound by theory, it is believed that administering an anti-sortilin antibody of the present disclosure can treat and/or delay the progression of dementia. In some embodiments, administering an anti-sortilin antibody of the present disclosure may treat and/or delay the progression of dementia in an individual. In some embodiments, administering an anti-sortilin antibody of the present disclosure to an individual having or at risk for having dementia may increase levels of progranulin in the individual, e.g., in plasma and/or cerebrospinal fluid; and/or decrease levels of sortilin; and/or inhibit the interaction between sortilin and one or more proteins; and/or inhibit the interaction between sortilin and progranulin, thereby treating and/or delaying progression of dementia.

### Seizures, Retinal Dystrophy, Traumatic Brain Injuries, and Spinal Cord Injuries

As used herein, retinal dystrophy refers to any disease or condition that involves the degeneration of the retina. Such diseases or conditions may lead to loss of vision or complete blindness.

As used herein, seizures also include epileptic seizures, and refer to a transient symptom of abnormal excessive or synchronous neuronal activity in the brain. The outward effect can be as dramatic as a wild thrashing movement or as mild as a brief loss of awareness. Seizures can manifest as an alteration in mental state, tonic or clonic movements, convulsions, and various other psychic symptoms.

Traumatic brain injuries (TBI), may also be known as intracranial injuries. Traumatic brain injuries occur when an external force traumatically injures the brain. Traumatic brain injuries can be classified based on severity, mechanism (closed or penetrating head injury), or other features (e.g., occurring in a specific location or over a widespread area).

Spinal cord injuries (SCI) include any injury to the spinal cord that is caused by trauma instead of disease. Depending on where the spinal cord and nerve roots are damaged, the symptoms can vary widely, from pain to paralysis to incontinence. Spinal cord injuries are described at various levels of “incomplete”, which can vary from having no effect on the patient to a “complete” injury which means a total loss of function.

It has been shown that pro-neurotrophins (e.g., pro-neurotrophin-4/5, neurotrophin-4/5, pro-NGF, pro-BDNF, etc.) play a role in seizures, retinal dystrophy, traumatic brain injury, and spinal cord injury.

Accordingly, and without wishing to be bound by theory, it is believed that anti-sortilin antibodies of the present disclosure that inhibit the interaction between sortilin and neurotrophins of the present disclosure (e.g., pro-neurotrophins, pro-neurotrophin-3, pro-neurotrophin-4/5, pro-NGF, pro-BDNF, neurotrophin-3, neurotrophin-4/5, NGF, BDNF, etc.); or that inhibit one or more activities of sortilin can be utilized to prevent, reduce the risk of, or treat seizures, retinal dystrophy, traumatic brain injuries, and/or spinal cord injuries in individuals in need thereof.

In some embodiments, administering an anti-sortilin antibody of the present disclosure may treat and/or delay the progression of retinal dystrophy, traumatic brain injuries, and/or spinal cord injuries in an individual. In some embodiments, administering an anti-sortilin antibody of the present disclosure to an individual having or at risk for having retinal dystrophy, traumatic brain injuries, and/or spinal cord injuries may increase levels of progranulin in the individual, e.g., in plasma and/or cerebrospinal fluid; and/or decrease levels of sortilin; and/or inhibit the interaction between sortilin and one or more proteins; and/or inhibit the interaction between sortilin and progranulin, thereby treating and/or delaying progression of retinal dystrophy, traumatic brain injuries, and/or spinal cord injuries.

### Undesirable Symptoms of Aging

As used herein, undesirable symptoms of aging include, without limitation, memory loss, behavioral changes, dementia, Alzheimer's disease, retinal degeneration, atherosclerotic vascular diseases, hearing loss, and cellular break-down.

In some embodiments, and without wishing to be bound by theory, it is believed that anti-sortilin antibodies of the present disclosure that inhibit the interaction between sortilin and progranulin, neurotrophins of the present disclosure (e.g., pro-neurotrophins, pro-neurotrophin-3, pro-neurotrophin-4/5, pro-NGF, pro-BDNF, neurotrophin-3, neurotrophin-4/5, NGF, BDNF, etc.), neurotensin, p75, lipoprotein lipase (LpL), apolipoprotein AV (APOA5), and/or receptor associated protein (RAP); or that inhibit one or more activities of sortilin can be utilized to prevent, reduce the risk of, or treat one or more undesirable symptoms of aging.

In some embodiments, administering an anti-sortilin antibody of the present disclosure may treat and/or delay the progression of one or more undesirable symptoms of aging in an individual. In some embodiments, administering an anti-sortilin antibody of the present disclosure to an individual having or at risk for having one or more undesirable symptoms of aging may increase levels of progranulin in the individual, e.g., in plasma and/or cerebrospinal fluid; and/or decrease levels of sortilin; and/or inhibit the interaction between sortilin and one or more proteins; and/or inhibit the interaction between sortilin and progranulin, thereby treating and/or delaying progression of one or more undesirable symptoms of aging.

### Amyotrophic Lateral Sclerosis (ALS)

As used herein, amyotrophic lateral sclerosis (ALS), motor neuron disease, or Lou Gehrig's disease are used interchangeably and refer to a debilitating disease with varied etiology characterized by rapidly progressive weakness, muscle atrophy and fasciculations, muscle spasticity, difficulty speaking (dysarthria), difficulty swallowing (dysphagia), and difficulty breathing (dyspnea).

Progranulin haploinsufficiency due to heterozygous loss-of-function mutations in the GRN gene results in a reduction of CSF progranulin levels and is causal for the development of frontotemporal dementia (FTD) with TDP-43 pathology (Sleegers et al., (2009) Ann Neurol 65:603; Smith et al., (2012) Am J Hum Genet 90:1102). TDP-43 has also been identified as a major pathological protein in ALS, suggesting a similarity between ALS and FTD.

For example, over twenty dominant mutations in TDP-43 have been identified in sporadic and familial ALS patients (Lagier-Tourenne et al., (2009) Cell 136:1001) and TDP-43 positive aggregates are found in approximately 95% of ALS cases (Prasad et al., (2019) Front Mol Neurosci 12:25). Furthermore, ALS risk genes, such as MOBP, C9ORF72, MOBKL2B, NSF and FUS, can also cause FTD (Karch et al., (2018) JAMA Neurol 75:860). In addition, both progranulin and C9ORF72 mutations are associated with abnormal microglial activation, which appears to be another common pathology of FTD and ALS (Haukedal et al., (2019) J Mol Biol 431:1818). Other evidence also suggests that ALS and FTD are closely related conditions with overlapping genetic, neuropathological, and clinical features (Weishaupt et al., (2016) Trends Mol Med 22:769; McCauley et al., (2018) Acta Neuropathol 137:715). Taken together, these results suggest that both diseases could benefit from shared treatments and that progranulin genetic variability acts as a modifier of the course of ALS.

Moreover, aside from demonstrations that loss of progranulin is detrimental in multiple models of acute and chronic neurodegeneration (Boddaert et al., (2018) Methods Mol Biol 1806:233), overexpression of progranulin has been found to be protective in many animal models of ALS (Laird et al., (2010) PLoS One 5:e13368; Tauffenberger et al., (2013) Hum Mol Genet 22:782; Beel et al., (2018) Mol Neurodegener 13:55; Chang et al., (2017) J Exp Med 214:2611). In addition, common variants in GRN are significantly associated with a reduction in age at onset and a shorter survival after onset in ALS patients (Sleegers et al., (2008) Neurology 71:253).

In summary, both human genetics and data from disease models support a protective function for progranulin in reducing pathology in ALS cases that are associated with TDP-43 pathology.

In some embodiments, and without wishing to be bound by theory, it is believed that anti-sortilin antibodies of the present disclosure that inhibit the interaction between sortilin and progranulin, neurotrophins of the present disclosure (e.g., pro-neurotrophins, pro-neurotrophin-3, pro-neurotrophin-4/5, pro-NGF, pro-BDNF, neurotrophin-3, neurotrophin-4/5, NGF, BDNF, etc.), neurotensin, p75, lipoprotein lipase (LpL), apolipoprotein AV (APOA5), and/or receptor associated protein (RAP); or that inhibit one or more activities of sortilin can be utilized to prevent, or treat one or more undesirable symptoms of ALS.

In some embodiments, administering an anti-sortilin antibody of the present disclosure may treat and/or delay the progression of ALS in an individual. In some embodiments, administering an anti-sortilin antibody of the present disclosure to an individual having or at risk for having ALS may increase levels of progranulin in the individual, e.g., in plasma and/or cerebrospinal fluid; and/or decrease levels of sortilin; and/or inhibit the interaction between sortilin and one or more proteins; and/or inhibit the interaction between sortilin and progranulin, thereby treating and/or delaying progression of ALS.

In some embodiments, the individual is heterozygous for a C9orf72 hexanucleotide repeat expansion.

In some embodiments, treatment and/or delay of ALS progression is determined by a change from baseline in brain atrophy, brain connectivity, brain free water and/or brain inflammation. Any method known in the art including, without limitation, MRI, may be used to measure brain atrophy, brain connectivity, brain free water and/or brain inflammation. In certain embodiments, brain atrophy is measured using structural MRI. In certain embodiments, brain free water and/or brain inflammation are measured using diffusion tensor imaging (DTI).

In some embodiments, treatment and/or delay of ALS progression is determined by a change from baseline in progranulin, markers of neurodegeneration, markers of glial activation, and/or markers of TDP-43 pathology. In certain embodiments, progranulin is measured using an Adipogen immunoassay. In certain embodiments, markers of neurodegeneration include, without limitation, neurofilament light chain. Neurofilament light chain may be measured by any known methods in the art including, without limitation, assays from Quanterix and/or Roche Diagnostics. In certain embodiments, markers of glial activation include, without limitation, YKL-40 (CHI3L), IL-6, and/or GFAP. GFAP may be measured using any methods known in the art including, without limitation, assays from Roche Diagnostics.

### Multiple Sclerosis

Multiple sclerosis (MS) can also be referred to as disseminated sclerosis or encephalomyelitis disseminata. MS is an inflammatory disease in which the fatty myelin sheaths around the axons of the brain and spinal cord are damaged, leading to demyelination and scarring as well as a broad spectrum of signs and symptoms. See, e.g., www.ninds.nih.gov/Disorders/Patient-Caregiver-Education/Hope-Through-Research/Multiple-Sclerosis-Hope-Through-Research.

Symptoms of MS include, without limitation, changes in sensation, such as loss of sensitivity or tingling; pricking or numbness, such as hypoesthesia and paresthesia; muscle weakness; clonus; muscle spasms; difficulty in moving; difficulties with coordination and balance, such as ataxia; problems in speech, such as dysarthria, or in swallowing, such as dysphagia; visual problems, such as nystagmus, optic neuritis including phosphenes, and diplopia; fatigue; acute or chronic pain; bladder and bowel difficulties; cognitive impairment of varying degrees; emotional symptoms of depression or unstable mood; Uhthoffs phenomenon, which is an exacerbation of extant symptoms due to an exposure to higher than usual ambient temperatures; and Lhermitte's sign, which is an electrical sensation that runs down the back when bending the neck.

In some embodiments, administering an anti-sortilin antibody of the present disclosure may treat and/or delay the progression of MS in an individual. In some embodiments, administering an anti-sortilin antibody of the present disclosure to an individual having or at risk for having MS may increase levels of progranulin in the individual, e.g., in plasma and/or cerebrospinal fluid; and/or decrease levels of sortilin; and/or inhibit the interaction between sortilin and one or more proteins; and/or inhibit the interaction between sortilin and progranulin, thereby treating and/or delaying progression of MS.

### Glaucoma and Macular Degeneration

Glaucoma describes, without limitation, a group of diseases that are characterized by a damaged optic nerve, resulting in vision loss and blindness. Glaucoma is usually caused by increased fluid pressure (e.g., intraocular pressure) in the anterior chamber underneath the cornea. Glaucoma results in the successive loss of retinal ganglion cells that are important for vision. Age-related macular degeneration usually affects older people and primarily causes loss of vision in the macula, the central field of vision. Macular degeneration causes, without limitation, drusen, pigmentary changes, distorted vision, hemorrhages of the eye, atrophy, reduced visual acuity, blurred vision, central scotomas, reduced color vision and reduced contrast sensitivity.

In some embodiments, administering an anti-sortilin antibody of the present disclosure may treat and/or delay the progression of glaucoma or macular degeneration in an individual. In some embodiments, administering an anti-sortilin antibody of the present disclosure to an individual having or at risk for having glaucoma or macular degeneration may increase levels of progranulin in the individual, e.g., in plasma and/or cerebrospinal fluid; and/or decrease levels of sortilin; and/or inhibit the interaction between sortilin and one or more proteins; and/or inhibit the interaction between sortilin and progranulin, thereby treating and/or delaying progression of glaucoma or macular degeneration.

### Administration and Pharmaceutical Dosages

An anti-sortilin antibody provided herein (and any additional therapeutic agent) can be administered by any suitable means, including parenteral, intrapulmonary, intranasal, intralesional, intracerobrospinal, intracranial, intraspinal, intrasynovial, intrathecal, oral, topical, or inhalation routes. Parenteral administration includes intramuscular, intraarterial, intra-articular, intraperitoneal, subcutaneous or intravenous administration. Intravenous administration includes administration as a bolus or by continuous infusion over a period of time. In some embodiments, administration of an antibody of the disclosure is by intravenous administration. In some embodiments, administration of an antibody of the disclosure is by subcutaneous injection. Various dosing schedules including but not limited to single or multiple administrations over various time-points, bolus administration, and pulse infusion are contemplated herein.

Anti-sortilin antibodies provided herein are formulated, dosed, and administered in a fashion consistent with good medical practice. Factors for consideration in this context include the particular disease or disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disease or disorder in question. In some embodiments, an anti-sortilin antibody of the disclosure is formulated and/or administered in combination with one or more additional anti-sortilin agents, such as one or more anti-sortilin antibodies. The effective amount of such other agents depends on the amount of antibody present in the formulation, the type of disease, disorder or treatment, and other factors discussed above.

In some embodiments, dosages for a particular anti-sortilin antibody of the disclosure may be determined empirically in individuals who have been given one or more administrations of the anti-sortilin antibody. In some embodiments, individuals are given incremental doses of an anti-sortilin antibody of the disclosure. To assess efficacy of an anti-sortilin antibody of the disclosure, a clinical symptom of any of the diseases, disorders, or injuries of the present disclosure (e.g., frontotemporal dementia, progressive supranuclear palsy, Alzheimer's disease, vascular dementia, seizures, retinal dystrophy, amyotrophic lateral sclerosis, traumatic brain injury, a spinal cord injury, dementia, stroke, Parkinson's disease, limbic-predominant age-related TDP43 encephalopathy (LATE), acute disseminated encephalomyelitis, retinal degeneration, age related macular degeneration, glaucoma, multiple sclerosis, septic shock, bacterial infection, arthritis, or osteoarthritis) can be monitored.

### Intravenous Administration

In some embodiments, the methods provided herein comprise administering an anti-sortilin antibody of the disclosure by intravenous infusion to an individual having or at risk for having a disease, disorder or injury. In some embodiments, the disease, disorder or injury is frontotemporal dementia, progressive supranuclear palsy, Alzheimer's disease, vascular dementia, seizures, retinal dystrophy, amyotrophic lateral sclerosis, traumatic brain injury, a spinal cord injury, dementia, stroke, Parkinson's disease, acute disseminated encephalomyelitis, retinal degeneration, age related macular degeneration, glaucoma, multiple sclerosis, septic shock, bacterial infection, arthritis, or osteoarthritis. In some embodiments, the disease or disorder is Alzheimer's disease. In some embodiments, the disease or disorder is frontotemporal dementia. In some embodiments, the disease or disorder is Parkinson's disease.

In some embodiments, the anti-sortilin antibody is administered intravenously at a dose of at least about 6 mg/kg. In some embodiments, the anti-sortilin antibody is administered intravenously at a dose of between about 6 mg/kg and about 60 mg/kg. In some embodiments, the anti-sortilin antibody is administered intravenously at a dose of up to about 30 mg/kg. In some embodiments, the anti-sortilin antibody is administered intravenously at a dose of between about 15 mg/kg and about 30 mg/kg. In some embodiments, the anti-sortilin antibody is administered intravenously at a dose of up to about 60 mg/kg. In some embodiments, the dose is about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, or about 60 mg/kg. In some embodiments, the anti-sortilin antibody is administered intravenously at a dose of about 6 mg/kg. In some embodiments, the anti-sortilin antibody is administered intravenously at a dose of about 15 mg/kg. In some embodiments, the anti-sortilin antibody is administered intravenously at a dose of about 30 mg/kg. In some embodiments, the anti-sortilin antibody is administered intravenously at a dose of about 60 mg/kg.

Such doses may be administered intermittently. In certain embodiments, the dosing frequency is three times per day, twice per day, once per day, once every other day, once weekly, once every two weeks, once every three weeks, once every four weeks, once every five weeks, once every six weeks, once every seven weeks, once every eight weeks, once every nine weeks, once every ten weeks, or less frequently. In certain embodiments, the dosing frequency is once per month, once every two months, once every three months, once every four months, once every five months, once every six months, or less frequently.

In some embodiments, an anti-sortilin antibody of the disclosure is administered intravenously at any of the above doses using any of the dosing regimens described below.

In some embodiments, an anti-sortilin antibody of the disclosure is administered intravenously about once every four weeks, or more frequently. In some embodiments, an anti-sortilin antibody of the disclosure is administered intravenously about once every week (qlw). In some embodiments, an anti-sortilin antibody of the disclosure is administered intravenously about once every two weeks (q2w). In some embodiments, an anti-sortilin antibody of the disclosure is administered intravenously about once every three weeks (q3w). In some embodiments, an anti-sortilin antibody of the disclosure is administered intravenously about once every four weeks (q4w).

In some embodiments, an anti-sortilin antibody of the disclosure is administered intravenously about once every four weeks, or less frequently. In some embodiments, an anti-sortilin antibody of the disclosure is administered intravenously about once every four weeks (q4w). In some embodiments, an anti-sortilin antibody of the disclosure is administered intravenously about once every five weeks (q5w). In some embodiments, an anti-sortilin antibody of the disclosure is administered intravenously about once every six weeks (q6w). In some embodiments, an anti-sortilin antibody of the disclosure is administered intravenously about once every seven weeks (q7w). In some embodiments, an anti-sortilin antibody of the disclosure is administered intravenously about once every eight weeks (q8w). In some embodiments, an anti-sortilin antibody of the disclosure is administered intravenously about once every nine weeks (q9w). In some embodiments, an anti-sortilin antibody of the disclosure is administered intravenously about once every ten weeks (q10w).

In some embodiments, an anti-sortilin antibody of the disclosure is administered intravenously about once per month, or less frequently. In some embodiments, an anti-sortilin antibody of the disclosure is administered intravenously about once per month. In some embodiments, an anti-sortilin antibody of the disclosure is administered intravenously about once every two months. In some embodiments, an anti-sortilin antibody of the disclosure is administered intravenously about once every three months. In some embodiments, an anti-sortilin antibody of the disclosure is administered intravenously about once every four months. In some embodiments, an anti-sortilin antibody of the disclosure is administered intravenously about once every five months. In some embodiments, an anti-sortilin antibody of the disclosure is administered intravenously about once every six months.

In some embodiments, the methods provided herein comprise administering to an individual an anti-sortilin antibody of the disclosure intravenously at a dose of at least about 6 mg/kg about once every four weeks (q4w), i.e., about once every 28 days. In some embodiments, the methods provided herein comprise administering to an individual an anti-sortilin antibody of the disclosure intravenously at a dose of at least about 6 mg/kg about once per month. In some embodiments, the methods provided herein comprise administering to an individual an anti-sortilin antibody of the disclosure intravenously at a dose of at least about 6 mg/kg about once every six weeks (q6w). In some embodiments, the methods provided herein comprise administering to an individual an anti-sortilin antibody of the disclosure intravenously at a dose of at least about 6 mg/kg about once every eight weeks (q8w). In some embodiments, the methods provided herein comprise administering to an individual an anti-sortilin antibody of the disclosure intravenously at a dose of about 6 mg/kg about once every four weeks (q4w). In some embodiments, the methods provided herein comprise administering to an individual an anti-sortilin antibody of the disclosure intravenously at a dose of about 6 mg/kg about once every six weeks (q6w). In some embodiments, the methods provided herein comprise administering to an individual an anti-sortilin antibody of the disclosure intravenously at a dose of about 6 mg/kg about once every eight weeks (q8w).

In some embodiments, the methods provided herein comprise administering to an individual an anti-sortilin antibody of the disclosure intravenously at a dose of at least about 15 mg/kg about once every four weeks (q4w), i.e., about once every 28 days. In some embodiments, the methods provided herein comprise administering to an individual an anti-sortilin antibody of the disclosure intravenously at a dose of at least about 15 mg/kg about once per month. In some embodiments, the methods provided herein comprise administering to an individual an anti-sortilin antibody of the disclosure intravenously at a dose of at least about 15 mg/kg about once every six weeks (q6w). In some embodiments, the methods provided herein comprise administering to an individual an anti-sortilin antibody of the disclosure intravenously at a dose of at least about 15 mg/kg about once every eight weeks (q8w). In some embodiments, the methods provided herein comprise administering to an individual an anti-sortilin antibody of the disclosure intravenously at a dose of about 15 mg/kg about once every four weeks (q4w). In some embodiments, the methods provided herein comprise administering to an individual an anti-sortilin antibody of the disclosure intravenously at a dose of about 15 mg/kg about once every six weeks (q6w). In some embodiments, the methods provided herein comprise administering to an individual an anti-sortilin antibody of the disclosure intravenously at a dose of about 15 mg/kg about once every eight weeks (q8w).

In some embodiments, the methods provided herein comprise administering to an individual an anti-sortilin antibody of the disclosure intravenously at a dose of up to about 30 mg/kg about once every four weeks (q4w), i.e., about once every 28 days. In some embodiments, the methods provided herein comprise administering to an individual an anti-sortilin antibody of the disclosure intravenously at a dose of up to about 30 mg/kg about once per month.

In some embodiments, the methods provided herein comprise administering to an individual an anti-sortilin antibody of the disclosure intravenously at a dose of at least about 30 mg/kg about once every four weeks (q4w), i.e., once every 28 days. In some embodiments, the methods provided herein comprise administering to an individual an anti-sortilin antibody of the disclosure intravenously at a dose of at least about 30 mg/kg about once per month. In some embodiments, the methods provided herein comprise administering to an individual an anti-sortilin antibody of the disclosure intravenously at a dose of at least about 30 mg/kg about once every six weeks (q6w). In some embodiments, the methods provided herein comprise administering to an individual an anti-sortilin antibody of the disclosure intravenously at a dose of at least about 30 mg/kg about once every eight weeks (q8w).

In some embodiments, the methods provided herein comprise administering to an individual an anti-sortilin antibody of the disclosure intravenously at a dose of at least about 60 mg/kg about once every four weeks (q4w), i.e., once every 28 days. In some embodiments, the methods provided herein comprise administering to an individual an anti-sortilin antibody of the disclosure intravenously at a dose of at least about 60 mg/kg about once per month. In some embodiments, the methods provided herein comprise administering to an individual an anti-sortilin antibody of the disclosure intravenously at a dose of at least about 60 mg/kg about once every six weeks (q6w). In some embodiments, the methods provided herein comprise administering to an individual an anti-sortilin antibody of the disclosure intravenously at a dose of at least about 60 mg/kg about once every eight weeks (q8w). In some embodiments, the methods provided herein comprise administering to an individual an anti-sortilin antibody of the disclosure intravenously at a dose of about 60 mg/kg about once every four weeks (q4w). In some embodiments, the methods provided herein comprise administering to an individual an anti-sortilin antibody of the disclosure intravenously at a dose of about 60 mg/kg about once every six weeks (q6w). In some embodiments, the methods provided herein comprise administering to an individual an anti-sortilin antibody of the disclosure intravenously at a dose of about 60 mg/kg about once every eight weeks (q8w).

In some embodiments, the methods provided herein comprise administering to an individual an anti-sortilin antibody of the disclosure intravenously at a dose of between about 6 mg/kg and about 60 mg/kg about once every four weeks (q4w), i.e., about once every 28 days. In some embodiments, the methods provided herein comprise administering to an individual an anti-sortilin antibody of the disclosure intravenously at a dose of between about 6 mg/kg and about 60 mg/kg once per month.

In some embodiments, the methods provided herein comprise administering to an individual an anti-sortilin antibody of the disclosure intravenously at a dose of between about 6 mg/kg and about 60 mg/kg about once every six weeks (q6w). In some embodiments, the methods provided herein comprise administering to an individual an anti-sortilin antibody of the disclosure intravenously at a dose of between about 6 mg/kg and about 60 mg/kg about once every eight weeks (q8w).

In some embodiments, the methods provided herein comprise administering to an individual an anti-sortilin antibody of the disclosure intravenously at a dose of about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, or about 60 mg/kg about once every four weeks (q4w), i.e., about once every 28 days. In some embodiments, the methods provided herein comprise administering to an individual an anti-sortilin antibody of the disclosure intravenously at a dose of about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, or about 60 mg/kg about once per month. In some embodiments, the methods provided herein comprise administering to an individual an anti-sortilin antibody of the disclosure intravenously at a dose of about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, or about 60 mg/kg about once every six weeks (q6w). In some embodiments, the methods provided herein comprise administering to an individual an anti-sortilin antibody of the disclosure intravenously at a dose of about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, or about 60 mg/kg about once every eight weeks (q8w).

In some embodiments, the methods provided herein comprise administering to an individual an anti-sortilin antibody of the disclosure intravenously at a dose of about 6 mg/kg about once every four weeks (q4w), i.e., about once every 28 days. In some embodiments, the methods provided herein comprise administering to an individual an anti-sortilin antibody of the disclosure intravenously at a dose of about 6 mg/kg about once per month. In some embodiments, the methods provided herein comprise administering to an individual an anti-sortilin antibody of the disclosure intravenously at a dose of about 6 mg/kg about once every six weeks (q6w). In some embodiments, the methods provided herein comprise administering to an individual an anti-sortilin antibody of the disclosure intravenously at a dose of about 6 mg/kg about once every eight weeks (q8w).

In some embodiments, the methods provided herein comprise administering to an individual an anti-sortilin antibody of the disclosure intravenously at a dose of about 15 mg/kg about once every four weeks (q4w), i.e., about once every 28 days. In some embodiments, the methods provided herein comprise administering to an individual an anti-sortilin antibody of the disclosure intravenously at a dose of about 15 mg/kg about once per month. In some embodiments, the methods provided herein comprise administering to an individual an anti-sortilin antibody of the disclosure intravenously at a dose of about 15 mg/kg about once every six weeks (q6w). In some embodiments, the methods provided herein comprise administering to an individual an anti-sortilin antibody of the disclosure intravenously at a dose of about 15 mg/kg about once every eight weeks (q8w).

In some embodiments, the methods provided herein comprise administering to an individual an anti-sortilin antibody of the disclosure intravenously at a dose of about 30 mg/kg about once every four weeks (q4w), i.e., about once every 28 days. In some embodiments, the methods provided herein comprise administering to an individual an anti-sortilin antibody of the disclosure intravenously at a dose of about 30 mg/kg about once per month. In some embodiments, the methods provided herein comprise administering to an individual an anti-sortilin antibody of the disclosure intravenously at a dose of about 30 mg/kg about once every six weeks (q6w). In some embodiments, the methods provided herein comprise administering to an individual an anti-sortilin antibody of the disclosure intravenously at a dose of about 30 mg/kg about once every eight weeks (q8w).

In some embodiments, the methods provided herein comprise administering to an individual an anti-sortilin antibody of the disclosure intravenously at a dose of about 60 mg/kg about once every four weeks (q4w), i.e., about once every 28 days. In some embodiments, the methods provided herein comprise administering to an individual an anti-sortilin antibody of the disclosure intravenously at a dose of about 60 mg/kg about once per month. In some embodiments, the methods provided herein comprise administering to an individual an anti-sortilin antibody of the disclosure intravenously at a dose of about 60 mg/kg about once every six weeks (q6w). In some embodiments, the methods provided herein comprise administering to an individual an anti-sortilin antibody of the disclosure intravenously at a dose of about 60 mg/kg about once every eight weeks (q8w).

In certain embodiments, a dose of an anti-sortilin antibody of the disclosure is administered to an individual intravenously, e.g., using an infusion pump, over about 60 minutes.

In certain embodiments, at least 1 dose, at least 2 doses, at least 3 doses, at least 4 doses, at least 5 doses, at least 6 doses, at least 7 doses, at least 8 doses, at least 9 doses, at least 10 doses, at least 11 doses, at least 12 doses, at least 13 doses, at least 14 doses, at least 15 doses, at least 16 doses, at least 17 doses, at least 18 doses, at least 19 doses, at least 20 doses, at least 21 doses, at least 22 doses, at least 23 doses, at least 24 doses, at least 25 doses, at least 26 doses, at least 27 doses, at least 28 doses, at least 29 doses, at least 30 doses, at least 31 doses, at least 32 doses, at least 33 doses, at least 34 doses, at least 35 doses, at least 36 doses, or more, of an anti-sortilin antibody of the disclosure are administered to the individual intravenously. In certain embodiments, at least 6 doses of the anti-sortilin antibody are administered to the individual.

In some embodiments, the first dose of the anti-sortilin antibody is administered on the first day of the treatment period and administration of the anti-sortilin antibody continues about once every four weeks, or more frequently, thereafter. In some embodiments, the first dose of the anti-sortilin antibody is administered on the first day of the treatment period and administration of the anti-sortilin antibody continues about once every week (qlw), about once every two weeks (q2w), about once every three weeks (q3w), or about once every four weeks (q4w) thereafter.

In some embodiments, the first dose of the anti-sortilin antibody is administered on the first day of the treatment period and administration of the anti-sortilin antibody continues about once every four weeks, or less frequently, thereafter. In some embodiments, the first dose of the anti-sortilin antibody is administered on the first day of the treatment period and administration of the anti-sortilin antibody continues about once every four weeks (q4w), about once every five weeks (q5w), about once every six weeks (q6w), about once every seven weeks (q7w), about once every eight weeks (q8w), about once every nine weeks (q9w), or about once every ten weeks (q10w) thereafter.

In some embodiments, the first dose of the anti-sortilin antibody is administered on the first day of the treatment period and administration of the anti-sortilin antibody continues about once per month, or less frequently, thereafter. In some embodiments, the first dose of the anti-sortilin antibody is administered on the first day of the treatment period and administration of the anti-sortilin antibody continues about once per month, about once every two months, about once every three months, about once every four months, about once every five months, or about once every six months thereafter.

In some embodiments, the individual is treated with an anti-Sortilin antibody of the disclosure for a treatment period of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, or more. In some embodiments, the individual is treated with an anti-Sortilin antibody of the disclosure for a treatment period of at least about 12 months, at least about 15 months, at least about 18 months, at least about 21 months, at least about 24 months, at least about 27 months, at least about 30 months, at least about 33 months, at least about 36 months, or more. In some embodiments, the individual is treated with an anti-Sortilin antibody of the disclosure for a treatment period of at least about 1 year, at least about 2 years, at least about 3 years, at least about 4 years, at least about 5 years, or more.

In some embodiments, an initial higher dose of the anti-sortilin antibody is administered to the individual, followed by one or more lower doses of the anti-sortilin antibody. In some embodiments, an initial dose of about 60 mg/kg of the anti-sortilin antibody is administered to the individual, followed by one or more lower doses of between about 6 mg/kg and about 59 mg/kg, or between about 6 mg/kg and about 30 mg/kg, or between about 6 mg/kg and about 15 mg/kg, of the anti-sortilin antibody. In some embodiments, an initial dose of about 30 mg/kg of the anti-sortilin antibody is administered to the individual, followed by one or more lower doses of between about 6 mg/kg and about 29 mg/kg, or between about 6 mg/kg and about 15 mg/kg, of the anti-sortilin antibody. In some embodiments, an initial dose of about 60 mg/kg of the anti-sortilin antibody is administered to the individual, followed by one or more lower doses of between about 6 mg/kg and about 59 mg/kg, between about 6 mg/kg and about 30 mg/kg, or between about 6 mg/kg and about 15 mg/kg, of the anti-sortilin antibody. In some embodiments, an initial lower dose of the anti-sortilin antibody is administered to the individual, followed by one or more higher doses of the anti-sortilin antibody. In some embodiments, an initial dose of about 6 mg/kg of the anti-sortilin antibody is administered to the individual, followed by one or more higher doses of between about 7 mg/kg and about 30 mg/kg, between about 15 mg/kg and about 30 mg/kg, or between about 30 mg/kg and about 60 mg/kg, of the anti-sortilin antibody. In some embodiments, an initial dose of about 15 mg/kg of the anti-sortilin antibody is administered to the individual, followed by one or more higher doses of between about 16 mg/kg and about 30 mg/kg, or between about 30 mg/kg and about 60 mg/kg, of the anti-sortilin antibody. In some embodiments, an initial dose of about 30 mg/kg of the anti-sortilin antibody is administered to the individual, followed by one or more higher doses of between about 31 mg/kg and about 60 mg/kg of the anti-sortilin antibody.

As will be understood by those of ordinary skill in the art, other dosage regimens may be used according to the methods provided herein, e.g., based on monitoring of the individual and/or the disease or disorder being treated according to methods known in the art, e.g., as described herein.

### Subcutaneous Administration

In some embodiments, the methods provided herein comprise administering an anti-sortilin antibody of the disclosure by subcutaneous administration to an individual having or at risk for having a disease, disorder or injury. In some embodiments, the disease, disorder or injury is frontotemporal dementia, progressive supranuclear palsy, Alzheimer's disease, vascular dementia, seizures, retinal dystrophy, amyotrophic lateral sclerosis, traumatic brain injury, a spinal cord injury, dementia, stroke, Parkinson's disease, acute disseminated encephalomyelitis, retinal degeneration, age related macular degeneration, glaucoma, multiple sclerosis, septic shock, bacterial infection, arthritis, or osteoarthritis. In some embodiments, the disease or disorder is Alzheimer's disease. In some embodiments, the disease or disorder is frontotemporal dementia. In some embodiments, the disease or disorder is Parkinson's disease.

In some embodiments, the methods provided herein comprise administering to an individual an anti-sortilin antibody of the disclosure subcutaneously at a dose of at least about 150 mg. In some embodiments, the methods provided herein comprise administering to an individual an anti-sortilin antibody of the disclosure subcutaneously at a dose of at least about 260 mg. In some embodiments, the methods provided herein comprise administering to the individual an anti-sortilin antibody of the disclosure subcutaneously at a dose of between about 260 mg and about 4000 mg. In some embodiments, the methods provided herein comprise administering to the individual an anti-sortilin antibody of the disclosure subcutaneously at a dose of any of about 260 mg, about 280 mg, about 300 mg, about 320 mg, about 340 mg, about 360 mg, about 380 mg, about 400 mg, about 420 mg, about 440 mg, about 460 mg, about 480 mg, about 500 mg, about 520 mg, about 540 mg, about 560 mg, about 580 mg, about 600 mg, about 620 mg, about 640 mg, about 660 mg, about 680 mg, about 700 mg, about 720 mg, about 740 mg, about 760 mg, about 780 mg, about 800 mg, about 820 mg, about 840 mg, about 860 mg, about 880 mg, about 900 mg, about 920 mg, about 940 mg, about 960 mg, about 980 mg, about 1000 mg, about 1020 mg, about 1040 mg, about 1060 mg, about 1080 mg, about 1100 mg, about 1120 mg, about 1140 mg, about 1160 mg, about 1180 mg, about 1200 mg, about 1220 mg, about 1240 mg, about 1260 mg, about 1280 mg, about 1300 mg, about 1320 mg, about 1340 mg, about 1360 mg, about 1380 mg, about 1400 mg, about 1420 mg, about 1440 mg, about 1460 mg, about 1480 mg, about 1500 mg, about 1520 mg, about 1540 mg, about 1560 mg, about 1580 mg, about 1600 mg, about 1620 mg, about 1640 mg, about 1660 mg, about 1680 mg, about 1700 mg, about 1720 mg, about 1740 mg, about 1760 mg, about 1780 mg, about 1800 mg, about 1820 mg, about 1840 mg, about 1860 mg, about 1880 mg, about 1900 mg, about 1920 mg, about 1940 mg, about 1960 mg, about 1980 mg, about 2000 mg, about 2020 mg, about 2040 mg, about 2060 mg, about 2080 mg, about 2100 mg, about 2120 mg, about 2140 mg, about 2160 mg, about 2180 mg, about 2200 mg, about 2220 mg, about 2240 mg, about 2260 mg, about 2280 mg, about 2300 mg, about 2320 mg, about 2340 mg, about 2360 mg, about 2380 mg, about 2400 mg, about 2420 mg, about 2440 mg, about 2460 mg, about 2480 mg, about 2500 mg, about 2520 mg, about 2540 mg, about 2560 mg, about 2580 mg, about 2600 mg, about 2620 mg, about 2640 mg, about 2660 mg, about 2680 mg, about 2700 mg, about 2720 mg, about 2740 mg, about 2760 mg, about 2780 mg, about 2800 mg, about 2820 mg, about 2840 mg, about 2860 mg, about 2880 mg, about 2900 mg, about 2920 mg, about 2940 mg, about 2960 mg, about 2980 mg, about 3000 mg, about 3020 mg, about 3040 mg, about 3060 mg, about 3080 mg, about 3100 mg, about 3120 mg, about 3140 mg, about 3160 mg, about 3180 mg, about 3200 mg, about 3220 mg, about 3240 mg, about 3260 mg, about 3280 mg, about 3300 mg, about 3320 mg, about 3340 mg, about 3360 mg, about 3380 mg, about 3400 mg, about 3420 mg, about 3440 mg, about 3460 mg, about 3480 mg, about 3500 mg, about 3520 mg, about 3540 mg, about 3560 mg, about 3580 mg, about 3600 mg, about 3620 mg, about 3640 mg, about 3660 mg, about 3680 mg, about 3700 mg, about 3720 mg, about 3740 mg, about 3760 mg, about 3780 mg, about 3800 mg, about 3820 mg, about 3840 mg, about 3860 mg, about 3880 mg, about 3900 mg, about 3920 mg, about 3940 mg, about 3960 mg, about 3980 mg, or about 4000 mg.

In some embodiments, the methods provided herein comprise administering to an individual an anti-sortilin antibody of the disclosure subcutaneously at a dose of between about 270 mg and about 600 mg, between about 600 mg and about 675 mg, between about 675 mg and about 720 mg, between about 720 mg and about 1350 mg, between about 1350 mg and about 1600 mg, between about 1600 mg and about 1800 mg, or between about 1800 mg and about 3600 mg. In some embodiments, the methods provided herein comprise administering to an individual an anti-sortilin antibody of the disclosure subcutaneously at a dose of about 270 mg, about 675 mg, about 720 mg, about 1350 mg, about 1800 mg, or about 3600 mg. In some embodiments, the anti-sortilin antibody is administered subcutaneously at a dose of about 150 mg. In some embodiments, the anti-sortilin antibody is administered subcutaneously at a dose of about 270 mg. In some embodiments, the anti-sortilin antibody is administered subcutaneously at a dose of about 300 mg. In some embodiments, the anti-sortilin antibody is administered subcutaneously at a dose of about 600 mg. In some embodiments, the anti-sortilin antibody is administered subcutaneously at a dose of about 675 mg. In some embodiments, the anti-sortilin antibody is administered subcutaneously at a dose of about 720 mg. In some embodiments, the anti-sortilin antibody is administered subcutaneously at a dose of about 1350 mg. In some embodiments, the anti-sortilin antibody is administered subcutaneously at a dose of about 1600 mg. In some embodiments, the anti-sortilin antibody is administered subcutaneously at a dose of about 1800 mg. In some embodiments, the anti-sortilin antibody is administered subcutaneously at a dose of about 3600 mg.

In some embodiments, the methods provided herein comprise administering to the individual an anti-sortilin antibody of the disclosure subcutaneously at a dose of between about 6 mg/kg and about 60 mg/kg. In some embodiments, the anti-sortilin antibody is administered subcutaneously at a dose of up to about 30 mg/kg. In some embodiments, the anti-sortilin antibody is administered subcutaneously at a dose of up to about 60 mg/kg. In some embodiments, the dose is about 6 mg/kg, about 7 mg/kg, about 8 mg/kg, about 9 mg/kg, about 10 mg/kg, about 11 mg/kg, about 12 mg/kg, about 13 mg/kg, about 13.3 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, or about 60 mg/kg. In some embodiments, the anti-sortilin antibody is administered subcutaneously at a dose of about 6 mg/kg. In some embodiments, the anti-sortilin antibody is administered subcutaneously at a dose of about 13.3 mg/kg. In some embodiments, the anti-sortilin antibody is administered subcutaneously at a dose of about 15 mg/kg. In some embodiments, the anti-sortilin antibody is administered subcutaneously at a dose of about 30 mg/kg. In some embodiments, the anti-sortilin antibody is administered subcutaneously at a dose of about 60 mg/kg.

Such doses may be administered intermittently. In certain embodiments, the dosing frequency is three times per day, twice per day, once per day, once every other day, once weekly, once every two weeks, once every three weeks, once every four weeks, once every five weeks, once every six weeks, once every seven weeks, once every eight weeks, once every nine weeks, once every ten weeks, or less frequently. In certain embodiments, the dosing frequency is once per month, once every two months, once every three months, once every four months, once every five months, once every six months, or less frequently.

In some embodiments, an anti-sortilin antibody of the disclosure is administered subcutaneously at any of the above doses using any of the dosing regimens described below.

In some embodiments, an anti-sortilin antibody of the disclosure is administered subcutaneously about once every four weeks (q4w), or more frequently. In some embodiments, an anti-sortilin antibody of the disclosure is administered subcutaneously about once every week (qlw). In some embodiments, an anti-sortilin antibody of the disclosure is administered subcutaneously about once every two weeks (q2w). In some embodiments, an anti-sortilin antibody of the disclosure is administered subcutaneously about once every three weeks (q3w). In some embodiments, an anti-sortilin antibody of the disclosure is administered subcutaneously about once every four weeks (q4w).

In some embodiments, an anti-sortilin antibody of the disclosure is administered subcutaneously about once every four weeks (q4w), or less frequently. In some embodiments, an anti-sortilin antibody of the disclosure is administered subcutaneously about once every four weeks (q4w). In some embodiments, an anti-sortilin antibody of the disclosure is administered subcutaneously about once every five weeks (q5w). In some embodiments, an anti-sortilin antibody of the disclosure is administered subcutaneously about once every six weeks (q6w). In some embodiments, an anti-sortilin antibody of the disclosure is administered subcutaneously about once every seven weeks (q7w). In some embodiments, an anti-sortilin antibody of the disclosure is administered subcutaneously about once every eight weeks (q8w). In some embodiments, an anti-sortilin antibody of the disclosure is administered subcutaneously about once every nine weeks (q9w). In some embodiments, an anti-sortilin antibody of the disclosure is administered subcutaneously about once every ten weeks (q10w).

In some embodiments, an anti-sortilin antibody of the disclosure is administered subcutaneously about once per month, or less frequently. In some embodiments, an anti-sortilin antibody of the disclosure is administered subcutaneously about once per month. In some embodiments, an anti-sortilin antibody of the disclosure is administered subcutaneously about once every two months. In some embodiments, an anti-sortilin antibody of the disclosure is administered subcutaneously about once every three months. In some embodiments, an anti-sortilin antibody of the disclosure is administered subcutaneously about once every four months. In some embodiments, an anti-sortilin antibody of the disclosure is administered subcutaneously about once every five months. In some embodiments, an anti-sortilin antibody of the disclosure is administered subcutaneously about once every six months.

In certain embodiments, a dose of the anti-sortilin antibody is administered to the individual subcutaneously as a subcutaneous injection. In certain embodiments, a dose of the anti-sortilin antibody is administered to the individual subcutaneously as a slow subcutaneous injection over about 15 minutes.

In certain embodiments, at least 1 dose, at least 2 doses, at least 3 doses, at least 4 doses, at least 5 doses, at least 6 doses, at least 7 doses, at least 8 doses, at least 9 doses, at least 10 doses, at least 11 doses, at least 12 doses, at least 13 doses, at least 14 doses, at least 15 doses, at least 16 doses, at least 17 doses, at least 18 doses, at least 19 doses, at least 20 doses, at least 21 doses, at least 22 doses, at least 23 doses, at least 24 doses, at least 25 doses, at least 26 doses, at least 27 doses, at least 28 doses, at least 29 doses, at least 30 doses, at least 31 doses, at least 32 doses, at least 33 doses, at least 34 doses, at least 35 doses, at least 36 doses, or more, of the anti-sortilin antibody are administered to the individual subcutaneously. In certain embodiments, at least 6 doses of the anti-sortilin antibody are administered to the individual.

In some embodiments, the first dose of the anti-sortilin antibody is administered on the first day of the treatment period and administration of the anti-sortilin antibody continues about once every four weeks (q4w), or more frequently, thereafter. In some embodiments, the first dose of the anti-sortilin antibody is administered on the first day of the treatment period and administration of the anti-sortilin antibody continues about once every week (qlw), about once every two weeks (q2w), about once every three weeks (q3w), or about once every four weeks (q4w) thereafter.

In some embodiments, the first dose of the anti-sortilin antibody is administered on the first day of the treatment period and administration of the anti-sortilin antibody continues about once every four weeks (q4w), or less frequently, thereafter. In some embodiments, the first dose of the anti-sortilin antibody is administered on the first day of the treatment period and administration of the anti-sortilin antibody continues about once every four weeks (q4w), about once every five weeks (q5w), about once every six weeks (q6w), about once every seven weeks (q7w), about once every eight weeks (q8w), about once every nine weeks (q9w), or about once every ten weeks (q10w) thereafter.

In some embodiments, the first dose of the anti-sortilin antibody is administered on the first day of the treatment period and administration of the anti-sortilin antibody continues about once per month, or less frequently, thereafter. In some embodiments, the first dose of the anti-sortilin antibody is administered on the first day of the treatment period and administration of the anti-sortilin antibody continues about once per month, about once every two months, about once every three months, about once every four months, about once every five months, or about once every six months thereafter.

In some embodiments, the individual is treated with an anti-Sortilin antibody of the disclosure for a treatment period of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, or more. In some embodiments, the individual is treated with an anti-Sortilin antibody of the disclosure for a treatment period of at least about 12 months, at least about 15 months, at least about 18 months, at least about 21 months, at least about 24 months, at least about 27 months, at least about 30 months, at least about 33 months, at least about 36 months, or more. In some embodiments, the individual is treated with an anti-Sortilin antibody of the disclosure for a treatment period of at least about 1 year, at least about 2 years, at least about 3 years, at least about 4 years, at least about 5 years, or more.

In some embodiments, an initial higher dose of the anti-sortilin antibody is administered to the individual, followed by one or more lower doses of the anti-sortilin antibody. In some embodiments, an initial dose of about 60 mg/kg of the anti-sortilin antibody is administered to the individual, followed by one or more lower doses of between about 6 mg/kg and about 59 mg/kg, or between about 6 mg/kg and about 30 mg/kg, or between about 6 mg/kg and about 15 mg/kg, of the anti-sortilin antibody In some embodiments, an initial dose of about 30 mg/kg of the anti-sortilin antibody is administered to the individual, followed by one or more lower doses of between about 6 mg/kg and about 29 mg/kg, or between about 6 mg/kg and about 15 mg/kg, of the anti-sortilin antibody. In some embodiments, an initial dose of about 60 mg/kg of the anti-sortilin antibody is administered to the individual, followed by one or more lower doses of between about 6 mg/kg and about 59 mg/kg, between about 6 mg/kg and about 30 mg/kg, or between about 6 mg/kg and about 15 mg/kg, of the anti-sortilin antibody. In some embodiments, an initial lower dose of the anti-sortilin antibody is administered to the individual, followed by one or more higher doses of the anti-sortilin antibody. In some embodiments, an initial dose of about 6 mg/kg of the anti-sortilin antibody is administered to the individual, followed by one or more higher doses of between about 7 mg/kg and about 30 mg/kg, between about 15 mg/kg and about 30 mg/kg, or between about 30 mg/kg and about 60 mg/kg, of the anti-sortilin antibody.

As will be understood by those of ordinary skill in the art, other dosage regimens may be used according to the methods provided herein, e.g., based on monitoring of the individual and/or the disease or disorder being treated according to methods known in the art, e.g., as described herein.

### Sortilin Antibodies

In some aspects, the present disclosure provides anti-sortilin antibodies that bind to sortilin, e.g., a human sortilin protein or a mammalian sortilin protein.

### Sortilin Proteins

In some aspects, the present disclosure provides anti-sortilin antibodies that bind to a sortilin protein. Sortilin is variously referred to as sortilin 1, sort1, SORT1, 100 kDa NT receptor, glycoprotein 95 (GP95), progranulin receptor (PGRN-R), and neurotensin receptor 3 (NT-3 or NTR-3). Sortilin is an 831 amino acid protein that encodes a type I membrane receptor. Various sortilin homologs are known, including without limitation, human sortilin, rat sortilin, cynomolgus monkey sortilin, and mouse sortilin. The amino acid sequence of human sortilin is set forth below as SEQ ID NO: 1 (with key amino acid residues predicted to participate in progranulin binding depicted in bold, and the predicted pro-NGF binding region underlined):

```
                10 20 30 40 50
MERPWGAADG LSRWPHGLGL LLLLQLLPPS TLSQDRLDAP PPPAAPLPRW
                60 70 80 90 100
SGPIGVSWGL RAAAAGGAFP RGGRWRRSAP GEDEECGRVR DFVAKLANNT
              110 120 130 140 150
HQHVFDDLRG SVSLSWVGDS TGVILVLTTF HVPLVIMTFG QSKLYRSEDY
              160 170 180 190 200
GKNFKDITDL INNTFIRTEF GMAIGPENSG KVVLTAEVSG GSRGGRIFRS
              210 220 230 240 250
SDFAKNFVQT DLPFHPLTQM MYSPQNSDYL LALSTENGLW VSKNFGGKWE
              260 270 280 290 300
EIHKAVCLAK WGSDNTIFFT TYANGSCKAD LGALELWRTS DLGKSFKTIG
              310 320 330 340 350
VKIYSFGLGG RFLFASVMAD KDTTRRIHVS TDQGDTWSMA QLPSVGQEQF
              360 370 380 390 400
YSILAANDDM VFMHVDEPGD TGFGTIFTSD DRGIVYSKSL DRHLYTTTGG
              410 420 430 440 450
ETDFTNVTSL RGVYITSVLS EDNSIQTMIT FDQGGRWTHL RKPENSECDA
              460 470 480 490 500
TAKNKNECSL HIHASYSISQ KLNVPMAPLS EPNAVGIVIA HGSVGDAISV
              510 520 530 540 550
MVPDVYISDD GGYSWTKMLE GPHYYTILDS GGIIVAIEHS SRPINVIKFS
              560 570 580 590 600
TDEGQCWQTY TFTRDPIYFT GLASEPGARS MNISIWGFTE SFLTSQWVSY
              610 620 630 640 650
TIDFKDILER NCEEKDYTIW LAHSTDPEDY EDGCILGYKE QFLRLRKSSV
              660 670 680 690 700
CQNGRDYVVT KQPSICLCSL EDFLCDFGYY RPENDSKCVE QPELKGHDLE
              710 720 730 740 750
FCLYGREEHL TINGYRKIPG DKCQGGVNPV REVKDLKKKC TSNFLSPEKQ
              760 770 780 790 800
NSKSNSVPII LAIVGLMLVT VVAGVLIVKK YVCGGRFLVH RYSVLQQHAE
              810 820 830
ANGVDGVDAL DTASHINKSG YHDDSDEDLL E
Additionally, the amino acid sequence of mouse sortilin is set forth in
SEQ ID NO: 2:
MERPRGAADG LLRWPLGLLL LLQLLPPAAV GQDRLDAPPP PAPPLLRWAG PVGVSWGLRA
AAPGGPVPRA GRWRRGAPAE DQDCGRLPDF IAKLINNTHQ HVFDDLSGSV SLSWVGDSTG
VILVLTTFQV PLVIVSFGQS KLYRSEDYGK NEKDITNLIN NTFIRTEFGM AIGPENSGKV
ILTAEVSGGS RGGRVFRSSD FAKNFVQTDL PFHPLTQMMY SPQNSDYLLA LSTENGLWVS
KNFGEKWEEI HKAVCLAKWG PNNIIFFTTH VNGSCKADLG ALELWRTSDL GKTFKTIGVK
IYSFGLGGRF LFASVMADKD TTRRIHVSTD QGDTWSMAQL PSVGQEQFYS ILAANEDMVE
MHVDEPGDTG FGTIFTSDDR GIVYSKSLDR HLYTTTGGET DFTNVTSLRG VYITSTLSED
NSIQSMITFD QGGRWEHLRK PENSKCDATA KNKNECSLHI HASYSISQKL NVPMAPLSEP
NAVGIVIAHG SVGDAISVMV PDVYISDDGG YSWAKMLEGP HYYTILDSGG IIVAIEHSNR
PINVIKFSTD EGQCWQSYVF TQEPIYFTGL ASEPGARSMN ISIWGFTESF ITRQWVSYTV
DFKDILERNC EEDDYTTWLA HSTDPGDYKD GCILGYKEQF LRLRKSSVCQ NGRDYVVAKQ
PSVCPCSLED FLCDFGYFRP ENASECVEQP ELKGHELEFC LYGKEEHLTT NGYRKIPGDK
CQGGMNPARE VKDLKKKCTS NFLNPTKQNS KSNSVPIILA IVGLMLVTVV AGVLIVKKYV
CGGRFLVHRY SVLQQHAEAD GVEALDSTSH AKSGYHDDSD EDLLE
Additionally, the amino acid sequence of rat sortilin is set forth in
SEQ ID NO: 3:
MERPRGAADG LLRWPLGLLL LLQLLPPAAV GQDRLDAPPP PAPPLLRWAG PVGVSWGLRA
AAPGGPVPRA GRWRRGAPAE DQDCGRLPDF IAKLINNTHQ HVFDDLSGSV SLSWVGDSTG
VILVLTTFQV PLVIVSFGQS KLYRSEDYGK NEKDITNLIN NTFIRTEFGM AIGPENSGKV
ILTAEVSGGS RGGRVFRSSD FAKNFVQTDL PFHPLTQMMY SPQNSDYLLA LSTENGLWVS
KNFGEKWEEI HKAVCLAKWG PNNIIFFTTH VNGSCKADLG ALELWRTSDL GKTFKTIGVK
IYSFGLGGRF LFASVMADKD TTRRIHVSTD QGDTWSMAQL PSVGQEQFYS ILAANDDMVF
MHVDEPGDTG FGTIFTSDDR GIVYSKSLDR HLYTTTGGET DFTNVTSLRG VYITSTLSED
NSIQSMITFD QGGRWEHLQK PENSKCDATA KNKNECSLHI HASYSISQKL NVPMAPLSEP
NAVGIVIAHG SVGDAISVMV PDVYISDDGG YSWAKMLEGP HYYTILDSGG IIVAIEHSNR
PINVIKFSTD EGQCWQSYVF SQEPVYFTGL ASEPGARSMN ISIWGFTESF LTRQWVSYTI
DFKDILERNC EENDYTTWLA HSTDPGDYKD GCILGYKEQF LRLRKSSVCQ NGRDYVVAKQ
PSICPCSLED FLCDFGYFRP ENASECVEQP ELKGHELEFC LYGKEEHLTT NGYRKIPGDR
CQGGMNPARE VKDLKKKCTS NFLNPKKQNS KSSSVPIILA IVGLMLVTVV AGVLIVKKYV
CGGRFLVHRY SVLQQHAEAD GVEALDTASH AKSGYHDDSD EDLLE
Additionally, the amino acid sequence of cynomolgus monkey sortilin is
set forth in SEQ ID NO: 27:
MERPRGAADGLSRWPHGLGLLLLLQLLPPVTLGQDRLDAPPPPAAPLLRWSGPIGVSWGLRAAAAGGAFPR
GGRWRRSAPGEDEECGRVRDFVAKLANNTHQHVFDDLRGSVSLSWVGDSTGVILVLTTFHVPLVIMTFGQS
KLYRSEDYGKNFKDITNLINNTFIRTEFGMAIGPENSGKVVLTAEVSGGSRGGRIFRSSDFAKNFVQTDLP
FHPLTQMMYSPQNSDYLLALSTENGLWVSKNFGGKWEEIHKAVCLAKWGSDNTIFFTTYANGSCKADLGAL
ELWRTLDLGKSFKTIGVKIYSFGLGGRFLFASVMADKDTTRRIHVSTDQGDTWSMAQLPSVGQEQFYSILA
ANDDMVFMHVDEPGDTGFGTIFTSDDRGIVYSKSLDRHLYTTTGGETDFTNVTSLRGVYITSVLSEDNSIQ
TMITFDQGGRWKHLRKPENSECDATAKNKNECSLHIHASYSISQKLNVPMAPLSEPNAVGIVIAHGSVGDA
ISVMVPDVYISDDGGYSWTKMLEGPHYYTILDSGGIIVAIEHSSRPINVIKFSTDEGQCWQTYTFTRDPIY
FTGLASEPGARSMNISIWGFTESFLTSQWVSYTIDFKDILERNCEEKDYTIWLAHSTDPEDYEDGCILGYK
EQFLRLRKSSVCQNGRDYVVTKQPSICLCSLEDFLCDFGYYRPENDSKCVEQPELKGHDLEFCLYGREEHL
TTNGYRKIPGDKCQGGVNPVREVKDLKKKCTSNFLSPEKQNSKSNSVPIILAIVGLMLVTVIAGVLIVKKY
VCGGRFLVHRYSVLQQHAEANGMDGVDALDTASHTNKSGYHDDSDEDLLE
```

In some embodiments, an antibody of the disclosure binds to a mammalian sortilin protein, human sortilin protein, primate sortilin protein (e.g., cynomolgus monkey sortilin), mouse sortilin protein, and/or rat sortilin protein. In some embodiments, an antibody of the disclosure may bind an epitope within one or more of a mammalian sortilin protein, human sortilin protein, primate sortilin protein (e.g., cynomolgus monkey sortilin), mouse sortilin protein, and rat sortilin protein. In some embodiments, an antibody of the disclosure binds to human sortilin. In some embodiments, an antibody of the disclosure binds to mouse sortilin. In some embodiments, an antibody of the disclosure binds to cynomolgus monkey sortilin. In some embodiments, an antibody of the disclosure binds to human sortilin and mouse sortilin. In some embodiments, an antibody of the disclosure binds to human sortilin, mouse sortilin, and cynomolgus monkey.

In some embodiments, the sortilin protein is a preprotein that includes a signal sequence. In some embodiments, the sortilin protein is a mature protein. In some embodiments, the mature sortilin protein does not include a signal sequence. In some embodiments, the mature sortilin protein is expressed on a cell.

Sortilin proteins of the present disclosure include several domains, including without limitation, a signal sequence, a propeptide, a luminal domain, a Vps10p domain, e.g., including an Asp-box motif, a ten-bladed beta-propeller structure, and a hydrophobic loop, a 10 CC domain, a transmembrane domain and a cytoplasmic domain. Additionally, sortilin proteins of the present disclosure are expressed at high levels in a number of tissues, including without limitation, the brain, spinal cord, heart and skeletal muscle, thyroid, placenta, and testis. Sortilin is a member of the Vps10p family of sorting receptors, which also includes, without limitation, sorting protein-related receptor with A-type repeats (SorLA), sortilin-related receptor CNS expressed 1 (SorCS1), sortilin-related receptor CNS expressed 2 (SorCS2), and sortilin-related receptor CNS expressed 3 (SorCS3). The luminal region of sortilin aligns with each of the two luminal domains in yeast Vps10p (Vps10p domains). The hallmark of the Vps10p domain is an amino-terminal propeptide and a carboxy-terminal segment that contains 10 conserved cysteine (10CC) residues. Other receptors of the Vps10p family share a Vps10p domain, which is situated at the amino-terminus, and contain additional ectodomains.

The Vps10p family of sorting receptors has diverse functions both within the nervous system and elsewhere. The receptors have been shown to be multifunctional, binding several different ligands, including without limitation, progranulin (PGRN), pro-nerve growth factor (Pro-NGF), nerve growth factor (NGF), PCSK9, pro-neurotrophins, neurotrophins, pro-neurotrophin-3 (pro-NT3), pro-neurotrophin-4/5, pro-brain-derived neurotrophic factor (Pro-BDNF), brain-derived neurotrophic factor (BDNF), neurotrophin-3 (NT3), neurotrophin-4/5, neurotensin, p75NTR, sortilin propeptide (Sort-pro), amyloid precursor protein (APP), lipoprotein lipase (LpL), apolipoproteins, apolipoprotein AV (APOA5), apolipoprotein E (APOE 2, 3, 4), receptor-associated protein (RAP), and elements of the plasminogen activator system; and engaging in intracellular sorting, endocytosis, and signal transduction. Sortilin proteins of the present disclosure have been shown to mediate rapid endocytosis of lipoprotein lipase, neurotensin, and the pro-form of nerve growth factor; and to target proteins for transport from the Golgi to late endosomes. Further, sortilin proteins of the present disclosure have been shown to form a complex with p75 on the cell membrane and be essential to pro-nerve growth factor (NGF)-induced neuronal death. It has also been recently shown that members of the Vps10p receptor family interact with members of the neurotrophin family, which includes NGF, brain derived neurotrophic factor, neurotrophin-3, and neurotrophin-4/5, or the pro-domain form of a neurotrophin (pro-neurotrophin). Sortilin proteins of the present disclosure have also been shown to bind to and regulate extracellular levels of PCSK9, which targets low-density lipoprotein receptor for degradation in lysosomes, resulting in increased levels of LDL cholesterol.

As disclosed herein, interactions between sortilin proteins of the present disclosure and pro-neurotrophins or neurotrophins are mediated by the Vps10p domain that contains a ten-bladed beta-propeller structure and an Asp-box motif. In certain embodiments, sortilin proteins of the present disclosure contain a VpsOp domain that includes a ten-bladed beta-propeller structure and is located within amino acid residues 78-611 of human sortilin (SEQ ID NO: 1) or amino acid residues of a mammalian sortilin that correspond to amino acid residues 78-611 of SEQ ID NO: 1. In certain embodiments, amino acid residues 190-220 of human sortilin (SEQ ID NO: 1) or amino acid residues of a mammalian sortilin that correspond to amino acid residues 190-220 of SEQ ID NO: 1 are located within the VpsOp domain.

Vps10p domains of the present disclosure may include an Asp-box motif. As used herein, Asp-box motifs have the following sequence: (S/T)-X-(D/N)-X-X-X-X-(W/F/Y) (SEQ ID NO: 22), or X-X-(S/T)-X-(D/N)-X-G-X-(T/S)-(W/F/Y)-X (SEQ ID NO: 23), where X represents any amino acid. In human sortilin, the Asp-box motif is located at amino acid residues 200-207 (SSDFAKNF (SEQ ID NO: 24)). Accordingly, in certain embodiments, an Asp-box motif is located at amino acid residues 200-207 of human sortilin (SEQ ID NO:1) or amino acid residues of a mammalian sortilin that correspond to amino acid residues 200-207 of SEQ ID NO: 1.

As disclosed herein, interactions between sortilin proteins of the present disclosure and p75 are mediated by the 10CC domain of the hydrophobic loop of the Vps10p domain.

In certain embodiments, sortilin proteins of the present disclosure contain a 10CC domain that is located within amino acid residues 610-757 of human sortilin (SEQ ID NO: 1) or amino acid residues of a mammalian sortilin that correspond to amino acid residues 610-757 of SEQ ID NO: 1. In some embodiments, amino acid residues 592-593, 610-660, and/or 667-749 of human sortilin (SEQ ID NO: 1) or amino acid residues of a mammalian sortilin that correspond to amino acid residues 592-593, 610-660, and/or 667-749 of SEQ ID NO: 1 are located within the 10CC domain of sortilin.

In other embodiments, sortilin proteins of the present disclosure contain a hydrophobic loop within the Vps10p domain that is located within amino acid residues 130-141 of human sortilin (SEQ ID NO: 1) or amino acid residues of a mammalian sortilin that correspond to amino acid residues 130-141 of SEQ ID NO: 1.

As one of skill in the art will appreciate, the beginning and ending residues of the domains of the present disclosure may vary depending upon the computer modeling program used or the method used for determining the domain.

### Exemplary Anti-Sortilin Antibodies

In some embodiments, anti-sortilin antibodies to be used in the methods of the present disclosure are described in WO2016164637A1, which is hereby incorporated by reference herein.

In some embodiments, anti-sortilin antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 6; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 7; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 8; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 9; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 10; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 11.

In some embodiments, anti-sortilin antibodies of the present disclosure comprise a light chain variable domain and a heavy chain variable domain, wherein the heavy chain variable domain comprises an HVR-H1, HVR-H2, and HVR-H3, and wherein the light chain variable domain comprises an HVR-L1, HVR-L2, and HVR-L3. In some embodiments, the anti-sortilin antibody comprises an HVR-H1 comprising an amino acid sequence containing substitutions (e.g., conservative substitutions, insertions, or deletions) relative to the amino acid sequence YTFTKYYMS (SEQ ID NO:6), but retains the ability to bind to sortilin. In certain embodiments, up to 1, up to 2, up to 3, up to 4, up to 5, or up to 6 amino acids have been substituted, inserted, and/or deleted in the amino acid sequence YTFTKYYMS (SEQ ID NO:6). In some embodiments, the anti-sortilin antibody comprises an HVR-H2 comprising an amino acid sequence containing substitutions (e.g., conservative substitutions, insertions, or deletions) relative to the amino acid sequence IINPIGGSTSYAQKFQG (SEQ ID NO: 7), but retains the ability to bind to sortilin. In certain embodiments, up to 1, up to 2, up to 3, up to 4, up to 5, or up to 6 amino acids have been substituted, inserted, and/or deleted in the amino acid sequence IINPIGGSTSYAQKFQG (SEQ ID NO: 7). In some embodiments, the anti-sortilin antibody comprises an HVR-H3 comprising an amino acid sequence containing substitutions (e.g., conservative substitutions, insertions, or deletions) relative to the amino acid sequence ARDPSGIALAGPASRGYQGMDV (SEQ ID NO:8), but retains the ability to bind to sortilin. In certain embodiments, up to 1, up to 2, up to 3, up to 4, up to 5, or up to 6 amino acids have been substituted, inserted, and/or deleted in the amino acid sequence ARDPSGIALAGPASRGYQGMDV (SEQ ID NO:8). In some embodiments, the anti-sortilin antibody comprises an HVR-L1 comprising an amino acid sequence containing substitutions (e.g., conservative substitutions, insertions, or deletions) relative to the amino acid sequence RASQSVSSNLA (SEQ ID NO:9), but retains the ability to bind to sortilin. In certain embodiments, up to 1, up to 2, up to 3, up to 4, up to 5, or up to 6 amino acids have been substituted, inserted, and/or deleted in the amino acid sequence RASQSVSSNLA (SEQ ID NO:9). In some embodiments, the anti-sortilin antibody comprises an HVR-L2 comprising an amino acid sequence containing substitutions (e.g., conservative substitutions, insertions, or deletions) relative to the amino acid sequence GASTRAT (SEQ ID NO:10), but retains the ability to bind to sortilin. In certain embodiments, up to 1, up to 2, up to 3, up to 4, up to 5, or up to 6 amino acids have been substituted, inserted, and/or deleted in the amino acid sequence GASTRAT (SEQ ID NO:10). In some embodiments, the anti-sortilin antibody comprises an HVR-L3 comprising an amino acid sequence containing substitutions (e.g., conservative substitutions, insertions, or deletions) relative to the amino acid sequence QQARLGPWT (SEQ ID NO: 11), but retains the ability to bind to sortilin. In certain embodiments, up to 1, up to 2, up to 3, up to 4, up to 5, or up to 6 amino acids have been substituted, inserted, and/or deleted in the amino acid sequence QQARLGPWT (SEQ ID NO: 11).

In some embodiments, anti-sortilin antibodies of the present disclosure comprise a light chain variable domain and a heavy chain variable domain, wherein the heavy chain variable domain comprises an HVR-H1 comprising the amino acid sequence YTFTKYYMS (SEQ ID NO:6), an HVR-H2 comprising the amino acid sequence IINPIGGSTSYAQKFQG (SEQ ID NO: 7), and an HVR-H3 comprising the amino acid sequence ARDPSGIALAGPASRGYQGMDV (SEQ ID NO:8), and the light chain variable domain comprises an HVR-L1 comprising the amino acid sequence RASQSVSSNLA (SEQ ID NO:9), an HVR-L2 comprising the amino acid sequence GASTRAT (SEQ ID NO: 10), and an HVR-L3 comprising the amino acid sequence QQARLGPWT (SEQ ID NO: 11).

In some embodiments, anti-sortilin antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the HVR-H1 amino acid sequence of antibody S-15-10-7; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the HVR-H2 amino acid sequence of antibody S-15-10-7; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the HVR-H3 amino acid sequence of antibody S-15-10-7; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the HVR-L1 amino acid sequence of antibody S-15-10-7; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the HVR-L2 amino acid sequence of antibody S-15-10-7; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the HVR-L3 amino acid sequence of antibody S-15-10-7.

In some embodiments, anti-sortilin antibodies of the present disclosure comprise a light chain variable domain and a heavy chain variable domain, wherein the heavy chain variable domain comprises an HVR-H1, HVR-H2, and HVR-H3, and wherein the light chain variable domain comprises an HVR-L1, HVR-L2, and HVR-L3. In some embodiments, the anti-sortilin antibody comprises an HVR-H1 comprising an amino acid sequence containing substitutions (e.g., conservative substitutions, insertions, or deletions) relative to the HVR-H1 amino acid sequence of antibody S-15-10-7, but retains the ability to bind to sortilin. In certain embodiments, up to 1, up to 2, up to 3, up to 4, up to 5, or up to 6 amino acids have been substituted, inserted, and/or deleted in the HVR-H1 amino acid sequence of antibody S-15-10-7. In some embodiments, the anti-sortilin antibody comprises an HVR-H2 comprising an amino acid sequence containing substitutions (e.g., conservative substitutions, insertions, or deletions) relative to the HVR-H2 amino acid sequence of antibody S-15-10-7, but retains the ability to bind to sortilin. In certain embodiments, up to 1, up to 2, up to 3, up to 4, up to 5, or up to 6 amino acids have been substituted, inserted, and/or deleted in the HVR-H2 amino acid sequence of antibody S-15-10-7. In some embodiments, the anti-sortilin antibody comprises an HVR-H3 comprising an amino acid sequence containing substitutions (e.g., conservative substitutions, insertions, or deletions) relative to the HVR-H3 amino acid sequence of antibody S-15-10-7, but retains the ability to bind to sortilin. In certain embodiments, up to 1, up to 2, up to 3, up to 4, up to 5, or up to 6 amino acids have been substituted, inserted, and/or deleted in the HVR-H3 amino acid sequence of antibody S-15-10-7. In some embodiments, the anti-sortilin antibody comprises an HVR-L1 comprising an amino acid sequence containing substitutions (e.g., conservative substitutions, insertions, or deletions) relative to the HVR-L1 amino acid sequence of antibody S-15-10-7, but retains the ability to bind to sortilin. In certain embodiments, up to 1, up to 2, up to 3, up to 4, up to 5, or up to 6 amino acids have been substituted, inserted, and/or deleted in the HVR-L1 amino acid sequence of antibody S-15-10-7. In some embodiments, the anti-sortilin antibody comprises an HVR-L2 comprising an amino acid sequence containing substitutions (e.g., conservative substitutions, insertions, or deletions) relative to the HVR-L2 amino acid sequence of antibody S-15-10-7, but retains the ability to bind to sortilin. In certain embodiments, up to 1, up to 2, up to 3, up to 4, up to 5, or up to 6 amino acids have been substituted, inserted, and/or deleted in the HVR-L2 amino acid sequence of antibody S-15-10-7. In some embodiments, the anti-sortilin antibody comprises an HVR-L3 comprising an amino acid sequence containing substitutions (e.g., conservative substitutions, insertions, or deletions) relative to the HVR-L3 amino acid sequence of antibody S-15-10-7, but retains the ability to bind to sortilin. In certain embodiments, up to 1, up to 2, up to 3, up to 4, up to 5, or up to 6 amino acids have been substituted, inserted, and/or deleted in the HVR-L3 amino acid sequence of antibody S-15-10-7.

In some embodiments, anti-sortilin antibodies of the present disclosure comprise a light chain variable domain and a heavy chain variable domain, wherein the heavy chain variable domain comprises an HVR-H1 comprising the HVR-H1 amino acid sequence of antibody S-15-10-7, an HVR-H2 comprising the HVR-H2 amino acid sequence of antibody S-15-10-7, and an HVR-H3 comprising the HVR-H3 amino acid sequence of antibody S-15-10-7, and the light chain variable domain comprises an HVR-L1 comprising the HVR-L1 amino acid sequence of antibody S-15-10-7, an HVR-L2 comprising the HVR-L2 amino acid sequence of antibody S-15-10-7, and an HVR-L3 comprising the HVR-L3 amino acid sequence of antibody S-15-10-7.

In some embodiments, anti-sortilin antibodies of the present disclosure comprise a heavy chain variable domain and a light chain variable domain, wherein the heavy chain variable domain comprises one, two, three or four framework regions (FR) selected from VH FR1, VH FR2, VH FR3, and VH FR4, wherein: the VH FR1 comprises an amino acid sequence of SEQ ID NO: 12, the VH FR2 comprises an amino acid sequence of SEQ ID NO: 13, the VH FR3 comprises an amino acid sequence of SEQ ID NO: 14, and the VH FR4 comprises an amino acid sequence of SEQ ID NO: 15; and/or the light chain variable domain comprises one, two, three or four framework regions selected from VL FR1, VL FR2, VL FR3, and VL FR4, wherein: the VL FR1 comprises an amino acid sequence of SEQ ID NO: 16, the VL FR2 comprises an amino acid sequence of SEQ ID NO: 17, the VL FR3 comprises an amino acid sequence of SEQ ID NO: 18, and the VL FR4 comprises an amino acid sequence of SEQ ID NO: 19.

In some embodiments, anti-sortilin antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 20; and/or wherein the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 21.

In some embodiments, anti-sortilin antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 20, wherein the heavy chain variable domain comprises an HVR-H1 comprising the amino acid sequence of SEQ ID NO: 6, an HVR-H2 comprising the amino acid sequence of SEQ ID NO: 7, and an HVR-H3 comprising the amino acid sequence of SEQ ID NO: 8; and/or wherein the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 21, wherein the light chain variable domain comprises an HVR-L1 comprising the amino acid sequence of SEQ ID NO: 9, an HVR-L2 comprising the amino acid sequence of SEQ ID NO: 10, and an HVR-L3 comprising the amino acid sequence of SEQ ID NO: 11.

In some embodiments, anti-sortilin antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 20, wherein the heavy chain variable domain comprises an HVR-H1 comprising the HVR-H1 amino acid sequence of antibody S-15-10-7, an HVR-H2 comprising the HVR-H2 amino acid sequence of antibody S-15-10-7, and an HVR-H3 comprising the HVR-H3 amino acid sequence of antibody S-15-10-7; and/or wherein the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 21, wherein the light chain variable domain comprises an HVR-L1 comprising the HVR-L1 amino acid sequence of antibody S-15-10-7, an HVR-L2 comprising the HVR-L2 amino acid sequence of antibody S-15-10-7, and an HVR-L3 comprising the HVR-L3 amino acid sequence of antibody S-15-10-7.

In some embodiments, anti-sortilin antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain variable domain amino acid sequence of antibody S-15-10-7; and/or wherein the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain variable domain amino acid sequence of antibody S-15-10-7.

In some embodiments, anti-sortilin antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the heavy chain variable domain amino acid sequence of antibody S-15-10-7, wherein the heavy chain variable domain comprises an HVR-H1 comprising the HVR-H1 amino acid sequence of antibody S-15-10-7, an HVR-H2 comprising the HVR-H2 amino acid sequence of antibody S-15-10-7, and an HVR-H3 comprising the HVR-H3 amino acid sequence of antibody S-15-10-7; and/or wherein the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the light chain variable domain amino acid sequence of antibody S-15-10-7, wherein the light chain variable domain comprises an HVR-L1 comprising the HVR-L1 amino acid sequence of antibody S-15-10-7, an HVR-L2 comprising the HVR-L2 amino acid sequence of antibody S-15-10-7, and an HVR-L3 comprising the HVR-L3 amino acid sequence of antibody S-15-10-7.

In some embodiments, an anti-sortilin antibody of the present disclosure comprises a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence of SEQ ID NO: 20 and contains substitutions (e.g., conservative substitutions, insertions, or deletions relative to SEQ ID NO: 20), but the anti-sortilin antibody comprising that sequence retains the ability to bind to sortilin. In certain embodiments, a total of between 1 and 10 amino acids have been substituted, inserted, and/or deleted in the amino acid sequence of SEQ ID NO: 20. In certain embodiments, a total of between 1 and 6 amino acids have been substituted, inserted, and/or deleted in the amino acid sequence of SEQ ID NO: 20. In certain embodiments, substitutions, insertions, or deletions occur in regions outside the HVRs (i.e., in the FR regions). In some embodiments, the substitutions, insertions, or deletions occur in one or more FR regions (i.e., in VH FR1, VH FR2, VH FR3, and/or VH FR4). In certain embodiments, substitutions, insertions, or deletions occur in one or more HVRs (i.e., in HVR-H1, HVR-H2, and/or HVR-H3). In some embodiments, the anti-sortilin antibody comprises a heavy chain variable domain comprising the amino acid sequence of SEQ ID NO: 20, including post-translational modifications of that sequence.

In some embodiments, an anti-sortilin antibody of the present disclosure comprises a light chain variable domain and a heavy chain variable domain, wherein the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence of SEQ ID NO: 21 and contains substitutions (e.g., conservative substitutions, insertions, or deletions relative to SEQ ID NO: 21), but the anti-sortilin antibody comprising that sequence retains the ability to bind to sortilin. In certain embodiments, a total of between 1 and 10 amino acids have been substituted, inserted, and/or deleted in the amino acid sequence of SEQ ID NO: 21. In certain embodiments, a total of between 1 and 6 amino acids have been substituted, inserted, and/or deleted in the amino acid sequence of SEQ ID NO: 21. In certain embodiments, substitutions, insertions, or deletions occur in regions outside the HVRs (i.e., in the FR regions). In some embodiments, the substitutions, insertions, or deletions occur in one or more FR regions (i.e., in VL FR1, VL FR2, VL FR3, and/or VL FR4). In certain embodiments, substitutions, insertions, or deletions occur in one or more HVRs (i.e., in HVR-L1, HVR-L2, and/or HVR-L3). In some embodiments, the anti-sortilin antibody comprises a light chain variable domain comprising the amino acid sequence of SEQ ID NO: 21, including post-translational modifications of that sequence.

In some embodiments, an anti-sortilin antibody of the present disclosure comprises a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the heavy chain variable domain amino acid sequence of antibody S-15-10-7 and contains substitutions (e.g., conservative substitutions, insertions, or deletions relative to the heavy chain variable domain amino acid sequence of antibody S-15-10-7), but the anti-sortilin antibody comprising that sequence retains the ability to bind to sortilin. In certain embodiments, a total of between 1 and 10 amino acids have been substituted, inserted, and/or deleted in the heavy chain variable domain amino acid sequence of antibody S-15-10-7. In certain embodiments, a total of between 1 and 6 amino acids have been substituted, inserted, and/or deleted in the heavy chain variable domain amino acid sequence of antibody S-15-10-7. In certain embodiments, substitutions, insertions, or deletions occur in regions outside the HVRs (i.e., in the FR regions). In some embodiments, the substitutions, insertions, or deletions occur in one or more FR regions (i.e., in VH FR1, VH FR2, VH FR3, and/or VH FR4). In certain embodiments, substitutions, insertions, or deletions occur in one or more HVRs (i.e., in HVR-H1, HVR-H2, and/or HVR-H3). In some embodiments, the anti-sortilin antibody comprises a heavy chain variable domain comprising the heavy chain variable domain amino acid sequence of antibody S-15-10-7, including post-translational modifications of that sequence.

In some embodiments, an anti-sortilin antibody of the present disclosure comprises a light chain variable domain and a heavy chain variable domain, wherein the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the light chain variable domain amino acid sequence of antibody S-15-10-7 and contains substitutions (e.g., conservative substitutions, insertions, or deletions relative to the light chain variable domain amino acid sequence of antibody S-15-10-7), but the anti-sortilin antibody comprising that sequence retains the ability to bind to sortilin. In certain embodiments, a total of between 1 and 10 amino acids have been substituted, inserted, and/or deleted in the light chain variable domain amino acid sequence of antibody S-15-10-7. In certain embodiments, a total of between 1 and 6 amino acids have been substituted, inserted, and/or deleted in the light chain variable domain amino acid sequence of antibody S-15-10-7. In certain embodiments, substitutions, insertions, or deletions occur in regions outside the HVRs (i.e., in the FR regions). In some embodiments, the substitutions, insertions, or deletions occur in one or more FR regions (i.e., in VL FR1, VL FR2, VL FR3, and/or VL FR4). In certain embodiments, substitutions, insertions, or deletions occur in one or more HVRs (i.e., in HVR-L1, HVR-L2, and/or HVR-L3). In some embodiments, the anti-sortilin antibody comprises a light chain variable domain comprising the light chain variable domain amino acid sequence of antibody S-15-10-7, including post-translational modifications of that sequence.

In some embodiments, an anti-sortilin antibody of the disclosure comprises a heavy chain variable domain comprising the amino acid sequence of SEQ ID NO: 20 and a light chain variable domain comprising the amino acid sequence of SEQ ID NO: 21.

In some embodiments, an anti-sortilin antibody of the disclosure comprises a heavy chain variable domain comprising the heavy chain variable domain amino acid sequence of antibody S-15-10-7 and a light chain variable domain comprising the light chain variable domain amino acid sequence of antibody S-15-10-7.

In some embodiments, an anti-sortilin antibody of the disclosure comprises a heavy chain comprising the amino acid sequence of SEQ ID NO: 31 or 32 and a light chain comprising the amino acid sequence of SEQ ID NO: 30. In some embodiments, an anti-sortilin antibody of the disclosure comprises a heavy chain comprising the amino acid sequence of SEQ ID NO: 31 and a light chain comprising the amino acid sequence of SEQ ID NO: 30. In some embodiments, an anti-sortilin antibody of the disclosure comprises a heavy chain comprising the amino acid sequence of SEQ ID NO: 32 and a light chain comprising the amino acid sequence of SEQ ID NO: 30.

In some embodiments, an anti-sortilin antibody of the present disclosure is anti-sortilin antibody S-15-10-7, e.g., as described in WO2016164637A1, which is hereby incorporated by reference in its entirety.

TABLE 1
Anti-Sortilin Antibody Sequences.

| SEQ ID NO | Sequence | Description |
|---|---|---|
| 6 | YTFTKYYMS | S-15-10-7 |
| | | HVR-H1 |
| 7 | IINPIGGSTSYAQKFQG | S-15-10-7 |
| | | HVR-H2 |
| 8 | ARDPSGIALAGPASRGYQGMDV | S-15-10-7 |
| | | HVR-H3 |
| 9 | RASQSVSSNLA | S-15-10-7 |
| | | HVR-L1 |
| 10 | GASTRAT | S-15-10-7 |
| | | HVR-L2 |
| 11 | QQARLGPWT | S-15-10-7 |
| | | HVR-L3 |
| 12 | QVQLVQSGAEVKKPGASVKVSCKASG | VH FR1 |
| 13 | WVRQAPGQGLEWMG | VH FR2 |
| 14 | RVTMTRDTSTSTVYMELSSLRSEDTAVYYC | VH FR3 |
| 15 | WGQGTTVTVSS | VH FR4 |
| 16 | EIVMTQSPATLSVSPGERATLSC | VL FR1 |
| 17 | WYQQKPGQAPRLLIY | VL FR2 |
| 18 | GIPARFSGSGSGTEFTLTISSLQSEDFAVYYC | VL FR3 |
| 19 | FGGGTKVEIK | VL FR4 |
| 20 | QVQLVQSGAEVKKPGASVKVSCKASGYTFTKYYMSWV | S-15-10-7 |
| | RQAPGQGLEWMGIINPIGGSTSYAQKFQGRVTMTRDTST | Heavy chain variable |
| | STVYMELSSLRSEDTAVYYCARDPSGIALAGPASRGYQG | (VH) domain |
| | MDVWGQGTTVTVSS | |
| 21 | EIVMTQSPATLSVSPGERATLSCRASQSVSSNLAWYQQKP | S-15-10-7 |
| | GQAPRLLIYGASTRATGIPARFSGSGSGTEFTLTISSLQSED | Light chain variable |
| | FAVYYCQQARLGPWTFGGGTKVEIK | (VL) domain |
| 30 | E I V M T Q S P A T L S V S P G E R A T | Full-length light |
| | L S C R A S Q S V S S N L A W Y Q Q K P | chain amino acid |
| | G Q A P R L L I Y G A S T R A T G I P A | sequence of antibody |
| | R F S G S G S G T E F T L T I S S L Q S | ALX (containing VL |
| | E D F A Y Y C Q Q A R L R G P W T F G G | of antibody S-15-10-7) |
| | G T K V E I K R T V A A P S V F I F P P | |
| | S D E Q L K S G T A S V V C L L N N F Y | |
| | P R E A K V Q W K V D N A L Q S G N S Q | |
| | E S V T E Q D S K D S T Y S L S S T L T | |
| | L S K A D K Y E K H V Y A C E V T H Q G | |
| | L S S P V T K S F N R G E C | |
| 31 | Q V Q L V Q S G A E V K K P G A S V K V | Full-length heavy |
| | S C K A S G Y T F T K Y Y M S W V R Q A | chain amino acid |
| | P G Q G L E W M G I I N P I G G S T S Y | sequence of antibody |
| | A Q K F Q G R V T M T R D T S T S T V Y | ALX with C-terminal |
| | M E L S S L R S E D T A V Y Y C A R D P | lysine (containing |
| | S G I A L A G P A S R G Y Q G M D V W G | VH of antibody S-15- |
| | Q G T T V T V S S A S T K G P S V F P L | 10-7) |
| | A P S S K S T S G G T A A L G C L V K D | |
| | Y F P E P V T V S W N S G A L T S G V H | |
| | T F P A V L Q S S G L Y S L S S V V T V | |
| | P S S S L G T Q T Y I C N V N H K P S N | |
| | T K V D K K V E P K S C D K T H T C P P | |
| | C P A A E A A G G P S V F L F P P K P K | |
| | D T L M I S R T P E V T C V V V D V S H | |
| | E D P E V K F N W Y V D G V E V H N A K | |
| | T K P R E E Q Y N S T Y R V V S V L T V | |
| | L H Q D W L N G K E Y K C K V S N K A L | |
| | P A S I E K T I S K A K G Q P R E P Q V | |
| | Y T L P P S R D E L T K N Q V S L T C L | |
| | V K G Y P S D I A V E W E S N Q G Q P E | |
| | N N Y K T T P P V L D S D G S F F L Y S | |
| | K L T V D K S R W Q Q G N V F S C S V M | |
| | H E A L H N H Y T Q K S L S L S P G K | |
| 32 | Q V Q L V Q S G A E V K K P G A S V K V | Full-length heavy |
| | S C K A S G Y T F T K Y Y M S W V R Q A | chain amino acid |
| | P G Q G L E W M G I I N P I G G S T S Y | sequence of antibody |
| | A Q K F Q G R V T M T R D T S T S T V Y | ALX without C- |
| | M E L S S L R S E D T A V Y Y C A R D P | terminal lysine |
| | S G I A L A G P A S R G Y Q G M D V W G | (containing VH of |
| | Q G T T V T V S S A S T K G P S V F P L | antibody S-15-10-7) |
| | A P S S K S T S G G T A A L G C L V K D | |
| | Y F P E P V T V S W N S G A L T S G V H | |
| | T F P A V L Q S S G L Y S L S S V V T V | |
| | P S S S L G T Q T Y I C N V N H K P S N | |
| | T K V D K K V E P K S C D K T H T C P P | |
| | C P A P E A A G G P S V F L F P P K P K | |
| | D T L M I S R T P E V T C V V V D V S H | |
| | E D P E V K F N W Y V D G V E V H N A K | |
| | T K P R E E Q Y N S T Y R V V S V L T V | |
| | L H Q D W L N G K E Y K C K V S N K A L | |
| | P A S I E K T I S K A K G Q P R E P Q V | |
| | Y T L P P S R D E L T K N Q V S L T C L | |
| | V K G F Y P S D I A V E W E S N G Q P E | |
| | N N Y K T T P P V L D S D G S F F L Y S | |
| | K L T V D K S R W Q Q G N V F S C S V M | |
| | H E A L H N H Y T Q K S L S L S P G | |

In some aspects, the present disclosure also provides antibodies that bind to a sortilin protein. In some embodiments, an antibody of the disclosure is any of the anti-sortilin antibodies described in the “Exemplary Anti-Sortilin Antibodies” section herein. In some embodiments, the antibody binds to human sortilin and/or to a mammalian sortilin protein. In some embodiments, the antibody comprises a heavy chain variable domain comprising the amino acid sequence of SEQ ID NO: 20 and a light chain variable domain comprising the amino acid sequence of SEQ ID NO: 21. In some embodiments, the antibody has a human IgG1 isotype. In some embodiments, the antibody comprises an Fc region comprising amino acid substitutions L234A, L235A, and P331S, wherein the numbering of the residue position is according to EU numbering. In some embodiments, the antibody comprises a light chain comprising the amino acid sequence of SEQ ID NO: 30, and a heavy chain comprising the amino acid sequence of SEQ ID NO: 31. In some embodiments, the antibody comprises a light chain comprising the amino acid sequence of SEQ ID NO: 30, and a heavy chain comprising the amino acid sequence of SEQ ID NO: 32.

### Anti-Sortilin Antibody Binding Regions

In some embodiments, anti-sortilin antibodies of the present disclosure bind to one or more amino acids within amino acid residues 237-247 of human sortilin, or amino acid residues on a sortilin protein corresponding to amino acid residues 237-247 of human sortilin. In some embodiments, anti-sortilin antibodies of the present disclosure bind to one or more amino acids within amino acid residues 237-247 of SEQ ID NO: 1, or amino acid residues on a protein corresponding to amino acid residues 237-247 of SEQ ID NO: 1.

In some embodiments, anti-sortilin antibodies of the present disclosure bind to one or more amino acids within amino acid residues 314-338 of human sortilin, or amino acid residues on a sortilin protein corresponding to amino acid residues 314-338 of human sortilin. In some embodiments, anti-sortilin antibodies of the present disclosure bind to one or more amino acids within amino acid residues 314-338 of SEQ ID NO: 1, or amino acid residues on a protein corresponding to amino acid residues 314-338 of SEQ ID NO: 1.

In some embodiments, anti-sortilin antibodies of the present disclosure bind to one or more amino acids within amino acid residues 237-247 and 314-338 of human sortilin, or amino acid residues on a sortilin protein corresponding to amino acid residues 237-247 and 314-338 of human sortilin. In some embodiments, anti-sortilin antibodies of the present disclosure bind to one or more amino acids within amino acid residues 237-247 and 314-338 of SEQ ID NO: 1, or amino acid residues on a protein corresponding to amino acid residues 237-247 and 314-338 of SEQ ID NO: 1.

In some embodiments, anti-sortilin antibodies of the present disclosure bind to one or more amino acids within the amino acid sequence NGLWVSKNFGG (SEQ ID NO: 4) of a sortilin protein, such as human sortilin. In some embodiments, anti-sortilin antibodies of the present disclosure bind to one or more amino acids within the amino acid sequence FASVMADKDTTRRIHVSTDQGDTWS (SEQ ID NO: 5) of a sortilin protein, such as human sortilin. In some embodiments, anti-sortilin antibodies of the present disclosure bind to one or more amino acids within the amino acid sequence NGLWVSKNFGG (SEQ ID NO: 4) and within the amino acid sequence FASVMADKDTTRRIHVSTDQGDTWS (SEQ ID NO: 5) of a sortilin protein, such as human sortilin.

In some embodiments, anti-sortilin antibodies of the present disclosure bind to one or more amino acids within the beta propeller domain of a sortilin protein, such as a human sortilin protein.

### Anti-Sortilin Antibody Binding Affinity

Anti-sortilin antibodies of the present disclosure may have micromolar, nanomolar, or picomolar affinities for the target antigen (e.g., human sortilin or mammalian sortilin).

In certain embodiments, the binding affinity of an anti-sortilin antibody of the present disclosure for target antigen (e.g., human sortilin or mammalian sortilin) is measured by the dissociation constant, K_{D}. Dissociation constants may be determined through any analytical technique known in the art, including biochemical or biophysical techniques such as fluorescent activated cell sorting (FACS), flow cytometry, enzyme-linked immunosorbent assay (ELISA), surface plasmon resonance (SPR), BioLayer interferometry (see, e.g., Octet System by ForteBio), meso scale discover assays (see, e.g., MSD-SET), isothermal titration calorimetry (ITC), differential scanning calorimetry (DSC), circular dichroism (CD), stopped-flow analysis, and colorimetric or fluorescent protein melting analyses; or a cell binding assay. In some embodiments, the dissociation constant (K_{D}) for sortilin (e.g., human sortilin or mammalian sortilin) is determined at a temperature of approximately 25° C. In some embodiments, the dissociation constant (K_{D}) for sortilin (e.g., human sortilin or mammalian sortilin) is determined at a temperature of approximately 4° C. In some embodiments, the dissociation constant (K_{D}) for sortilin (e.g., human sortilin or mammalian sortilin) is measured at 4° C. or room temperature utilizing, for example, FACS or BioLayer interferometry assay. In some embodiments, the dissociation constant (K_{D}) for sortilin (e.g., human sortilin or mammalian sortilin) is measured at 4° C. or room temperature utilizing, for example, a cell binding assay, ForteBio assay, or MSD-SET assay.

In some embodiments of any of the antibodies provided herein, the antibody has a dissociation constant (K_{D}) of <1 μM, <100 nM, <10 nM, <1 nM, <0.1 nM, <0.01 nM, or <0.001 nM (e.g., 10⁻⁸ M or less, e.g., from 10⁻⁸ M to 10⁻¹³ M, e.g., from 10⁻⁹ M to 10⁻¹³ M) for the target antigen (e.g., human sortilin or mammalian sortilin).

In certain embodiments, the K_{D} of an anti-sortilin antibody of the present disclosure for human sortilin and/or mammalian sortilin, e.g., mouse sortilin or cynomolgus monkey sortilin, is less than 100 nM, less than 90 nM, less than 80 nM, less than 70 nM, less than 60 nM, less than 50 nM, less than 40 nM, less than 30 nM, less than 20 nM, less than 10 nM, less than 9 nM, less than 8 nM, less than 7 nM, less than 6 nM, less than 5 nM, less than 4 nM, less than 3 nM, less than 2 nM, less than 1 nM, less than 0.5 nM, less than 0.1 nM, less than 0.09 nM, less than 0.08 nM, less than 0.07 nM, less than 0.06 nM, less than 0.05 nM, less than 0.04 nM, less than 0.03 nM, less than 0.02 nM, less than 0.01 nM, less than 0.009 nM, less than 0.008 nM, less than 0.007 nM, less than 0.006 nM, less than 0.005 nM, less than 0.004 nM, less than 0.003 nM, less than 0.002 nM, less than 0.001 nM, or less than 0.001 nM.

In some embodiments, an antibody of the disclosure binds with a dissociation constant (K_{D}) for human sortilin that ranges from about 0.005 nM to about 100 nM. In some embodiments, an antibody of the disclosure binds with a dissociation constant (K_{D}) for human sortilin that ranges from about 0.005 nM to about 1 nM, e.g., any of about 0.005 nM, about 0.006 nM, about 0.007 nM, about 0.008 nM, about 0.009 nM, about 0.01 nM, about 0.02 nM, about 0.03 nM, about 0.04 nM, about 0.05 nM, about 0.06 nM, about 0.07 nM, about 0.08 nM, about 0.09 nM, about 0.1 nM, about 0.2 nM, about 0.3 nM, about 0.4 nM, about 0.5 nM, about 0.6 nM, about 0.7 nM, about 0.8 nM, about 0.9 nM, or about 1 nM. In some embodiments, an antibody of the disclosure binds with a dissociation constant (K_{D}) for human sortilin that ranges from about 1 nM to about 10 nM, e.g., any of about 1 nM, about 1.2 nM, about 1.4 nM, about 1.6 nM, about 1.8 nM, about 2 nM, about 2.2 nM, about 2.4 nM, about 2.6 nM, about 2.8 nM, about 3 nM, about 3.2 nM, about 3.4 nM, about 3.6 nM, about 3.8 nM, about 4 nM, about 4.2 nM, about 4.4 nM, about 4.6 nM, about 4.8 nM, about 5 nM, about 5.2 nM, about 5.4 nM, about 5.6 nM, about 5.8 nM, about 6 nM, about 6.2 nM, about 6.4 nM, about 6.6 nM, about 6.8 nM, about 7 nM, about 7.2 nM, about 7.4 nM, about 7.6 nM, about 7.8 nM, about 8 nM, about 8.2 nM, about 8.4 nM, about 8.6 nM, about 8.8 nM, about 9 nM, about 9.2 nM, about 9.4 nM, about 9.6 nM, about 9.8 nM, or about 10 nM. In some embodiments, an antibody of the disclosure binds with a dissociation constant (K_{D}) for human sortilin of about 0.029 nM. In some embodiments, an antibody of the disclosure binds with a dissociation constant (K_{D}) for human sortilin of about 1.3 nM. In some embodiments, an antibody of the disclosure binds with a dissociation constant (K_{D}) for human sortilin of about 1.32 nM. In some embodiments, an antibody of the disclosure binds with a dissociation constant (K_{D}) for human sortilin of about 2.3 nM. In some embodiments, an antibody of the disclosure binds with a dissociation constant (K_{D}) for human sortilin of about 3.16 nM. In some embodiments, an antibody of the disclosure binds with a dissociation constant (K_{D}) for human sortilin of about 3.3 nM.

In some embodiments, an antibody of the disclosure binds with a dissociation constant (K_{D}) for mouse sortilin that ranges from about 0.005 nM to about 100 nM. In some embodiments, an antibody of the disclosure binds with a dissociation constant (K_{D}) for mouse sortilin that ranges from about 1 nM to about 10 nM, e.g., any of about 1 nM, about 1.2 nM, about 1.4 nM, about 1.6 nM, about 1.8 nM, about 2 nM, about 2.2 nM, about 2.4 nM, about 2.6 nM, about 2.8 nM, about 3 nM, about 3.2 nM, about 3.4 nM, about 3.6 nM, about 3.8 nM, about 4 nM, about 4.2 nM, about 4.4 nM, about 4.6 nM, about 4.8 nM, about 5 nM, about 5.2 nM, about 5.4 nM, about 5.6 nM, about 5.8 nM, about 6 nM, about 6.2 nM, about 6.4 nM, about 6.6 nM, about 6.8 nM, about 7 nM, about 7.2 nM, about 7.4 nM, about 7.6 nM, about 7.8 nM, about 8 nM, about 8.2 nM, about 8.4 nM, about 8.6 nM, about 8.8 nM, about 9 nM, about 9.2 nM, about 9.4 nM, about 9.6 nM, about 9.8 nM, or about 10 nM. In some embodiments, an antibody of the disclosure binds with a dissociation constant (K_{D}) for mouse sortilin of about 1.2 nM. In some embodiments, an antibody of the disclosure binds with a dissociation constant (K_{D}) for mouse sortilin of about 1.23 nM. In some embodiments, an antibody of the disclosure binds with a dissociation constant (K_{D}) for mouse sortilin of about 1.4 nM. In some embodiments, an antibody of the disclosure binds with a dissociation constant (K_{D}) for mouse sortilin of about 6.4 nM. In some embodiments, an antibody of the disclosure binds with a dissociation constant (K_{D}) for mouse sortilin of about 7.42 nM.

In some embodiments, the dissociation constant (K_{D}) of an anti-sortilin antibody of the disclosure for sortilin (e.g., human sortilin or mammalian sortilin) is determined using a ForteBio binding assay. In an exemplary ForteBio binding assay, affinity measurements are performed generally as previously described (Estep et al., MAbs. 2013 March-April; 5(2):270-8). Briefly, ForteBio affinity measurements are performed by loading anti-sortilin antibodies on-line onto AHQ sensors. Sensors are equilibrated off-line in assay buffer for 30 minutes and then monitored on-line for 60 seconds for baseline establishment. For avid binding measurement, sensors with loaded anti-sortilin antibodies are exposed to 100 nM antigen (e.g., human sortilin or mammalian sortilin, such as a human or mouse sortilin Fc fusion protein), afterwards they are transferred to assay buffer for off-rate measurements. Additional avid binding may be determined by loading biotinylated sortilin protein, e.g., human or mouse sortilin monomer, on SA sensors and exposing to about 100 nM anti-sortilin antibody in solution. Monovalent binding measurements may be obtained by loading sortilin, such as a human or mouse sortilin Fc fusion protein, to AHQ sensors followed by exposure to about 100 nM of anti-sortilin antibody Fab. Additional monovalent measurements may be made by loading biotinylated sortilin protein, e.g., human or mouse sortilin monomer, to SA sensors followed by exposure to about 100 nM anti-sortilin antibody Fab in solution. Kinetics data may be fit using a 1:1 binding, e.g., using a model in the data analysis software provided by ForteBio.

In some embodiments, an antibody of the disclosure binds with a dissociation constant (K_{D}) for human sortilin of between about 0.5 nM and about 2 nM, e.g., as measured using a ForteBio assay, e.g. as described herein. In some embodiments, an antibody of the disclosure binds with a dissociation constant (K_{D}) for human sortilin of between about 2 nM and about 4 nM, e.g., as measured using a ForteBio assay, e.g. as described herein. In some embodiments, an antibody of the disclosure binds with a dissociation constant (K_{D}) for human sortilin of about 0.9 nM, about 0.96 nM, about 0.98 nM, about lnM, about 1.18 nM, about 1.2 nM, or about 1.24 nM, e.g., as measured using a ForteBio assay, e.g. as described herein. In some embodiments, an antibody of the disclosure binds with a dissociation constant (K_{D}) for human sortilin of about 2.3 nM or about 3.3 nM, e.g., as measured using a ForteBio assay, e.g. as described herein. In some embodiments, an antibody of the disclosure binds with a dissociation constant (K_{D}) for mouse sortilin of between about 0.5 nM and about 2 nM, e.g., as measured using a ForteBio assay, e.g. as described herein. In some embodiments, an antibody of the disclosure binds with a dissociation constant (K_{D}) for mouse sortilin of between about lnM and about 8 nM, e.g., as measured using a ForteBio assay, e.g. as described herein. In some embodiments, an antibody of the disclosure binds with a dissociation constant (K_{D}) for mouse sortilin of about 0.6 nM, about 0.68 nM, about 0.7 nM, about 0.76 nM, about 0.77 nM, about 0.8 nM, about 0.9 nM, about 1 nM, about 1.11 nM, or about 1.2 nM, e.g., as measured using a ForteBio assay, e.g. as described herein. In some embodiments, an antibody of the disclosure binds with a dissociation constant (K_{D}) for mouse sortilin of about 1.4 nM or about 6.4 nM, e.g., as measured using a ForteBio assay, e.g. as described herein.

In some embodiments, the dissociation constant (K_{D}) of an anti-sortilin antibody of the disclosure for sortilin (e.g., human sortilin or mammalian sortilin) is determined using a meso scale discover assay (see, e.g., MSD-SET). In an exemplary MSD-SET assay, equilibrium affinity measurements are performed as previously described (Estep et al, (2013) MAbs 5(2):270-8). Solution equilibrium titrations (SET) are performed in PBS+0.1% IgG-Free BSA (PBSF) with antigen (e.g., human sortilin or mammalian sortilin, such as biotinylated human sortilin or mouse sortilin) held constant at 50 μM and incubated with 3- to 5-fold serial dilutions of anti-sortilin antibody starting at 10 nM. Anti-sortilin antibodies (20 nM in PBS) are coated onto standard bind MSD-ECL plates overnight at 4° C. or at room temperature for 30 minutes. Plates are then blocked for 30 minutes with shaking at 700 rpm, followed by three washes with wash buffer (PBSF+0.05% Tween 20). SET samples are applied and incubated on the plates for 150 seconds with shaking at 700 rpm followed by one wash. Antigen (e.g., human sortilin or mammalian sortilin, such as biotinylated human sortilin or mouse sortilin) captured on a plate is detected with 250 ng/mL sulfotag-labeled streptavidin in PBSF by incubation on the plate for 3 minutes. The plates are washed three times with wash buffer and then read on the MSD Sector Imager 2400 instrument using 1× Read Buffer T with surfactant. The percent free antigen is plotted as a function of titrated antibody in Prism and fit to a quadratic equation to extract the K_{D}.

In some embodiments, an antibody of the disclosure binds with a dissociation constant (K_{D}) for human sortilin of between about 0.02 nM and about 0.3 nM, e.g., as measured using a MSD-SET assay, e.g. as described herein. In some embodiments, an antibody of the disclosure binds with a dissociation constant (K_{D}) for human sortilin of between about 1 nM and about 5 nM, e.g., as measured using a MSD-SET assay, e.g. as described herein. In some embodiments, an antibody of the disclosure binds with a dissociation constant (K_{D}) for human sortilin of about 0.023 nM, about 0.032 nM, about 0.076 nM, about 0.19 nM, about 0.23 nM, or about 0.26 nM, e.g., as measured using a MSD-SET assay, e.g. as described herein. In some embodiments, an antibody of the disclosure binds with a dissociation constant (K_{D}) for human sortilin of about 2.3 nM or about 3.3 nM, e.g., as measured using a MSD-SET assay, e.g. as described herein. In some embodiments, an antibody of the disclosure binds with a dissociation constant (K_{D}) for mouse sortilin of between about 0.05 nM and about 0.2 nM, e.g., as measured using a MSD-SET assay, e.g. as described herein. In some embodiments, an antibody of the disclosure binds with a dissociation constant (K_{D}) for mouse sortilin of between about 1 nM and about 8 nM, e.g., as measured using a MSD-SET assay, e.g. as described herein. In some embodiments, an antibody of the disclosure binds with a dissociation constant (K_{D}) for mouse sortilin of about 0.07 nM, about 0.1 nM, or about 0.11 nM, e.g., as measured using a MSD-SET assay, e.g. as described herein. In some embodiments, an antibody of the disclosure binds with a dissociation constant (K_{D}) for mouse sortilin of about 1.4 nM, or about 6.4 nM e.g., as measured using a MSD-SET assay, e.g. as described herein.

In some embodiments, the dissociation constant (K_{D}) of an anti-sortilin antibody of the disclosure for sortilin (e.g., human sortilin or mammalian sortilin) is determined using a cell-based assay, such as a cell binding assay. In an exemplary cell binding assay, cell binding affinity measurements are performed at 4° C. using cells expressing human or mouse sortilin, such as HEK293T cells transiently transfected with mouse sortilin or stably expressing human sortilin. Cells are harvested, washed in PBS and incubated with an amount of antibody close to the K_{D} of the antibody. Antibodies are diluted in FACS buffer (PBS+2% FBS+0.01% NaAzide). After incubation on ice for 1 hour, cells are washed three times in FACS buffer and incubated with anti-human PE conjugated secondary antibody (BD Biosciences, 1:100 dilution) for 30 minutes on ice. Then the cells are washed twice in 200 μl FACS buffer, and subsequently analyzed on a FACS Canto or iQE FACS screening instrument (Intellicyt Corp). Binding to human and/or mouse sortilin is measured as the median fluorescent intensity (MFI) of PE. For determination of apparent affinity to cell-expressed sortilin, antibodies are added to cells in a titration, e.g., from 0.16-40 nM for human sortilin and 0.39-50 nM for mouse sortilin, and their binding K_{D}'s are determined by non-linear curve fitting (e.g., using modified OneSiteTotal, Graph Pad Prism).

In some embodiments, an antibody of the disclosure binds with a dissociation constant (K_{D}) for human sortilin expressed on a cell of between about 1 nM and about 10 nM (e.g., any of about 1 nM, about 2 nM, about 3 nM, about 4 nM, about 5 nM, about 6 nM, about 7 nM, about 8 nM, about 9 nM, or about 10 nM), e.g., as measured using a cell binding assay, e.g. as described herein. In some embodiments, an antibody of the disclosure binds with a dissociation constant (K_{D}) for human sortilin expressed on a cell of about 2.17 nM, e.g., as measured using a cell binding assay, e.g. as described herein. In some embodiments, an antibody of the disclosure binds with a dissociation constant (K_{D}) for human sortilin expressed on a cell of about 3.16 nM, e.g., as measured using a cell binding assay, e.g. as described herein. In some embodiments, an antibody of the disclosure binds with a dissociation constant (K_{D}) for mouse sortilin expressed on a cell of between about 1 nM and about 24 nM (e.g., any of about 1 nM, about 2 nM, about 3 nM, about 4 nM, about 5 nM, about 6 nM, about 7 nM, about 8 nM, about 9 nM, about 10 nM, about 11 nM, about 12 nM, about 13 nM, about 14 nM, about 15 nM, about 16 nM, about 17 nM, about 18 nM, about 19 nM, about 20 nM, about 21 nM, about 22 nM, about 23 nM, or about 24 nM), e.g., as measured using a cell binding assay, e.g. as described herein. In some embodiments, an antibody of the disclosure binds with a dissociation constant (K_{D}) for mouse sortilin expressed on a cell of about 7.42 nM, e.g., as measured using a cell binding assay, e.g. as described herein.

In some embodiments, the K_{D} of an anti-sortilin antibody of the disclosure for sortilin is determined using a monovalent antibody (e.g., a Fab) or a full-length antibody in a monovalent form, e.g., as described herein. In some embodiments, the K_{D} is determined using a bivalent antibody and monomeric recombinant sortilin protein, e.g., as described herein.

### Anti-Sortilin Antibody Activities

In certain aspects, anti-sortilin antibodies of the disclosure decrease cellular levels of sortilin (e.g., cell surface levels of sortilin, intracellular levels of sortilin, and/or total levels of sortilin); increase progranulin levels (e.g., extracellular levels of progranulin and/or cellular levels of progranulin); and/or inhibit the interaction (e.g., binding) between progranulin and sortilin. In certain aspects, anti-sortilin antibodies of the disclosure increase extracellular levels of progranulin and decrease cellular levels of sortilin. In some embodiments, the decrease in cellular levels of sortilin is a decrease in cell surface levels of sortilin, a decrease in intracellular levels of sortilin, a decrease in total levels of sortilin, or any combination thereof. In some embodiments, an anti-sortilin antibody of the disclosure (a) induces sortilin degradation, sortilin cleavage, sortilin internalization, sortilin down regulation, or any combination thereof; (b) inhibits interaction between sortilin and one or more proteins by: reducing the effective levels of sortilin available for interacting with the one or more proteins, inducing degradation of sortilin, or both; (c) inhibits interaction between sortilin and progranulin; (d) inhibits interaction between sortilin and pro-nerve growth factor (pro-NGF); (e) binds specifically to human sortilin; (f) binds specifically to human sortilin and mouse sortilin or binds specifically to human sortilin, mouse sortilin, and cynomolgus monkey sortilin; (g) induces one or more progranulin activities; (h) reduces endosomal internalization of progranulin, or fragments thereof; (i) increases the effective concentration of progranulin; j) inhibits the interaction between sortilin expressed on a cell and progranulin; (k) decreases expression of one or more pro-inflammatory mediators; and any combination thereof.

As contemplated herein, anti-sortilin antibodies of the present disclosure may also inhibit interaction (e.g., binding) of sortilin with one or more proteins including, without limitation, neurotrophins, such as pro-neurotrophins, pro-neurotrophin-3, neurotrophin-3, pro-neurotrophin-4/5, neurotrophin-4/5, pro-nerve growth factor (Pro-NGF), nerve growth factor (NGF), pro-brain-derived neurotrophic factor (Pro-BDNF), brain-derived neurotrophic factor (BDNF), and neurotrophin-4 (NT-4), neurotensin, low affinity nerve growth factor (NGF) receptor (p75), lipoprotein lipase (LpL), apolipoprotein AV (APOA5), apolipoprotein E (APOE), such as APOE2, APOE3, and APOE4, amyloid precursor protein (APP), A beta peptide, PCSK9, p75NTR, and receptor associated protein (RAP).

Anti-sortilin antibodies of the present disclosure may also decrease expression of one or more pro-inflammatory mediators including, without limitation, cytokines, such as type I and II interferons, IL-6, IL12p70, IL12p40, IL-1β, TNF-α, IL-8, CRP, IL-20 family members, IL-33, LIF, OSM, CNTF, GM-CSF, IL-11, IL-12, IL-17, IL-18, CRP, and chemokines, such as CXCL1, CCL2, CCL3, CCL4, and CCL5.

Inhibiting the Interaction Between Sortilin and Progranulin

Sortilin has been shown to interact (e.g., bind to) directly with progranulin and mediate the degradation of progranulin (e.g., Zheng, Y et al., (2011) PLoS ONE 6(6): e21023). In some embodiments, antibodies of the present disclosure decrease, block, or inhibit the interaction between sortilin and progranulin.

In some embodiments, an anti-sortilin antibody of the disclosure inhibits the interaction (e.g., binding) between sortilin and progranulin if it decreases progranulin binding to sortilin by 20% or more at saturating antibody concentrations, assessed using any method known in the art, such as any in vitro assay or cell-based assay described herein or known in the art. In some embodiments, an anti-sortilin antibody of the disclosure inhibits the interaction (e.g., binding) between sortilin and progranulin if it decreases progranulin binding to sortilin relative to a control antibody at saturating antibody concentrations, assessed using any in vitro assay or cell-based assay described herein or known in the art.

In some embodiments, an anti-sortilin antibody of the disclosure decreases binding of progranulin to sortilin by at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, or at least about 50%. In some embodiments, an anti-sortilin antibody of the disclosure decreases binding of progranulin to sortilin by at least about 50%, at least about 55%, at least about 60%, at least about 65%, at least about 70%, at least about 75%, at least about 80%, at least about 85%, at least about 90%, at least about 95%, at least about 99%, or 100%. In some embodiments, an anti-sortilin antibody of the disclosure decreases binding of human progranulin to human sortilin by between about 20% and about 50%, e.g., any of about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%. In some embodiments, an anti-sortilin antibody of the disclosure decreases binding of human progranulin to human sortilin by about 21%. In some embodiments, an anti-sortilin antibody of the disclosure decreases binding of human progranulin to human sortilin by about 36%. In some embodiments, an anti-sortilin antibody of the disclosure decreases binding of mouse progranulin to mouse sortilin by between about 20% and about 50%, e.g., any of about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%. In some embodiments, an anti-sortilin antibody of the disclosure decreases binding of mouse progranulin to mouse sortilin by about 21%. In some embodiments, an anti-sortilin antibody of the disclosure decreases binding of mouse progranulin to mouse sortilin by about 24%. In some embodiments, the binding of progranulin to sortilin, e.g., binding of human progranulin to human sortilin or mouse progranulin to mouse sortilin, may be assessed using any method known in the art, such as any in vitro assay or cell-based assay described herein or known in the art.

In some embodiments, the interaction, e.g., binding, between sortilin and progranulin may be assessed using a cell-based assay. In an exemplary cell-based assay, recombinant human or mouse progranulin (Adipogen) is biotinylated with an EZ-Link Micro NHS-PEG4 kit from ThermoScientific/Pierce according to the manufacturer's instructions. Cells that express human or mouse sortilin may be generated as is known in the art. Sortilin-expressing cells or control cells are harvested and washed in PBS. Biotinylated human or mouse progranulin is added in PBS+2% FBS with or without anti-sortilin antibodies (10 μg/ml), e.g., an anti-sortilin antibody of the disclosure, or control isotype antibodies, and incubated on ice for 2 hours. After washing cells 3 times in PBS+2% FBS, cells are incubated in Streptavidin-APC (BD Biosciences, 1:100) on ice for 30 minutes. Then cells are washed again, resuspended in PBS+2% FBS and analyzed by flow cytometry, e.g., on a FACSCanto™ flow cytometer (BD Biosciences, Mississauga, ON). Progranulin binding is measured as the median fluorescence intensity of APC of the sortilin expressing cell population. In some embodiments of the cell-based assay described herein, human or mouse biotinylated progranulin is used at increasing concentrations of between 0 nM and about 100 nM. In some embodiments of the cell-based assay described herein, human or mouse sortilin-expressing cells are incubated with 15 nM biotinylated human or mouse progranulin together with 67 nM of anti-sortilin antibodies, e.g., anti-sortilin antibodies of the disclosure.

In some embodiments, an anti-sortilin antibody of the disclosure decreases binding of human progranulin to human sortilin by between about 20% and about 50%, e.g., any of about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%, assessed using a cell-based assay, such as a cell-based assay described herein. In some embodiments, an anti-sortilin antibody of the disclosure decreases binding of human progranulin to human sortilin by about 21%, assessed using a cell-based assay, such as a cell-based assay described herein. In some embodiments, an anti-sortilin antibody of the disclosure decreases binding of human progranulin to human sortilin by about 36%, assessed using a cell-based assay, such as a cell-based assay described herein. In some embodiments, an anti-sortilin antibody of the disclosure decreases binding of mouse progranulin to mouse sortilin by between about 20% and about 50%, e.g., any of about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%, assessed using a cell-based assay, such as a cell-based assay described herein. In some embodiments, an anti-sortilin antibody of the disclosure decreases binding of mouse progranulin to mouse sortilin by about 21%, assessed using a cell-based assay, such as a cell-based assay described herein. In some embodiments, an anti-sortilin antibody of the disclosure decreases binding of mouse progranulin to mouse sortilin by about 24%, assessed using a cell-based assay, such as a cell-based assay described herein.

In some embodiments, the interaction between sortilin and progranulin may be assessed using an in vitro assay. For example, the interaction between sortilin and progranulin may be assessed using ForteBio and surface plasmon resonance analysis (e.g., Skeldal, S et al., (2012) J Biol Chem., 287:43798; and Andersen, O S et al., (2010) THE JOURNAL OF BIOLOGICAL CHEMISTRY, 285, 12210-12222). In an exemplary assay, the interaction between sortilin and progranulin is characterized using surface plasmon resonance (SPR) analysis. Determination of direct binding of human or mouse progranulin to immobilized sortilin, as well as binding of human or mouse sortilin to immobilized progranulin, in the presence or absence of an anti-sortilin antibody of the disclosure may be performed on a Biacore2000 instrument (Biacore, Sweden) using CaHBS as standard running buffer (10 mM HEPES, pH 7.4, 140 mM NaCl, 2 mM CaCl₂, 1 mM EGTA, and 0.005% Tween 20). A biosensor chip from Biacore (CM5, catalog no. BR-1000-14) may be activated using the NHS/EDC, e.g., method as described by the supplier, followed by coating with sortilin to a protein density of 79 fmol/mm² and used for affinity measurements of native progranulin protein. Regeneration of the flow cell after each cycle of ligand binding experiment is done by two 10-g1 pulses of regeneration buffer (10 mM glycine-HCl, pH 4.0, 500 mM NaCl, 20 mM EDTA, and 0.005% Tween 20) and a single injection of 0.001% SDS. Fitting of sensorgrams for affinity estimations may be done using BIAevaluation version 3.1. Following similar protocols, immobilization of His-progranulin may also be done on a CM5 biosensor chip using the NHS/EDC coupling kit according to the manufacturer's instructions (Biacore, Sweden), giving similar surface densities of immobilized protein (˜300 fmol/mm²). A biosensor chip with immobilized progranulin may also be used to examine the binding of sortilin in the absence or presence of an anti-sortilin antibody of the disclosure.

In some embodiments, the interaction between sortilin and progranulin may be assessed using an immunoassay such as an ELISA assay. In an exemplary assay, human or mouse sortilin (R&D Systems) is immobilized overnight on an ELISA plate (2 μg/ml in PBS). The plates are washed in wash buffer (PBS+0.05% TWEEN20) and blocked for one hour at 37° C. with binding buffer (PBS+1% BSA). Recombinant human or mouse progranulin (Adipogen) is biotinylated with an EZ-Link Micro NHS-PEG4 kit from ThermoScientific/Pierce. Biotinylated progranulin is added at various concentrations to immobilized sortilin and incubated at room temperature for 30 minutes. Plates are washed thrice in wash buffer and incubated with Streptavidin-HRP (1:200 in binding buffer, R&D Systems) for 20 minutes. Plates are washed thrice again and incubated with TMB substrate solution until color develops. The reaction is stopped by adding 50 ul of 2N sulfuric acid and color is quantified using a Biotek Synergy H1 plate reader. Data is analyzed and fitted in Prism.

In some embodiments, inhibition of the interaction between progranulin and sortilin by an anti-sortilin antibody of the disclosure is assessed using a competition assay, such as an in vitro competition assay. In an exemplary competition assay, a Forte Bio Octet Red384 system (Pall Forte Bio Corporation, Menlo Park, CA) using a standard sandwich format binning is used. Control anti-target IgG is loaded onto AHQ sensors and unoccupied Fc-binding sites on the sensor are blocked with a non-relevant IgG1 antibody. The sensors are then exposed to 100 nM target antigen followed by a second anti-target antibody. Data processing is performed using ForteBio's Data Analysis Software 7.0. Additional binding by the second antibody after antigen association indicates an unoccupied epitope (non-competitor), while no binding indicates epitope blocking (competitor).

In some embodiments, anti-sortilin antibodies of the present disclosure inhibit or reduce interaction (e.g., binding) between sortilin and progranulin by reducing sortilin levels or expression (e.g., as described herein).

In some embodiments, inhibition of the interaction (e.g., binding) between sortilin and progranulin by an antibody of the disclosure results in an increase in the level of progranulin, e.g., as described below. In some embodiments, inhibition of the interaction (e.g., binding) between sortilin and progranulin by an antibody of the disclosure results in an increase in the level of extracellular progranulin, e.g., as described below.

### Decreasing Sortilin Levels

In some embodiments, anti-sortilin antibodies of the present disclosure decrease cellular levels of sortilin. In some embodiments, anti-sortilin antibodies of the present disclosure decrease cellular levels of sortilin by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%, e.g., assessed using any in vitro, cell-based, or in vivo method known in the art or described herein. In some embodiments, the decrease in cellular levels of sortilin is a decrease in cell surface levels of sortilin, a decrease in intracellular levels of sortilin, a decrease in total levels of sortilin, or any combination thereof.

In some embodiments, anti-sortilin antibodies of the present disclosure decrease cell surface levels of sortilin. Cell surface levels of sortilin may be measured using any method known in the art, such as an in vitro cell-based assay or a suitable in vivo method. In some embodiments, an anti-sortilin antibody of the disclosure decreases cell surface levels of sortilin if it induces a decrease of 20% or more in cell surface levels of sortilin, e.g., as measured by any in vitro cell-based assays or suitable in vivo method described herein or known in the art. In some embodiments, an anti-sortilin antibody of the disclosure decreases cell surface levels of sortilin if it induces a decrease, at saturating antibody concentrations and/or relative to a control antibody, in cell surface levels of sortilin as measured by any in vitro cell-based assays or suitable in vivo method described herein or known in the art.

In an exemplary cell-based assay, cells that express sortilin, such as human U-251 cells or murine Neuro-2A (N2A) cells, are incubated with 50 nM anti-sortilin antibodies for 72 hours. Cells are then harvested with Trypsin, washed in PBS and labeled with another primary anti-sortilin antibody (e.g., at a concentration of about 5 μg/ml). After cells are incubated with primary antibody for one hour on ice, cells are washed three times in PBS+2% FBS and then incubated with secondary antibody, such as 5 μg/ml anti-human PE secondary antibody (Southern Biotech) or another suitable secondary antibody. Cells are then washed again, and cell surface sortilin levels are quantified using flow cytometry, e.g., using a FACSCanto™ system, and measured as median fluorescence intensity of PE.

In some embodiments, an antibody of the disclosure induces a decrease of at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% in the cell surface level of sortilin, e.g., assessed using an in vitro cell-based assay, e.g., as described herein. In some embodiments, an antibody of the disclosure induces a decrease of at least about 51.6% in the cell surface level of sortilin, e.g., assessed using an in vitro cell-based assay, e.g., as described herein. In some embodiments, an antibody of the disclosure induces a decrease of at least about 87.82% in the cell surface level of sortilin, e.g., assessed using an in vitro cell-based assay, e.g., as described herein. In some embodiments, an antibody of the disclosure induces a decrease of at least about 89% in the cell surface level of sortilin, e.g., assessed using an in vitro cell-based assay, e.g., as described herein using a concentration of anti-sortilin antibody of about 0.63 nM.

In some embodiments, cell surface levels of sortilin may be assessed in vivo, e.g., in macrophages, dendritic cells, and/or white blood cells obtained from an individual treated with an anti-sortilin antibody of the disclosure. In some cases, cell surface levels of human sortilin may be assessed in vivo, e.g., in macrophages, dendritic cells, and/or white blood cells obtained from a human treated with an anti-sortilin antibody of the disclosure. In some cases, cell surface levels of mouse sortilin may be assessed in vivo, e.g., in macrophages, dendritic cells, and/or white blood cells obtained from a mouse treated with an anti-sortilin antibody of the disclosure. In some cases, cell surface levels of cynomolgus monkey sortilin may be assessed in vivo, e.g., in macrophages, dendritic cells, and/or white blood cells obtained from a cynomolgus monkey treated with an anti-sortilin antibody of the disclosure. In some embodiments, an anti-sortilin antibody of the disclosure induces a decrease of at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% in the cell surface level of sortilin, e.g., as measured by any in vivo method described herein or known in the art.

In some embodiments, anti-sortilin antibodies of the present disclosure decrease intracellular levels of sortilin. Intracellular levels of sortilin may be measured using any method known in the art, such as an in vitro cell-based assay or a suitable in vivo method. In some embodiments, an anti-sortilin antibody of the disclosure decreases intracellular levels of sortilin if it induces a decrease of 20% or more in intracellular levels of sortilin as measured by any in vitro cell-based assays or suitable in vivo method described herein or known in the art. In certain embodiments, an anti-sortilin antibody of the disclosure decreases intracellular levels of sortilin if it induces a decrease at saturating antibody concentrations and/or relative to a control antibody in intracellular levels of sortilin as measured by any in vitro cell-based assays or suitable in vivo method described herein or known in the art. For example, intracellular levels of sortilin may be assessed by an in vitro cell based assay using cells that express sortilin, e.g., human U251 cells expressing human sortilin, or mouse Neuro2A cells expressing mouse sortilin. In some cases, intracellular levels of sortilin may be assessed by an in vitro cell based assay, e.g., using macrophages, dendritic cells, and/or white blood cells. In some cases, intracellular levels of human sortilin may be assessed by an in vitro cell based assay, e.g., using human macrophages, human dendritic cells, and/or human white blood cells. In some cases, intracellular levels of sortilin may be assessed in vivo, e.g., in macrophages, dendritic cells, and/or white blood cells obtained from an individual treated with an anti-sortilin antibody of the disclosure. In some cases, intracellular levels of human sortilin may be assessed in vivo e.g., in macrophages, dendritic cells, and/or white blood cells obtained from a human treated with an anti-sortilin antibody of the disclosure. In some cases, intracellular levels of mouse sortilin may be assessed in vivo e.g., in macrophages, dendritic cells, and/or white blood cells obtained from a mouse treated with an anti-sortilin antibody of the disclosure. In some cases, intracellular levels of cynomolgus monkey sortilin may be assessed in vivo e.g., in macrophages, dendritic cells, and/or white blood cells obtained from a cynomolgus monkey treated with an anti-sortilin antibody of the disclosure. In some embodiments, an antibody of the disclosure induces a decrease of at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% in the intracellular level of sortilin, e.g., as measured by any in vitro or in vivo method described herein or known in the art. In some embodiments, an antibody of the disclosure induces a decrease of at least about 51.6% in the intracellular level of sortilin, e.g., as measured by an in vitro cell-based assay described herein or known in the art. In some embodiments, an antibody of the disclosure induces a decrease of at least about 89% in the intracellular level of sortilin, e.g., as measured by an in vitro cell-based assay described herein or known in the art.

In some embodiments, anti-sortilin antibodies of the present disclosure decrease total levels of sortilin. Total levels of sortilin may be measured using any method known in the art, such as an in vitro cell-based assay or a suitable in vivo method. In some embodiments, an anti-sortilin antibody of the disclosure decreases total levels of sortilin if it induces a decrease of 20% or more in total levels of sortilin as measured by any in vitro cell-based assay or suitable in vivo method described herein or known in the art. In certain embodiments, an anti-sortilin antibody of the disclosure decreases total levels of sortilin if it induces a decrease at saturating antibody concentrations and/or relative to a control antibody in total levels of sortilin as measured by any in vitro cell-based assay or suitable in vivo method described herein or known in the art. For example, total levels of sortilin may be assessed by an in vitro cell based assay using cells that express sortilin, e.g., human U251 cells expressing human sortilin or mouse Neuro2A cells expressing mouse sortilin. In some cases, total levels of sortilin may be assessed by an in vitro cell based assay, e.g., using macrophages, dendritic cells, and/or white blood cells. In some cases, total levels of human sortilin may be assessed in vivo e.g., in macrophages, dendritic cells, and/or white blood cells obtained from a human treated with an anti-sortilin antibody of the disclosure. In some cases, total levels of mouse sortilin may be assessed in vivo e.g., in macrophages, dendritic cells, and/or white blood cells obtained from a mouse treated with an anti-sortilin antibody of the disclosure. In some cases, total levels of cynomolgus monkey sortilin may be assessed in vivo e.g., in macrophages, dendritic cells, and/or white blood cells obtained from a cynomolgus monkey treated with an anti-sortilin antibody of the disclosure. In some embodiments, an antibody of the disclosure induces a decrease of at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% in the total level of sortilin, e.g., as measured by any in vitro or in vivo method described herein or known in the art. In some embodiments, an antibody of the disclosure induces a decrease of at least about 51.6% in the total level of sortilin, e.g., as measured by an in vitro cell-based assay described herein or known in the art. In some embodiments, an antibody of the disclosure induces a decrease of at least about 89% in the total level of sortilin, e.g., as measured by an in vitro cell-based assay described herein or known in the art.

In some embodiments, total levels of sortilin may be assessed by an in vitro cell based assay. In an exemplary assay, white blood cells, such as primary monocytes, e.g., human primary monocytes, are isolated from blood, e.g., from heparinized human blood (Blood Centers of the Pacific) using RosetteSep Human Monocyte Enrichment Cocktail (STEMCELL Technologies), according to the manufacturer's protocol. Monocytes are seeded in RPMI (Invitrogen) containing 10% Fetal Calf Serum (Hyclone) and either 50 μg/ml M-CSF (Peprotech) to induce differentiation to macrophages or 100 μg/ml IL-4+100 μg/ml GM-CSF (Peprotech) to induce differentiation to dendritic cells. After 5 days, cells are harvested. For macrophages, only cells attached to the plate are harvested using a cell scraper. For dendritic cells, cells in suspension are collected. After washes in PBS, cells are plated at 0.4 Mi/well in 12-well plates. Fifty nanomolar control antibodies or Fabs, or full length anti-sortilin antibodies (IgG) or anti-sortilin antibody Fabs, e.g., anti-sortilin antibodies of the present disclosure or anti-sortilin antibody Fabs of the present disclosure, are added to each well and incubated for 48 hours. Cells are then lysed on ice using RIPA buffer (Thermo Fisher Scientific) with protease inhibitors (Life Technologies). Lysates are collected and centrifuged at 10,000×g for 10 minutes at 4° C. Supernatants are collected and protein concentration is measured using a BCA kit according to the manufacturer's instructions (Thermo Fisher Pierce). The cell lysates are then analyzed by immunoblot. In some embodiments, an anti-sortilin antibody of the disclosure decreases the total level of sortilin in white blood cells by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%, e.g., assessed using an in vitro cell based assay, e.g., as described herein. In some embodiments, an antibody of the disclosure induces a decrease of at least about 51.6% in the total level of sortilin in white blood cells, e.g., assessed using an in vitro cell based assay, e.g., as described herein. In some embodiments, an antibody of the disclosure induces a decrease of at least about 89% in the total level of sortilin in white blood cells, e.g., assessed using an in vitro cell based assay, e.g., as described herein.

As used herein, levels of sortilin may refer to expression levels of the gene encoding sortilin; to expression levels of one or more transcripts encoding sortilin; to expression levels of sortilin protein; and/or to the amount of sortilin protein present within cells and/or on the cell surface. In some embodiments, levels of sortilin refers to the effective concentration of sortilin. Any methods known in the art for measuring levels of gene expression, transcription, translation, and/or protein abundance or localization may be used to determine the levels of sortilin. In some embodiments, cellular levels of sortilin protein, such as cell surface levels of sortilin protein, intracellular levels of sortilin protein, and/or total levels of sortilin protein are measured using any method known in the art, such as flow cytometry-based methods, immunoblotting methods such as Western blot, mass spectrometry, immunohistochemistry, imaging methods such as fluorescence microscopy, and immunoassays such as ELISA.

In certain embodiments, anti-sortilin antibodies of the present disclosure may decrease cellular levels of sortilin (e.g., cell surface levels of sortilin, intracellular levels of sortilin, and/or total levels of sortilin) by inducing sortilin degradation. Accordingly, in some embodiments, anti-sortilin antibodies of the present disclosure induce sortilin degradation. In certain embodiments, anti-sortilin antibodies of the present disclosure may decrease cellular levels of sortilin (e.g., cell surface levels of sortilin, intracellular levels of sortilin, and/or total levels of sortilin) by inducing downregulation of sortilin. Accordingly, in some embodiments, anti-sortilin antibodies of the present disclosure induce sortilin downregulation. In certain embodiments, anti-sortilin antibodies of the present disclosure may decrease cellular levels of sortilin (e.g., cell surface levels of sortilin, intracellular levels of sortilin, and/or total levels of sortilin) by inducing cleavage of sortilin. Accordingly, in some embodiments, anti-sortilin antibodies of the present disclosure induce sortilin cleavage. In certain embodiments, anti-sortilin antibodies of the present disclosure may decrease cellular levels of sortilin (e.g., cell surface levels of sortilin, intracellular levels of sortilin, and/or total levels of sortilin) by inducing internalization of sortilin. Accordingly, in some embodiments, anti-sortilin antibodies of the present disclosure induce sortilin internalization. In certain embodiments, anti-sortilin antibodies of the present disclosure may decrease cellular levels of sortilin (e.g., cell surface levels of sortilin, intracellular levels of sortilin, and/or total levels of sortilin) by inducing shedding of sortilin. Accordingly, in some embodiments, anti-sortilin antibodies of the present disclosure induce sortilin shedding.

In some embodiments, an anti-sortilin antibody of the present disclosure induces desensitization of sortilin. In some embodiments, an anti-sortilin antibody of the present disclosure acts as a ligand mimetic to transiently activate sortilin. In some embodiments, an anti-sortilin antibody of the present disclosure acts as a ligand mimetic and transiently activates sortilin before inducing a decrease in cellular levels of sortilin and/or inhibition of interaction (e.g., binding) between sortilin and one or more sortilin ligands. In some embodiments, an anti-sortilin antibody of the present disclosure acts as a ligand mimetic and transiently activates sortilin before inducing degradation of sortilin. In some embodiments, an anti-sortilin antibody of the present disclosure acts as a ligand mimetic and transiently activates sortilin before inducing cleavage of sortilin. In some embodiments, an anti-sortilin antibody of the present disclosure acts as a ligand mimetic and transiently activates sortilin before inducing internalization of sortilin. In some embodiments, an anti-sortilin antibody of the present disclosure acts as a ligand mimetic and transiently activates sortilin before inducing shedding of sortilin. In some embodiments, an anti-sortilin antibody of the present disclosure acts as a ligand mimetic and transiently activates sortilin before inducing downregulation of sortilin expression. In some embodiments, an anti-sortilin antibody of the present disclosure acts as a ligand mimetic and transiently activates sortilin before inducing desensitization of sortilin.

Anti-sortilin antibodies of the present disclosure may decrease cellular levels of sortilin with a half-maximal effective concentration (EC₅₀) (e.g., when measured in vitro) in the micromolar, nanomolar, or picomolar range. In certain embodiments, the EC₅₀ of the antibody is any of <1 μM, <100 nM, <10 nM, <1 nM, <0.1 nM, <0.01 nM, or <0.001 nM. In certain embodiments, the EC₅₀ of the antibody is less than 100 nM, less than 90 nM, less than 80 nM, less than 70 nM, less than 60 nM, less than 50 nM, less than 40 nM, less than 30 nM, less than 20 nM, less than 10 nM, less than 9 nM, less than 8 nM, less than 7 nM, less than 6 nM, less than 5 nM, less than 4 nM, less than 3 nM, less than 2 nM, less than 1 nM, less than 0.5 nM, less than 0.1 nM, less than 0.09 nM, less than 0.08 nM, less than 0.07 nM, less than 0.06 nM, less than 0.05 nM, less than 0.04 nM, less than 0.03 nM, less than 0.02 nM, less than 0.01 nM, less than 0.009 nM, less than 0.008 nM, less than 0.007 nM, less than 0.006 nM, less than 0.005 nM, less than 0.004 nM, less than 0.003 nM, less than 0.002 nM, less than 0.001 nM, or less than 0.0009 nM. In certain embodiments, the EC₅₀ of the antibody is about 0.086 nM, e.g., when measured in vitro. In certain embodiments, the EC₅₀ of the antibody is less than any of about 1000 pM, about 950 pM, about 900 pM, about 850 pM, about 800 pM, about 750 pM, about 700 pM, about 650 pM, about 600 pM, about 550 pM, about 500 pM, about 450 pM, about 400 pM, about 350 pM, about 300 pM, about 250 pM, about 200 pM, about 150 pM, about 100 pM, about 50 pM, about 40 pM, about 30 pM, about 20 pM, about 10 pM, about 1 pM, about 0.5 pM, about 0.1 pM, or about 0.05 pM. In some embodiments, the EC₅₀ of the antibody can be any of a range having an upper limit of about 1000 pM, about 950 pM, about 900 pM, about 850 pM, about 800 pM, about 750 pM, about 700 pM, about 650 pM, about 600 pM, about 550 pM, about 500 pM, about 450 pM, about 400 pM, about 350 pM, about 300 pM, about 250 pM, about 200 pM, about 150 pM, about 100 pM, about 50 pM, about 40 pM, about 30 pM, about 20 pM, about 10 pM, about 1 pM, about 0.5 pM, or about 0.1 pM, and an independently selected lower limit of about 0.05 pM, about 0.1 pM, about 0.5 pM, about 1 pM, about 10 pM, about 20 pM, about 30 pM, about 40 pM, about 50 pM, about 100 pM, about 150 pM, about 200 pM, about 250 pM, about 300 pM, about 350 pM, about 400 pM, about 450 pM, about 500 pM, about 550 pM, about 600 pM, about 650 pM, about 700 pM, about 750 pM, about 800 pM, about 850 pM, about 900 pM, or about 950 pM, wherein the lower limit is less than the upper limit. In some embodiments, the EC₅₀ of the antibody is any of about 1 pM, about 2 pM, about 3 pM, about 4 pM, about 5 pM, about 6 pM, about 7 pM, about 8 pM, about 9 pM, about 10 pM, about 15 pM, about 20 pM, about 25 pM, about 30 pM, about 35 pM, about 40 pM, about 45 pM, about 50 pM, about 55 pM, about 60 pM, about 65 pM, about 70 pM, about 75 pM, about 80 pM, about 85 pM, about 90 pM, about 95 pM, about 100 pM, about 105 pM, about 110 pM, about 115 pM, about 120 pM, about 125 pM, about 130 pM, about 135 pM, about 140 pM, about 145 pM, about 150 pM, about 155 pM, about 160 pM, about 165 pM, about 170 pM, about 175 pM, about 180 pM, about 185 pM, about 190 pM, about 195 pM, or about 200 pM. In certain embodiments, the EC₅₀ of the antibody is about 86 pM.

Various methods of measuring antibody EC₅₀ values are known in the art, including, for example, by flow cytometry. In some embodiments, the EC₅₀ is measured in vitro using cells that express sortilin, e.g., that are engineered to express sortilin, e.g., human sortilin or mammalian sortilin. In some embodiments, the EC₅₀ is measured at a temperature of approximately 4° C. In some embodiments, the EC₅₀ is measured at a temperature of approximately 25° C. In some embodiments, the EC₅₀ is measured at a temperature of approximately 35° C. In some embodiments, the EC₅₀ is measured at a temperature of approximately 37° C. In some embodiments, the EC₅₀ is determined using a monovalent antibody (e.g., a Fab) or a full-length antibody in a monovalent form. In some embodiments, the EC₅₀ is determined using antibodies containing constant regions that demonstrate enhanced Fc receptor binding. In some embodiments, the EC₅₀ is determined using antibodies containing constant regions that demonstrate reduced Fc receptor binding.

Increasing Progranulin Levels

In some embodiments, anti-sortilin antibodies of the present disclosure increase the levels of progranulin. In some embodiments, progranulin levels may refer to, without limitation, extracellular levels of progranulin, intracellular levels of progranulin, and total levels of progranulin. In some embodiments, an increase in the levels of progranulin comprises an increase in extracellular levels of progranulin. In some embodiments, an increase in the levels of progranulin comprises an increase in intracellular levels of progranulin. In some embodiments, an increase in the levels of progranulin comprises an increase in total levels of progranulin.

In some embodiments, anti-sortilin antibodies of the present disclosure increase the levels of progranulin in vitro or in vivo (e.g., in the brain, blood, and/or peripheral organs of an individual). In some embodiments, an anti-sortilin antibody of the disclosure increases the levels of progranulin if it induces an increase of 20% or more in the levels of progranulin, e.g., as measured by any in vitro cell-based assays or in tissue-based (such as brain tissue-based) assays described herein or known in the art. In some embodiments, an anti-sortilin antibody of the disclosure increases the levels of progranulin if it induces an increase at saturating antibody concentrations and/or relative to a control antibody in the levels of progranulin as measured by any in vitro cell-based assays or in tissue-based (such as brain tissue-based) assays described herein or known in the art.

In some embodiments, anti-sortilin antibodies of the present disclosure increase extracellular levels of progranulin in vitro. In some embodiments, anti-sortilin antibodies of the present disclosure increase extracellular levels of progranulin in vitro by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. In some embodiments, anti-sortilin antibodies of the present disclosure increase extracellular levels of progranulin in vitro by at least about 100%, at least about 125%, at least about 150%, at least about 175%, at least about 200%, at least about 225%, at least about 250%, at least about 275%, or at least about 300%.

In some embodiments, anti-sortilin antibodies of the present disclosure increase extracellular levels of progranulin in vivo (e.g., in the brain, blood, and/or peripheral organs of an individual). In some embodiments, anti-sortilin antibodies of the present disclosure increase extracellular levels of progranulin in vivo by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. In some embodiments, anti-sortilin antibodies of the present disclosure increase extracellular levels of progranulin in vivo by at least about 100%, at least about 125%, at least about 150%, at least about 175%, at least about 200%, at least about 225%, at least about 250%, at least about 275%, or at least about 300%.

In some embodiments, anti-sortilin antibodies of the present disclosure increase intracellular levels of progranulin in vitro. In some embodiments, anti-sortilin antibodies of the present disclosure increase intracellular ‘levels of progranulin in vitro by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. In some embodiments, anti-sortilin antibodies of the present disclosure increase intracellular levels of progranulin in vitro by at least about 100%, at least about 125%, at least about 150%, at least about 175%, at least about 200%, at least about 225%, at least about 250%, at least about 275%, or at least about 300%.

In some embodiments, anti-sortilin antibodies of the present disclosure increase intracellular levels of progranulin in vivo (e.g., in the brain, blood, and/or peripheral organs of an individual). In some embodiments, anti-sortilin antibodies of the present disclosure increase intracellular levels of progranulin in vivo by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. In some embodiments, anti-sortilin antibodies of the present disclosure increase intracellular levels of progranulin in vivo by at least about 100%, at least about 125%, at least about 150%, at least about 175%, at least about 200%, at least about 225%, at least about 250%, at least about 275%, or at least about 300%.

In some embodiments, anti-sortilin antibodies of the present disclosure increase total levels of progranulin in vitro. In some embodiments, anti-sortilin antibodies of the present disclosure increase total levels of progranulin in vitro by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. In some embodiments, anti-sortilin antibodies of the present disclosure increase total levels of progranulin in vitro by at least about 100%, at least about 125%, at least about 150%, at least about 175%, at least about 200%, at least about 225%, at least about 250%, at least about 275%, or at least about 300%.

In some embodiments, anti-sortilin antibodies of the present disclosure increase total levels of progranulin in vivo (e.g., in the brain, blood, and/or peripheral organs of an individual). In some embodiments, anti-sortilin antibodies of the present disclosure increase total levels of progranulin in vivo by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. In some embodiments, anti-sortilin antibodies of the present disclosure increase total levels of progranulin in vivo by at least about 100%, at least about 125%, at least about 150%, at least about 175%, at least about 200%, at least about 225%, at least about 250%, at least about 275%, or at least about 300%.

As used herein, levels of progranulin may refer to expression levels of the gene encoding progranulin; to expression levels of one or more transcripts encoding progranulin; to expression levels of progranulin protein; and/or to the amount of progranulin protein secreted from cells and/or present within cells. In some embodiments, levels of progranulin refer to the effective concentration of progranulin. Any methods known in the art for measuring levels of gene expression, transcription, translation, protein abundance, protein secretion, and/or protein localization may be used to determine the levels of progranulin.

In some embodiments, an anti-sortilin antibody of the present disclosure increases progranulin secretion by any of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. In some embodiments, an anti-sortilin antibody of the present disclosure increases progranulin secretion by any of at least about 100%, at least about 125%, at least about 150%, at least about 175%, at least about 200%, at least about 225%, at least about 250%, at least about 275%, or at least about 300%. In some embodiments, an anti-sortilin antibody of the present disclosure increases progranulin secretion by any of at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.1 fold, at least about 2.2 fold, at least about 2.3 fold, at least about 2.4 fold, at least about 2.5 fold, at least about 2.6 fold, at least about 2.7 fold, at least about 2.8 fold, at least about 2.9 fold, at least about 3 fold, at least about 3.1 fold, at least about 3.2 fold, at least about 3.3 fold, at least about 3.4 fold, at least about 3.5 fold, at least about 3.6 fold, at least about 3.7 fold, at least about 3.8 fold, at least about 3.9 fold, or at least about 4 fold. In some embodiments, an anti-sortilin antibody of the present disclosure increases progranulin secretion by about 1.9-fold.

Various methods of measuring progranulin secretion are known in the art, including, for example, by ELISA. In an exemplary assay, U-251 human astrocytoma cells are seeded in 96-well dishes and incubated overnight. The next morning, anti-sortilin antibodies (e.g., an anti-sortilin antibody of the disclosure), as well as control antibodies (e.g., a positive control antibody, such as goat anti-human sortilin (gtSort) from R&D Systems, AF3154, and/or an isotype control antibody, such as goat IgG, ADI-88 (human IgG1), and ADI-89 (human IgG1)), are added at 50 nM or 5 nM final dilution and the cells are thus incubated for about 72 hours. The cell culture media is then collected and the concentration of progranulin in the media samples is measured using an R&D Systems human progranulin Duoset ELISA kit. In some embodiments, the EC₅₀ is measured in vitro using cells expressing human sortilin. In some embodiments, progranulin secretion is determined using a monovalent antibody (e.g., a Fab) or a full-length antibody in a monovalent form. In some embodiments, progranulin secretion is determined using antibodies containing constant regions that demonstrate enhanced Fc receptor binding. In some embodiments, progranulin secretion is determined using antibodies containing constant regions that demonstrate reduced Fc receptor binding.

In some embodiments, anti-sortilin antibodies of the present disclosure increase the effective concentration of progranulin in vitro. In some embodiments, anti-sortilin antibodies of the present disclosure increase the effective concentration of progranulin in vitro by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. In some embodiments, anti-sortilin antibodies of the present disclosure increase the effective concentration of progranulin in vitro by at least about 100%, at least about 125%, at least about 150%, at least about 175%, at least about 200%, at least about 225%, at least about 250%, at least about 275%, or at least about 300%. In some embodiments, anti-sortilin antibodies of the present disclosure increase the effective concentration of progranulin in vivo (e.g., in the brain, blood, and/or peripheral organs of an individual). In some embodiments, anti-sortilin antibodies of the present disclosure increase the effective concentration of progranulin in vivo by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. In some embodiments, anti-sortilin antibodies of the present disclosure increase the effective concentration of progranulin in vivo by at least about 100%, at least about 125%, at least about 150%, at least about 175%, at least about 200%, at least about 225%, at least about 250%, at least about 275%, or at least about 300%. In some embodiments, anti-sortilin antibodies of the present disclosure increase the effective concentration of progranulin in vitro or in vivo by increasing extracellular levels of progranulin, intracellular levels of progranulin, total levels of progranulin, expression levels of the gene encoding progranulin, expression levels of one or more transcripts encoding progranulin, expression levels of progranulin protein, the amount of progranulin protein secreted from cells, and/or the amount of progranulin protein present within cells. Any method known in the art to measure the effective concentration of progranulin may be used, such as any in vitro, in vivo, or cell-based assay described herein, e.g., ELISA, Western blot, flow cytometry, and mass spectrometry.

In some embodiments, anti-sortilin antibodies of the present disclosure reduce endosomal internalization of progranulin, or fragments thereof. In some embodiments, anti-sortilin antibodies of the present disclosure reduce endosomal internalization and degradation of progranulin, or fragments thereof. In some embodiments, anti-sortilin antibodies of the present disclosure reduce endosomal internalization of progranulin, or fragments thereof, by reducing, inhibiting, or blocking the interaction between sortilin and progranulin. In some embodiments, anti-sortilin antibodies of the present disclosure reduce endosomal internalization of progranulin, or fragments thereof, and increase the levels or progranulin, e.g., extracellular levels of progranulin. Methods and reagents for measuring internalization (e.g., endosomal internalization) of proteins, such as progranulin, are known in the art, including, without limitation, imaging methods, such as fluorescence microscopy, confocal microscopy, or total internal reflection imaging (TIRF), or flow cytometry.

In some embodiments, anti-sortilin antibodies of the present disclosure increase one or more progranulin activities. In some embodiments, anti-sortilin antibodies of the present disclosure increase one or more progranulin activities by increasing levels of progranulin (e.g., as described above), such as the effective concentration of progranulin, extracellular levels of progranulin, intracellular levels of progranulin, total levels of progranulin, expression levels of the gene encoding progranulin, expression levels of one or more transcripts encoding progranulin, expression levels of progranulin protein, the amount of progranulin protein secreted from cells, and/or the amount of progranulin protein present within cells.

In some embodiments, an anti-sortilin antibody according to any of the above embodiments may incorporate any of the features, singly or in combination.

### Antibody Fragments

In some embodiments of any of the antibodies provided herein, the antibody is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP404097; WO 1993/01161; Hudson et al. Nat. Med. 9:129-134 (2003). Triabodies and tetrabodies are also described in Hudson et al. Nat. Med. 9:129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

In some embodiments, the antibody fragment is used in combination with a second sortilin antibody and/or with one or more antibodies that specifically bind a disease-causing protein selected from: amyloid beta or fragments thereof, Tau, IAPP, alpha-synuclein, TDP-43, FUS protein, prion protein, PrPSc, huntingtin, calcitonin, superoxide dismutase, ataxin, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, proline-arginine (PR) repeat peptides, and any combination thereof.

### Chimeric and Humanized Antibodies

In some embodiments of any of the antibodies provided herein, the antibody is a chimeric antibody. Certain chimeric antibodies are described, e.g., in U.S. Pat. No. 4,816,567. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments of any of the antibodies provided herein, the antibody is a humanized antibody. Typically, a non-human antibody is humanized to reduce immunogenicity to humans, while retaining the specificity and affinity of the parental non-human antibody. In certain embodiments, a humanized antibody is substantially non-immunogenic in humans. In certain embodiments, a humanized antibody has substantially the same affinity for a target as an antibody from another species from which the humanized antibody is derived. See, e.g., U.S. Pat. Nos. 5,530,101, 5,693,761; 5,693,762; and 5,585,089. In certain embodiments, amino acids of an antibody variable domain that can be modified without diminishing the native affinity of the antigen binding domain while reducing its immunogenicity are identified. See, e.g., U.S. Pat. Nos. 5,766,886 and 5,869,619. Generally, a humanized antibody comprises one or more variable domains in which HVRs (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), for example, to restore or improve antibody specificity or affinity.

Humanized antibodies and methods of making them are reviewed, for example, in Almagro et al. Front. Biosci. 13:161 9-1633 (2008), and are further described, e.g., in U.S. Pat. Nos. 5,821,337, 7,527,791, 6,982,321, and 7,087,409. Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA 89:4285 (1992); and Presta et al., J. Immunol. 151:2623 (1993)); human mature (somatically mutated) framework regions or human germline framework regions (see, e.g., Almagro and Fransson Front. Biosci. 13:1619-1633 (2008)); and framework regions derived from screening FR libraries (see, e.g., Baca et al. J. Biol. Chem. 272:10678-10684 (1997) and Rosok et al. J. Biol. Chem. 271:22611-22618 (1996)).

### Human Antibodies

In some embodiments of any of the antibodies provided herein, the antibody is a human antibody. Human antibodies can be produced using various techniques known in the art. Human antibodies are described generally in van Dijk et al. Curr. Opin. Pharmacol. 5:368-74 (2001) and Lonberg Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. One can engineer mouse strains deficient in mouse antibody production with large fragments of the human Ig loci in anticipation that such mice would produce human antibodies in the absence of mouse antibodies. Large human Ig fragments can preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains can yield high affinity fully human antibodies against any antigen of interest, including human antigens. Using the hybridoma technology, antigen-specific human MAbs with the desired specificity can be produced and selected. Certain exemplary methods are described in U.S. Pat. No. 5,545,807, EP 546073, and EP 546073. See also, for example, U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol. 133:3001 (1984) and Boerner et al. J. Immunol. 147:86 (1991)). Human antibodies generated via human B-cell hybridoma technology are also described in Li et al. Proc. Natl. Acad. Sci. USA, 1 03:3557-3562 (2006). Additional methods include those described, for example, in U.S. Pat. No. 7,189,826 (describing production of monoclonal human IgM antibodies from hybridoma cell lines). Human hybridoma technology (Trioma technology) is also described in Vollmers et al. Histology and Histopathology 20(3):927-937 (2005) and Vollmers et al. Methods and Findings in Experimental and Clinical Pharmacology 27(3):185-91 (2005). Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

In some embodiments of any of the antibodies provided herein, the antibody is a human antibody isolated by in vitro methods and/or screening combinatorial libraries for antibodies with the desired activity or activities. Suitable examples include but are not limited to phage display (CAT, Morphosys, Dyax, Biosite/Medarex, Xoma, Symphogen, Alexion (formerly Proliferon), Affimed), ribosome display (CAT), yeast-based platforms (Adimab), and the like. In certain phage display methods, repertoires of VH and VL genes are separately cloned by polymerase chain reaction (PCR) and recombined randomly in phage libraries, which can then be screened for antigen-binding phage as described in Winter et al. Ann. Rev. Immunol. 12: 433-455 (1994). For example, a variety of methods are known in the art for generating phage display libraries and screening such libraries for antibodies possessing the desired binding characteristics. See also Sidhu et al. J. Mol. Biol. 338(2): 299-310, 2004; Lee et al. J. Mol. Biol. 340(5): 1073-1093, 2004; Fellouse Proc. Natl. Acad. Sci. USA 101(34):12467-12472 (2004); and Lee et al. J. Immunol. Methods 284(2):1 19-132 (2004). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al. EMBO J. 12: 725-734 (1993). Finally, naive libraries can also be made synthetically by cloning unrearranged V-gene segments from stem cells, and using PCR primers comprising random sequence to encode the highly variable HVR3 regions and to accomplish rearrangement in vitro, as described by Hoogenboom et al. J. Mol. Biol., 227: 381-388, 1992. Patent publications describing human antibody phage libraries include, for example: U.S. Pat. No. 5,750,373, and US Patent Publication Nos. 2007/0292936 and 2009/0002360. Antibodies isolated from human antibody libraries are considered human antibodies or human antibody fragments herein.

### Constant Regions Including Fc Regions

In some embodiments of any of the antibodies provided herein, the antibody comprises an Fc region. In some embodiments, the Fc region is a human IgG1, IgG2, IgG3, and/or IgG4 isotype. In some embodiments, the antibody is of the IgG class, the IgM class, or the IgA class.

In certain embodiments of any of the antibodies provided herein, the antibody has an IgG2 isotype. In some embodiments, the antibody contains a human IgG2 constant region. In some embodiments, the human IgG2 constant region includes an Fc region. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has an IgG1 isotype. In some embodiments, the antibody contains a mouse IgG1 constant region. In some embodiments, the antibody contains a human IgG1 constant region. In some embodiments, the human IgG1 constant region includes an Fc region. An exemplary human IgG1 heavy chain constant region is provided in SEQ ID NO: 28 or SEQ ID NO: 29. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has an IgG4 isotype. In some embodiments, the antibody contains a human IgG4 constant region. In some embodiments, the human IgG4 constant region includes an Fc region. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has a hybrid IgG2/4 isotype. In some embodiments, the antibody includes an amino acid sequence comprising amino acids 118 to 260 according to EU numbering of human IgG2 and amino acids 261-447 according to EU numbering of human IgG4 (WO 1997/11971; WO 2007/106585).

In some embodiments, the Fc region increases clustering without activating complement as compared to a corresponding antibody comprising an Fc region that does not comprise the amino acid substitutions.

It may also be desirable to modify an anti-sortilin antibody of the present disclosure to modify effector function and/or to increase serum half-life of the antibody. For example, the Fc receptor binding site on the constant region may be modified or mutated to remove or reduce binding affinity to certain Fc receptors, such as FcγRI, FcγRII, and/or FcγRIII to reduce antibody-dependent cell-mediated cytotoxicity. In some embodiments, the effector function is impaired by removing N-glycosylation of the Fc region (e.g., in the CH2 domain of IgG) of the antibody. In some embodiments, the effector function is impaired by modifying regions such as 233-236, 297, and/or 327-331 of human IgG as described in WO 99/58572 and Armour et al. Molecular Immunology 40: 585-593 (2003); Reddy et al. J. Immunology 164:1925-1933 (2000). In other embodiments, it may also be desirable to modify an anti-sortilin antibody of the present disclosure to modify effector function to increase binding selectivity toward the ITIM-containing FcgRIIb (CD32b), e.g., to increase clustering of anti-sortilin antibodies on adjacent cells without activating humoral responses including antibody-dependent cell-mediated cytotoxicity and antibody-dependent cellular phagocytosis.

To increase the serum half-life of the antibody, one may incorporate a salvage receptor binding epitope into the antibody (especially an antibody fragment), e.g., as described in U.S. Pat. No. 5,739,277. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

### Multispecific Antibodies

Multispecific antibodies are antibodies that have binding specificities for at least two different epitopes, including those on the same or another polypeptide (e.g., one or more sortilin polypeptides of the present disclosure). In some embodiments, the multispecific antibody can be a bispecific antibody. In some embodiments, the multispecific antibody can be a trispecific antibody. In some embodiments, the multispecific antibody can be a tetraspecific antibody. Such antibodies can be derived from full-length antibodies or antibody fragments (e.g., F(ab′)₂ bispecific antibodies). In some embodiments, the multispecific antibody comprises a first antigen binding region which binds to a first site on sortilin and comprises a second antigen binding region which binds to a second site on sortilin. In some embodiments, the multispecific antibodies comprise a first antigen binding region which binds to sortilin and a second antigen binding region that binds to a second polypeptide.

Provided herein are multispecific antibodies comprising a first antigen binding region, wherein the first antigen binding region comprises the six HVRs of an antibody described herein (e.g., antibody S-15-10-7), which binds to sortilin and a second antigen binding region that binds to a second polypeptide. In some embodiments, the first antigen binding region comprises the V_{H} or V_{L} of an antibody described herein (e.g., antibody S-15-10-7).

In some embodiments of any of the multispecific antibodies provided herein, the second polypeptide is a) an antigen facilitating transport across the blood-brain-barrier; (b) an antigen facilitating transport across the blood-brain-barrier selected from transferrin receptor (TR), insulin receptor (HIR), insulin-like growth factor receptor (IGFR), low-density lipoprotein receptor related proteins 1 and 2 (LPR-1 and 2), diphtheria toxin receptor, CRM197, a llama single domain antibody, TMEM 30(A), a protein transduction domain, TAT, Syn-B, penetratin, a poly-arginine peptide, an angiopep peptide, and ANG1005; (c) a disease-causing protein selected from amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides; (d) ligands and/or proteins expressed on immune cells, wherein the ligands and/or proteins are selected from CD40, OX40, ICOS, CD28, CD137/4-1BB, CD27, GITR, PD-L1, CTLA-4, PD-L2, PD-1, B7-H3, B7-H4, HVEM, BTLA, KIR, GAL9, TIM3, A2AR, LAG-3, and phosphatidylserine; and/or (e) a protein, lipid, polysaccharide, or glycolipid expressed on one or more tumor cells, and any combination thereof.

Numerous antigens are known in the art that facilitate transport across the blood-brain barrier (see, e.g., Gabathuler R. Neurobiol. Dis. 37:48-57 (2010)). Such second antigens include, without limitation, transferrin receptor (TR), insulin receptor (HIR), Insulin-like growth factor receptor (IGFR), low-density lipoprotein receptor related proteins 1 and 2 (LPR-1 and 2), diphtheria toxin receptor, including CRM197 (a non-toxic mutant of diphtheria toxin), llama single domain antibodies such as TMEM 30(A) (Flippase), protein transduction domains such as TAT, Syn-B, or penetratin, poly-arginine or generally positively charged peptides, Angiopep peptides such as ANG1005 (see, e.g., Gabathuler, 2010), and other cell surface proteins that are enriched on blood-brain barrier endothelial cells (see, e.g., Daneman et al. PLoS One 5(10):ei3741 (2010)).

The multivalent antibodies may recognize the sortilin antigen as well as without limitation an additional antigen, such as an Aβ peptide antigen or an α-synuclein protein antigen, a Tau protein antigen, a TDP-43 protein antigen, a prion protein antigen, a huntingtin protein antigen, a RAN translation product antigen, including DiPeptide Repeats (DPRs peptides) composed of glycine-alanine (GA), glycine-proline (GP), glycine-arginine (GR), proline-alanine (PA), or proline-arginine (PR), an insulin receptor antigen, or an insulin like growth factor receptor antigen, a transferrin receptor antigen, or any other antigen that facilitates antibody transfer across the blood brain barrier. In some embodiments, the second polypeptide is transferrin. In some embodiments, the second polypeptide is Tau. In some embodiments, the second polypeptide is Ap. In some embodiments, the second polypeptide is TREM2. In some embodiments, the second polypeptide is α-synuclein.

The multivalent antibody contains at least one polypeptide chain (and preferably two polypeptide chains), wherein the polypeptide chain or chains comprise two or more variable domains. For instance, the polypeptide chain or chains may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. Similarly, the polypeptide chain or chains may comprise V_{H}-C_{H}1-flexible linker-V_{H}-C_{H}1-Fc region chain; or V_{H}-C_{H}1-V_{H}-C_{H}1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

Techniques for making multispecific antibodies include, but are not limited to, recombinant co-expression of two immunoglobulin heavy chain-light chain pairs having different specificities (see Milstein and Cuello Nature 305: 537 (1983), WO 93/08829, and Traunecker et al. EMBO J. 10:3655 (1991)), and “knob-in-hole” engineering (see, e.g., U.S. Pat. No. 5,731,168). See also WO 2013/026833 (CrossMab). Multi-specific antibodies may also be made by engineering electrostatic steering effects for making antibody Fc-heterodimeric molecules (WO 2009/089004A1); cross-linking two or more antibodies (see, e.g., U.S. Pat. No. 4,676,980); using leucine; using “diabody” technology for making bispecific antibody fragments (see, e.g., Hollinger et al. Proc. Natl. Acad. Sci. USA 90:6444-6448 (1993)); and using single-chain Fv (scFv) dimers (see, e.g., Gruber et al. J. Immunol. 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576). The antibody herein also includes a “Dual Acting FAb” or “DAF” comprising an antigen binding site that binds to multiple sortilin antigens (see, US 2008/0069820, for example).

Antibodies with Improved Stability

Amino acid sequence modifications of anti-sortilin antibodies of the present disclosure, or antibody fragments thereof, to improve stability during manufacturing, storage, and in vivo administration, are also contemplated. For example, it may be desirable to reduce degradation of the antibodies or antibody fragments of the present disclosure through multiple pathways, including without limitation, oxidation and deamidation. Amino acid sequence variants of the antibodies or antibody fragments are prepared by introducing appropriate nucleotide changes into the nucleic acid encoding the antibodies or antibody fragments, or by peptide synthesis. Such modifications include, for example, deletions from, and/or insertions into, and/or substitutions of, residues within the amino acid sequences of the antibody. Any combination of deletion, insertion, and substitution can be made to arrive at the final construct, provided that the final construct possesses the desired characteristics (i.e., reduced susceptibility to degradation, and one or more of the characteristics of the anti-sortilin antibodies of the disclosure described herein).

### Antibody Variants

In some embodiments of any of the antibodies provided herein, amino acid sequence variants of the antibodies are contemplated. For example, it may be desirable to improve the binding affinity and/or other biological properties of the antibody.

Substitution. Insertion, and Deletion Variants

In some embodiments of any of the antibodies provided herein, antibody variants having one or more amino acid substitutions are provided. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody.

TABLE 2
Amino Acid Substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Tyr |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro; and
(6) aromatic: Trp, Tyr, Phe.

For example, non-conservative substitutions can involve the exchange of a member of one of these classes for a member from another class. Such substituted residues can be introduced, for example, into regions of a human antibody that are homologous with non-human antibodies, or into the non-homologous regions of the molecule.

In making changes to the polypeptide or antibody described herein, according to certain embodiments, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al. J. Mol. Biol., 157:105-131 (1982). It is known that certain amino acids can be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within ±1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments. In certain embodiments, the greatest local average hydrophilicity of an amino acid, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0±1); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within +2 is included, in certain embodiments, those which are within +1 are included, and in certain embodiments, those within +0.5 are included. One can also identify epitopes from primary amino acid sequences on the basis of hydrophilicity. These regions are also referred to as “epitopic core regions”.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two, three, four, five, or six amino acid substitutions.

Amino acid sequence insertions include amino- and/or carboxyl-terminal fusions ranging in length from one residue to polypeptides comprising a hundred or more residues, as well as intrasequence insertions of single or multiple amino acid residues. Examples of terminal insertions include an antibody with an N-terminal methionyl residue. Other insertional variants of the antibody molecule include fusion to the N- or C-terminus of the antibody to an enzyme (e.g., for ADEPT) or a polypeptide which increases the serum half-life of the antibody.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment, such as an Fv fragment).

Glycosylation Variants

In some embodiments of any of the antibodies provided herein, the antibody is altered to increase or decrease the extent to which the antibody is glycosylated. Addition or deletion of glycosylation sites to an antibody may be conveniently accomplished by altering the amino acid sequence such that one or more glycosylation sites is created or removed.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to an antibody of the disclosure may be conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where an antibody of the disclosure comprises an Fc region, the carbohydrate attached thereto may be altered. Native antibodies produced by mammalian cells typically comprise a branched, biantennary oligosaccharide that is generally attached by an N-linkage to Asn297, according to Kabat numbering of the CH2 domain of the Fc region. The oligosaccharide may include various carbohydrates, for example, mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the disclosure may be made in order to create antibody variants with certain improved properties.

In one embodiment, antibody variants are provided having a carbohydrate structure that lacks fucose attached (directly or indirectly) to an Fc region. See, e.g., US Patent Publication Nos. 2003/0157108 and 2004/0093621. Examples of publications related to “defucosylated” or “fucose-deficient” antibody variants include: US 2003/0157108; US 2003/0115614; US 2002/0164328; US 2004/0093621; US 2004/0132140; US 2004/0110704; US 2004/0110282; US 2004/0109865; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Led 3 CHO cells deficient in protein fucosylation (Ripka et al. Arch. Biochem. Biophys. 249:533-545 (1986); US 2003/0157108), and knockout cell lines, such as alpha-1,6-fucosyltransferase gene, FUT8, knockout CHO cells (see, e.g., Yamane-Ohnuki et al. Biotech. Bioeng. 87: 614 (2004) and Kanda et al. Biotechnol. Bioeng. 94(4):680-688 (2006)).

Modified Constant Regions

In some embodiments of any of the antibodies provided herein, the antibody comprises an Fc region comprising one or more modifications, e.g., relative to a wild type or unmodified Fc region of the same isotype. In some embodiments, the Fc region is capable of binding to Fc gamma receptor.

In some embodiments of any of the antibodies provided herein, the modified antibody Fc region is an IgG1 modified Fc region. In some embodiments, the IgG1 modified Fc region comprises one or more modifications. For example, in some embodiments, the IgG1 modified Fc region comprises one or more amino acid substitutions (e.g., relative to a wild-type Fc region of the same isotype). In some embodiments, the one or more amino acid substitutions are selected from N297A (Bolt S et al. (1993) Eur J Immunol 23:403-411), D265A (Shields et al. (2001) R. J. Biol. Chem. 276, 6591-6604), L234A, L235A (Hutchins et al. (1995) Proc Natl Acad Sci USA, 92:11980-11984; Alegre et al., (1994) Transplantation 57:1537-1543. 31; Xu et al., (2000) CellImmunol, 200:16-26), G237A (Alegre et al. (1994) Transplantation 57:1537-1543. 31; Xu et al. (2000) CellImmunol, 200:16-26), C226S, C229S, E233P, L234V, L234F, L235E (McEarchern et al., (2007) Blood, 109:1185-1192), P331S (Sazinsky et al., (2008) Proc Natl Acad Sci USA 2008, 105:20167-20172), S267E, L328F, A330L, M252Y, S254T, and/or T256E, where the amino acid position is according to the EU numbering convention.

In some embodiments of any of the IgG1 modified Fc regions provided herein, the Fc comprises an N297A mutation according to EU numbering. In some embodiments of any of the IgG1 modified Fc regions provided herein, the Fc comprises D265A and N297A mutations according to EU numbering. In some embodiments of any of the IgG1 modified Fc regions provided herein, the Fc comprises a D270A mutation according to EU numbering. In some embodiments, the IgG1 modified Fc region provided herein comprises L234A and L235A mutations according to EU numbering. In some embodiments of any of the IgG1 modified Fc regions provided herein, the Fc comprises L234A and G237A mutations according to EU numbering. In some embodiments of any of the IgG1 modified Fc regions provided herein, the Fc comprises L234A, L235A and G237A mutations according to EU numbering. In some embodiments of any of the IgG1 modified Fc regions provided herein, the Fc comprises one or more (including all) of P238D, L328E, E233, G237D, H268D, P271G and A330R mutations according to EU numbering. In some embodiments of any of the IgG1 modified Fc regions provided herein, the Fc comprises one or more of S267E/L328F mutations according to EU numbering. In some embodiments of any of the IgG1 modified Fc regions provided herein, the Fc comprises P238D, L328E, E233D, G237D, H268D, P271G and A330R mutations according to EU numbering. In some embodiments of any of the IgG1 modified Fc regions provided herein, the Fc comprises P238D, L328E, G237D, H268D, P271G and A330R mutations according to EU numbering. In some embodiments of any of the IgG1 modified Fc regions provided herein, the Fc comprises P238D, S267E, L328E, E233D, G237D, H268D, P271G and A330R mutations according to EU numbering. In some embodiments of any of the IgG1 modified Fc regions provided herein, the Fc comprises P238D, S267E, L328E, G237D, H268D, P271G and A330R mutations according to EU numbering. In some embodiments of any of the IgG1 modified Fc regions provided herein, the Fc comprises C226S, C229S, E233P, L234V, and L235A mutations according to EU numbering. In some embodiments of any of the IgG1 modified Fc regions provided herein, the Fc comprises L234F, L235E, and P331S mutations according to EU numbering. In some embodiments of any of the IgG1 modified Fc regions provided herein, the Fc comprises S267E and L328F mutations according to EU numbering. In some embodiments of any of the IgG1 modified Fc regions provided herein, the Fc comprises a S267E mutation according to EU numbering. In some embodiments of any of the IgG1 modified Fc regions provided herein, the Fc comprises a substitution of the constant heavy 1 (CH1) and hinge region of IgG1 with CH1 and hinge region of IgG2 (amino acids 118-230 of IgG2 according to EU numbering) with a Kappa light chain.

In some embodiments of any of the IgG1 modified Fc regions provided herein, the Fc includes two or more amino acid substitutions that increase antibody clustering without activating complement as compared to a corresponding antibody having an Fc region that does not include the two or more amino acid substitutions. Accordingly, in some embodiments of any of the IgG1 modified Fc regions provided herein, the Fc region comprises an amino acid substitution at position E430G and one or more amino acid substitutions in the Fc region at a residue position selected from: L234F, L235A, L235E, S267E, K322A, L328F, A330S, P331S, and any combination thereof according to EU numbering. In some embodiments, the IgG1 modified Fc region comprises an amino acid substitution at positions E430G, L243A, L235A, and P331S according to EU numbering. In some embodiments, the IgG1 modified Fc region comprises an amino acid substitution at positions E430G and P331S according to EU numbering. In some embodiments, the IgG1 modified Fc region comprises an amino acid substitution at positions E430G and K322A according to EU numbering. In some embodiments, the IgG1 modified Fc region comprises an amino acid substitution at positions E430G, A330S, and P331S according to EU numbering. In some embodiments, the IgG1 modified Fc region comprises an amino acid substitution at positions E430G, K322A, A330S, and P331S according to EU numbering. In some embodiments, the IgG1 modified Fc region comprises an amino acid substitution at positions E430G, K322A, and A330S according to EU numbering. In some embodiments, the IgG1 modified Fc region comprises an amino acid substitution at positions E430G, K322A, and P331S according to EU numbering.

In some embodiments of any of the IgG1 modified Fc regions provided herein, the IgG1 modified Fc may further comprise an A330L mutation (Lazar et al. Proc Natl Acad Sci USA, 103:4005-4010 (2006)), or one or more of L234F, L235E, and/or P331S mutations (Sazinsky et al. Proc Natl Acad Sci USA, 105:20167-20172 (2008)), according to the EU numbering convention, to eliminate complement activation. In some embodiments of any of the IgG1 modified Fc regions provided herein, the IgG1 modified Fc may further comprise one or more of A330L, A330S, L234F, L235E, and/or P331S amino acid substitutions according to EU numbering. In some embodiments of any of the IgG1 modified Fc regions provided herein, the IgG1 modified Fc may further comprise one or more mutations to enhance the antibody half-life in human serum (e.g., one or more (including all) of M252Y, S254T, and T256E mutations according to the EU numbering convention). In some embodiments of any of the IgG1 modified Fc regions provided herein, the IgG1 modified Fc may further comprise one or more of E430G, E430S, E430F, E430T, E345K, E345Q, E345R, E345Y, S440Y, and/or S440W amino acid substitutions according to EU numbering.

In some embodiments of any of the antibodies provided herein, the antibody has a human IgG1 isotype (huIgG1) and the Fc region comprises one or more amino acid substitutions selected from N297A, N297Q, D265A, L234A, L235A, C226S, C229S, P238S, E233P, L234V, P238A, A327Q, A327G, P329A, K322A, L234F, L235E, P331S, T394D, A330L, M252Y, S254T, or T256E, and any combination thereof, wherein the numbering of the residues is according to EU numbering. In some embodiments of any of the antibodies provided herein, an antibody comprising an IgG1 Fc region further comprises one or more amino acid substitutions selected from A330L, L234F, L235E, or P331S, and any combination thereof, wherein the numbering of the residues is according to EU numbering. In some embodiments of any of the antibodies provided herein, an antibody comprising an IgG1 Fc region further comprises one or more amino acid substitutions selected from M252Y, S254T, or T256E, and any combination thereof, wherein the numbering of the residues is according to EU numbering.

In some embodiments of any of the antibodies provided herein, the antibody has a human IgG4 isotype (huIgG4) and the Fc region comprises one or more amino acid substitutions selected from E233P, F234V, L234A/F234A, L235A, G237A, E318A, S228P, L236E, S241P, L248E, T394D, M252Y, S254T, T256E, N297A, N297Q, and any combination thereof, wherein the numbering of the residues is according to EU numbering. In some embodiments of any of the antibodies provided herein, an antibody comprising an IgG4 Fc region further comprises one or more amino acid substitutions selected from M252Y, S254T, or T256E, and any combination thereof, wherein the numbering of the residues is according to EU numbering. In some embodiments of any of the antibodies provided herein, an antibody comprising an IgG4 Fc region further comprises a S228P amino acid substitution, wherein the numbering of the residues is according to EU numbering.

In some embodiments of any of the antibodies provided herein, the antibody has a human IgG1 isotype (huIgG1) and the Fc region comprises amino acid substitutions at positions L234A, L235A, and P331S, wherein the numbering of the residue position is according to EU numbering. In some embodiments, the antibody comprises a huIgG1 Fc region comprising a heavy chain constant region comprising the amino acid sequence of SEQ ID NO: 25. In some embodiments, the antibody comprises a huIgG1 Fc region comprising a heavy chain constant region comprising the amino acid sequence of SEQ ID NO: 26. In some embodiments, the antibody comprises a heavy chain comprising the amino acid sequence of SEQ ID NO: 31 and a light chain comprising the amino acid sequence of SEQ ID NO: 30. In some embodiments, the antibody comprises a heavy chain comprising the amino acid sequence of SEQ ID NO: 32 and a light chain comprising the amino acid sequence of SEQ ID NO: 30.

In some embodiments of any of the antibodies provided herein, the modified antibody Fc region is an IgG2 modified Fc region. In some embodiments, the IgG2 modified Fc region comprises one or more modifications. For example, in some embodiments, the IgG2 modified Fc region comprises one or more amino acid substitutions (e.g., relative to a wild-type Fc region of the same isotype). In some embodiments of any of the IgG2 modified Fc regions provided herein, the one or more amino acid substitutions are selected from V234A (Alegre et al. Transplantation 57:1537-1543 (1994); Xu et al. CellImmunol, 200:16-26 (2000)); G237A (Cole et al. Transplantation, 68:563-571 (1999)); H268Q, V309L, A330S, P331S (US 2007/0148167; Armour et al. Eur J Immunol 29: 2613-2624 (1999); Armour et al. The Haematology Journal 1(Suppl.1):27 (2000); Armour et al. The Haematology Journal 1(Suppl.1):27 (2000)); C219S, and/or C220S (White et al. Cancer Cell 27, 138-148 (2015)); S267E, L328F (Chu et al. MolImmunol, 45:3926-3933 (2008)); or M252Y, S254T, and/or T256E, according to the EU numbering convention. In some embodiments of any of the IgG2 modified Fc regions, the Fc comprises an amino acid substitution at positions V234A and G237A according to EU numbering. In some embodiments of any of the IgG2 modified Fc regions, the Fc comprises an amino acid substitution at positions C219S or C220S according to EU numbering. In some embodiments of any of the IgG2 modified Fc regions, the Fc comprises an amino acid substitution at positions A330S and P331S according to EU numbering. In some embodiments of any of the IgG2 modified Fc regions, the Fc comprises an amino acid substitution at positions S267E and L328F according to EU numbering.

In some embodiments of any of the IgG2 modified Fc regions provided herein, the Fc comprises a C127S amino acid substitution according to the EU numbering convention (White et al., (2015) Cancer Cell 27, 138-148; Lightle et al. Protein Sci. 19:753-762 (2010); and WO 2008/079246). In some embodiments of any of the IgG2 modified Fc regions, the antibody has an IgG2 isotype with a Kappa light chain constant domain that comprises a C214S amino acid substitution according to the EU numbering convention (White et al. Cancer Cell 27:138-148 (2015); Lightle et al. Protein Sci. 19:753-762 (2010); and WO 2008/079246).

In some embodiments of any of the IgG2 modified Fc regions provided herein, the Fc comprises a C220S amino acid substitution according to the EU numbering convention. In some embodiments of any of the IgG2 modified Fc regions, the antibody has an IgG2 isotype with a Kappa light chain constant domain that comprises a C214S amino acid substitution according to the EU numbering convention.

In some embodiments of any of the IgG2 modified Fc regions provided herein, the Fc comprises a C219S amino acid substitution according to the EU numbering convention. In some embodiments of any of the IgG2 modified Fc regions, the antibody has an IgG2 isotype with a Kappa light chain constant domain that comprises a C214S amino acid substitution according to the EU numbering convention.

In some embodiments of any of the IgG2 modified Fc regions provided herein, the Fc includes an IgG2 isotype heavy chain constant domain 1 (CH1) and hinge region (White et al. Cancer Cell 27:138-148 (2015)). In certain embodiments of any of the IgG2 modified Fc regions provided herein, the IgG2 isotype CH1 and hinge region comprise the amino acid sequence of amino acids 118-230 according to EU numbering. In some embodiments of any of the IgG2 modified Fc regions, the antibody Fc region comprises a S267E amino acid substitution, a L328F amino acid substitution, or both, and/or a N297A or N297Q amino acid substitution according to the EU numbering convention.

In some embodiments of any of the IgG2 modified Fc regions provided herein, the Fc further comprises one or more amino acid substitution at positions E430G, E430S, E430F, E430T, E345K, E345Q, E345R, E345Y, S440Y, and S440W according to EU numbering. In some embodiments of any of the IgG2 modified Fc regions, the Fc may further comprise one or more mutations to enhance the antibody half-life in human serum (e.g., one or more (including all) of M252Y, S254T, and T256E mutations according to the EU numbering convention). In some embodiments of any of the IgG2 modified Fc regions, the Fc may further comprise A330S and P331S amino acid substitutions.

In some embodiments of any of the IgG2 modified Fc regions provided herein, the Fc is an IgG2/4 hybrid Fc. In some embodiments, the IgG2/4 hybrid Fc comprises IgG2 amino acids 118 to 260 and IgG4 amino acids 261 to 447. In some embodiments of any IgG2 modified Fc region provided herein, the Fc comprises one or more amino acid substitutions at positions H268Q, V309L, A330S, and P331S according to EU numbering.

In some embodiments of any of the IgG1 and/or IgG2 modified Fc regions provided herein, the Fc comprises one or more additional amino acid substitutions selected from A330L, L234F, L235E, or P331S, and any combination thereof, according to EU numbering.

In certain embodiments of any of the IgG1 and/or IgG2 modified Fc regions provided herein, the Fc comprises one or more amino acid substitutions at a residue position selected from C127S, L234A, L234F, L235A, L235E, S267E, K322A, L328F, A330S, P331S, E345R, E430G, S440Y, and any combination thereof according to EU numbering. In some embodiments of any of the IgG1 and/or IgG2 modified Fc regions, the Fc comprises an amino acid substitution at positions E430G, L243A, L235A, and P331S according to EU numbering. In some embodiments of any of the IgG1 and/or IgG2 modified Fc regions, the Fc comprises an amino acid substitution at positions E430G and P331S according to EU numbering. In some embodiments of any of the IgG1 and/or IgG2 modified Fc regions, the Fc comprises an amino acid substitution at positions E430G and K322A according to EU numbering. In some embodiments of any of the IgG1 and/or IgG2 modified Fc regions, the Fc comprises an amino acid substitution at positions E430G, A330S, and P331S according to EU numbering. In some embodiments of any of the IgG1 and/or IgG2 modified Fc regions, the Fc comprises an amino acid substitution at positions E430G, K322A, A330S, and P331S according to EU numbering. In some embodiments of any of the IgG1 and/or IgG2 modified Fc regions, the Fc comprises an amino acid substitution at positions E430G, K322A, and A330S according to EU numbering. In some embodiments of any of the IgG1 and/or IgG2 modified Fc regions, the Fc comprises an amino acid substitution at positions E430G, K322A, and P331S according to EU numbering. In some embodiments of any of the IgG1 and/or IgG2 modified Fc regions, the Fc comprises an amino acid substitution at positions S267E and L328F according to EU numbering. In some embodiments of any of the IgG1 and/or IgG2 modified Fc regions, the Fc comprises an amino acid substitution at position C127S according to EU numbering. In some embodiments of any of the IgG1 and/or IgG2 modified Fc regions, the Fc comprises an amino acid substitution at positions E345R, E430G and S440Y according to EU numbering.

In some embodiments of any of the antibodies provided herein, the antibody has an IgG4 isotype and comprises a modified Fc region. In some embodiments, the IgG4 modified Fc region comprises one or more modifications. For example, in some embodiments, the IgG4 modified Fc region comprises one or more amino acid substitutions (e.g., relative to a wild-type Fc region of the same isotype). In some embodiments of any of the IgG4 modified Fc regions provided herein, the one or more amino acid substitutions are selected from L235A, G237A, S229P, L236E (Reddy et al. J Immunol 164:1925-1933(2000)), S267E, E318A, L328F, M252Y, S254T, and/or T256E according to the EU numbering convention. In some embodiments of any of the IgG4 modified Fc regions, the Fc may further comprise L235A, G237A, and E318A amino acid substitutions according to the EU numbering convention. In some embodiments of any of the IgG4 modified Fc regions, the Fc may further comprise S228P and L235E amino acid substitutions according to the EU numbering convention. In some embodiments of any of the IgG4 modified Fc regions, the IgG4 modified Fc may further comprise S267E and L328F amino acid substitutions according to the EU numbering convention.

In some embodiments of any of the IgG4 modified Fc regions provided herein, the IgG4 modified Fc comprises an S228P mutation according to the EU numbering convention (Angal et al. Mol Immunol. 30:105-108 (1993)) and/or one or more mutations described in (Peters et al. J Biol Chem. 287(29):24525-33 (2012)) to enhance antibody stabilization.

In some embodiments of any of the IgG4 modified Fc regions provided herein, the IgG4 modified Fc may further comprise one or more mutations to enhance the antibody half-life in human serum (e.g., one or more (including all) of M252Y, S254T, and T256E mutations according to the EU numbering convention).

In some embodiments of any of the IgG4 modified Fc regions provided herein, the Fc comprises a L235E amino acid substitution according to EU numbering. In certain embodiments of any of the IgG4 modified Fc regions, the Fc comprises one or more amino acid substitutions at a residue position selected from C127S, F234A, L235A, L235E, S267E, K322A, L328F, E345R, E430G, S440Y, and any combination thereof, according to EU numbering. In some embodiments of any of the IgG4 modified Fc regions, the Fc comprises an amino acid substitution at positions E430G, L243A, L235A, and P331S according to EU numbering. In some embodiments of any of the IgG4 modified Fc regions, the Fc comprises an amino acid substitution at positions E430G and P331S according to EU numbering. In some embodiments of any of the IgG4 modified Fc regions, the Fc comprises an amino acid substitution at positions E430G and K322A according to EU numbering. In some embodiments of any of the IgG4 modified Fc regions, the Fc comprises an amino acid substitution at position E430 according to EU numbering. In some embodiments of any of the IgG4 modified Fc regions, the Fc region comprises an amino acid substitution at positions E430G and K322A according to EU numbering. In some embodiments of any of the IgG4 modified Fc regions, the Fc comprises an amino acid substitution at positions S267E and L328F according to EU numbering. In some embodiments of any of the IgG4 modified Fc regions, the Fc comprises an amino acid substitution at position C127S according to EU numbering. In some embodiments of any of the IgG4 modified Fc regions, the Fc comprises an amino acid substitution at positions E345R, E430G and S440Y according to EU numbering.

### Nucleic Acids, Vectors, and Host Cells

Anti-sortilin antibodies of the present disclosure may be produced using recombinant methods and compositions, e.g., as described in U.S. Pat. No. 4,816,567. In some embodiments, isolated nucleic acids having a nucleotide sequence encoding any of the anti-sortilin antibodies of the present disclosure are provided. Such nucleic acids may encode an amino acid sequence comprising the light chain variable domain (V_{L}) and/or an amino acid sequence comprising the heavy chain variable domain (V_{H}) of the anti-sortilin antibody (e.g., the light and/or heavy chains of the antibody). In some embodiments, one or more vectors (e.g., expression vectors) comprising such nucleic acids are provided. In some embodiments, a host cell comprising such nucleic acids or vectors is also provided. In some embodiments, the host cell comprises (e.g., has been transduced with): (1) a vector comprising a nucleic acid that encodes an amino acid sequence comprising the V_{L} of the antibody and an amino acid sequence comprising the V₁ of the antibody, or (2) a first vector comprising a nucleic acid that encodes an amino acid sequence comprising the V_{L} of the antibody and a second vector comprising a nucleic acid that encodes an amino acid sequence comprising the V_{H} of the antibody. In some embodiments, the host cell is eukaryotic, e.g., a Chinese Hamster Ovary (CHO) cell or lymphoid cell (e.g., Y0, NS0, Sp20 cell). Host cells of the present disclosure also include, without limitation, isolated cells, in vitro cultured cells, and ex vivo cultured cells.

Methods of making an anti-sortilin antibody of the present disclosure are provided. In some embodiments, the method includes culturing a host cell of the present disclosure comprising a nucleic acid encoding the anti-sortilin antibody, under conditions suitable for expression of the antibody. In some embodiments, the antibody is subsequently recovered from the host cell (or host cell culture medium).

For recombinant production of an anti-sortilin antibody of the present disclosure, a nucleic acid encoding the anti-sortilin antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors comprising a nucleic acid sequence encoding any of the anti-sortilin antibodies of the present disclosure include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones comprising the vector. Suitable examples include plasmids and bacterial viruses, e.g., pUC18, pUC19, Bluescript (e.g., pBS SK+) and its derivatives, mpl8, mpl9, pBR322, pMB9, ColEl, pCR1, RP4, phage DNAs, and shuttle vectors such as pSA3 and pAT28. These and many other cloning vectors are available from commercial vendors such as BioRad, Strategene, and Invitrogen.

Suitable host cells for cloning or expression of antibody-encoding vectors include prokaryotic or eukaryotic cells. For example, anti-sortilin antibodies of the present disclosure may be produced in bacteria, in particular when glycosylation and Fc effector function are not needed. For information about the expression of antibody fragments and polypeptides in bacteria, see, e.g., U.S. Pat. Nos. 5,648,237, 5,789,199, and 5,840,523. After expression, the antibody may be isolated from the bacterial cell paste in a soluble fraction and can be further purified.

In addition to prokaryotes, eukaryotic microorganisms, such as filamentous fungi or yeast, are also suitable cloning or expression hosts for antibody-encoding vectors, including fungi and yeast strains whose glycosylation pathways have been “humanized,” resulting in the production of an antibody with a partially or fully human glycosylation pattern (e.g., Gerngross Nat. Biotech. 22:1409-1414 (2004); and Li et al. Nat. Biotech. 24:210-215 (2006)).

Suitable host cells for the expression of glycosylated antibodies can also be derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts (e.g., U.S. Pat. Nos. 5,959,177, 6,040,498, 6,420,548, 7,125,978, and 6,417,429, describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are the monkey kidney CV1 line transformed by SV40 (COS-7); human embryonic kidney line (293 or 293 cells as described, e.g., in Graham et al. J. Gen Virol. 36:59 (1977)); baby hamster kidney cells (BHK); mouse sertoli cells (TM4 cells as described, e.g., in Mather, Biol. Reprod. 23:243-251 (1980)); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562) cells; TRI cells, as described, e.g., in Mather et al. Annals N.Y. Acad. Sci. 383:44-68 (1982); MRC 5 cells; and FS4 cells. Other useful mammalian host cell lines include Chinese hamster ovary (CHO) cells, including DHFR− CHO cells (Urlaub et al. Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### Diagnostic Uses

The anti-sortilin antibodies of the present disclosure also have diagnostic utility. This disclosure therefore provides for methods of using the antibodies of this disclosure, or functional fragments thereof, for diagnostic purposes, such as the detection of a sortilin protein in an individual or in tissue samples derived from an individual.

In some embodiments, the individual is a human. In some embodiments, the individual is a human patient suffering from, or at risk for developing a disease, disorder, or injury of the present disclosure. In some embodiments, the diagnostic methods involve detecting a sortilin protein in a biological sample, such as a biopsy specimen, a tissue, or a cell. In some embodiments, an anti-sortilin antibody described herein is contacted with the biological sample, and antigen-bound antibody is detected. For example, a biopsy specimen may be stained with an anti-sortilin antibody described herein in order to detect and/or quantify disease-associated cells or the level of sortilin. The detection method may involve quantification of the antigen-bound antibody. Antibody detection in biological samples may occur with any method known in the art, including immunofluorescence microscopy, immunocytochemistry, immunohistochemistry, ELISA, FACS analysis, immunoprecipitation, or micro-positron emission tomography. In certain embodiments, the antibody is radiolabeled, for example with ¹⁸F, and subsequently detected utilizing micro-positron emission tomography analysis. Antibody-binding may also be quantified in an individual by non-invasive techniques such as positron emission tomography (PET), X-ray computed tomography, single-photon emission computed tomography (SPECT), computed tomography (CT), and computed axial tomography (CAT).

In other embodiments, an anti-sortilin antibody of the present disclosure may be used to detect and/or quantify, for example, microglia in a brain specimen taken from a preclinical disease model (e.g., a non-human disease model, such as a mouse or a cynomolgus monkey disease model). As such, an anti-sortilin antibody of the present disclosure may be useful in evaluating therapeutic response after treatment in a model for a disease, disorder, or injury such as frontotemporal dementia, progressive supranuclear palsy, Alzheimer's disease, vascular dementia, seizures, retinal dystrophy, amyotrophic lateral sclerosis, traumatic brain injury, a spinal cord injury, dementia, stroke, Parkinson's disease, acute disseminated encephalomyelitis, retinal degeneration, age related macular degeneration, glaucoma, multiple sclerosis, septic shock, bacterial infection, arthritis, or osteoarthritis, e.g., as compared to a control. In some embodiments, an anti-sortilin antibody of the present disclosure may be useful in evaluating therapeutic response after treatment in a model for a neurodegenerative or nervous system disease or disorder, such as frontotemporal dementia, Alzheimer's disease, or Parkinson's disease, e.g., as compared to a control.

### Biomarkers

### Progranulin

In some embodiments, the methods of treating or delaying progression of a disease or disorder provided herein further comprise measuring the level of progranulin protein in a sample of blood (e.g., plasma or serum) or cerebrospinal fluid obtained from an individual before and after the individual has received one or more doses of an anti-sortilin antibody of the disclosure.

In some embodiments, administration of an anti-sortilin antibody of the disclosure to an individual according to the methods provided herein results in an increase in the level of progranulin protein in the plasma and/or in the cerebrospinal fluid of the individual compared to prior to administration of the anti-sortilin antibody. In some embodiments, administration of an anti-sortilin antibody of the disclosure to an individual according to the methods provided herein results in an increase in the level of progranulin protein in the plasma and/or in the cerebrospinal fluid of the individual of at least about 1.4-fold, at least about 1.8-fold, at least about 2-fold, at least about 2.2-fold, at least about 2.4-fold, at least about 2.6-fold, at least about 2.8-fold, at least about 3-fold, at least about 3.2-fold, at least about 3.4-fold, at least about 3.6-fold, at least about 3.8-fold, at least about 4-fold, at least about 4.2-fold, at least about 4.4-fold, at least about 4.6-fold, at least about 4.8-fold, at least about 5-fold, at least about 5.2-fold, at least about 5.4-fold, at least about 5.6-fold, at least about 5.8-fold, at least about 6-fold, at least about 6.2-fold, at least about 6.4-fold, at least about 6.6-fold, at least about 6.8-fold, at least about 7-fold, at least about 7.2-fold, at least about 7.4-fold, at least about 7.6-fold, at least about 7.8-fold, at least about 8-fold, at least about 8.2-fold, at least about 8.4-fold, at least about 8.6-fold, at least about 8.8-fold, at least about 9-fold, at least about 9.2-fold, at least about 9.4-fold, at least about 9.6-fold, at least about 9.8-fold, at least about 10-fold, or more, compared to prior to administration of the anti-sortilin antibody.

In some embodiments, administration of an anti-sortilin antibody of the disclosure to an individual according to the methods provided herein results in an increase in the level of progranulin protein in the plasma and/or in the cerebrospinal fluid of the individual of at least about any of 5%, 6%, 7%, 8%, 9%, 10%, 11%, 12%1, 3%, 14%, 15%, 16%, 17%, 18%, 19%, 20%, or more, compared to prior to administration of the anti-sortilin antibody. In some embodiments, administration of an anti-sortilin antibody of the disclosure to an individual according to the methods provided herein results in an increase in the level of progranulin protein in the plasma and/or in the cerebrospinal fluid of the individual of at least about any of 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or more, compared to prior to administration of the anti-sortilin antibody.

In some embodiments, the increase in the level of progranulin protein in the plasma and/or in the cerebrospinal fluid of the individual is present at any of about 1 hour, about 2 hours, about 4 hours, about 6 hours, about 8 hours, about 10 hours, about 12 hours, about 14 hours, about 16 hours, about 18 hours, about 20 hours, about 22 hours, about 24 hours, or more after administration of the anti-sortilin antibody. In some embodiments, the increase in the level of progranulin protein in the plasma and/or in the cerebrospinal fluid of the individual is present at any of about 24 hours, about 36 hours, about 48 hours, about 60 hours, about 72 hours, or more after administration of the anti-sortilin antibody.

In some embodiments, the increase in the level of progranulin protein in the plasma and/or in the cerebrospinal fluid of the individual is present at any of about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, about 50 days, about 51 days, about 52 days, about 53 days, about 54 days, about 55 days, about 56 days, about 57 days, about 58 days, about 59 days, about 60 days, about 61 days, about 62 days, about 63 days, about 64 days, about 65 days, about 66 days, about 67 days, about 68 days, about 69 days, about 70 days, about 71 days, about 72 days, about 73 days, about 74 days, about 75 days, about 76 days, about 77 days, about 78 days, about 79 days, about 80 days, about 81 days, about 82 days, about 83 days, about 84 days, about 85 days, about 86 days, about 87 days, about 88 days, about 89 days, about 90 days, about 91 days, about 92 days, about 93 days, about 94 days, about 95 days, about 96 days, about 97 days, about 98 days, about 99 days, about 100 days, about 101 days, about 102 days, about 103 days, about 104 days, about 105 days, about 106 days, about 107 days, about 108 days, about 109 days, about 110 days, about 111 days, about 112 days, about 113 days, about 114 days, about 115 days, about 116 days, about 117 days, about 118 days, about 119 days, about 120 days, about 121 days, about 122 days, about 123 days, about 124 days, about 125 days, about 126 days, about 127 days, about 1280 days, about 129 days, about 130 days, about 131 days, about 132 days, about 133 days, about 134 days, about 135 days, about 136 days, about 137 days, about 138 days, about 139 days, about 140 days, about 141 days, about 142 days, about 143 days, about 144 days, about 145 days, about 146 days, about 147 days, about 148 days, about 149 days, about 150 days, about 151 days, about 152 days, about 153 days, about 154 days, about 155 days, about 156 days, about 157 days, about 158 days, about 159 days, about 160 days, about 161 days, about 162 days, about 163 days, about 164 days, about 165 days, about 166 days, about 167 days, about 168 days, about 169 days, about 170 days, about 171 days, about 172 days, about 173 days, about 174 days, about 175 days, about 176 days, about 177 days, about 178 days, about 179 days, about 180 days, about 181 days, about 182 days, about 183 days, about 184 days, about 185 days, about 186 days, about 187 days, about 188 days, about 189 days, about 190 days, about 191 days, about 192 days, about 193 days, about 194 days, about 195 days, about 196 days, about 197 days, about 198 days, about 199 days, about 200 days, about 201 days, or more after administration of the anti-sortilin antibody.

In some embodiments, intravenous administration of a dose of about 6 mg/kg of an anti-sortilin antibody of the disclosure to an individual according to the methods provided herein results in an increase in the level of progranulin protein in the plasma of the individual of at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.1-fold, at least about 2.2-fold, at least about 2.3-fold, at least about 2.4-fold, at least about 2.5-fold, at least about 2.6-fold, at least about 2.7-fold, at least about 2.8-fold, at least about 2.9-fold, at least about 3-fold, at least about 3.1-fold, at least about 3.2-fold, at least about 3.3-fold, at least about 3.4-fold, at least about 3.5-fold, at least about 3.6-fold, at least about 3.7-fold, at least about 3.8-fold, at least about 3.9-fold, at least about 4-fold, at least about 4.1-fold, at least about 4.2-fold, at least about 4.3-fold, at least about 4.4-fold, at least about 4.5-fold, at least about 4.6-fold, at least about 4.7-fold, at least about 4.8-fold, at least about 4.9-fold, at least about 5-fold, or more, compared to the level of progranulin protein in the plasma of the individual prior to administration of the anti-sortilin antibody. In some embodiments, intravenous administration of a dose of about 6 mg/kg of an anti-sortilin antibody of the disclosure to an individual according to the methods provided herein results in an increase in the level of progranulin protein in the plasma of the individual of at least about 2-fold or more compared to the level of progranulin protein in the plasma of the individual prior to administration of the anti-sortilin antibody. In some embodiments, intravenous administration of a dose of about 6 mg/kg of an anti-sortilin antibody of the disclosure to an individual according to the methods provided herein results in an increase in the level of progranulin protein in the plasma of the individual of at least about 3-fold or more compared to the level of progranulin protein in the plasma of the individual prior to administration of the anti-sortilin antibody. In some embodiments, intravenous administration of a dose of about 6 mg/kg of an anti-sortilin antibody of the disclosure to an individual according to the methods provided herein results in an increase in the level of progranulin protein in the plasma of the individual of at least about any of 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, or more, compared to the level of progranulin protein in the plasma of the individual prior to administration of the anti-sortilin antibody. In some embodiments, the increase in the level of progranulin protein in the plasma of the individual is present at about 1 day, at about 2 days, at about 3 days, at about 6 days, at about 7 days, at about 8 days, at about 13 days, at about 14 days, at about 18 days, at about 21 days, at about 23 days, at about 24 days, at about 25 days, at about 26 days, at about 27 days, at about 28 days, at about 29 days, at about 30 days, at about 31 days, at about 32 days, at about 33 days, at about 34 days, at about 35 days, at about 36 days, at about 37 days, at about 38 days, at about 39 days, at about 40 days, at about 41 days, at about 42 days, at about 43 days, at about 44 days, at about 45 days, or more, after administration of the anti-sortilin antibody. In some embodiments, the increase in the level of progranulin protein in the plasma of the individual is present from within about 1 day after administration of the anti-sortilin antibody to about 43 days or more after administration of the anti-sortilin antibody. In some embodiments, the increase in the level of progranulin protein in the plasma of the individual is present at about 42 days or more after administration of the anti-sortilin antibody. In some embodiments, the increase in the level of progranulin protein in the plasma of the individual is present at about 43 days or more after administration of the anti-sortilin antibody. In some embodiments, the increase in the level of progranulin protein in the plasma of the individual is present at about 28 days or more after administration of the anti-sortilin antibody.

In some embodiments, intravenous administration of a dose of about 15 mg/kg of an anti-sortilin antibody of the disclosure to an individual according to the methods provided herein results in an increase in the level of progranulin protein in the plasma of the individual of at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.1-fold, at least about 2.2-fold, at least about 2.3-fold, at least about 2.4-fold, at least about 2.5-fold, at least about 2.6-fold, at least about 2.7-fold, at least about 2.8-fold, at least about 2.9-fold, at least about 3-fold, at least about 3.1-fold, at least about 3.2-fold, at least about 3.3-fold, at least about 3.4-fold, at least about 3.5-fold, at least about 3.6-fold, at least about 3.7-fold, at least about 3.8-fold, at least about 3.9-fold, at least about 4-fold, at least about 4.1-fold, at least about 4.2-fold, at least about 4.3-fold, at least about 4.4-fold, at least about 4.5-fold, at least about 4.6-fold, at least about 4.7-fold, at least about 4.8-fold, at least about 4.9-fold, at least about 5-fold, or more, compared to the level of progranulin protein in the plasma of the individual prior to administration of the anti-sortilin antibody. In some embodiments, intravenous administration of a dose of about 15 mg/kg of an anti-sortilin antibody of the disclosure to an individual according to the methods provided herein results in an increase in the level of progranulin protein in the plasma of the individual of at least about 2-fold or more compared to the level of progranulin protein in the plasma of the individual prior to administration of the anti-sortilin antibody. In some embodiments, intravenous administration of a dose of about 15 mg/kg of an anti-sortilin antibody of the disclosure to an individual according to the methods provided herein results in an increase in the level of progranulin protein in the plasma of the individual of at least about 3-fold or more compared to the level of progranulin protein in the plasma of the individual prior to administration of the anti-sortilin antibody. In some embodiments, intravenous administration of a dose of about 15 mg/kg of an anti-sortilin antibody of the disclosure to an individual according to the methods provided herein results in an increase in the level of progranulin protein in the plasma of the individual of at least about any of 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, or more, compared to the level of progranulin protein in the plasma of the individual prior to administration of the anti-sortilin antibody. In some embodiments, the increase in the level of progranulin protein in the plasma of the individual is present at about 1 day, at about 2 days, at about 3 days, at about 6 days, at about 7 days, at about 8 days, at about 13 days, at about 14 days, at about 18 days, at about 21 days, at about 23 days, at about 24 days, at about 25 days, at about 26 days, at about 27 days, at about 28 days, at about 29 days, at about 30 days, at about 31 days, at about 32 days, at about 33 days, at about 34 days, at about 35 days, at about 36 days, at about 37 days, at about 38 days, at about 39 days, at about 40 days, at about 41 days, at about 42 days, at about 43 days, at about 44 days, at about 45 days, at about 46 days, at about 47 days, at about 48 days, at about 49 days, at about 50 days, at about 51 days, at about 52 days, at about 53 days, at about 54 days, at about 55 days, at about 56 days, at about 57 days, at about 58 days, at about 59 days, at about 60 days, or more, after administration of the anti-sortilin antibody. In some embodiments, the increase in the level of progranulin protein in the plasma of the individual is present from within about 1 day after administration of the anti-sortilin antibody to about 57 days or more after administration of the anti-sortilin antibody. In some embodiments, the increase in the level of progranulin protein in the plasma of the individual is present at about 57 days or more after administration of the anti-sortilin antibody. In some embodiments, the increase in the level of progranulin protein in the plasma of the individual is present at about 42 days or more after administration of the anti-sortilin antibody.

In some embodiments, intravenous administration of a dose of about 30 mg/kg of an anti-sortilin antibody of the disclosure to an individual according to the methods provided herein results in an increase in the level of progranulin protein in the plasma of the individual of at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.1-fold, at least about 2.2-fold, at least about 2.3-fold, at least about 2.4-fold, at least about 2.5-fold, at least about 2.6-fold, at least about 2.7-fold, at least about 2.8-fold, at least about 2.9-fold, at least about 3-fold, at least about 3.1-fold, at least about 3.2-fold, at least about 3.3-fold, at least about 3.4-fold, at least about 3.5-fold, at least about 3.6-fold, at least about 3.7-fold, at least about 3.8-fold, at least about 3.9-fold, at least about 4-fold, at least about 4.1-fold, at least about 4.2-fold, at least about 4.3-fold, at least about 4.4-fold, at least about 4.5-fold, at least about 4.6-fold, at least about 4.7-fold, at least about 4.8-fold, at least about 4.9-fold, at least about 5-fold, or more, compared to the level of progranulin protein in the plasma of the individual prior to administration of the anti-sortilin antibody. In some embodiments, intravenous administration of a dose of about 30 mg/kg of an anti-sortilin antibody of the disclosure to an individual according to the methods provided herein results in an increase in the level of progranulin protein in the plasma of the individual of at least about 2-fold or more compared to the level of progranulin protein in the plasma of the individual prior to administration of the anti-sortilin antibody. In some embodiments, intravenous administration of a dose of about 30 mg/kg of an anti-sortilin antibody of the disclosure to an individual according to the methods provided herein results in an increase in the level of progranulin protein in the plasma of the individual of at least about 3-fold or more compared to the level of progranulin protein in the plasma of the individual prior to administration of the anti-sortilin antibody. In some embodiments, intravenous administration of a dose of about 30 mg/kg of an anti-sortilin antibody of the disclosure to an individual according to the methods provided herein results in an increase in the level of progranulin protein in the plasma of the individual of at least about any of 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or more, compared to the level of progranulin protein in the plasma of the individual prior to administration of the anti-sortilin antibody. In some embodiments, the increase in the level of progranulin protein in the plasma of the individual is present at about 1 day, at about 2 days, at about 3 days, at about 6 days, at about 7 days, at about 8 days, at about 13 days, at about 18 days, at about 21 days, at about 23 days, at about 24 days, at about 25 days, at about 26 days, at about 27 days, at about 28 days, at about 29 days, at about 30 days, at about 31 days, at about 32 days, at about 33 days, at about 34 days, at about 35 days, at about 36 days, at about 37 days, at about 38 days, at about 39 days, at about 40 days, at about 41 days, at about 42 days, at about 43 days, at about 44 days, at about 45 days, at about 46 days, at about 47 days, at about 48 days, at about 49 days, at about 50 days, at about 51 days, at about 52 days, at about 53 days, at about 54 days, at about 55 days, at about 56 days, at about 57 days, at about 58 days, at about 59 days, at about 60 days, or more, after administration of the anti-sortilin antibody. In some embodiments, the increase in the level of progranulin protein in the plasma of the individual is present from within about 1 day after administration of the anti-sortilin antibody to about 57 days or more after administration of the anti-sortilin antibody. In some embodiments, the increase in the level of progranulin protein in the plasma of the individual is present at about 56 days or more after administration of the anti-sortilin antibody. In some embodiments, the increase in the level of progranulin protein in the plasma of the individual is present at about 57 days or more after administration of the anti-sortilin antibody.

In some embodiments, intravenous administration of a dose of about 60 mg/kg of an anti-sortilin antibody of the disclosure to an individual according to the methods provided herein results in an increase in the level of progranulin protein in the plasma of the individual of at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.1-fold, at least about 2.2-fold, at least about 2.3-fold, at least about 2.4-fold, at least about 2.5-fold, at least about 2.6-fold, at least about 2.7-fold, at least about 2.8-fold, at least about 2.9-fold, at least about 3-fold, at least about 3.1-fold, at least about 3.2-fold, at least about 3.3-fold, at least about 3.4-fold, at least about 3.5-fold, at least about 3.6-fold, at least about 3.7-fold, at least about 3.8-fold, at least about 3.9-fold, at least about 4-fold, at least about 4.1-fold, at least about 4.2-fold, at least about 4.3-fold, at least about 4.4-fold, at least about 4.5-fold, at least about 4.6-fold, at least about 4.7-fold, at least about 4.8-fold, at least about 4.9-fold, at least about 5-fold, or more, compared to the level of progranulin protein in the plasma of the individual prior to administration of the anti-sortilin antibody. In some embodiments, intravenous administration of a dose of about 60 mg/kg of an anti-sortilin antibody of the disclosure to an individual according to the methods provided herein results in an increase in the level of progranulin protein in the plasma of the individual of at least about 2-fold or more compared to the level of progranulin protein in the plasma of the individual prior to administration of the anti-sortilin antibody. In some embodiments, intravenous administration of a dose of about 60 mg/kg of an anti-sortilin antibody of the disclosure to an individual according to the methods provided herein results in an increase in the level of progranulin protein in the plasma of the individual of at least about 3-fold or more compared to the level of progranulin protein in the plasma of the individual prior to administration of the anti-sortilin antibody. In some embodiments, intravenous administration of a dose of about 60 mg/kg of an anti-sortilin antibody of the disclosure to an individual according to the methods provided herein results in an increase in the level of progranulin protein in the plasma of the individual of at least about any of 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or more, compared to the level of progranulin protein in the plasma of the individual prior to administration of the anti-sortilin antibody. In some embodiments, the increase in the level of progranulin protein in the plasma of the individual is present at about 1 day, at about 2 days, at about 3 days, at about 6 days, at about 7 days, at about 8 days, at about 13 days, at about 14 days, at about 18 days, at about 21 days, at about 23 days, at about 24 days, at about 25 days, at about 26 days, at about 27 days, at about 28 days, at about 29 days, at about 30 days, at about 31 days, at about 32 days, at about 33 days, at about 34 days, at about 35 days, at about 36 days, at about 37 days, at about 38 days, at about 39 days, at about 40 days, at about 41 days, at about 42 days, at about 43 days, at about 44 days, at about 45 days, at about 46 days, at about 47 days, at about 48 days, at about 49 days, at about 50 days, at about 51 days, at about 52 days, at about 53 days, at about 54 days, at about 55 days, at about 56 days, at about 57 days, at about 58 days, at about 59 days, at about 60 days, at about 61 days, at about 62 days, at about 63 days, at about 64 days, at about 65 days, at about 66 days, at about 67 days, at about 68 days, at about 69 days, at about 70 days, at about 71 days, at about 72 days, at about 73 days, at about 74 days, at about 75 days, at about 76 days, at about 77 days, at about 78 days, at about 79 days, at about 80 days, at about 81 days, at about 82 days, at about 83 days, at about 84 days, at about 85 days, at about 86 days, at about 87 days, at about 88 days, at about 89 days, at about 90 days, at about 91 days, at about 92 days, at about 93 days, at about 94 days, at about 95 days, at about 96 days, at about 97 days, at about 98 days, at about 99 days, at about 100 days, at about 101 days, at about 102 days, at about 103 days, at about 104 days, at about 105 days, at about 106 days, at about 107 days, at about 108 days, at about 109 days, at about 110 days, at about 111 days, at about 112 days, at about 113 days, at about 114 days, at about 115 days, at about 116 days, at about 117 days, at about 118 days, at about 119 days, at about 120 days, or more, after administration of the anti-sortilin antibody. In some embodiments, the increase in the level of progranulin protein in the plasma of the individual is present from within about 1 day after administration of the anti-sortilin antibody to about 57 days or more after administration of the anti-sortilin antibody. In some embodiments, the increase in the level of progranulin protein in the plasma of the individual is present from within about 1 day after administration of the anti-sortilin antibody to about 84 days or more after administration of the anti-sortilin antibody. In some embodiments, the increase in the level of progranulin protein in the plasma of the individual is present at about 57 days or more after administration of the anti-sortilin antibody. In some embodiments, the increase in the level of progranulin protein in the plasma of the individual is present at about 84 days or more after administration of the anti-sortilin antibody.

In some embodiments, subcutaneous administration of a dose of about 150 mg of an anti-sortilin antibody of the disclosure to an individual according to the methods provided herein results in an increase in the level of progranulin protein in the plasma of the individual of at least about any of 5%, 10%, 15%, 20%, 25%, 30%, 35%, 40%, 45%, 50%, 55% 60%, 65%, 70%, 75%, 80%, 85%, 90%, 95%, 100%, 110%, 120%, 130%, 140%, 150%, 160%, 170%, 180%, 190%, 200%, or more, compared to the level of progranulin protein in the plasma of the individual prior to administration of the anti-sortilin antibody. In some embodiments, the increase in the level of progranulin protein in the plasma of the individual is present at about 1 day, at about 2 days, at about 3 days, at about 6 days, at about 7 days, at about 8 days, at about 13 days, at about 14 days, at about 18 days, at about 21 days, at about 23 days, at about 24 days, at about 25 days, at about 26 days, at about 27 days, at about 28 days, or more, after administration of the anti-sortilin antibody. In some embodiments, the increase in the level of progranulin protein in the plasma of the individual is present from within about 1 day after administration of the anti-sortilin antibody to about 28 days or more after administration of the anti-sortilin antibody. In some embodiments, the increase in the level of progranulin protein in the plasma of the individual is present at about 28 days or more after administration of the anti-sortilin antibody.

In some embodiments, subcutaneous administration of a dose of about 300 mg of an anti-sortilin antibody of the disclosure to an individual according to the methods provided herein results in an increase in the level of progranulin protein in the plasma of the individual of at least about any of 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, or more, compared to the level of progranulin protein in the plasma of the individual prior to administration of the anti-sortilin antibody. In some embodiments, the increase in the level of progranulin protein in the plasma of the individual is present at about 1 day, at about 2 days, at about 3 days, at about 6 days, at about 7 days, at about 8 days, at about 13 days, at about 14 days, at about 18 days, at about 21 days, at about 23 days, at about 24 days, at about 25 days, at about 26 days, at about 27 days, at about 28 days, or more, after administration of the anti-sortilin antibody. In some embodiments, the increase in the level of progranulin protein in the plasma of the individual is present from within about 1 day after administration of the anti-sortilin antibody to about 28 days or more after administration of the anti-sortilin antibody. In some embodiments, the increase in the level of progranulin protein in the plasma of the individual is present at about 28 days or more after administration of the anti-sortilin antibody.

In some embodiments, subcutaneous administration of a dose of about 600 mg of an anti-sortilin antibody of the disclosure to an individual according to the methods provided herein results in an increase in the level of progranulin protein in the plasma of the individual of at least about any of 5%, 10%, 15%, 20%, 25%, 30%, 35%, 40%, 45%, 50%, 55%, 60%, 6%, 70%, 75%, 80%, 85%, 90%, 95%, 100%, 110%, 120%, 130%, 140%, 150%, 160%, 170%, 180%, 190%, 200%, or more, compared to the level of progranulin protein in the plasma of the individual prior to administration of the anti-sortilin antibody. In some embodiments, the increase in the level of progranulin protein in the plasma of the individual is present at about 1 day, at about 2 days, at about 3 days, at about 6 days, at about 7 days, at about 8 days, at about 13 days, at about 14 days, at about 18 days, at about 21 days, at about 23 days, at about 24 days, at about 25 days, at about 26 days, at about 27 days, at about 28 days, or more, after administration of the anti-sortilin antibody. In some embodiments, the increase in the level of progranulin protein in the plasma of the individual is present from within about 1 day after administration of the anti-sortilin antibody to about 28 days or more after administration of the anti-sortilin antibody. In some embodiments, the increase in the level of progranulin protein in the plasma of the individual is present at about 28 days or more after administration of the anti-sortilin antibody.

In some embodiments, intravenous administration of a dose of about 6 mg/kg of an anti-sortilin antibody of the disclosure to an individual according to the methods provided herein results in an increase in the level of progranulin protein in the cerebrospinal fluid of the individual of at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, or more, compared to the level of progranulin protein in the cerebrospinal fluid of the individual prior to administration of the anti-sortilin antibody. In some embodiments, intravenous administration of a dose of about 6 mg/kg of an anti-sortilin antibody of the disclosure to an individual according to the methods provided herein results in an increase in the level of progranulin protein in the cerebrospinal fluid of the individual of at least about 5% or more compared to the level of progranulin protein in the cerebrospinal fluid of the individual prior to administration of the anti-sortilin antibody. In some embodiments, intravenous administration of a dose of about 6 mg/kg of an anti-sortilin antibody of the disclosure to an individual according to the methods provided herein results in an increase in the level of progranulin protein in the cerebrospinal fluid of the individual of at least about 10% or more compared to the level of progranulin protein in the cerebrospinal fluid of the individual prior to administration of the anti-sortilin antibody. In some embodiments, intravenous administration of a dose of about 6 mg/kg of an anti-sortilin antibody of the disclosure to an individual according to the methods provided herein results in an increase in the level of progranulin protein in the cerebrospinal fluid of the individual of at least about 12% or more compared to the level of progranulin protein in the cerebrospinal fluid of the individual prior to administration of the anti-sortilin antibody. In some embodiments, intravenous administration of a dose of about 6 mg/kg of an anti-sortilin antibody of the disclosure to an individual according to the methods provided herein results in an increase in the level of progranulin protein in the cerebrospinal fluid of the individual of at least about 15% or more compared to the level of progranulin protein in the cerebrospinal fluid of the individual prior to administration of the anti-sortilin antibody. In some embodiments, intravenous administration of a dose of about 6 mg/kg of an anti-sortilin antibody of the disclosure to an individual according to the methods provided herein results in an increase in the level of progranulin protein in the cerebrospinal fluid of the individual of at least about 18% or more compared to the level of progranulin protein in the cerebrospinal fluid of the individual prior to administration of the anti-sortilin antibody. In some embodiments, the increase in the level of progranulin protein in the cerebrospinal fluid of the individual is present at about 1 day, at about 2 days, at about 3 days, at about 6 days, at about 7 days, at about 8 days, at about 13 days, at about 14 days, at about 18 days, at about 21 days, at about 23 days, at about 24 days, at about 25 days, at about 26 days, at about 27 days, at about 28 days, at about 29 days, at about 30 days, at about 31 days, at about 32 days, at about 33 days, at about 34 days, at about 35 days, at about 36 days, at about 37 days, at about 38 days, at about 39 days, at about 40 days, at about 41 days, at about 42 days, at about 43 days, at about 44 days, at about 45 days, at about 46 days, at about 47 days, at about 48 days, at about 49 days, at about 50 days, at about 51 days, at about 52 days, at about 53 days, at about 54 days, at about 55 days, at about 56 days, at about 57 days, at about 58 days, at about 59 days, at about 60 days, or more, after administration of the anti-sortilin antibody. In some embodiments, the increase in the level of progranulin protein in the cerebrospinal fluid of the individual is present from within about 1 day after administration of the anti-sortilin antibody to about 57 days or more after administration of the anti-sortilin antibody. In some embodiments, the increase in the level of progranulin protein in the cerebrospinal fluid of the individual is present at about 25 days or more after administration of the anti-sortilin antibody. In some embodiments, the increase in the level of progranulin protein in the cerebrospinal fluid of the individual is present at about 43 days or more after administration of the anti-sortilin antibody. In some embodiments, the increase in the level of progranulin protein in the cerebrospinal fluid of the individual is present at about 57 days or more after administration of the anti-sortilin antibody.

In some embodiments, intravenous administration of a dose of about 15 mg/kg of an anti-sortilin antibody of the disclosure to an individual according to the methods provided herein results in an increase in the level of progranulin protein in the cerebrospinal fluid of the individual of at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, or more, compared to the level of progranulin protein in the cerebrospinal fluid of the individual prior to administration of the anti-sortilin antibody. In some embodiments, intravenous administration of a dose of about 15 mg/kg of an anti-sortilin antibody of the disclosure to an individual according to the methods provided herein results in an increase in the level of progranulin protein in the cerebrospinal fluid of the individual of at least about 15% or more compared to the level of progranulin protein in the cerebrospinal fluid of the individual prior to administration of the anti-sortilin antibody. In some embodiments, intravenous administration of a dose of about 15 mg/kg of an anti-sortilin antibody of the disclosure to an individual according to the methods provided herein results in an increase in the level of progranulin protein in the cerebrospinal fluid of the individual of at least about 60% or more compared to the level of progranulin protein in the cerebrospinal fluid of the individual prior to administration of the anti-sortilin antibody. In some embodiments, intravenous administration of a dose of about 15 mg/kg of an anti-sortilin antibody of the disclosure to an individual according to the methods provided herein results in an increase in the level of progranulin protein in the cerebrospinal fluid of the individual of at least about 80% or more compared to the level of progranulin protein in the cerebrospinal fluid of the individual prior to administration of the anti-sortilin antibody. In some embodiments, the increase in the level of progranulin protein in the cerebrospinal fluid of the individual is present at about 1 day, at about 2 days, at about 3 days, at about 6 days, at about 7 days, at about 8 days, at about 13 days, at about 14 days, at about 18 days, at about 21 days, at about 23 days, at about 24 days, at about 25 days, at about 26 days, at about 27 days, at about 28 days, at about 29 days, at about 30 days, at about 31 days, at about 32 days, at about 33 days, at about 34 days, at about 35 days, at about 36 days, at about 37 days, at about 38 days, at about 39 days, at about 40 days, at about 41 days, at about 42 days, at about 43 days, at about 44 days, at about 45 days, at about 46 days, at about 47 days, at about 48 days, at about 49 days, at about 50 days, at about 51 days, at about 52 days, at about 53 days, at about 54 days, at about 55 days, at about 56 days, at about 57 days, at about 58 days, at about 59 days, at about 60 days, or more, after administration of the anti-sortilin antibody. In some embodiments, the increase in the level of progranulin protein in the cerebrospinal fluid of the individual is present from within about 1 day after administration of the anti-sortilin antibody to about 57 days or more after administration of the anti-sortilin antibody. In some embodiments, the increase in the level of progranulin protein in the cerebrospinal fluid of the individual is present at about 25 days or more after administration of the anti-sortilin antibody. In some embodiments, the increase in the level of progranulin protein in the cerebrospinal fluid of the individual is present at about 43 days or more after administration of the anti-sortilin antibody. In some embodiments, the increase in the level of progranulin protein in the cerebrospinal fluid of the individual is present at about 57 days or more after administration of the anti-sortilin antibody.

In some embodiments, intravenous administration of a dose of about 30 mg/kg of an anti-sortilin antibody of the disclosure to an individual according to the methods provided herein results in an increase in the level of progranulin protein in the cerebrospinal fluid of the individual of at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, or more, compared to the level of progranulin protein in the cerebrospinal fluid of the individual prior to administration of the anti-sortilin antibody. In some embodiments, intravenous administration of a dose of about 30 mg/kg of an anti-sortilin antibody of the disclosure to an individual according to the methods provided herein results in an increase in the level of progranulin protein in the cerebrospinal fluid of the individual of at least about 15% or more compared to the level of progranulin protein in the cerebrospinal fluid of the individual prior to administration of the anti-sortilin antibody. In some embodiments, intravenous administration of a dose of about 30 mg/kg of an anti-sortilin antibody of the disclosure to an individual according to the methods provided herein results in an increase in the level of progranulin protein in the cerebrospinal fluid of the individual of at least about 60% or more compared to the level of progranulin protein in the cerebrospinal fluid of the individual prior to administration of the anti-sortilin antibody. In some embodiments, intravenous administration of a dose of about 30 mg/kg of an anti-sortilin antibody of the disclosure to an individual according to the methods provided herein results in an increase in the level of progranulin protein in the cerebrospinal fluid of the individual of at least about 80% or more compared to the level of progranulin protein in the cerebrospinal fluid of the individual prior to administration of the anti-sortilin antibody. In some embodiments, the increase in the level of progranulin protein in the cerebrospinal fluid of the individual is present at about 1 day, at about 2 days, at about 3 days, at about 6 days, at about 7 days, at about 8 days, at about 13 days, at about 14 days, at about 18 days, at about 21 days, at about 23 days, at about 24 days, at about 25 days, at about 26 days, at about 27 days, at about 28 days, at about 29 days, at about 30 days, at about 31 days, at about 32 days, at about 33 days, at about 34 days, at about 35 days, at about 36 days, at about 37 days, at about 38 days, at about 39 days, at about 40 days, at about 41 days, at about 42 days, at about 43 days, at about 44 days, at about 45 days, at about 46 days, at about 47 days, at about 48 days, at about 49 days, at about 50 days, at about 51 days, at about 52 days, at about 53 days, at about 54 days, at about 55 days, at about 56 days, at about 57 days, at about 58 days, at about 59 days, at about 60 days, at about 61 days, at about 62 days, at about 63 days, at about 64 days, at about 65 days, at about 66 days, at about 67 days, at about 68 days, at about 69 days, at about 70 days, at about 71 days, at about 72 days, at about 73 days, at about 74 days, at about 75 days, at about 76 days, at about 77 days, at about 78 days, at about 79 days, at about 80 days, at about 81 days, at about 82 days, at about 83 days, at about 84 days, or more, after administration of the anti-sortilin antibody. In some embodiments, the increase in the level of progranulin protein in the cerebrospinal fluid of the individual is present from within about 1 day after administration of the anti-sortilin antibody to about 57 days or more after administration of the anti-sortilin antibody. In some embodiments, the increase in the level of progranulin protein in the cerebrospinal fluid of the individual is present at about 25 days or more after administration of the anti-sortilin antibody. In some embodiments, the increase in the level of progranulin protein in the cerebrospinal fluid of the individual is present at about 43 days or more after administration of the anti-sortilin antibody. In some embodiments, the increase in the level of progranulin protein in the cerebrospinal fluid of the individual is present at about 57 days or more after administration of the anti-sortilin antibody.

In some embodiments, intravenous administration of a dose of about 60 mg/kg of an anti-sortilin antibody of the disclosure to an individual according to the methods provided herein results in an increase in the level of progranulin protein in the cerebrospinal fluid of the individual of at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, or more, compared to the level of progranulin protein in the cerebrospinal fluid of the individual prior to administration of the anti-sortilin antibody. In some embodiments, intravenous administration of a dose of about 60 mg/kg of an anti-sortilin antibody of the disclosure to an individual according to the methods provided herein results in an increase in the level of progranulin protein in the cerebrospinal fluid of the individual of at least about 15% or more compared to the level of progranulin protein in the cerebrospinal fluid of the individual prior to administration of the anti-sortilin antibody. In some embodiments, intravenous administration of a dose of about 60 mg/kg of an anti-sortilin antibody of the disclosure to an individual according to the methods provided herein results in an increase in the level of progranulin protein in the cerebrospinal fluid of the individual of at least about 60% or more compared to the level of progranulin protein in the cerebrospinal fluid of the individual prior to administration of the anti-sortilin antibody. In some embodiments, intravenous administration of a dose of about 60 mg/kg of an anti-sortilin antibody of the disclosure to an individual according to the methods provided herein results in an increase in the level of progranulin protein in the cerebrospinal fluid of the individual of at least about 80% or more compared to the level of progranulin protein in the cerebrospinal fluid of the individual prior to administration of the anti-sortilin antibody. In some embodiments, the increase in the level of progranulin protein in the cerebrospinal fluid of the individual is present at about 1 day, at about 2 days, at about 3 days, at about 6 days, at about 8 days, at about 13 days, at about 18 days, at about 23 days, at about 24 days, at about 25 days, at about 26 days, at about 27 days, at about 28 days, at about 29 days, at about 30 days, at about 31 days, at about 32 days, at about 33 days, at about 34 days, at about 35 days, at about 36 days, at about 37 days, at about 38 days, at about 39 days, at about 40 days, at about 41 days, at about 42 days, at about 43 days, at about 44 days, at about 45 days, at about 46 days, at about 47 days, at about 48 days, at about 49 days, at about 50 days, at about 51 days, at about 52 days, at about 53 days, at about 54 days, at about 55 days, at about 56 days, at about 57 days, at about 58 days, at about 59 days, at about 60 days, at about 61 days, at about 62 days, at about 63 days, at about 64 days, at about 65 days, at about 66 days, at about 67 days, at about 68 days, at about 69 days, at about 70 days, at about 71 days, at about 72 days, at about 73 days, at about 74 days, at about 75 days, at about 76 days, at about 77 days, at about 78 days, at about 79 days, at about 80 days, at about 81 days, at about 82 days, at about 83 days, at about 84 days, at about 85 days, at about 86 days, at about 87 days, at about 88 days, at about 89 days, at about 90 days, at about 91 days, at about 92 days, at about 93 days, at about 94 days, at about 95 days, at about 96 days, at about 97 days, at about 98 days, at about 99 days, at about 100 days, at about 101 days, at about 102 days, at about 103 days, at about 104 days, at about 105 days, at about 106 days, at about 107 days, at about 108 days, at about 109 days, at about 110 days, at about 111 days, at about 112 days, at about 113 days, at about 114 days, at about 115 days, at about 116 days, at about 117 days, at about 118 days, at about 119 days, at about 120 days, or more, after administration of the anti-sortilin antibody. In some embodiments, the increase in the level of progranulin protein in the cerebrospinal fluid of the individual is present from within about 1 day after administration of the anti-sortilin antibody to about 57 days or more after administration of the anti-sortilin antibody. In some embodiments, the increase in the level of progranulin protein in the cerebrospinal fluid of the individual is present at about 25 days or more after administration of the anti-sortilin antibody. In some embodiments, the increase in the level of progranulin protein in the cerebrospinal fluid of the individual is present at about 43 days or more after administration of the anti-sortilin antibody. In some embodiments, the increase in the level of progranulin protein in the cerebrospinal fluid of the individual is present at about 57 days or more after administration of the anti-sortilin antibody. In some embodiments, the increase in the level of progranulin protein in the cerebrospinal fluid of the individual is present at about 84 days or more after administration of the anti-sortilin antibody.

In some embodiments, subcutaneous administration of a dose of about 150 mg of an anti-sortilin antibody of the disclosure to an individual according to the methods provided herein results in an increase in the level of progranulin protein in the cerebrospinal fluid of the individual of at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 30%, at least about 35%, at least about 40%, at least about 45%, at least about 50%, at least about 55%, at least about 60%, at least about 65%, at least about 70%, or more, compared to the level of progranulin protein in the cerebrospinal fluid of the individual prior to administration of the anti-sortilin antibody. In some embodiments, subcutaneous administration of a dose of about 300 mg of an anti-sortilin antibody of the disclosure to an individual according to the methods provided herein results in an increase in the level of progranulin protein in the cerebrospinal fluid of the individual of at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 30%, at least about 35%, at least about 40%, at least about 45%, at least about 50%, at least about 55%, at least about 60%, at least about 65%, at least about 70%, or more, compared to the level of progranulin protein in the cerebrospinal fluid of the individual prior to administration of the anti-sortilin antibody. In some embodiments, the increase in the level of progranulin protein in the cerebrospinal fluid of the individual is present at about 1 day, at about 2 days, at about 3 days, at about 6 days, at about 8 days, at about 13 days, at about 18 days, at about 23 days, at about 24 days, at about 25 days, at about 26 days, at about 27 days, at about 28 days, at about 29 days, at about 30 days, at about 31 days, at about 32 days, at about 33 days, at about 34 days, at about 35 days, at about 36 days, at about 37 days, at about 38 days, at about 39 days, at about 40 days, at about 41 days, at about 42 days, or more after administration of the anti-sortilin antibody. In some embodiments, the increase in the level of progranulin protein in the cerebrospinal fluid of the individual is present from within about 1 day after administration of the anti-sortilin antibody to about 21 days or more after administration of the anti-sortilin antibody. In some embodiments, the increase in the level of progranulin protein in the cerebrospinal fluid of the individual is present from within about 1 day after administration of the anti-sortilin antibody to about 42 days or more after administration of the anti-sortilin antibody. In some embodiments, the increase in the level of progranulin protein in the cerebrospinal fluid of the individual is present at about 7 days or more after administration of the anti-sortilin antibody. In some embodiments, the increase in the level of progranulin protein in the cerebrospinal fluid of the individual is present at about 14 days or more after administration of the anti-sortilin antibody. In some embodiments, the increase in the level of progranulin protein in the cerebrospinal fluid of the individual is present at about 27 days or more after administration of the anti-sortilin antibody.

In some embodiments, subcutaneous administration of a dose of about 600 mg of an anti-sortilin antibody of the disclosure to an individual according to the methods provided herein results in an increase in the level of progranulin protein in the cerebrospinal fluid of the individual of at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 30%, at least about 35%, at least about 40%, at least about 45%, at least about 50%, at least about 55%, at least about 60%, at least about 65%, at least about 70%, or more, compared to the level of progranulin protein in the cerebrospinal fluid of the individual prior to administration of the anti-sortilin antibody. In some embodiments, subcutaneous administration of a dose of about 600 mg of an anti-sortilin antibody of the disclosure to an individual according to the methods provided herein results in an increase in the level of progranulin protein in the cerebrospinal fluid of the individual of at least about 40% or more compared to the level of progranulin protein in the cerebrospinal fluid of the individual prior to administration of the anti-sortilin antibody. In some embodiments, subcutaneous administration of a dose of about 600 mg of an anti-sortilin antibody of the disclosure to an individual according to the methods provided herein results in an increase in the level of progranulin protein in the cerebrospinal fluid of the individual of at least about 35% or more compared to the level of progranulin protein in the cerebrospinal fluid of the individual prior to administration of the anti-sortilin antibody. In some embodiments, subcutaneous administration of a dose of about 600 mg of an anti-sortilin antibody of the disclosure to an individual according to the methods provided herein results in an increase in the level of progranulin protein in the cerebrospinal fluid of the individual of at least about 20% or more compared to the level of progranulin protein in the cerebrospinal fluid of the individual prior to administration of the anti-sortilin antibody. In some embodiments, the increase in the level of progranulin protein in the cerebrospinal fluid of the individual is present at about 1 day, at about 2 days, at about 3 days, at about 6 days, at about 8 days, at about 13 days, at about 18 days, at about 23 days, at about 24 days, at about 25 days, at about 26 days, at about 27 days, at about 28 days, at about 29 days, at about 30 days, at about 31 days, at about 32 days, at about 33 days, at about 34 days, at about 35 days, at about 36 days, at about 37 days, at about 38 days, at about 39 days, at about 40 days, at about 41 days, at about 42 days, or more after administration of the anti-sortilin antibody. In some embodiments, the increase in the level of progranulin protein in the cerebrospinal fluid of the individual is present from within about 1 day after administration of the anti-sortilin antibody to about 21 days or more after administration of the anti-sortilin antibody. In some embodiments, the increase in the level of progranulin protein in the cerebrospinal fluid of the individual is present from within about 1 day after administration of the anti-sortilin antibody to about 42 days or more after administration of the anti-sortilin antibody. In some embodiments, the increase in the level of progranulin protein in the cerebrospinal fluid of the individual is present at about 7 days or more after administration of the anti-sortilin antibody. In some embodiments, the increase in the level of progranulin protein in the cerebrospinal fluid of the individual is present at about 14 days or more after administration of the anti-sortilin antibody. In some embodiments, the increase in the level of progranulin protein in the cerebrospinal fluid of the individual is present at about 27 days or more after administration of the anti-sortilin antibody.

In some embodiments, the level of progranulin protein in the plasma or the cerebrospinal fluid of the individual is measured in a sample obtained from the individual. In some embodiments, the level of progranulin protein in the plasma of the individual is measured in a sample of blood obtained from the individual. In some embodiments, the level of progranulin protein in the cerebrospinal fluid of the individual is measured in a sample of cerebrospinal fluid obtained from the individual. In some embodiments, the level of progranulin protein in the plasma or the cerebrospinal fluid of the individual is determined using any method of quantifying proteins known in the art. Non-limiting examples of methods that may be used to quantify progranulin protein include SOMASCAN assay (see, e.g., Candia et al. (2017) Sci Rep 7, 14248), Western blots, mass spectrometry, flow cytometry, and enzyme-linked immunosorbent assay (ELISA). In certain embodiments, the level of progranulin protein in the plasma or the cerebrospinal fluid of the individual is determined using ELISA assays.

### Sortilin

In some embodiments, the methods of treating or delaying progression of a disease or disorder provided herein further comprise measuring the level of sortilin protein in white blood cells. In some embodiments, the level of sortilin protein in white blood cells is measured in a sample of blood obtained from an individual before and after the individual has received one or more doses of an anti-sortilin antibody of the disclosure.

In some embodiments, administration to an individual of an anti-sortilin antibody of the disclosure according to the methods provided herein results in a reduction in the level of sortilin protein in white blood cells of the individual compared to the level of sortilin protein in white blood cells of the individual before administration of the anti-sortilin antibody. In some embodiments, the level of sortilin protein in white blood cells of the individual after administration of the anti-sortilin antibody is reduced by at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to the level of sortilin protein in white blood cells of the individual before administration of the anti-sortilin antibody. In some embodiments, the reduction in the level of sortilin in white blood cells of the individual is present at any of about 1 hour, about 2 hours, about 4 hours, about 6 hours, about 8 hours, about 10 hours, about 12 hours, about 14 hours, about 16 hours, about 18 hours, about 20 hours, about 22 hours, about 24 hours, or more after administration of the anti-sortilin antibody. In some embodiments, the reduction in the level of sortilin in white blood cells of the individual is present at any of about 24 hours, about 36 hours, about 48 hours, about 60 hours, about 72 hours, or more after administration of the anti-sortilin antibody. In some embodiments, the reduction in the level of sortilin in white blood cells of the individual is present at any of about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, about 50 days, about 51 days, about 52 days, about 53 days, about 54 days, about 55 days, about 56 days, about 57 days, about 58 days, about 59 days, about 60 days, about 61 days, about 62 days, about 63 days, about 64 days, about 65 days, about 66 days, about 67 days, about 68 days, about 69 days, about 70 days, about 71 days, about 72 days, about 73 days, about 74 days, about 75 days, about 76 days, about 77 days, about 78 days, about 79 days, about 80 days, about 81 days, about 82 days, about 83 days, about 84 days, about 85 days, about 86 days, about 87 days, about 88 days, about 89 days, about 90 days, about 91 days, about 92 days, about 93 days, about 94 days, about 95 days, about 96 days, about 97 days, about 98 days, about 99 days, about 100 days, about 101 days, about 102 days, about 103 days, about 104 days, about 105 days, about 106 days, about 107 days, about 108 days, about 109 days, about 110 days, about 111 days, about 112 days, about 113 days, about 114 days, about 115 days, about 116 days, about 117 days, about 118 days, about 119 days, about 120 days, about 121 days, about 122 days, about 123 days, about 124 days, about 125 days, about 126 days, about 127 days, about 1280 days, about 129 days, about 130 days, about 131 days, about 132 days, about 133 days, about 134 days, about 135 days, about 136 days, about 137 days, about 138 days, about 139 days, about 140 days, about 141 days, about 142 days, about 143 days, about 144 days, about 145 days, about 146 days, about 147 days, about 148 days, about 149 days, about 150 days, about 151 days, about 152 days, about 153 days, about 154 days, about 155 days, about 156 days, about 157 days, about 158 days, about 159 days, about 160 days, about 161 days, about 162 days, about 163 days, about 164 days, about 165 days, about 166 days, about 167 days, about 168 days, about 169 days, about 170 days, about 171 days, about 172 days, about 173 days, about 174 days, about 175 days, about 176 days, about 177 days, about 178 days, about 179 days, about 180 days, about 181 days, about 182 days, about 183 days, about 184 days, about 185 days, about 186 days, about 187 days, about 188 days, about 189 days, about 190 days, about 191 days, about 192 days, about 193 days, about 194 days, about 195 days, about 196 days, about 197 days, about 198 days, about 199 days, about 200 days, about 201 days, or more after administration of the anti-sortilin antibody.

In some embodiments, the level of sortilin protein in white blood cells of the individual is determined using any method of quantifying proteins known in the art. Non-limiting examples of methods that may be used to quantify sortilin protein include SOMASCAN assay (see, e.g., Candia et al. (2017) Sci Rep 7, 14248), Western blots, mass spectrometry, flow cytometry, and enzyme-linked immunosorbent (ELISA) assays. In certain embodiments, the level of sortilin protein in white blood cells is determined using ELISA assays.

### Disease and Neuroinflammation Biomarkers

In some embodiments, the methods of treating or delaying progression of a disease or disorder provided herein further comprise measuring the level of neurofilament light chain (NF-L). In some embodiments, the level of NF-L is measured in a sample of blood (e.g., plasma) or cerebrospinal fluid obtained from an individual before and after the individual has received one or more doses of an anti-sortilin antibody of the disclosure. Non-limiting examples of methods that may be used to measure the levels of NF-L in a sample obtained from an individual include SOMASCAN assay (see, e.g., Candia et al. (2017) Sci Rep 7, 14248), Western blots, mass spectrometry, flow cytometry, and enzyme-linked immunosorbent assay (ELISA).

In some embodiments, the methods of treating or delaying progression of a disease or disorder provided herein further comprise measuring the level of Tau. In some embodiments, the level of Tau is measured in a sample of blood or cerebrospinal fluid obtained from an individual before and after the individual has received one or more doses of an anti-sortilin antibody of the disclosure. Non-limiting examples of methods that may be used to measure the levels of Tau in a sample obtained from the individual include SOMASCAN assay (see, e.g., Candia et al. (2017) Sci Rep 7, 14248), Western blots, mass spectrometry, flow cytometry, and enzyme-linked immunosorbent assay (ELISA).

In some embodiments, the methods of treating or delaying progression of a disease or disorder provided herein further comprise measuring the levels of one or more biomarkers of neuroinflammation. In some embodiments, the levels of the one or more biomarkers of neuroinflammation are measured in a sample of blood or cerebrospinal fluid obtained from an individual before and after the individual has received one or more doses of an anti-sortilin antibody of the disclosure. Biomarkers of neuroinflammation include, without limitation, IL-6, SPP1, IFI2712A, CHIT1, YKL-40, GFAP, YWHAE, CSF1, AIF1, LY86, CD86, and TOP2A. Non-limiting examples of methods that may be used to measure the levels of the one or more biomarkers in a sample obtained from the individual include SOMASCAN assay (see, e.g., Candia et al. (2017) Sci Rep 7, 14248), Western blots, mass spectrometry, flow cytometry, and enzyme-linked immunosorbent assay (ELISA).

### Methods of Monitoring Treatment

Also provided herein are methods of monitoring the treatment of an individual being administered an anti-sortilin antibody of the present disclosure.

In some embodiments, the methods of monitoring treatment comprise measuring the level of one or more biomarkers selected from progranulin protein, GCase protein, neurofilament light chain (NF-L), Tau, one or more markers of neuroinflammation, or alpha-synuclein protein in a sample of plasma or cerebrospinal fluid obtained from an individual before and after the individual has received one or more doses of an anti-Sortilin antibody of the disclosure. In some embodiments, the method further comprises a step of assessing the activity of the anti-sortilin antibody in the individual based on the level of the one or more biomarkers in the sample. In some embodiments, the sample is from the cerebrospinal fluid of the individual or the blood, e.g., plasma, of the individual. In some embodiments, the sample is from the cerebrospinal fluid of the individual. In some embodiments, the sample is from the blood, e.g., plasma, of the individual. Non-limiting examples of methods that may be used to measure the levels of the one or more biomarkers in a sample obtained from the individual include SOMASCAN assay (see, e.g., Candia et al. (2017) Sci Rep 7, 14248), Western blots, mass spectrometry, flow cytometry, and enzyme-linked immunosorbent assay (ELISA).

In some embodiments, the methods of monitoring treatment comprise measuring one or more biomarkers of the cerebrospinal fluid proteome in a sample of cerebrospinal fluid obtained from an individual before and after the individual has received one or more doses of an anti-Sortilin antibody of the disclosure. In some embodiments, the method further comprises a step of assessing the activity of the anti-sortilin antibody in the individual based on the level of the one or more biomarkers in the sample. Non-limiting examples of methods that may be used to measure the levels of the one or more biomarkers in a sample obtained from the individual include SOMASCAN assay (see, e.g., Candia et al. (2017) Sci Rep 7, 14248), mass spectrometry, Western blots, flow cytometry, and enzyme-linked immunosorbent assay (ELISA).

In some embodiments, the methods of monitoring treatment comprise measuring the level of one or more biomarkers of lysosomal function in a sample obtained from an individual before and after the individual has received one or more doses of an anti-Sortilin antibody of the disclosure. In some embodiments, the one or more biomarkers of lysosomal function are selected from GCase protein, GCase activity, lyso-Gb1, or glucosylceramide. In some embodiments, the method further comprises a step of assessing the activity of the anti-sortilin antibody in the individual based on the level of the one or more biomarkers in the sample. In some embodiments, the sample is from the cerebrospinal fluid of the individual or the blood, e.g., plasma, of the individual. In some embodiments, the sample is from the cerebrospinal fluid of the individual. In some embodiments, the sample is from the blood, e.g., plasma, of the individual. Non-limiting examples of methods that may be used to measure the levels of the one or more biomarkers in a sample obtained from the individual include SOMASCAN assay (see, e.g., Candia et al. (2017) Sci Rep 7, 14248), Western blots, mass spectrometry, flow cytometry, and enzyme-linked immunosorbent assay (ELISA).

### Pharmaceutical Compositions

Provided herein are pharmaceutical compositions and/or pharmaceutical formulations comprising the anti-sortilin antibodies of the present disclosure and a pharmaceutically acceptable carrier.

In some embodiments, pharmaceutically acceptable carriers preferably are nontoxic to recipients at the dosages and concentrations employed. The antibodies described herein may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols. Pharmaceutically acceptable carriers can include, depending on the formulation desired, pharmaceutically-acceptable, non-toxic carriers of diluents, which are vehicles commonly used to formulate pharmaceutical compositions for animal or human administration. In certain embodiments, the pharmaceutical composition can comprise formulation materials for modifying, maintaining or preserving, for example, the pH, osmolarity, viscosity, clarity, color, isotonicity, odor, sterility, stability, rate of dissolution or release, adsorption or penetration of the composition.

In certain embodiments, pharmaceutically acceptable carriers include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. Further examples of formulations that are suitable for various types of administration can be found in Remington: The Science and Practice of Pharmacy, Pharmaceutical Press 22nd ed. (2013). For a brief review of methods for drug delivery, see, Langer, Science 249:1527-1533 (1990).

Formulations suitable for parenteral administration include aqueous and non-aqueous, isotonic sterile injection solutions, which can comprise antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Formulations may be optimized for retention and stabilization in the brain or central nervous system. When the agent is administered into the cranial compartment, it is desirable for the agent to be retained in the compartment, and not to diffuse or otherwise cross the blood brain barrier. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the antibody, such as an anti-sortilin antibody of the present disclosure, in a biodegradable or bioerodible implant. The rate of release of the antibody is controlled by the rate of transport through the polymeric matrix, and the biodegradation of the implant. Implants may be particles, sheets, patches, plaques, fibers, microcapsules and the like and may be of any size or shape compatible with the selected site of insertion. Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers may be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the disclosure. Hydrogels are typically a copolymer material, characterized by the ability to imbibe a liquid.

In some embodiments, a pharmaceutical composition or formulation provided herein comprises an anti-Sortilin antibody of the disclosure and is suitable for administration to a human subject by intravenous infusion and/or subcutaneous injection.

### Kits/Articles of Manufacture

Provided herein are articles of manufacture (e.g., kits) comprising an anti-sortilin antibody of the disclosure. Articles of manufacture may include one or more containers comprising an antibody described herein. Containers may be any suitable packaging including, but not limited to, vials, bottles, jars, flexible packaging (e.g., sealed Mylar or plastic bags), and the like. The containers may be unit doses, bulk packages (e.g., multi-dose packages) or sub-unit doses.

In some embodiments, the kits may further include a second agent. In some embodiments, the second agent is a pharmaceutically-acceptable buffer or diluting agent including, but not limited to, bacteriostatic water for injection (BWFI), phosphate-buffered saline, Ringer's solution and dextrose solution. In some embodiments, the second agent is a pharmaceutically active agent.

In some embodiments of any of the articles of manufacture, the articles of manufacture further include instructions for use in accordance with the methods of this disclosure. The instructions generally include information as to dosage, dosing schedule, and route of administration for the intended treatment. In some embodiments, these instructions comprise a description of administration of an antibody of the present disclosure (e.g., an anti-sortilin antibody described herein) to prevent, reduce risk, or treat an individual having a disease, disorder, or injury selected from frontotemporal dementia, progressive supranuclear palsy, Alzheimer's disease, vascular dementia, seizures, retinal dystrophy, amyotrophic lateral sclerosis, traumatic brain injury, a spinal cord injury, dementia, stroke, Parkinson's disease, acute disseminated encephalomyelitis, retinal degeneration, age related macular degeneration, glaucoma, multiple sclerosis, septic shock, bacterial infection, arthritis, or osteoarthritis, according to any methods of this disclosure. In some embodiments, the disease, disorder, or injury is frontotemporal dementia. In some embodiments, the disease, disorder, or injury is Alzheimer's disease. In some embodiments, the disease, disorder, or injury is Parkinson's disease. In some embodiments, the instructions include instructions for use of the anti-sortilin antibody and the second agent (e.g., second pharmaceutically active agent).

The present disclosure will be more fully understood by reference to the following Examples. They should not, however, be construed as limiting the scope of the present disclosure. All citations throughout the disclosure are hereby expressly incorporated by reference.

### EXAMPLES

### Example 1: A Phase I Study in Healthy Volunteers to Assess the Safety, Tolerability, Pharmacokinetics, Pharmacodynamics, and Bioavailability of Intravenous and Subcutaneous Anti-Sortilin Antibody ALX

This Example describes a Phase I study in healthy volunteers to assess the safety, tolerability, pharmacokinetics, pharmacodynamics, and bioavailability of anti-sortilin antibody ALX administered intravenously or subcutaneously.

Anti-sortilin antibody ALX comprises a heavy chain comprising the amino acid sequence of SEQ ID NO: 31 or 32 and a light chain comprising the amino acid sequence of SEQ ID NO: 30. In this study, anti-sortilin antibody ALX was produced by expressing one or more nucleic acids that encode a heavy chain comprising the amino acid sequence of SEQ ID NO: 31 and a light chain comprising the amino acid sequence of SEQ ID NO: 30.

### I. Study Objectives

The primary objective of this study is to evaluate the safety, tolerability, pharmacokinetics (PK), pharmacodynamics (PD), and bioavailability of antibody ALX when administered at escalating single doses by intravenous (IV) infusion or as a single subcutaneous (SC) administration in healthy human volunteers (HV). This study is also designed to evaluate administration of multiple doses of antibody ALX by IV infusion or as SC administration in HVs.

A. Primary Outcome Measures

The primary outcome measure of this study is evaluation of safety and tolerability of antibody ALX, assessed based on the number of subjects that experience adverse events (AEs) and dose-limiting adverse events (DLAEs).

B. Secondary Outcome Measures

The secondary outcome measures of this study include:
Pharmacokinetics (PK) of antibody ALX, assessed based on serum and cerebrospinal fluid (CSF) concentrations of antibody ALX.
Maximum concentration (Cₘₐₓ) for antibody ALX in serum or plasma and CSF.
Area under the curve (AUC) for antibody ALX based on serum and CSF concentration of antibody ALX.

### II. Study Participants

Healthy male and female human volunteers are included in this study.

A. Inclusion Criteria

Healthy human volunteers that meet the following criteria are included in this study.
Male or female, 18-65 years of age, inclusive, at screening.
Body mass index 18.0-35.0 kg/m², inclusive.
Body weight 45-120 kg, inclusive.
In good physical health on the basis of no clinically significant findings from medical history, physical examination, laboratory tests, 12-lead electrocardiogram (ECG), and vital signs.

B. Exclusion Criteria

Subjects who meet any of the following criteria are not included in this study.
Known history of severe allergic, anaphylactic, or other hypersensitivity reactions to chimeric, human, or humanized antibodies or fusion proteins.
Past history of seizures, with the exception of childhood febrile seizures.
Serious infection requiring oral or IV antibiotics within 30 days prior to screening.
Clinically significant systemic immunocompromised condition because of continuing effects of immune-suppressing medication.
History of major depression, schizophrenia, schizoaffective disorder, or bipolar disorder.
History of cancer, except if considered likely to be cured, is not being actively treated with anticancer therapy or radiotherapy and is not likely to require treatment in the ensuing 3 years, and is considered to have low probability of recurrence.
Positive for hepatitis B surface antigen, anti-hepatitis C virus antibodies, or anti-human immunodeficiency virus (HIV)-1 and -2 antibodies or antigen, or history of spirochete infection of the central nervous system (e.g., syphilis, Lyme disease, or borreliosis).
Chronic kidney disease as indicated by a screening creatinine clearance (CL) of <30 mL/min, as calculated using the Cockcroft-Gault formula, that remains <30 mL/min if retested.
Impaired hepatic function as indicated by screening aspartate aminotransferase (AST) or alanine aminotransferase (ALT) of >2×the upper limit of normal or total bilirubin of >1.5×the upper limit of normal, and that remains above these limits if retested due to a slightly elevated initial result or abnormalities in synthetic function tests that are clinically significant.
Within the last 2 years, unstable or clinically significant cardiovascular disease (e.g., myocardial infarction, angina pectoris, or New York Heart Association Class II or higher cardiac failure).
Uncontrolled hypertension (i.e., sustained resting blood pressure of >140 mmHg systolic or >90 mmHg diastolic).
History or presence of an abnormal ECG that is clinically significant, including complete left bundle branch block, second- or third-degree heart block, or evidence of prior myocardial infarction.
QT interval corrected using Fridericia's formula of >450 ms for male participants and >470 ms for female participants (average of triplicate screening measurements).
History of ventricular dysrhythmias or risk factors for ventricular dysrhythmias, such as structural heart disease (e.g., severe left ventricular systolic dysfunction, or left ventricular hypertrophy), coronary heart disease (symptomatic or with ischemia demonstrated by diagnostic testing), clinically significant electrolyte abnormalities (e.g., hypokalemia, hypomagnesemia, or hypocalcemia), or family history of sudden unexplained death or long QT syndrome.

### III. Study Design

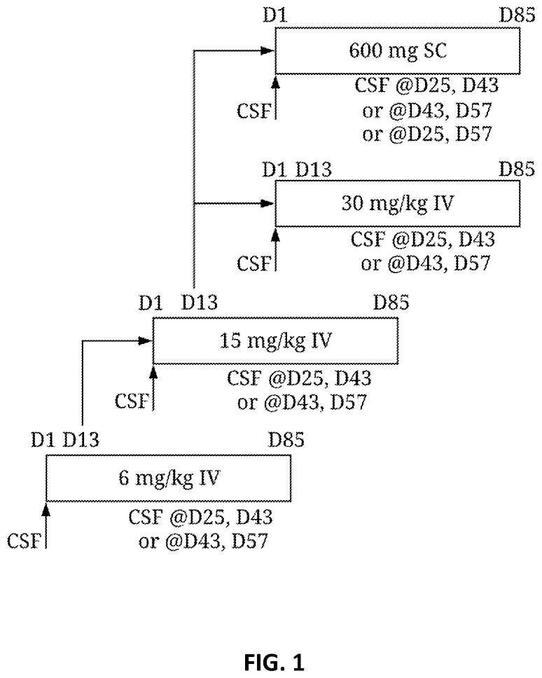

This study is a first-in-human, Phase I study designed to investigate the safety, tolerability, PK, PD, and bioavailability of single doses of anti-sortilin antibody ALX administered via IV and SC routes. An overview of the study design is provided in FIG. 1. This study also evaluates administration of multiple doses of antibody ALX via IV and SC routes.

This study includes IV administration of single ascending doses (SAD) of antibody ALX in approximately 3 cohorts of 11 HV participants. The dose levels for the first 3 cohorts are 6 mg/kg, 15 mg/kg, and 30 mg/kg. In some embodiments, an additional IV SAD cohort is included at 60 mg/kg.

In addition to the SAD IV cohorts, one single dose (SD) cohort assesses the bioavailability and tolerability of SC administration of antibody ALX. The SD SC cohort includes 9 HV participants administered antibody ALX at the fixed dose of 600 mg. One additional SD SC cohort is enrolled if dosing flexibility is required. The following cohorts are also assessed: a single dose SC 150 mg cohort, a multiple-dose (MD) SC cohort, and a multiple-dose IV cohort, as described below.

All cohorts require cerebrospinal fluid (CSF) sampling pre-dose (baseline) and at two time points after dosing to assess PK and PD in the brain. Subgroups within each cohort have CSF sampled at different post-dose time points to allow for CSF sampling at more time points across the cohort (a total of three time points after dosing). The CSF subgroups are described below for both the IV and SC cohorts.

All participants in this study are followed-up for 12 weeks after their single dose of antibody ALX for evaluation of safety, PK, PD, and bioavailability. In some embodiments, the participants are followed-up for 16 weeks after their single dose of antibody ALX.

A. Single-Ascending-Dose IV Cohorts

In each of the SAD IV cohorts, 11 HV participants are randomized to receive antibody ALX or placebo (PBO) in a ratio of 8:3 (antibody ALX:PBO) in up to 3 dose cohorts (cohorts 1, 2, and 3). In total, approximately 33 HV participants are included. The first SAD IV cohort is dosed at 6 mg/kg of antibody ALX. The dose levels for the subsequent SAD IV cohorts are 15 mg/kg and 30 mg/kg of antibody ALX. An additional SAD IV dose of 60 mg/kg of antibody ALX may be added (not shown in FIG. 1). In some embodiments, an additional SAD cohort is included at 60 mg/kg. An overview of the SAD IV cohorts is provided in Table 3.

TABLE 3
Single Ascending Dose IV Cohorts.

| | Number of Participants | |
|---|---|---|
| Cohort | (Antibody ALX:PBO Ratio) | Dose (mg/kg) |
| 1 | 8:3 | 6 |
| 2 | 8:3 | 15 |
| 3 | 8:3 | 30 |
| 4 | 8:3 | 60 |

The first 6 participants in each SAD IV cohort have CSF sampled at baseline, Day 25, and Day 43. The remaining 5 participants in each SAD IV cohort have CSF sampled at baseline, Day 43, and Day 57. The overall randomization of the 11 participants in each SAD IV cohort is 8:3 (antibody ALX:PBO). Within each SAD IV cohort, CSF is sampled for 4 participants dosed with antibody ALX at Day 25 and Day 57, and for 8 participants dosed with antibody ALX at baseline and Day 43. Post-dose CSF time points are altered if warranted by emerging PK and PD data.

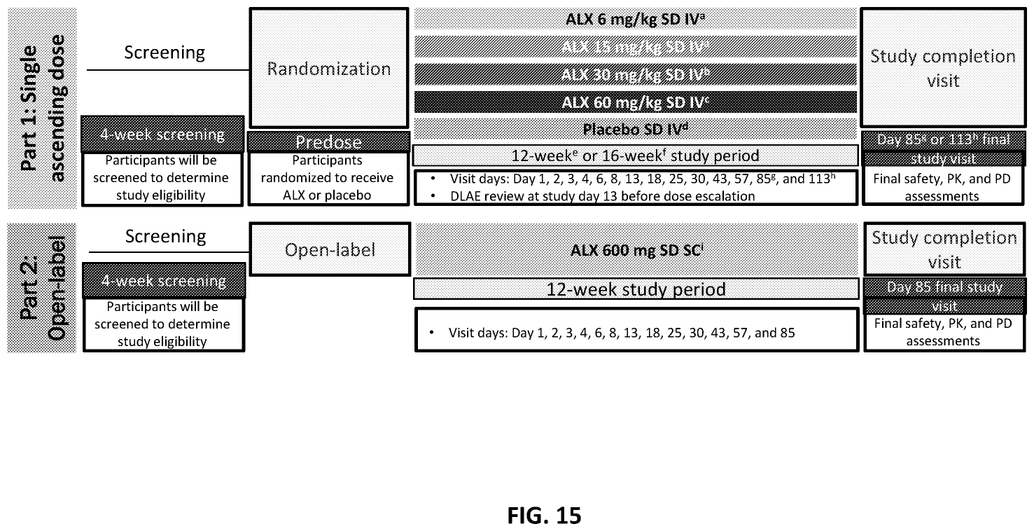

In another embodiment, for the 6 mg/kg IV cohort, CSF was sampled at baseline and at 2 time points on days 25, 43, or 57 (FIG. 15). In another embodiment, for the 15 mg/kg IV cohort, CSF was sampled at baseline and at 2 points on days 25, 43, or 57 (FIG. 15). In another embodiment, for the 30 mg/kg IV cohort, CSF was sampled at baseline and at 2 time points on days 25, 43, 57, or 85 (FIG. 15). In another embodiment, for the 60 mg/kg IV cohort, CSF was sampled at baseline and at 2 time points on days 43, 57, or 85 (FIG. 15). In another embodiment, for the placebo SD IV cohort, CSF was sampled at baseline and at 2 time points on days 25, 43, 57, or 85 (FIG. 15).

Based on emerging safety, tolerability, PK, and PD data from prior cohorts, dose levels may be adjusted, 1 or more cohorts may be omitted or expanded, or cohorts at intermediate doses may be added. Expanded cohorts or cohorts at intermediate doses are open-label and include up to 9 participants dosed with antibody ALX.

B. Single-Dose SC Cohort—600 mg

The SD SC 600 mg cohort is an open-label cohort of 9 HV. This cohort is enrolled if the 15 mg/kg dose of antibody ALX is generally safe and tolerable based on clinical safety data up to and including the Day 13 visit for SAD IV Cohort 2 (15 mg/kg). The SD SC cohort is administered a single dose of 600 mg of antibody ALX, which corresponds to a maximum dose level of 13.3 mg/kg for the lowest body weight permitted in this study (45 kg). There is no PBO group for the SD SC cohort.

The first 3 participants have CSF sampled at baseline, Day 25, and Day 43. The next 3 participants have CSF sampled at baseline, Day 25, and Day 57. The remaining 3 participants have CSF sampled at baseline, Day 43, and Day 57. Each post-dose CSF time point (Day 25, Day 43, and Day 57) includes CSF sampling for 6 participants. Post-dose CSF time points are altered if warranted by emerging PK and PD data.

In another embodiment, the first participant in the SD SC 600 mg cohort has CSF sampled at baseline, Day 25, and Day 43. The second participant has CSF sampled at baseline, Day 13 and Day 25. The third participant has CSF sampled at baseline, Day 18, and Day 25. The next 3 participants have CSF sampled at baseline, Day 8 and Day 13. The next 3 participants have CSF sampled at baseline, Day 8, and Day 18. In one embodiment, the CSF samples are collected at the time points shown below in Table 4.

TABLE 4
CSF Sample Collection Time Points for
Single-Dose SC 600 mg Cohort.

| SD SC 600 mg Cohort | |
|---|---|
| Subjects by Enrollment Sequence | CSF Draw |
| (Enrollment Sequence) | Time Points |
| 1st (Sentinel-Dose Subject) | Baseline, Day 25 |
| | (D25), D43 |
| 2nd through 9th subjects | |
| enrolled in the SC cohort | |
| 2^{nd} | Baseline, D13, D25 |
| 3^{rd} | Baseline, D18, D25 |
| 4^{th} | Baseline, D8, D13 |
| 5^{th} | Baseline, D8, D13 |
| 6^{th} | Baseline, D8, D13 |
| 7^{th} | Baseline, D8, D18 |
| 8^{th} | Baseline, D8, D18 |
| 9^{th} | Baseline, D8, D18 |

C. Single Dose SC Cohort—150 mg

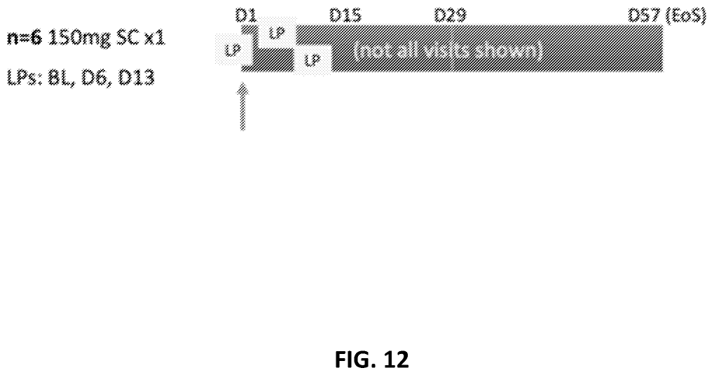

The single dose SC 150 mg cohort is a cohort of 6 HV participants and is administered a single dose of 150 mg of antibody ALX on Day 1. CSF samples (LP, lumbar puncture) are taken at baseline, Day 6, and Day 13 (FIG. 12).

D. Multiple-Dose SC Cohort

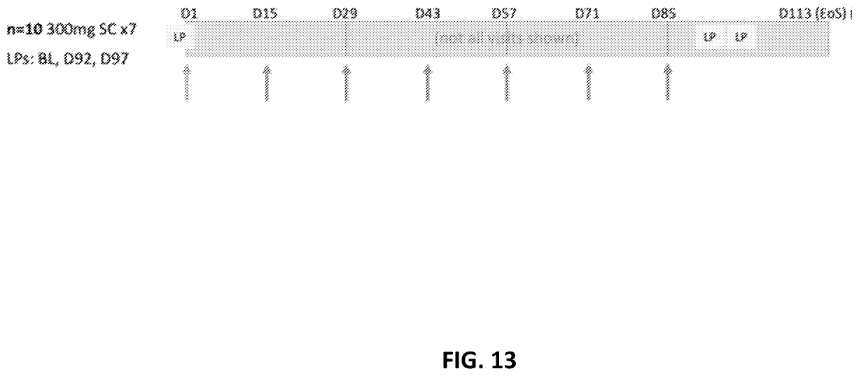

The multiple-dose (MD) SC cohort is administered seven doses of 300 mg of antibody ALX in a cohort of 10 HV participants (FIG. 13). The 300 mg antibody ALX dose is administered to subjects on Day 1, Day 15, Day 29, Day 43, Day 57, Day 71, and Day 85. CSF samples (LP) are taken at baseline, Day 92 and Day 97.

E. Multiple-Dose IV Cohort

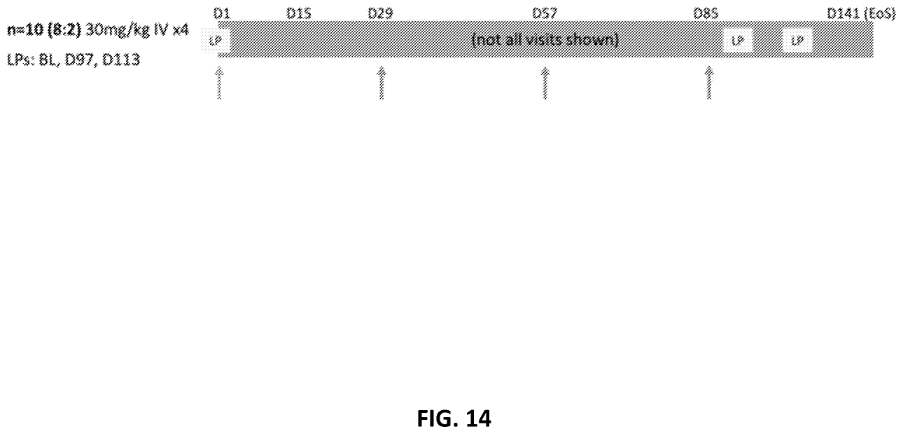

In the MD IV cohort, 10 HV participants are randomized to receive antibody ALX or placebo (PBO) in a ratio of 8:2 (antibody ALX:PBO) (FIG. 14). The MD IV cohort is administered 30 mg/kg of antibody ALX or placebo on Day 1, Day 29, Day 57, and Day 85. CSF samples (LP) are taken at baseline and at Day 97 and Day 113.

F. Investigational Medicinal Product, Dose, and Route of Administration

For IV cohorts, antibody ALX is administered as a single peripheral IV infusion using an infusion pump over approximately 60 minutes.

For the SC cohorts, antibody ALX is administered as a slow injection over 15 minutes. A total volume of 12 mL for the 600 mg dose of antibody ALX is administered.

G. Placebo Control, Dose and Route of Administration

For IV cohorts, normal saline (0.9% NaCl) is administered as PBO as a single peripheral IV infusion using an infusion pump over approximately 60 minutes.

For the SC cohort, there is no PBO group.

H. Duration of Study

The total study duration for each participant is approximately 4 months. In some embodiments, the total study duration for each participant is approximately 3-6 months. This includes a screening period of up to 28 days prior to study drug administration, administration of a single IV infusion or SC administration of antibody ALX or PBO (IV cohorts only) on Day 1, or multiple doses of IV or SC antibody ALX or PBO (IV cohorts only), each according to the dosing schedule of each cohort, and follow-up through Day 57 (8 weeks post-study drug administration), Day 85 (12 weeks post study drug administration), Day 113 (16 weeks post study drug administration), or Day 141 (20 weeks post study drug administration), according to the follow-up periods designated for each cohort.

### IV. Study Procedures

A. Pre-Dose Procedures and Assessments

Prior to administration of antibody ALX or PBO, the following assessments are performed:
Physical examination or limited, symptom-driven examination.
Neurological examination.
Height and body weight.
Vital signs, including blood pressure (BP), pulse, body temperature, and respiratory rate.
Triplicate 12-lead ECG.
Blood and urine samples for chemistry, hematology, coagulation, serology, and urinalysis.
Serum samples for anti-drug antibodies (ADA) and PK analysis; plasma samples for progranulin and exploratory biomarkers; and whole blood samples for white blood cell analysis.
Whole blood sample for exploratory biomarkers.
Whole genome sequencing (WGS).
CSF samples via lumbar puncture.
Concomitant medications review.
Serious adverse events (SAEs) and AEs recorded.

B. Dosing

Antibody ALX or PBO are administered to participants as described above. No fasting is applicable on the dosing days, unless clinical laboratory tests are also being performed.

C. Post-Dose

The following assessments are performed after administration of antibody ALX or PBO:
Serum samples for PK analysis; plasma samples for progranulin and biomarkers; and whole blood samples for white blood cell analysis.
Triplicate 12-lead ECG.
Vital signs, including BP, pulse, body temperature, and respiratory rate.
Concomitant medications review.
SAEs and AEs recorded.

### V. Study Assessments

A. Clinical Assessments

Any prior medications received within 30 days before screening are recorded. All concomitant medications and concurrent therapies are recorded from screening through the follow-up period.

From the time of obtaining informed consent through the first administration of study drug, all SAEs as well as any AEs related to protocol procedures are recorded. From the time of first administration of study drug through the follow-up period, any SAEs and AEs, including exacerbation or changes in medical history, are recorded.

Demographic information (year of birth, gender, race) is recorded.

All relevant medical history, including history or current disease, other pertinent history, and information regarding underlying diseases is recorded at screening prior to study drug administration.

A complete physical examination (PE) is performed at screening and study completion or early termination (ET). A complete PE includes evaluation of the head, eyes, ears, nose, and throat, and of the cardiovascular, dermatological, musculoskeletal, respiratory, and gastrointestinal systems. Abnormalities observed at baseline are recorded. Body height (in cm) and weight (in kg) are measured at screening, and BMI is calculated.

A complete neurologic examination includes the evaluation of consciousness, orientation, cranial nerves, motor and sensory system, coordination and gait, and reflexes. Changes from baseline abnormalities are recorded at each subsequent neurologic examination. New or worsened abnormalities are recorded as AEs if considered clinically significant.

Vital signs are assessed and abnormalities observed at baseline are recorded. At subsequent visits, new or worsened abnormalities are recorded as AEs if considered clinically significant. All ECGs are analyzed from a clinical safety basis (without intensive QT analysis). The clinical significance of ECG changes is determined after review of the ECG report in relation to the participant's medical history, PE, and concomitant medications.

Blood and urine samples are collected for clinical safety laboratory tests (chemistry, hematology, urinalysis, serology, drug and alcohol screen and pregnancy testing). Out-of-range values are recorded as AEs after start of study drug if confirmed and considered clinically significant, or if they require a participant to be discontinued from the study or to receive treatment.

Blood serum samples are collected for determination of anti-drug antibodies (ADA). Additional ADA samples are collected in participants with signs and symptoms of infusion-related reactions. A corresponding additional PK sample is obtained at the same time point.

B. Safety Assessments

Adverse events (AEs) are recorded and graded according to the World Health Organization (WHO) Toxicity Grading Scale. If an AE is not specified within the WHO Toxicity Grading Scale, then the AE is graded as follows: Grade 1 (mild: transient or mild discomfort; no limitation in activity; no medical intervention or therapy required; participant may be aware of the sign or symptom but tolerates it reasonably well); Grade 2 (moderate: mild to moderate limitation in activity; no or minimal medical intervention/therapy required); Grade 3 (severe: marked limitation in activity; medical intervention/therapy required, e.g., hospitalization); Grade 4 (life-threatening: risk of death due to the adverse experience as it occurred); or Grade 5 (death).

An AE is related to study drug (i.e., antibody ALX) if the AE is considered definitely, probably, or possibly related to study treatment, e.g., there is clear evidence that the event is related to the use of study drug, or the event cannot be explained by the participant's medical condition, concomitant therapy, or other causes, and there is a plausible temporal relationship between the event and study drug administration. An AE is not related to study drug if the relatedness to study drug is considered unlikely or definitively not related to study treatment, e.g., the event can be readily explained by the participant's underlying medical condition, concomitant therapy, or other causes, or an event for which an alternative explanation is more likely (e.g., concomitant medications or ongoing medical conditions) or the temporal relationship to study drug administration and/or exposure suggests that a causal relationship is unlikely.

A serious adverse event (SAE) is any AE occurring at any dose that results in death, a life-threatening AE, inpatient hospitalization, prolongation of existing hospitalization, persistent or significant disability/incapacity, or a congenital anomaly/birth defect. Other important medical events are also considered an SAE when they jeopardize the participant or require intervention to prevent one of the outcomes listed.

A dose-limiting AE (DLAE) is an AE that is assessed as related to study drug, it is confirmed that the participant received study drug, and is: a SAE that has no other clearly attributable cause beyond study drug, an AE Grade 3 or higher that has no other clearly attributable cause beyond study drug, or a Grade 2 or higher infusion related toxicity (e.g., allergic reaction/hypersensitivity, drug fever, urticaria, dyspnea, flushing, bronchospasm, wheezing, hypoxia, or infusion site pain) occurring during the infusion or within 24 hours after completion of an infusion that does not resolve promptly with supportive care and/or a reduced infusion rate.

C. Pharmacokinetics Assessments

Blood serum samples are collected for assessment of serum concentrations of antibody ALX.

Cerebrospinal fluid samples are also assessed for concentration of antibody ALX.

D. Pharmacodynamics Assessments

Blood plasma samples are collected for evaluation of levels of progranulin. Whole blood samples are collected for evaluation of sortilin expression in white blood cells and for evaluation of other analytes.

Cerebrospinal fluid samples are evaluated for levels of progranulin.

E. Biomarker Assessments

Plasma samples are collected for evaluation of biomarkers, including levels of neurofilament light chain (NF-L), Tau, markers of neuroinflammation, and other analytes relevant to disease biology and response to antibody ALX.

Cerebrospinal fluid samples are evaluated for biomarkers, including levels of NF-L, Tau, markers of neuroinflammation, and other analytes relevant to disease biology and response to antibody ALX.

A blood sample is collected at screening for DNA extraction to enable analysis via whole genome sequencing to identify common and rare genetic variants that are predictive of response to antibody ALX, are associated with progression to a more severe disease state, are associated with susceptibility to developing AEs, or can increase the knowledge and understanding of disease biology.

### VI. Study Endpoints and Statistics

Summary statistics are provided (e.g., number of non-missing values, mean, median, standard deviation, minimum and maximum for continuous variables, and number and percentage for categorical variables) for all measures, including demographic and baseline values, safety endpoints, PK, PD, and PK/PD correlations. No formal statistical inferences are made for safety parameters. No imputation is used for missing data.

A. Safety Analysis and Endpoints

The safety population includes all enrolled participants who receive any amount of study drug (antibody ALX or PBO). The safety population is described and summarized by treatment dose level/cohort.

The safety endpoints of this study include:
Incidence, nature, and severity of AEs.
Incidence of dose-limiting AEs (DLAEs).
Incidence of treatment discontinuations due to AEs.
Mean changes in clinical laboratory test results from baseline; incidence of treatment-emergent abnormal laboratory values and abnormal laboratory values reported as AEs.
Mean changes in ECG assessments from baseline and incidence of abnormal ECG assessments.
Mean changes and incidence of abnormal blood pressure and heart rate.
Mean changes in vital signs from baseline and incidence of abnormal vital sign measurements.
Physical and neurologic examination abnormalities.
Incidence of anti-drug antibodies (ADA) during the study relative to the prevalence of ADA at baseline.

All treatment emergent AEs that occur on or after dosing are summarized by the Medical Dictionary for Regulatory Activities (MedDRA) coded term, appropriate thesaurus level, and severity. In addition, all serious AEs (SAEs), including deaths, and events leading to discontinuation, are listed separately and summarized. DLAEs are listed and summarized by treatment dose level/cohort. All AEs are coded using the latest version of the MedDRA and are classified by MedDRA system organ class and preferred term.

B. Pharmacokinetic Analysis and Endpoints

The PK population includes all participants in the safety population who have adequate assessments for determination of PK parameters.

The PK endpoints of this study include:
Serum and cerebrospinal fluid concentration of antibody ALX.
Relationship between antibody ALX serum concentration or PK parameters and safety endpoints. PK parameters include: Cₘₐₓ, time to maximum observed concentration (Tₘₐₓ), AUC from time zero to time t, AUC from time zero to infinity, percentage of AUC extrapolated to infinity, AUC from time zero to tau, elimination rate constant, terminal half-life (tr), volume of distribution during the terminal phase (Vₛ.), and clearance (CL).
Relationship between serum concentration, CSF concentration, or PK parameters for antibody ALX and activity or PD endpoints.
Bioavailability of SC administered antibody ALX.

Individual and mean plasma antibody ALX concentration-time data is tabulated and plotted by treatment dose level/cohort. As applicable, the serum PK of antibody ALX is summarized by estimation of total exposure as indicated by AUC, Cₘₐₓ, total serum CL, V_{z}, and t_{1/2} on the basis of results obtained following single or multiple doses. Estimates for PK parameters are tabulated and summarized by descriptive statistics (mean, standard deviation, minimum, maximum, geometric mean, and coefficient of variation); dose proportionality is explored using a regression (power) model relating log transformed Cₘₐₓ and designated AUC parameters to the log-transformed dose. Individual and mean antibody ALX CSF concentration-time data are tabulated by treatment dose level/cohort and plotted over time. Bioavailability of SC administered antibody ALX is estimated. Potential correlations of relevant PK parameters with dose, demographics, safety (including QT changes), and PD measures are explored. Additional modeling to characterize these correlations is performed, including population PK analysis.

C. Pharmacodynamic Analysis and Endpoints

The PD population includes all participants in the safety population who have both a baseline PD assessment and at least one post-dose PD assessment.

The PD endpoints of this study include changes in the levels of progranulin in plasma and CSF after dosing relative to baseline.

PD endpoints are described and summarized by treatment dose level/cohort.

D. Biomarker Analysis and Endpoints

The biomarker population includes all participants in the safety population who have the necessary baseline and post-dose measurements to provide interpretable results for specific parameters of interest.

The biomarker endpoints of this study include:
Changes in the levels of markers of neuroinflammation, and other analytes in blood and CSF after dosing relative to baseline.
Relationship between biomarkers at baseline and safety, PK, activity, immunogenicity, or other biomarker endpoints.

Biomarkers evaluated in blood and in CSF samples include NF-L, as well as other blood/CSF markers implicated in frontotemporal dementia (FTD) or other neurodegenerative conditions. In addition, biomarkers include rare genetic variants identified through whole genome sequencing performed on DNA extracted from blood.

### Example 2: Results of a Phase I Study in Healthy Volunteers to Assess the Safety, Tolerability, Pharmacokinetics, Pharmacodynamics, and Bioavailability of Intravenous and Subcutaneous Anti-Sortilin Antibody ALX

This Example provides results from the single ascending dose (SAD) intravenous (IV) Cohorts 1 and 2 of the Phase I study described in Example 1.

As described in detail in Example 1, healthy volunteers (HVs) in SAD IV Cohort 1 were administered a single intravenous dose of 6 mg/kg of antibody ALX, and HVs in SAD IV Cohort 2 were administered a single intravenous dose of 15 mg/kg of antibody ALX.

Table 5 provides an overview of participant allocation at various time points during which progranulin plasma levels were determined.

TABLE 5
Healthy volunteer participant allocation and plasma
progranulin levels assessments in Cohorts 1 and 2.

| | Cohort 1 | | Cohort 2 | |
|---|---|---|---|---|
| | Antibody | | Antibody | |
| Visit | ALX | Placebo | ALX | Placebo |
| (Relative Day) | (N = 8) | (N = 3) | (N = 9*) | (N = 3) |
| Day 1: | 8 | 3 | 6 | 2 |
| Pre-Dose, EOI, | | | | |
| 4 h, 8 h, | | | | |
| and 12 h | | | | |
| Day 2 | 8 | 3 | 6 | 2 |
| Day 3 | 8 | 3 | 6 | 2 |
| Day 6 | 8 | 3 | 5 | 2 |
| Day 8 | 8 | 3 | 5 | 2 |
| Day 13 | 8 | 3 | 5 | 2 |
| Day 18 | 8 | 2 | 5 | 2 |
| Day 25 | 8 | 3 | 4 | 2 |
| Day 30 | 8 | 3 | 5 | 2 |
| Day 43 | 8 | 2 | 5 | 2 |
| Day 57 | 7 | 1 | 5 | 2 |
| Day 85 | 7 | 2 | 0 | 0 |

EOI = end of infusion;
h = hours after infusion;
Day = study day.
*One participant was assessed on Day 1, Day 2, and Day 3 and then withdrew; this participant was replaced.

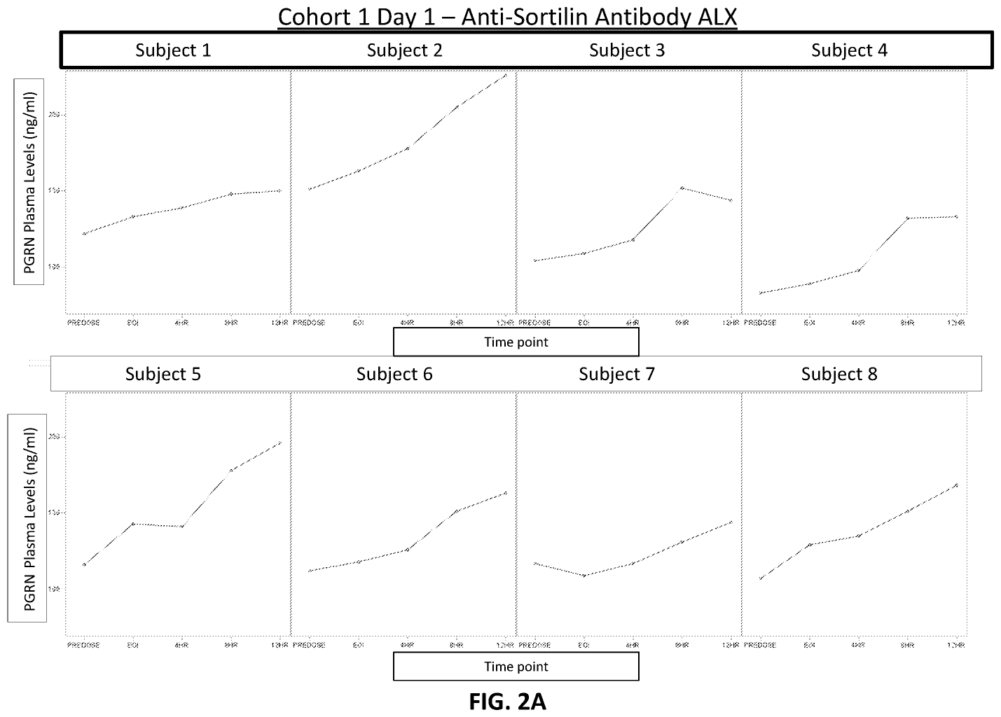

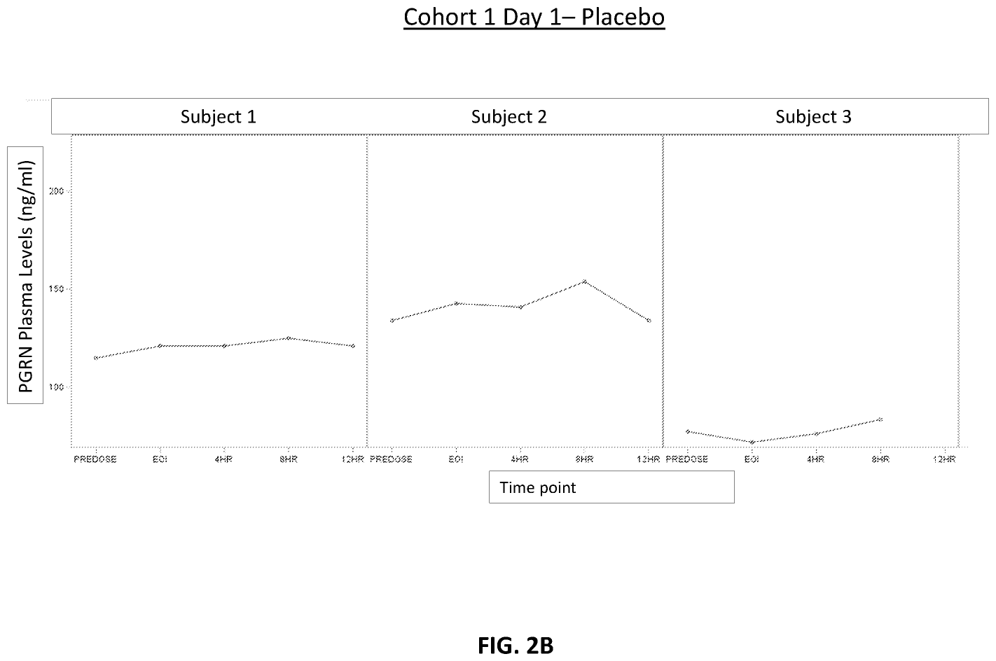

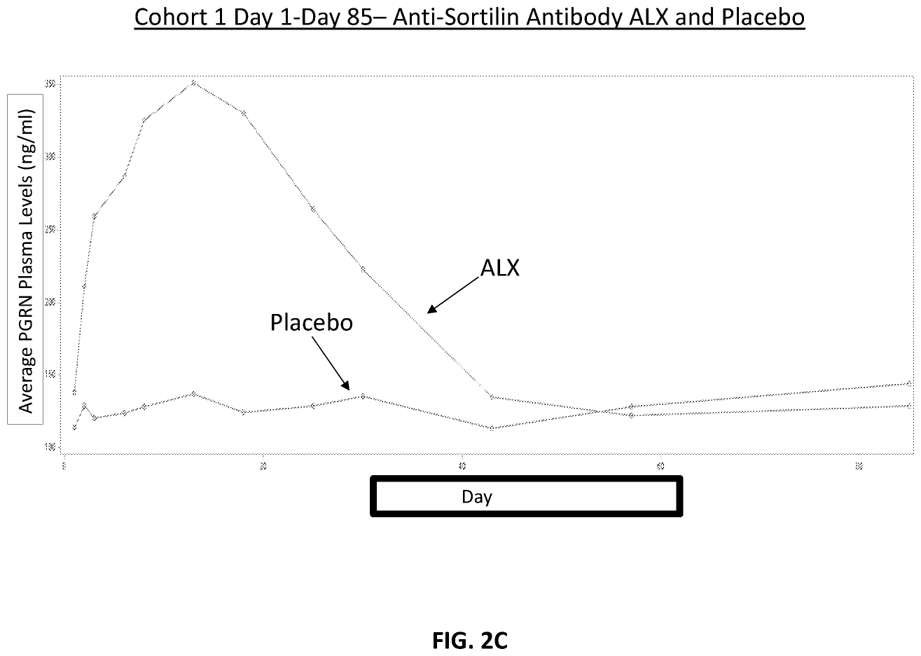

FIGS. 2A-2B show the levels of progranulin in the plasma of each subject in Cohort 1 at the indicated times on Day 1 of the study. FIG. 2A provides the plasma progranulin levels for subjects administered antibody ALX (n=8) and FIG. 2B provides the plasma progranulin levels for subjects administered placebo (n=3). FIG. 2C provides the average levels of progranulin in the plasma of subjects administered placebo or antibody ALX in Cohort 1 on the indicated days up to Day 85 of the study. These results showed that antibody ALX, administered at a dose of 6 mg/kg, resulted in an increase of plasma progranulin levels of about 2- to about 3-fold compared to baseline, and that the levels of plasma progranulin returned to baseline levels about 42 days after administration of antibody ALX.

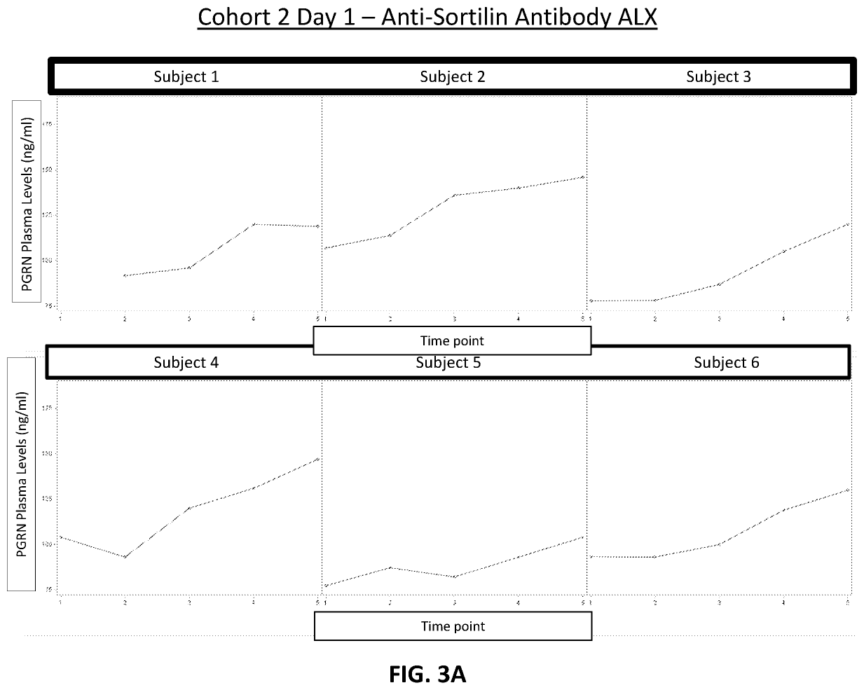

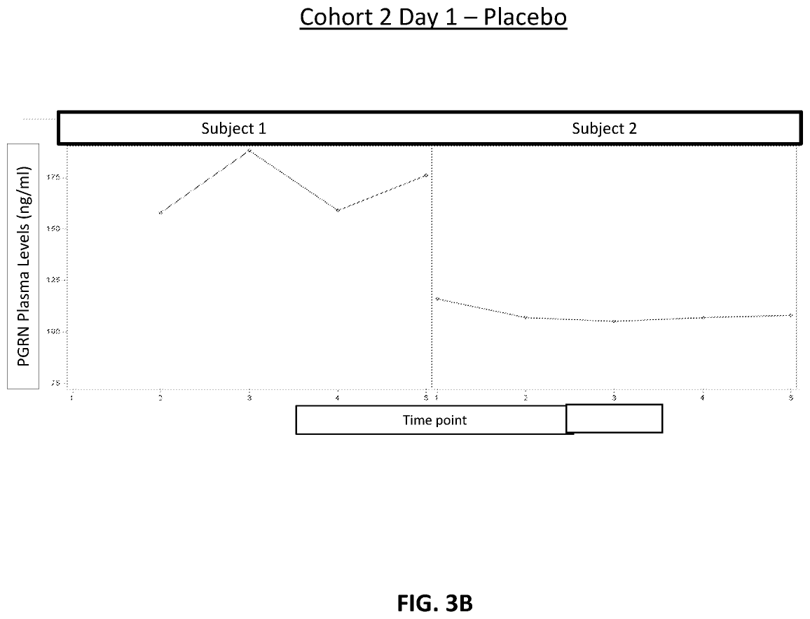

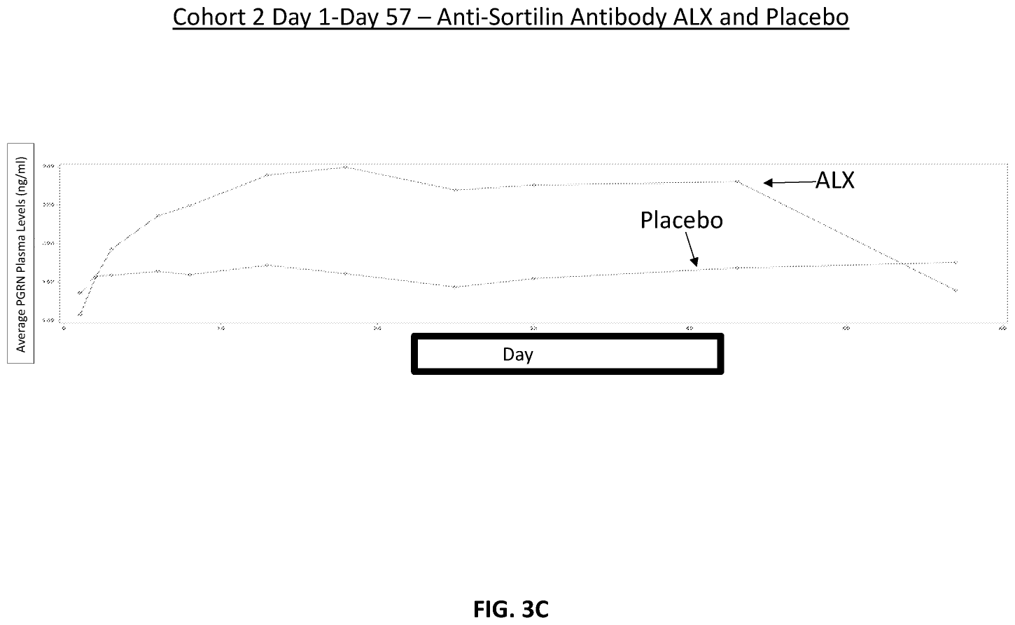

FIGS. 3A-3B show the levels of progranulin in the plasma of each subject in Cohort 2 at the indicated times on Day 1 of the study. FIG. 3A provides the plasma progranulin levels for subjects administered antibody ALX (n=6) and FIG. 3B provides the plasma progranulin levels for subjects administered placebo (n=2). FIG. 3C provides the average levels of progranulin in the plasma of subjects administered placebo or antibody ALX in Cohort 2 on the indicated days up to Day 57 of the study. Overall, these results showed that antibody ALX administered at a dose of 15 mg/kg resulted in an increase of plasma progranulin levels of about 2- to about 3-fold compared to baseline, and that the levels of plasma progranulin returned to baseline levels on about day 57 of the study.

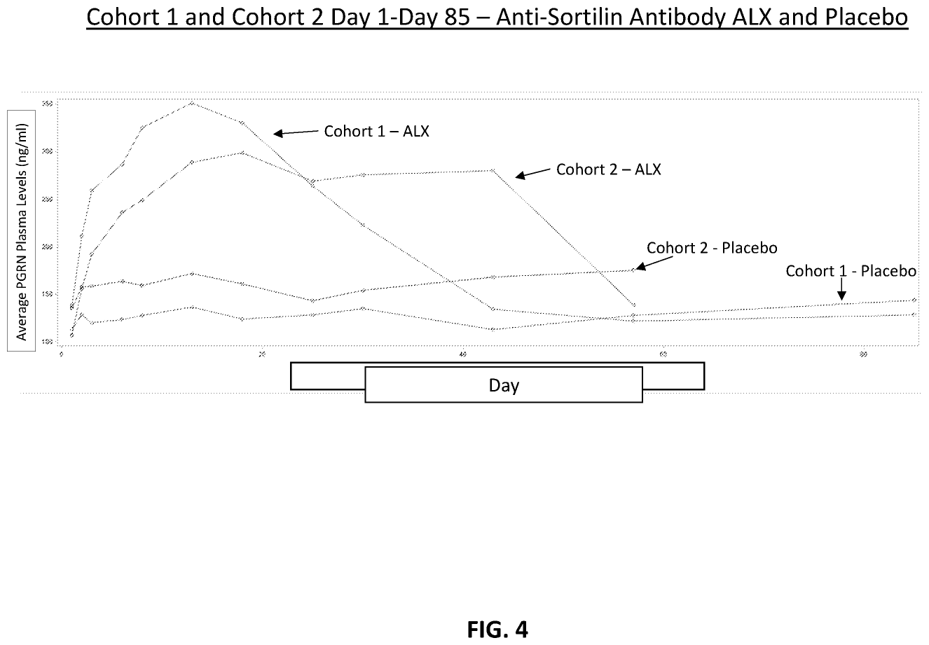

FIG. 4 provides a comparison of the levels of progranulin in the plasma of subjects administered antibody ALX or placebo in Cohorts 1 and 2 on the indicated days up to Day 57 (Cohort 2) or Day 85 (Cohort 1) of the study.

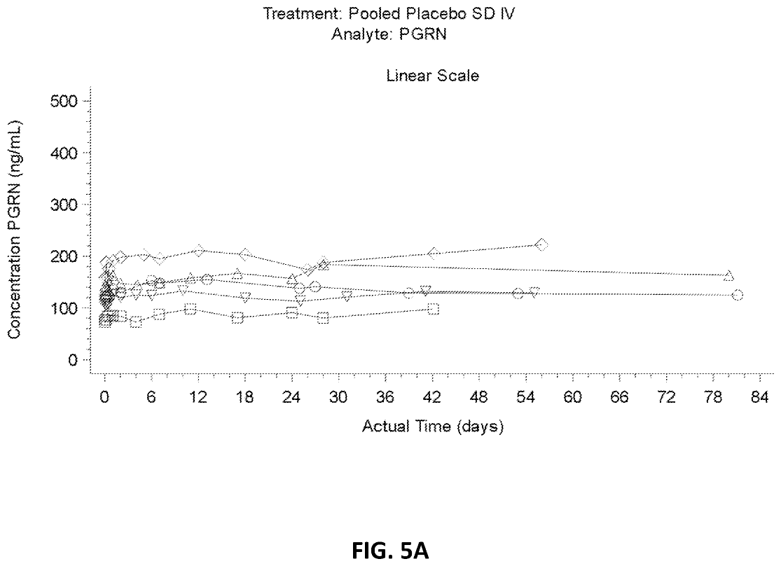

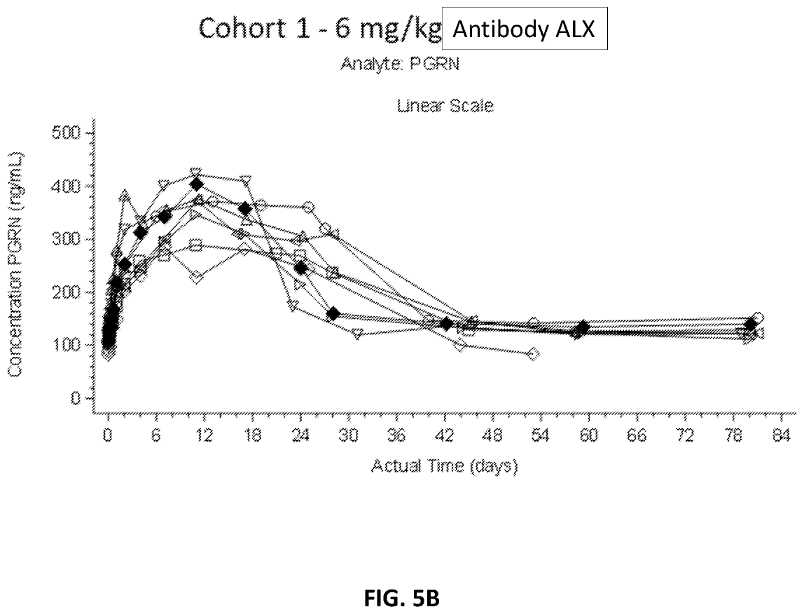

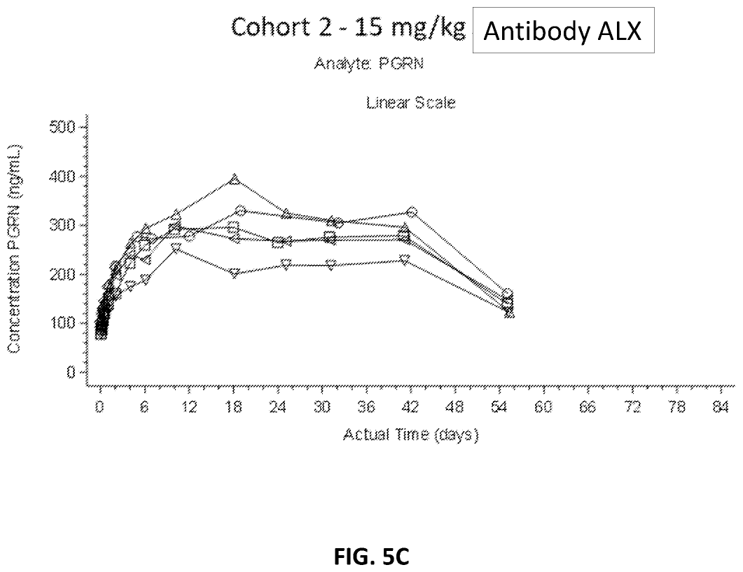

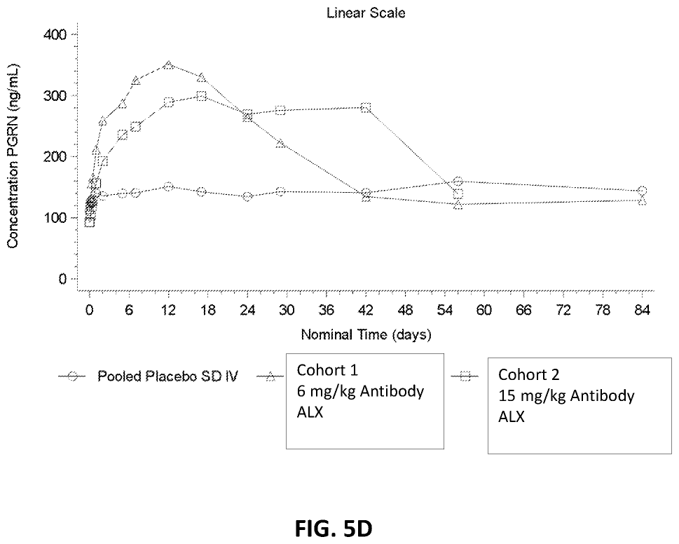

FIG. 5A shows the levels of progranulin in the plasma of subjects administered placebo in Cohorts 1 and 2 on the indicated days after administration of placebo. FIGS. 5B-5C show the levels of progranulin in the plasma of subjects administered antibody ALX in Cohort 1 (FIG. 5B) and Cohort 2 (FIG. 5C) on the indicated days after administration of antibody ALX. FIG. 5D provides a comparison of the levels of progranulin in the plasma of subjects administered antibody ALX or placebo in Cohorts 1 and 2 on the indicated days after administration of placebo or antibody ALX. In FIG. 5D, subjects administered placebo in Cohorts 1 and 2 were pooled. Overall, these results showed that administration of antibody ALX resulted in an increase of plasma progranulin levels of about 2- to about 3-fold over baseline, and that the levels of plasma progranulin returned to baseline levels about 42 days (Cohort 1) or about 56 days (Cohort 2) after administration of antibody ALX.

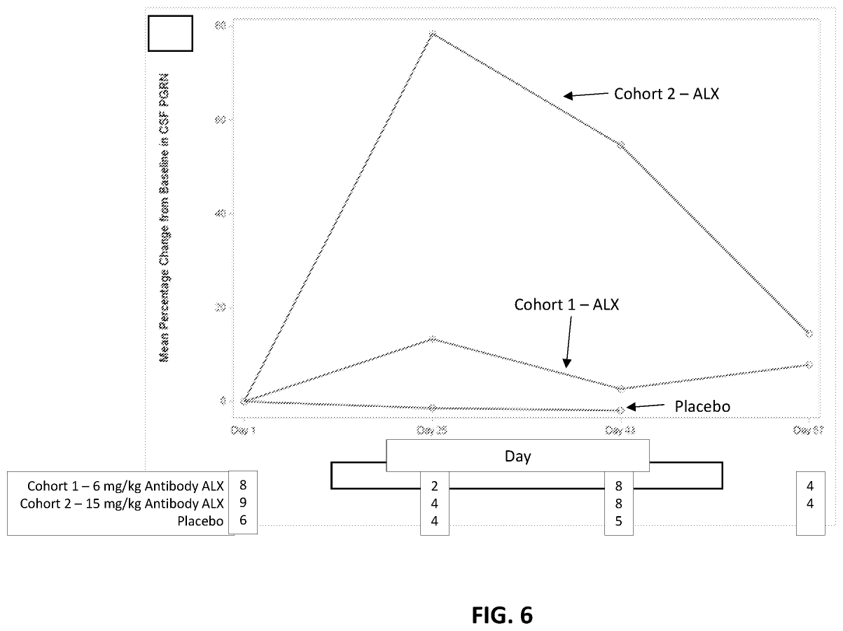

FIG. 6 shows the mean percentage change in the levels of progranulin in the CSF of subjects administered antibody ALX or placebo in Cohorts 1 and 2 on the indicated days of the study up to Day 57 compared to baseline. CSF progranulin levels increased about 18% from baseline for subjects in Cohort 1 (6 mg/kg) and about 80% from baseline for subjects in Cohort 2 (15 mg/kg) on about Day 25 of the study. Elevated CSF progranulin levels for subjects in Cohorts 1 and 2 administered antibody ALX were present up to Day 57 of the study.

Currently available safety results have demonstrated that antibody ALX appears to be well tolerated at single doses of up to 30 mg/kg.

Overall, the results described in these Examples show that administration of a single IV dose of antibody ALX resulted in a marked elevation of progranulin levels in plasma and CSF, and this elevation was sustained for at least 6 weeks.

### Example 3: A Phase IIa Study Evaluating the Safety, Tolerability, Pharmacokinetics, and Pharmacodynamics of Anti-Sortilin Antibody ALX in Patients with Parkinson's Disease

This Example describes a randomized, placebo-controlled, dose escalation Phase IIa study of anti-sortilin antibody ALX, evaluating the safety, tolerability, pharmacokinetics, and pharmacodynamic response of selected biomarkers in patients with Parkinson's disease (PD).

### I. Study Objectives

The objectives of this study are to evaluate the safety, tolerability, and effect on key biomarkers of a 6-month low-dose (15 mg/kg) and high-dose (30 mg/kg) regimen of antibody ALX in patients with sporadic (idiopathic) PD, as well as PD patients with at least one pathogenic mutation in the GBA1 gene. Key biomarkers analyzed include progranulin, GCase, and alpha-synuclein.

A. Primary Outcome Measures

The primary outcome measures of this study include the number of adverse events (AEs), serious adverse events (SAEs), and AEs leading to discontinuation in each treatment group over study duration. Safety and tolerability are based on additional safety assessments (e.g., clinical safety laboratory tests, vital signs, weight, electrocardiogram [ECG] parameters and physical examination). Further primary outcome measures of this study include changes in cerebrospinal fluid (CSF) and blood-based biomarkers (e.g., progranulin, GCase, and alpha-synuclein). Changes in CSF and blood-based biomarkers are assessed from baseline to the end of treatment (EOT).

B. Secondary Outcome Measures

The secondary outcome measures for this study include changes in motor examination, assessed by scores on the Movement Disorder Society (MDS)-sponsored revision of the Unified Parkinson's Disease Rating Scale (UPDRS) Part III (MDS-UPDRS Part III), and the UPDRS total scale. Changes in overall cognitive function, assessed by the Montreal Cognitive Assessment (MoCA) score, may also be measured. The secondary outcome measures are assessed from baseline to end of treatment (EOT).

### II. Study Participants

Forty-eight subjects with sporadic PD or PD patients with at least one pathogenic mutation in the GBA1 gene are included in this study.

A. Inclusion Criteria

Subjects that meet the following criteria are included in this study:
Adult males and females aged >40 years and <80 years.
Confirmed diagnosis of sporadic PD or PD with at least one pathogenic GBA1 mutation, and Hoehn and Yahr criteria with a confirmed stage of between I-III, inclusive.

Subjects who are taking PD medications prior to the start of this study, such as glutamate antagonists, anticholinergics, dopamine agonists, Levodopa (L-DOPA and decarboxylase [DDC] inhibitor), Monoamine oxidase B (MAO-B) inhibitors, catechol-O-methyltransferase (COMT) inhibitors, beta blockers, selective serotonin uptake inhibitors (SSRIs), tricyclic antidepressants (TCAs), or indomethacin, may continue taking the medications during this study.

B. Exclusion Criteria

Subjects that meet the following criteria are not included in this study:
Current treatment with anticoagulants (e.g., warfarin) that could preclude safe completion of a lumbar puncture.
History of known sensitivity to antibody ALX or its excipients.
Impaired renal function.
Active cancer diagnosis, except for skin cancer squamous cell carcinoma.
Moderate/severe hepatic impairment.
A major cardiovascular event (e.g., myocardial infarction, acute coronary syndrome, decompensated congestive heart failure, pulmonary embolism, or coronary revascularization) within six months of study screening.

### III. Study Design

This study is a randomized, placebo-controlled, sequential study of two dose levels of antibody ALX (15 mg/kg and 30 mg/kg), administered monthly via intravenous (IV) administration. Subjects are stratified by PD presentation (sporadic or GBA1 gene mutations).

Study subjects are randomized into 3 study arms: antibody ALX at a dose of 15 mg/kg, n=16; antibody ALX at a dose of 30 mg/kg, n=16; and placebo, n=16. Within each treatment arm, the randomization ratio of antibody ALX to placebo is 2:1. Study treatments are administered by intravenous infusion. Antibody ALX or placebo are administered once per month (i.e., once every 28 days) for a total of six administrations. The dose levels and frequency may be modified to remove or add dose levels, e.g., 60 mg/kg, and/or to alter the frequency of intravenous dosing (e.g., every six weeks (q6w) or every eight weeks (q8w)), and/or to include dosing by subcutaneous administration, as described herein. The placebo is saline, with appropriate procedures in place to ensure blinding.

### IV. Biomarkers and Target Engagement

As discussed above, the primary outcome measures of this study include changes in CSF and blood-based biomarkers (e.g., progranulin, GCase, and alpha-synuclein).

Plasma and CSF samples are collected for biomarker analyses. Changes in CSF and blood-based biomarkers are assessed from baseline to the end of treatment (EOT).

Plasma and CSF progranulin levels following antibody ALX treatment are measured as a marker of target engagement.

To monitor for lysosomal pharmacodynamic effects of antibody ALX, changes in GCase protein, GCase activity, and its substrates lyso-Gb1 and glucosylceramide in CSF and plasma are analyzed.

Alpha-synuclein and the CSF proteome are used to assess changes in disease status.

All assays are rigorously quality controlled and are validated.

### Example 4: A Study of Anti-Sortilin Antibody ALX in Cynomolgus Monkeys

This Example describes the results of a four-week toxicology study of anti-sortilin antibody ALX in cynomolgus monkeys.

Cynomolgus monkeys were administered antibody ALX intravenously at doses of 20, 60, or 200 mg/kg once weekly for four weeks, for a total of five doses. The study also included a six-week recovery period.

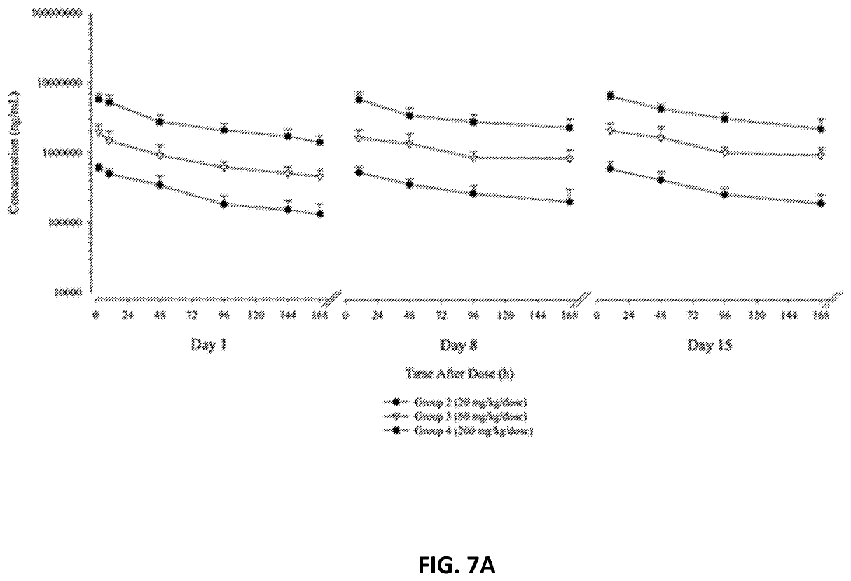

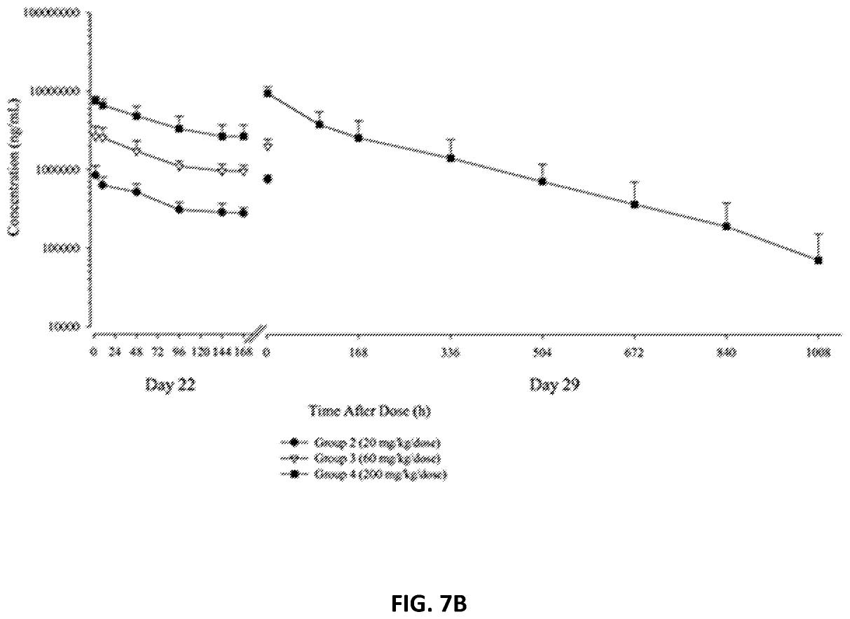

FIGS. 7A-7B show the mean concentration of antibody ALX in serum at the indicated times (hours) after administration of a dose of antibody ALX on Days 1, 8 and 15 (FIG. 7A) or Days 22 and 29 of the study (FIG. 7B). Exposure, assessed by maximum concentration (Cₘₐₓ) and area under the curve from time 0 to 168 hours (AUC₀₋₁₆₈), increased with the increase in dose level from 20 to 200 mg/kg/dose, and was generally dose-proportional. On Day 22, the gender-combined mean Cₘₐₓ and AUC₀₋₁₆₈ were 855 μg/mL and 70600 μg*h/mL, respectively, for the 20 mg/kg dose group; 2770 μg/mL and 248000 μg*h/mL, respectively, for the 60 mg/kg dose group; and 7960 μg/mL and 744000 μg*h/mL, respectively, for the 200 mg/kg dose group. Accumulation of antibody ALX was observed after multiple doses in monkeys. Gender combined mean Cₘₐₓ and AUC₀₋₁₆₈ ratios (Day 22/Day 1) ranged from 1.40 to 1.44 and 1.59 to 1.84 across doses.

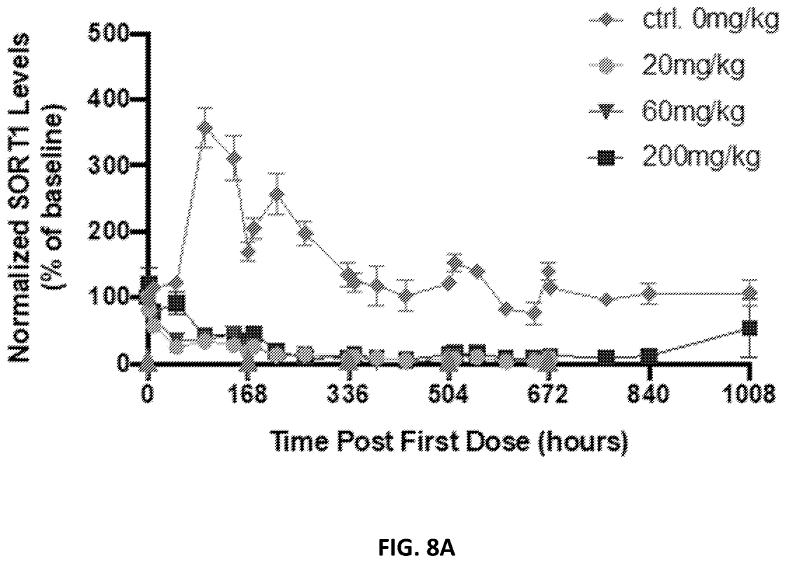

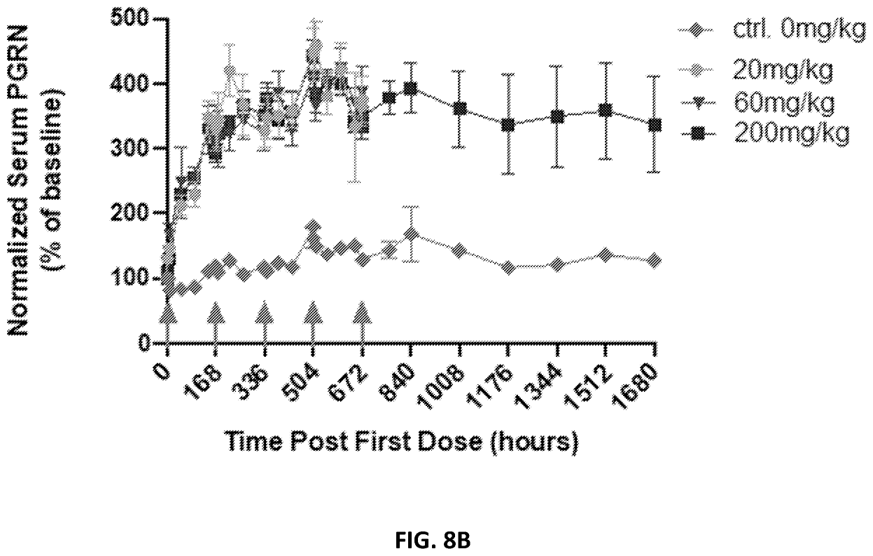

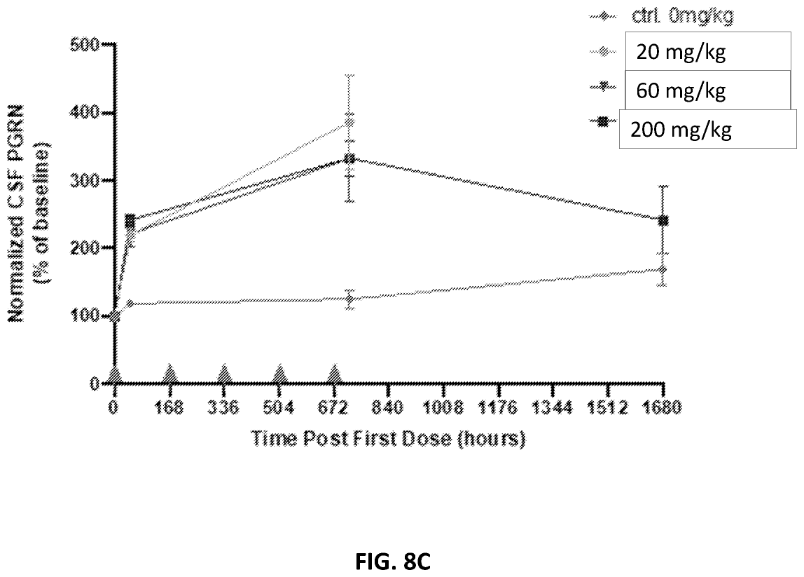

Sortilin expression in white blood cells (WBCs) was consistently down-regulated after weekly administration of antibody ALX, with no significant difference in down-regulation of sortilin observed across the dose levels of 20, 60, and 200 mg/kg (FIG. 8A). As shown in FIG. 8B, peripheral serum levels of progranulin increased concomitantly with down-regulation of sortilin and remained elevated more than 3-fold above baseline throughout the duration of the study. CSF progranulin levels were more than 2- to 3-fold above baseline at all time points sampled during the dosing phase (FIG. 8C). At the end of the 6-week recovery phase, both serum and CSF progranulin levels in 3 out of 4 animals in the 200 mg/kg group remained significantly elevated, indicating a long-lasting pharmacodynamic effect (FIGS. 8B-8C).

The results of this study also showed that antibody ALX administered at doses up to 200 mg/kg weekly in cynomolgus monkeys was well tolerated. Toxicokinetic analyses indicated exposure was maintained in antibody ALX-treated animals over the treatment period, and there were no antibody ALX-related adverse findings during the study. The no-observed-adverse-effect level (NOAEL) was 200 mg/kg for cynomolgus monkeys.

An additional toxicology study in RccHan:WIST rats administered antibody ALX at doses up to 300 mg/kg for 4 weeks showed that antibody ALX was well-tolerated. Toxicokinetic analyses indicated exposure was maintained in antibody ALX-treated animals over the treatment period and there were no antibody ALX-related adverse findings. The no-observed-adverse-effect level (NOAEL) was 300 mg/kg for rats.

### Example 5: Results of a Phase I Study in Healthy Volunteers to Assess the Safety, Tolerability, Pharmacokinetics, Pharmacodynamics, and Bioavailability of Intravenous and Subcutaneous Anti-Sortilin Antibody ALX

This Example provides additional results from the single-ascending dose (SAD) intravenous (IV) Cohorts 1-4 and the single dose 600 mg subcutaneous (SC) cohort of the Phase I study described in Example 1.

As described in detail in Example 1, HVs in an additional single-dose (SD) subcutaneous cohort were administered antibody ALX at the fixed dose of 600 mg. This dose corresponds to a maximum dose level of 13.3 mg/kg for the lowest body weight permitted in the study (45 kg). This cohort was enrolled after the Safety Review Committee determined that 15 mg/kg IV dose level was generally safe and tolerable.

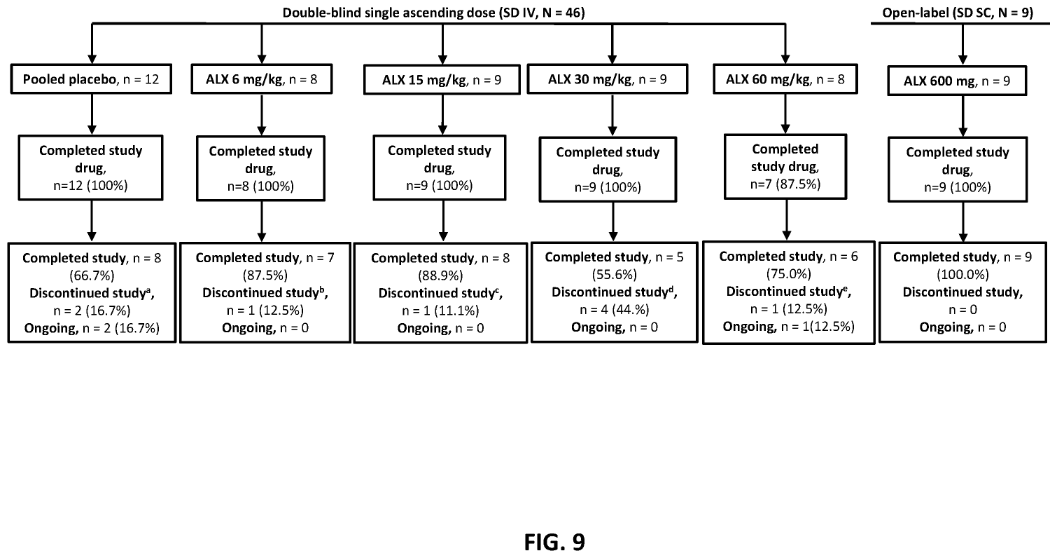

FIG. 9 provides an overview of participant disposition of the enrolled population at various time points during the study. Out of a total of 55 participants enrolled in the study, 54 participants (92.80%) completed drug treatment and 43 participants (78.20%) completed the study. As shown in Table 6, baseline demographics and characteristics were similar across groups. Across all groups, most participants were either White or Black, and the majority were not Hispanic or Latino.

TABLE 6
Baseline demographics and characteristics (safety population)

| | Double-blind single ascending dose IV | | | | | Open-label SC |
|---|---|---|---|---|---|---|
| | Pooled | ALX | ALX | ALX | ALX | ALX |
| | placebo | 6 mg/kg | 15 mg/kg | 30 mg/kg | 60 mg/kg | 600 mg |
| | (N = 12) | (N = 8) | (N = 9) | (N = 9) | (N = 8) | (N = 9) |
| Age (years), | 43.7 (10.9) | 39.0 (14.8) | 41.9 (13.1) | 40.0 (12.3) | 39.0 (14.0) | 44.4 (11.4) |
| mean (SD) | | | | | | |
| Sex, n (%) | | | | | | |
| Female | 6 (50.0) | 2 (25.0) | 5 (55.6) | 3 (33.3) | 5 (62.5) | 3 (33.3) |
| Male | 6 (50.0) | 6 (75.0) | 4 (44.4) | 6 (66.7) | 3 (37.5) | 6 (66.7) |
| Race, n (%) | | | | | | |
| White | 8 (66.7) | 6 (75.0) | 1 (11.1) | 5 (55.6) | 7 (87.5) | 3 (33.3) |
| Black or | 4 (33.3) | 2 (25.0) | 5 (55.6) | 4 (44.4) | 1 (12.5) | 5 (55.6) |
| African | | | | | | |
| American | | | | | | |
| Asian | 0 | 0 | 0 | 0 | 0 | 1 (11.1) |
| American | 0 | 0 | 1 (11.1) | 0 | 0 | 0 |
| Indian or | | | | | | |
| Alaska | | | | | | |
| Native | | | | | | |
| Native | 0 | 0 | 0 | 0 | 0 | 0 |
| Hawaiian | | | | | | |
| or other | | | | | | |
| Pacific | | | | | | |
| Islander | | | | | | |
| Multiracial | 0 | 0 | 1 (11.1) | 0 | 0 | 0 |
| Not | 0 | 0 | 1 (11.1) | 0 | 0 | 0 |
| reported | | | | | | |
| Ethnicity, n (%) | | | | | | |
| Hispanic | 4 (33.3) | 1 (12.5) | 3 (33.3) | 3 (33.3) | 4 (50.0) | 4 (44.5) |
| or Latino | | | | | | |
| Not | 8 (66.7) | 7 (87.5) | 6 (66.7) | 6 (66.7) | 4 (50.0) | 5 (55.6) |
| Hispanic | | | | | | |
| or Latino | | | | | | |
| Screening | 174.1 (11.5) | 172.6 (8.9) | 169.3 (6.8) | 174.3 (10.7) | 167.4 (8.3) | 169.0 (9.8) |
| height (cm), | | | | | | |
| mean (SD) | | | | | | |
| Screening | 86.5 (13.4) | 75.8 (13.8) | 75.6 (11.5) | 80.0 (15.5) | 79.3 (16.4) | 78.4 (12.9) |
| weight (kg), | | | | | | |
| mean (SD) | | | | | | |
| Predose | 86.1 (13.4) | 75.3 (13.6) | 75.4 (11.7) | 79.8 (15.0) | 78.3 (16.5) | 78.4 (12.9) |
| weight (kg), | | | | | | |
| mean (SD) | | | | | | |
| Screening | 28.6 (3.5) | 25.3 (3.1) | 26.3 (2.9) | 26.1 (2.7) | 28.1 (3.6) | 27.4 (3.3) |
| BMI (kg/m²), | | | | | | |
| mean (SD) | | | | | | |

BMI = body mass index; IV = intravenous; SC = subcutaneous; SD = standard deviation.

As shown in Table 7, most adverse events (AEs) were considered mild to moderate in severity, with the most frequent AEs being headache (25.6%), anemia (9.3%), and procedural pain (9.3%). One subject displayed one serious and severe event of infusion reaction (60 mg/kg IV group) considered related to study treatment, and thus discontinued study drug shortly after infusion but recovered within same day. Another subject displayed one serious and severe adverse event of Influenza A (placebo group), which was considered unrelated to study treatment. A last subject (30 mg/kg group), displayed concurrent severe decrease in glomerular filtration rate and elevated creatinine, but both were non-serious and considered unrelated to study treatment.

TABLE 7
TEAEs occurring in two or more subjects across all groups (safety population)

| | Double-blind single ascending dose IV | | | | | Open-label SD SC | |
|---|---|---|---|---|---|---|---|
| | Pooled | ALX | ALX | ALX | ALX | ALX | All Active |
| | placebo | 6 mg/kg | 15 mg/kg | 30 mg/kg | 60 mg/kg | 600 mg | ALX |
| n (%) [E] | (N = 12) | (N = 8) | (N = 9) | (N = 9) | (N = 8) | (N = 9) | (N = 43) |
| Any TEAE | 7 (58.3) [11] | 4 (50.0) [9] | 7 (77.8) [9] | 7 (77.8) [15] | 5 (62.5) [12] | 6 (66.7) [14] | 29 (67.4) [59] |
| Headache | 0 | 3 (37.5) [5] | 1 (11.1) [1] | 3 (33.3) [4] | 3 (37.5) [4] | 1 (11.1) [1] | 11 (25.6) [15] |
| Anemia | 0 | 1 (12.5) [1] | 2 (22.3) [3] | 1 (11.1) [1] | 0 | 0 | 4 (9.3) [5] |
| Procedural pain | 0 | 3 (37.5) [3] | 0 | 0 | 0 | 1 (11.1) [1] | 4 (9.3) [4] |
| Back pain | 0 | 0 | 0 | 1 (11.1) [1] | 2 (25.0) [2] | 0 | 3 (7.0) [3] |
| Injection site | 0 | 0 | 0 | 0 | 0 | 3 (33.3) [3] | 3 (7.0) [3] |
| erythema | | | | | | | |
| Injection site | 0 | 0 | 0 | 0 | 0 | 3 (33.3) [3] | 3 (7.0) [3] |
| induration | | | | | | | |
| Alanine | 1 (8.3) [1] | 0 | 1 (11.2) [1] | 1 (11.1) [1] | 0 | 0 | 2 (4.7) [2] |
| aminotransferase | | | | | | | |
| increased | | | | | | | |
| Post lumbar | 1 (8.3) [1] | 0 | 2 (22.2) [2] | 0 | 0 | 0 | 2 (4.7) [2] |
| puncture | | | | | | | |
| syndrome | | | | | | | |

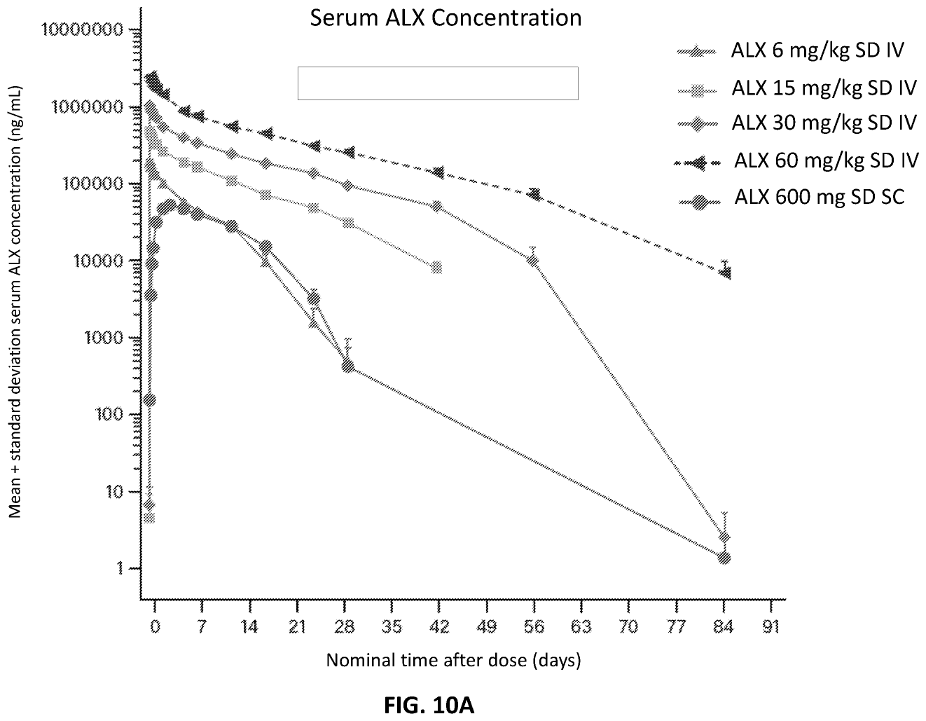

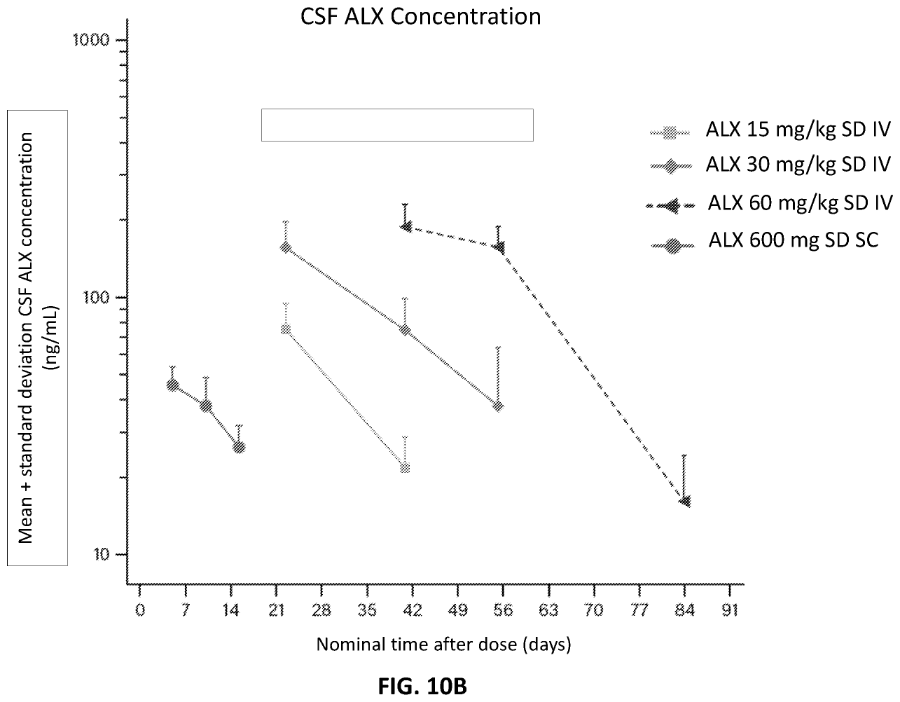

FIGS. 10A-10B show mean serum and cerebrospinal fluid (CSF) concentrations of ALX in each cohort after administration of a SAD IV dose or a single subcutaneous dose of ALX. FIG. 10A shows that mean serum concentrations of ALX increased in a dose-dependent manner after administration of a SAD IV dose of ALX and were detected for up to 30 days after a single SC dose of 600 mg of ALX. FIG. 10B shows CSF concentrations increased in a dose-dependent manner after administration of a SAD IV dose of ALX and were detected for up to 17 days after a single SC dose of 600 mg of ALX. Both mean serum and CSF concentrations of ALX were detected up to 84 days after a single IV dose of 60 mg/kg (FIGS. 10A-10B).

Table 8 provides a summary of mean serum pharmacokinetic (PK) parameters of ALX for Cohorts 1-4 and the SD SC Cohort. As shown in Table 8, the mean serum partition coefficient ranged from 0.0015 to 0.0007.

TABLE 8
Summary of mean (standard deviation) serum PK parameters of ALX (PK population)

| | AUC_{inf} | CL | Cₘₐₓ | tₘₐₓ | t_{1/2} | PC day | PC day | PC day | PC day |
|---|---|---|---|---|---|---|---|---|---|
| | (h*ug/mL) | (mL/h) | (ug/mL) | (h) | (h) | 13 | 25 | 43 | 57 |
| ALX 6 mg/kg | 18700 | 24.8 | 155 | 1.35 | 83.2 | NE^{a} | NE^{b} | NE^{b} | NE^{b} |
| IV | (4010) | (5.15) | (32.3) | (0.02) | (16.5) | | | | |
| ALX 15 | 74400 | 14.9 | 372 | 1.29 | 196 | NE^{a} | 0.00183 | 0.00347 | NE^{a} |
| mg/kg IV | (9830) | (1.23) | (49.7) | (0.04) | (44.0) | | (0.0008) | (0.003) | |
| ALX 30 | 178000 | 13.6 | 753 | 2.61 | 261 | NE^{a} | 0.00146 | 0.00114 | 0.00683 |
| mg/kg IV | (20600) | (3.65) | (87.3) | (2.73) | (75.2) | | (0.0007) | (0.0009) | (0.0047) |
| ALX 60 | 438000 | NE | 1740 | 1.81 | 294 | NE^{a} | NE^{a} | 0.0021 | 0.00194 |
| mg/kg IV | (85100) | | (270) | (1.44) | (68.1) | | | (0.0008) | (0.0005) |
| ALX 600 mg | 13500 | 47.5 | 48.0 | 82.26 | 95.8 | 0.00178 | NE^{a} | NE^{a} | NE^{a} |
| SC | (3280) | (15.0) | (9.86) | (27.38) | (58.8) | (0.0009) | | | |

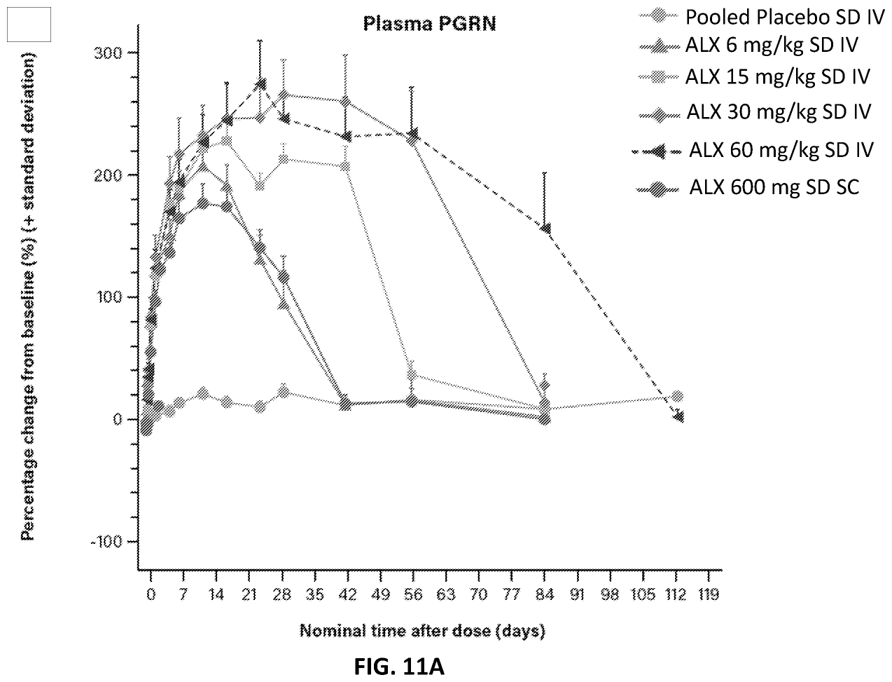

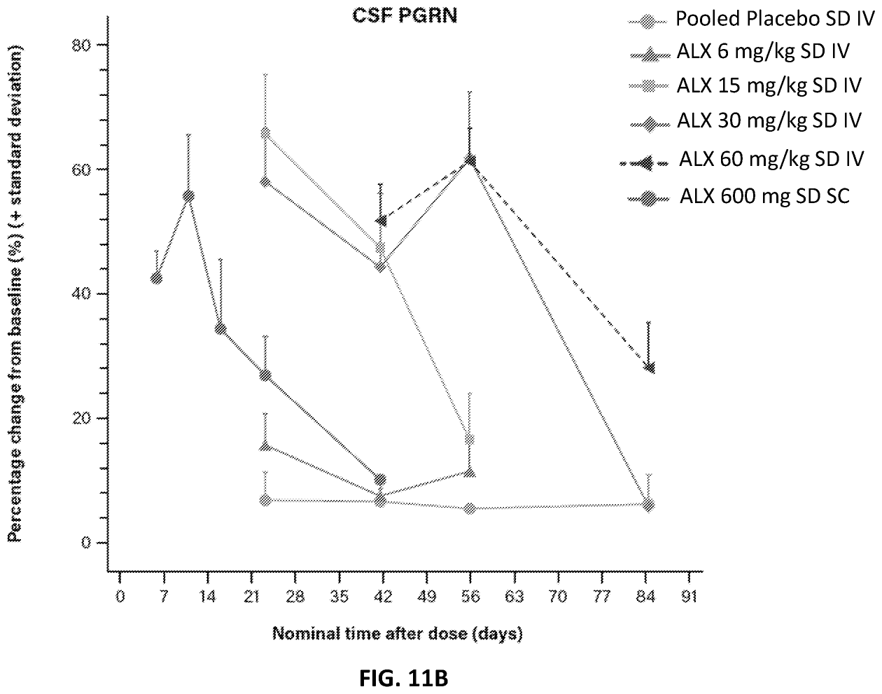

FIGS. 11A-11B show the percentage change from baseline in plasma and CSF concentrations of progranulin of each cohort at the indicated days of the study. A SAD IV dose of ALX caused an increase in progranulin (PGRN) levels in the periphery (FIG. 11A) and the brain (FIG. 11B), with a dose-dependent effect on the duration of the increase. FIG. 11A shows that ALX-induced increase of plasma PGRN levels was prolonged with increasing IV dose levels. SC administration of 600 mg ALX caused a robust increase in plasma PGRN that persisted up to 29 days after dosing. FIG. 11B shows a parallel increase in CSF PGRN that persisted up to 24 days after dosing.

Overall, the results described in these Examples show that ALX was found to be safe and well tolerated with single-dose IV or SC administrations. These results showed that ALX exposure increased in a dose-proportional manner and was distributed into the central nervous system, as evidenced by CSF ALX concentrations. These results demonstrate that ALX is a potent modulator of PGRN levels in the CSF, with a PK/pharmacodynamics (PD) profile that supports development of IV and SC ALX in chronic conditions.

TABLE 9
Sequences.

| | | SEQ ID |
|---|---|---|
| Description | Sequence | NO |
| Human | 10 20 30 40 50 | 1 |
| Sortilin | MERPWGAADG LSRWPHGLGL LLLLQLLPPS TLSQDRLDAP PPPAAPLPRW | |
| | 60 70 80 90 100 | |
| | SGPIGVSWGL RAAAAGGAFP RGGRWRRSAP GEDEECGRVR DFVAKLANNT | |
| | 120 130 140 150 160 | |
| | HQHVFDDLRG SVSLSWVGDS TGVILVLTTF HVPLVIMTFG QSKLYRSEDY | |
| | 170 180 190 200 210 | |
| | GKNFKDITDL INNTFIRTEF GMAIGPENSG KVVLTAEVSG GSRGGRIFRS | |
| | 220 230 240 250 260 | |
| | SDFAKNFVQT DLPFHPLTQM MYSPQNSDYL LALSTENGLW VSKNFGGKWE | |
| | 270 280 290 300 310 | |
| | EIHKAVCLAK WGSDNTIFFT TYANGSCKAD LGALELWRTS DLGKSFKTIG | |
| | 320 330 340 350 360 | |
| | VKIYSFGLGG RFLFASVMAD KDTTRRIHVS TDQGDTWSMA QLPSVGQEQF | |
| | 360 370 380 390 400 | |
| | YSILAANDDM VFMHVDEPGD TGFGTIFTSD DRGIVYSKSL DRHLYTTTGG | |
| | 410 420 430 440 450 | |
| | ETDFTNVTSL RGVYITSVLS EDNSIQTMIT FDQGGRWTHL RKPENSECDA | |
| | 460 470 480 490 500 | |
| | TAKNKNECSL HIHASYSISQ KLNVPMAPLS EPNAVGIVIA HGSVGDAISV | |
| | 510 520 530 540 550 | |
| | MVPDVYISDD GGYSWTKMLE GPHYYTILDS GGIIVAIEHS SRPINVIKFS | |
| | 560 570 580 590 600 | |
| | TDEGQCWQTY TFTRDPIYFT GLASEPGARS MNISIWGFTE SFLTSQWVSY | |
| | 610 620 630 640 650 | |
| | TIDFKDILER NCEEKDYTIW LAHSTDPEDY EDGCILGYKE QFLRLRKSSV | |
| | 660 670 680 690 700 | |
| | CQNGRDYVVT KQPSICLCSL EDFLCDFGYY RPENDSKCVE QPELKGHDLE | |
| | 710 720 730 740 750 | |
| | FCLYGREEHL TTNGYRKIPG DKCQGGVNPV REVKDLKKKC TSNFLSPEKQ | |
| | 760 770 780 790 800 | |
| | NSKSNSVPII LAIVGLMLVT VVAGVLIVKK YVCGGRFLVH RYSVLQQHAE | |
| | 810 820 830 | |
| | ANGVDGVDAL DTASHINKSG YHDDSDEDLL E | |
| Mouse | MERPRGAADG LLRWPLGLLL LLQLLPPAAV GQDRLDAPPP PAPPLLRWAG | 2 |
| Sortilin | PVGVSWGLRA AAPGGPVPRA GRWRRGAPAE DQDCGRLPDF IAKLTNNTHQ | |
| | HVFDDLSGSV SLSWVGDSTG VILVLTTFQV PLVIVSFGQS KLYRSEDYGK | |
| | NFKDITNLIN NTFIRTEFGM AIGPENSGKV ILTAEVSGGS RGGRVFRSSD | |
| | FAKNFVQTDL PFHPLTQMMY SPQNSDYLLA LSTENGLWVS KNFGEKWEEI | |
| | HKAVCLAKWG PNNIIFFTTH VNGSCKADLG ALELWRTSDL GKTFKTIGVK | |
| | IYSFGLGGRF LFASVMADKD TTRRIHVSTD QGDTWSMAQL PSVGQEQFYS | |
| | ILAANEDMVF MHVDEPGDTG FGTIFTSDDR GIVYSKSLDR HLYTTTGGET | |
| | DFTNVTSLRG VYITSTLSED NSIQSMITED QGGRWEHLRK PENSKCDATA | |
| | KNKNECSLHI HASYSISQKL NVPMAPLSEP NAVGIVIAHG SVGDAISVMV | |
| | PDVYISDDGG YSWAKMLEGP HYYTILDSGG IIVAIEHSNR PINVIKFSTD | |
| | EGQCWQSYVF TQEPIYFTGL ASEPGARSMN ISIWGFTESF ITRQWVSYTV | |
| | DFKDILERNC EEDDYTTWLA HSTDPGDYKD GCILGYKEQF LRLRKSSVCQ | |
| | NGRDYVVAKQ PSVCPCSLED FLCDFGYFRP ENASECVEQP ELKGHELEFC | |
| | LYGKEEHLTT NGYRKIPGDK CQGGMNPARE VKDLKKKCTS NFLNPTKQNS | |
| | KSNSVPIILA IVGLMLVTVV AGVLIVKKYV CGGRFLVHRY SVLQQHAEAD | |
| | GVEALDSTSH AKSGYHDDSD EDLLE | |
| Rat Sortilin | MERPRGAADG LLRWPLGLLL LLQLLPPAAV GQDRLDAPPP PAPPLLRWAG | 3 |
| | PVGVSWGLRA AAPGGPVPRA GRWRRGAPAE DQDCGRLPDF IAKLTNNTHQ | |
| | HVFDDLSGSV SLSWVGDSTG VILVLTTFQV PLVIVSFGQS KLYRSEDYGK | |
| | NEKDITNLIN NTFIRTEFGM AIGPENSGKV ILTAEVSGGS RGGRVFRSSD | |
| | FAKNFVQTDL PFHPLTQMMY SPQNSDYLLA LSTENGLWVS KNFGEKWEEI | |
| | HKAVCLAKWG PNNIIFFTTH VNGSCKADLG ALELWRTSDL GKTFKTIGVK | |
| | IYSFGLGGRF LFASVMADKD TTRRIHVSTD QGDTWSMAQL PSVGQEQFYS | |
| | ILAANDDMVF MHVDEPGDTG FGTIFTSDDR GIVYSKSLDR HLYTTTGGET | |
| | DFTNVTSLRG VYITSTLSED NSIQSMITFD QGGRWEHLQK PENSKCDATA | |
| | KNKNECSLHI HASYSISQKL NVPMAPLSEP NAVGIVIAHG SVGDAISVMV | |
| | PDVYISDDGG YSWAKMLEGP HYYTILDSGG IIVAIEHSNR PINVIKFSTD | |
| | EGQCWQSYVF SQEPVYFTGL ASEPGARSMN ISIWGFTESF LRLRKSSVCQ | |
| | DFKDILERNC EENDYTTWLA HSTDPGDYKD GCILGYKEQF NFLNPKKQNS | |
| | NGRDYVVAKQ PSICPCSLED FLCDFGYFRP ENASECVEQP LTRQWVSYTI | |
| | LYGKEEHLTT NGYRKIPGDR CQGGMNPARE VKDLKKKCTS ELKGHELEFC | |
| | KSSSVPIILA IVGLMLVTVV AGVLIVKKYV CGGRFLVHRY SVLQQHAEAD | |
| | GVEALDTASH AKSGYHDDSD EDLLE | |
| Anti-Sortilin | NGLWVSKNFGG | 4 |
| Antibody | | |
| Binding | | |
| Region | | |
| Anti-Sortilin | FASVMADKDTTRRIHVSTDQGDTWS | 5 |
| Antibody | | |
| Binding | | |
| Region | | |
| S-15-10-7 | YTFTKYYMS | 6 |
| HVR-H1 | | |
| S-15-10-7 | IINPIGGSTSYAQKFQG | 7 |
| HVR -H2 | | |
| S-15-10-7 | ARDPSGIALAGPASRGYQGMDV | 8 |
| HVR -H3 | | |
| S-15-10-7 | RASQSVSSNLA | 9 |
| HVR -L1 | | |
| S-15-10-7 | GASTRAT | 10 |
| HVR -L2 | | |
| S-15-10-7 | QQARLGPWT | 11 |
| HVR -L3 | | |
| VH FR1 | QVQLVQSGAEVKKPGASVKVSCKASG | 12 |
| VH FR2 | WVRQAPGQGLEWMG | 13 |
| VH FR3 | RVTMTRDTSTSTVYMELSSLRSEDTAVYYC | 14 |
| VH FR4 | WGQGTTVTVSS | 15 |
| VL FR1 | EIVMTQSPATLSVSPGERATLSC | 16 |
| VL FR2 | WYQQKPGQAPRLLIY | 17 |
| VL FR3 | GIPARFSGSGSGTEFTLTISSLQSEDFAVYYC | 18 |
| VL FR4 | FGGGTKVEIK | 19 |
| S-15-10-7 | QVQLVQSGAEVKKPGASVKVSCKASGYTFTKYYMSWVRQAPGQGLEWMGIINPI | 20 |
| Heavy chain | GGSTSYAQKFQGRVTMTRDTSTSTVYMELSSLRSEDTAVYYCARDPSGIALAGP | |
| variable | ASRGYQGMDVWGQGTTVTVSS | |
| (VH) | | |
| domain | | |
| S-15-10-7 | EIVMTQSPATLSVSPGERATLSCRASQSVSSNLAWYQQKPGQAPRLLIYGASTR | 21 |
| Light chain | ATGIPARFSGSGSGTEFTLTISSLQSEDFAVYYCQQARLGPWTFGGGTKVEIK | |
| variable | | |
| (VL) | | |
| domain | | |
| Asp-box | (S/T)-X-(D/N)-X-X-X-X-(W/F/Y) | 22 |
| motif; X | | |
| represents | | |
| any amino | | |
| acid | | |
| Asp-box | X-X-(S/T)-X-(D/N)-X-G-X-(T/S)-(W/F/Y)-X | 23 |
| motif; X | | |
| represents | | |
| any amino | | |
| acid | | |
| Asp-box | SSDFAKNF | 24 |
| motif | | |
| huIgG1 | ASTKGPSVFPLAPSSKSTSGGTAALGCLVKDYFPEPVTVSWNSGALTSGVHTFP | 25 |
| heavy chain | AVLQSSGLYSLSSVVTVPSSSLGTQTYICNVNHKPSNTKVDKKVEPKSCDKTHT | |
| constant | CPPCPAPEAAGGPSVFLFPPKPKDTLMISRTPEVTCVVVDVSHEDPEVKENWYV | |
| region | DGVEVHNAKTKPREEQYNSTYRVVSVLTVLHQDWLNGKEYKCKVSNKALPASIE | |
| (CH1, hinge, | KTISKAKGQPREPQVYTLPPSRDELTKNQVSLTCLVKGFYPSDIAVEWESNGQP | |
| CH2, CH3) | ENNYKTTPPVLDSDGSFFLYSKLTVDKSRWQQGNVFSCSVMHEALHNHYTQKSL | |
| LALAPS Fc | SLSPGK | |
| with C- | | |
| terminal | | |
| lysine | | |
| huIgG1 | ASTKGPSVFPLAPSSKSTSGGTAALGCLVKDYFPEPVTVSWNSGALTSGVHTFP | 26 |
| heavy chain | AVLQSSGLYSLSSVVTVPSSSLGTQTYICNVNHKPSNTKVDKKVEPKSCDKTHT | |
| constant | CPPCPAPEAAGGPSVFLFPPKPKDTLMISRTPEVTCVVVDVSHEDPEVKENWYV | |
| region | DGVEVHNAKTKPREEQYNSTYRVVSVLTVLHQDWLNGKEYKCKVSNKALPASIE | |
| (CH1, hinge, | KTISKAKGQPREPQVYTLPPSRDELTKNQVSLTCLVKGFYPSDIAVEWESNGQP | |
| CH2, CH3) | ENNYKTTPPVLDSDGSFFLYSKLTVDKSRWQQGNVFSCSVMHEALHNHYTQKSL | |
| LALAPS Fc | SLSPG | |
| without C- | | |
| terminal | | |
| lysine | | |
| Cynomolgus | MERPRGAADGLSRWPHGLGLLLLLQLLPPVTLGQDRLDAPPPPAAPLLRWSGPI | 27 |
| monkey | GVSWGLRAAAAGGAFPRGGRWRRSAPGEDEECGRVRDFVAKLANNTHQHVEDDL | |
| sortilin | RGSVSLSWVGDSTGVILVLTTFHVPLVIMTFGQSKLYRSEDYGKNFKDITNLIN | |
| | NTFIRTEFGMAIGPENSGKVVLTAEVSGGSRGGRIFRSSDFAKNFVQTDLPFHP | |
| | LTQMMYSPQNSDYLLALSTENGLWVSKNFGGKWEEIHKAVCLAKWGSDNTIFFT | |
| | TYANGSCKADLGALELWRTLDLGKSFKTIGVKIYSFGLGGRFLFASVMADKDTT | |
| | RRIHVSTDQGDTWSMAQLPSVGQEQFYSILAANDDMVFMHVDEPGDTGFGTIFT | |
| | SDDRGIVYSKSLDRHLYTTTGGETDFTNVTSLRGVYITSVLSEDNSIQTMITED | |
| | QGGRWKHLRKPENSECDATAKNKNECSLHIHASYSISQKLNVPMAPLSEPNAVG | |
| | IVIAHGSVGDAISVMVPDVYISDDGGYSWTKMLEGPHYYTILDSGGIIVAIEHS | |
| | SRPINVIKFSTDEGQCWQTYTFTRDPIYFTGLASEPGARSMNISIWGFTESFLT | |
| | SQWVSYTIDFKDILERNCEEKDYTIWLAHSTDPEDYEDGCILGYKEQFLRLRKS | |
| | SVCQNGRDYVVTKQPSICLCSLEDFLCDFGYYRPENDSKCVEQPELKGHDLEFC | |
| | LYGREEHLTTNGYRKIPGDKCQGGVNPVREVKDLKKKCTSNFLSPEKQNSKSNS | |
| | VPIILAIVGLMLVTVIAGVLIVKKYVCGGRFLVHRYSVLQQHAEANGMDGVDAL | |
| | DTASHTNKSGYHDDSDEDLLE | |
| huIgG1 | ASTKGPSVFPLAPSSKSTSGGTAALGCLVKDYFPEPVTVSWNSGALTSGVHTFP | 28 |
| heavy chain | AVLQSSGLYSLSSVVTVPSSSLGTQTYICNVNHKPSNTKVDKKVEPKSCDKTHT | |
| constant | CPPCPAPELLGGPSVFLFPPKPKDTLMISRTPEVTCVVVDVSHEDPEVKENWYV | |
| region | DGVEVHNAKTKPREEQYNSTYRVVSVLTVLHQDWLNGKEYKCKVSNKALPAPIE | |
| (CH1, hinge, | KTISKAKGQPREPQVYTLPPSRDELTKNQVSLTCLVKGFYPSDIAVEWESNGQP | |
| CH2, CH3) | ENNYKTTPPVLDSDGSFFLYSKLTVDKSRWQQGNVFSCSVMHEALHNHYTQKSL | |
| with C- | SLSPGK | |
| terminal | | |
| lysine | | |
| huIgG1 | ASTKGPSVFPLAPSSKSTSGGTAALGCLVKDYFPEPVTVSWNSGALTSGVHTFP | 29 |
| heavy chain | AVLQSSGLYSLSSVVTVPSSSLGTQTYICNVNHKPSNTKVDKKVEPKSCDKTHT | |
| constant | CPPCPAPELLGGPSVFLFPPKPKDTLMISRTPEVTCVVVDVSHEDPEVKENWYV | |
| region | DGVEVHNAKTKPREEQYNSTYRVVSVLTVLHQDWLNGKEYKCKVSNKALPAPIE | |
| (CH1, hinge, | KTISKAKGQPREPQVYTLPPSRDELTKNQVSLTCLVKGFYPSDIAVEWESNGQP | |
| CH2, CH3) | ENNYKTTPPVLDSDGSFFLYSKLTVDKSRWQQGNVFSCSVMHEALHNHYTQKSL | |
| without C- | SLSPG | |
| terminal | | |
| lysine | | |
| Full-length | E I V M T Q S P A T L S V S P G E R A T | 30 |
| light chain | L S C R A S Q S V S S N L A W Y Q Q K P | |
| amino acid | G Q A P R L L I Y G A S T R A T G I P A | |
| sequence of | R F S G S G S G T E F T L T I S S L Q S | |
| antibody | E D F A V Y Y C Q Q A R L G P W T F G G | |
| ALX | G T K V E I K R T V A A P S V F I F P P | |
| (containing | S D E Q L K S G T A S V V C L L N N F Y | |
| VL of | P R E A K V Q W K V D N A L Q S G N S Q | |
| antibody S- | E S V T E Q D S K D S T Y S L S S T L T | |
| 15-10-7) | L S K A D Y E K H K V Y A C E V T H Q G | |
| | L S S P V T K S F N R G E C | |
| Full-length | Q V Q L V Q S G A E V K K P G A S V K V | 31 |
| heavy chain | S C K A S G Y T F T K Y Y M S W V R Q A | |
| amino acid | P G Q G L E W M G I I N P I G G S T S Y | |
| sequence of | A Q K F Q G R V T M T R D T S T S T V Y | |
| antibody | M E L S S L R S E D T A V Y Y C A R D P | |
| ALX with | S G I A L A G P A S R G Y Q G M D V W G | |
| C-terminal | Q G T H V H V S S A S T K G P S V F P L | |
| lysine | A P S S K S T S G G T A A L G C L V K D | |
| (containing | Y F P E P V T V S W N S G A L T S G V H | |
| VH of | T F P A V L Q S S G L Y S L S S V V T V | |
| antibody S- | P S S S L G T Q T Y I C N V N H K P S N | |
| 15-10-7) | T K V D K K V E P K S C D K T H T C P P | |
| | C P A P E A A G G P S V F L F P P K P K | |
| | D T L M I S R T P E V T C V V V D V S H | |
| | E D P E V K F N W Y V D G V E V H N A K | |
| | T K P R E E Q Y N S T Y R V V S V L T V | |
| | L H Q D W L N G K E Y K C K V S N K A L | |
| | P A S I E K T I S K A K G Q P R E P Q V | |
| | Y T L P P S R D E L T K N Q V S L T C L | |
| | V K G F Y P S D I A V E W E S N G Q P E | |
| | N N Y K T T P P V L D S D G S F F L Y S | |
| | K L T V D K S R W Q Q G N V F S C S V M | |
| | H E A L H N H Y T Q K S L S L S P G K | |
| Full-length | Q V Q L V Q S G A E V K K P G A S V K V | 32 |
| heavy chain | S C K A S G Y T F T K Y Y M S W V R Q A | |
| amino acid | P G Q G L E W M G I I N P I G G S T S Y | |
| sequence of | A Q K F Q G R V T M T R D T S T S T V Y | |
| antibody | M E L S S L R S E D T A V Y Y C A R D P | |
| ALX | S G I A L A G P A S R G Y Q G M D V W G | |
| without C- | Q G T T V T V S S A S T K G P S V F P L | |
| terminal | A P S S K S T S G G T A A L G C L V K D | |
| lysine | Y F P E P V T V S W N S G A L T S G V H | |
| (containing | T F P A V L Q S S G L Y S L S S V V T V | |
| VH of | P S S S L G T Q T Y I C N V N H K P S N | |
| antibody S- | T K V D K K V E P K S C D K T H T C P P | |
| 15-10-7) | C P A P E A A G G P S V F L F P P K P K | |
| | D T L M I S R T P E V T C V V V D V S H | |
| | E D P E V K F N W Y V D G V E V H N A K | |
| | T K P R E E Q Y N S T Y R V V S V L T V | |
| | L H Q D W L N G K E Y K C K V S N K A L | |
| | P A S I E K T I S K A K G Q P R E P Q V | |
| | Y T L P P S R D E L T K N Q V S L T C L | |
| | V K G F Y P S D I A V E W E S N G Q P E | |
| | N N Y K T T P P V L D S D G S F F L Y S | |
| | K L T V D K S R W Q Q G N V F S C S V M | |
| | H E A L H N H Y T Q K S L S L S P G | |

What is claimed is:

1. A method of treating or delaying the progression of a disease, disorder, or injury in an individual, comprising administering to the individual an anti-sortilin antibody at a dose of at least about 6 mg/kg, wherein the antibody comprises a light chain variable domain and a heavy chain variable domain, wherein the heavy chain variable domain comprises an HVR-H1 comprising the amino acid sequence of SEQ ID NO:6, an HVR-H2 comprising the amino acid sequence of SEQ ID NO: 7, and an HVR-H3 comprising the amino acid sequence of SEQ ID NO:8, and the light chain variable domain comprises an HVR-L1 comprising the amino acid sequence of SEQ ID NO:9, an HVR-L2 comprising the amino acid sequence of SEQ ID NO: 10, and an HVR-L3 comprising the amino acid sequence of SEQ ID NO:11.

2. A method of increasing progranulin levels in an individual having a disease, disorder, or injury, comprising administering to the individual an anti-sortilin antibody at a dose of at least about 6 mg/kg, wherein the antibody comprises a light chain variable domain and a heavy chain variable domain, wherein the heavy chain variable domain comprises an HVR-H1 comprising the amino acid sequence of SEQ ID NO:6, an HVR-H2 comprising the amino acid sequence of SEQ ID NO: 7, and an HVR-H3 comprising the amino acid sequence of SEQ ID NO:8, and the light chain variable domain comprises an HVR-L1 comprising the amino acid sequence of SEQ ID NO:9, an HVR-L2 comprising the amino acid sequence of SEQ ID NO: 10, and an HVR-L3 comprising the amino acid sequence of SEQ ID NO:11.

3. The method of claim 2, wherein the increase in progranulin levels comprises an increase in the level of progranulin in the cerebrospinal fluid of the individual, the plasma of the individual, or both.

4. The method of any one of claims 1-3, comprising administering the anti-sortilin antibody by intravenous infusion or by subcutaneous injection.

5. The method of any one of claims 1-4, comprising administering the anti-sortilin antibody by intravenous infusion at a dose of at least about 6 mg/kg, at least about 15 mg/kg, at least about 30 mg/kg, or at least about 60 mg/kg.

6. The method of any one of claims 1-5, comprising administering the anti-sortilin antibody by intravenous infusion at a dose of between about 6 mg/kg and about 30 mg/kg.

7. The method of any one of claims 1-5, comprising administering the anti-sortilin antibody by intravenous infusion at a dose of about 6 mg/kg, about 15 mg/kg, about 30 mg/kg, or about 60 mg/kg.

8. The method of any one of claims 1-4, comprising:
(a) administering the anti-sortilin antibody by intravenous infusion at an initial dose of about 60 mg/kg, followed by one or more lower doses of between about 6 mg/kg and about 59 mg/kg, or between about 6 mg/kg and about 30 mg/kg, or between about 6 mg/kg and about 15 mg/kg, of the anti-sortilin antibody;
(b) administering the anti-sortilin antibody by intravenous infusion at an initial dose of about 30 mg/kg, followed by one or more lower doses of between about 6 mg/kg and about 29 mg/kg, or between about 6 mg/kg and about 15 mg/kg, of the anti-sortilin antibody;
(c) administering the anti-sortilin antibody by intravenous infusion at an initial dose of about 15 mg/kg, followed by one or more lower doses of between about 6 mg/kg and about 14 mg/kg of the anti-sortilin antibody;
(d) administering the anti-sortilin antibody by intravenous infusion at an initial dose of about 6 mg/kg, followed by one or more higher doses of between about 7 mg/kg and about 30 mg/kg, between about 15 mg/kg and about 30 mg/kg, or between about 30 mg/kg and about 60 mg/kg, of the anti-sortilin antibody;
(e) administering the anti-sortilin antibody by intravenous infusion at an initial dose of about 15 mg/kg, followed by one or more higher doses of between about 16 mg/kg and about 30 mg/kg, or between about 30 mg/kg and about 60 mg/kg, of the anti-sortilin antibody; or
(f) administering the anti-sortilin antibody by intravenous infusion at an initial dose of about 30 mg/kg, followed by one or more higher doses of between about 31 mg/kg and about 60 mg/kg of the anti-sortilin antibody.

9. The method of any one of claims 5-8, comprising administering the anti-sortilin antibody about once every four weeks or less frequently.

10. The method of claim 9, comprising administering the anti-sortilin antibody about once every four weeks, about once every five weeks, about once every six weeks, about once every seven weeks, about once every eight weeks, about once every nine weeks, or about once every ten weeks.

11. The method of claim 1 or claim 2, comprising administering the anti-sortilin antibody by intravenous infusion at a dose of about 15 mg/kg about once every four weeks, about once every six weeks, or about once every eight weeks.

12. The method of claim 1 or claim 2, comprising administering the anti-sortilin antibody by intravenous infusion at a dose of about 15 mg/kg about once every six weeks or about once every eight weeks.

13. The method of claim 1 or claim 2, comprising administering the anti-sortilin antibody by intravenous infusion at a dose of about 30 mg/kg about once every four weeks, about once every six weeks, or about once every eight weeks.

14. The method of claim 1 or claim 2, comprising administering the anti-sortilin antibody by intravenous infusion at a dose of about 30 mg/kg about once every six weeks or about once every eight weeks.

15. The method of claim 1 or claim 2, comprising administering the anti-sortilin antibody by intravenous infusion at a dose of about 60 mg/kg about once every four weeks, about once every six weeks or about once every eight weeks.

16. The method of claim 1 or claim 2, comprising administering the anti-sortilin antibody by intravenous infusion at a dose of about 60 mg/kg about once every six weeks or about once every eight weeks.

17. The method of any one of claims 1-4, comprising administering the anti-sortilin antibody by subcutaneous injection at a dose of at least about 270 mg.

18. The method of any one of claims 1-4, comprising administering the anti-sortilin antibody by subcutaneous injection at a dose of between about 150 mg and about 600 mg.

19. The method of claim 18, comprising administering the anti-sortilin antibody by subcutaneous injection at a dose of any of about 150 mg, about 270 mg, about 300 mg, or about 600 mg.

20. The method of claim 18, comprising administering the anti-sortilin antibody by subcutaneous injection at a dose of about 150 mg.

21. The method of claim 18, comprising administering the anti-sortilin antibody by subcutaneous injection at a dose of about 270 mg.

22. The method of claim 18, comprising administering the anti-sortilin antibody by subcutaneous injection at a dose of about 300 mg.

23. The method of claim 18, comprising administering the anti-sortilin antibody by subcutaneous injection at a dose of about 600 mg.

24. The method of any one of claims 17-23, comprising administering the anti-sortilin antibody by subcutaneous injection any of about every two weeks, about every four weeks, about every six weeks, or about every eight weeks.

25. The method of any one of claims 17-23, comprising administering the anti-sortilin antibody by subcutaneous injection about once every four weeks.

26. The method of any one of claims 1-25, wherein the anti-sortilin antibody comprises a heavy chain variable domain comprising the amino acid sequence of SEQ ID NO: 20 and a light chain variable domain comprising the amino acid sequence of SEQ ID NO: 21.

27. The method of any one of claims 1-26, wherein the anti-sortilin antibody has a human IgG1 isotype.

28. The method of claim 27, wherein the anti-sortilin antibody comprises an Fc region comprising amino acid substitutions L234A, L235A, and P331S, wherein the numbering of the residue position is according to EU numbering.

29. The method of any one of claims 1-28, wherein the anti-sortilin antibody comprises a light chain comprising the amino acid sequence of SEQ ID NO: 30, and a heavy chain comprising the amino acid sequence of SEQ ID NO: 31 or 32.

30. The method of any one of claims 1-29, wherein the anti-sortilin antibody comprises a light chain comprising the amino acid sequence of SEQ ID NO: 30, and a heavy chain comprising the amino acid sequence of SEQ ID NO: 32.

31. The method of any one of claims 1-29, wherein the anti-sortilin antibody comprises a light chain comprising the amino acid sequence of SEQ ID NO: 30, and a heavy chain comprising the amino acid sequence of SEQ ID NO: 31.

32. The method of any one of claims 1-31, further comprising measuring the level of progranulin in a sample of blood or cerebrospinal fluid obtained from the individual before and after the individual has received one or more doses of the anti-sortilin antibody.

33. The method of any one of claims 1-32, further comprising measuring the level of sortilin in white blood cells in a sample of blood obtained from the individual before and after the individual has received one or more doses of the anti-sortilin antibody.

34. The method of any one of claims 1-33, further comprising measuring the level of neurofilament light chain (NF-L), Tau, one or more biomarkers of neuroinflammation, one or more inflammatory biomarkers, one or more biomarkers of complement function, and/or one or more biomarkers of microglial activity in a sample of blood or cerebrospinal fluid obtained from the individual before and after the individual has received one or more doses of the anti-sortilin antibody.

35. The method of claim 34, wherein:
(a) the one or more biomarkers of neuroinflammation are selected from the group consisting of IL-6, SPP1, IFI2712A, CHIT1, YKL-40, GFAP, YWHAE, CSF1, AIF1, LY86, CD86, and TOP2A;
(b) the one or more inflammatory biomarkers are selected from the group consisting of osteopontin (SPP1), YWHAE (14-3-3 protein epsilon), allograft inflammatory factor 1 (AIF1), colony stimulating factor 1 (CSF1), chitinase 1 (CHIT1), lymphocyte antigen 86 (LY86), and CD86;
(c) the one or more biomarkers of complement function are selected from the group consisting of C1qb and C1qc; and/or
(d) the one or more biomarkers of microglial activity are selected from the group consisting of YKL-40, GFAP and interleukin-6.

36. The method of any one of claims 1-7 and 26-35, wherein administration to the individual of one dose of 6 mg/kg or 15 mg/kg of the anti-sortilin antibody by intravenous infusion results in an increase in the level of progranulin in the plasma of the individual of at least about 1.4-fold, at least about 1.8-fold, at least about 2-fold, at least about 2.2-fold, at least about 2.4-fold, at least about 2.6-fold, at least about 2.8-fold, or at least about 3-fold, compared to the level of progranulin in the plasma of the individual prior to administration of the anti-sortilin antibody.

37. The method of claim 36, wherein the increase in the level of progranulin in the plasma of the individual is present within about 1 day after administration of the anti-sortilin antibody.

38. The method of claim 36 or claim 37, wherein the dose of the anti-sortilin antibody is 6 mg/kg, and wherein the increase in the level of progranulin in the plasma of the individual is present at about 1 day, at about 2 days, at about 5 days, at about 7 days, at about 12 days, at about 17 days, at about 24 days, at about 29 days, or at about 42 days after administration of the anti-sortilin antibody.

39. The method of claim 36 or claim 37, wherein the dose of the anti-sortilin antibody is 15 mg/kg, and wherein the increase in the level of progranulin in the plasma of the individual is present at about 1 day, at about 2 days, at about 5 days, at about 7 days, at about 12 days, at about 17 days, at about 24 days, at about 29 days, at about 42 days, or at about 56 days after administration of the anti-sortilin antibody.

40. The method of any one of claims 1-7 and 26-35, wherein administration to the individual of one dose of 6 mg/kg, 15 mg/kg, 30 mg/kg or 60 mg/kg of the anti-sortilin antibody by intravenous infusion results in an increase in the level of progranulin in the plasma of the individual of at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300%, compared to the level of progranulin in the plasma of the individual prior to administration of the anti-sortilin antibody.

41. The method of claim 40, wherein the dose of the anti-sortilin antibody is 6 mg/kg, and wherein the increase in the level of progranulin in the plasma of the individual is present at about 1 day, at about 7 days, at about 14 days, at about 21 days, or at about 28 days after administration of the anti-sortilin antibody.

42. The method of claim 40, wherein the dose of the anti-sortilin antibody is 15 mg/kg, and wherein the increase in the level of progranulin in the plasma of the individual is present at about 1 day, at about 7 days, at about 14 days, at about 21 days, at about 28 days, at about 35 days, or at about 42 days after administration of the anti-sortilin antibody.

43. The method of claim 40, wherein the dose of the anti-sortilin antibody is 30 mg/kg, and wherein the increase in the level of progranulin in the plasma of the individual is present at about 1 day, at about 7 days, at about 14 days, at about 21 days, at about 28 days, at about 35 days, at about 42 days, at about 49 days, or at about 56 days after administration of the anti-sortilin antibody.

44. The method of claim 40, wherein the dose of the anti-sortilin antibody is 60 mg/kg, and wherein the increase in the level of progranulin in the plasma of the individual is present at about 1 day, at about 7 days, at about 14 days, at about 21 days, at about 28 days, at about 35 days, at about 42 days, at about 49 days, at about 56 days, at about 63 days, at about 70 days, at about 77 days, or at about 84 days after administration of the anti-sortilin antibody.

45. The method of any one of claims 1-4, 18-20, and 26-35, wherein administration to the individual of one dose of about 150 mg of the anti-sortilin antibody by subcutaneous injection results in an increase in the level of progranulin in the plasma of the individual of at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, or at least about 200%, compared to the level of progranulin in the plasma of the individual prior to administration of the anti-sortilin antibody.

46. The method of any one of claims 1-4, 17-19, 22, and 26-35, wherein administration to the individual of one dose of about 300 mg of the anti-sortilin antibody by subcutaneous injection results in an increase in the level of progranulin in the plasma of the individual of at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, or at least about 200%, compared to the level of progranulin in the plasma of the individual prior to administration of the anti-sortilin antibody.

47. The method of any one of claims 1-4, 17-19, 23, and 26-35, wherein administration to the individual of one dose of about 600 mg of the anti-sortilin antibody by subcutaneous injection results in an increase in the level of progranulin in the plasma of the individual of at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, or at least about 200%, compared to the level of progranulin in the plasma of the individual prior to administration of the anti-sortilin antibody.

48. The method of any one of claims 45-47, wherein the increase in the level of progranulin in the plasma of the individual is present at about 1 day, at about 7 days, at about 14 days, at about 21 days, or at about 28 days after administration of the anti-sortilin antibody.

49. The method of any one of claims 1-7, 26-38 and 40-41, wherein administration to the individual of one dose of 6 mg/kg of the anti-sortilin antibody by intravenous infusion results in an increase in the level of progranulin in the cerebrospinal fluid of the individual of at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, or at least about 20%, compared to the level of progranulin in the cerebrospinal fluid of the individual prior to administration of the anti-sortilin antibody.

50. The method of any one of claims 1-7, 26-37, 39-40 and 42, wherein administration to the individual of one dose of 15 mg/kg of the anti-sortilin antibody by intravenous infusion results in an increase in the level of progranulin in the cerebrospinal fluid of the individual of at least about 5%, at least about 10%, at least about 20%, at least about 30%, at least about 40%, at least about 50%, at least about 60%, at least about 70%, or at least about 80%, compared to the level of progranulin in the cerebrospinal fluid of the individual prior to administration of the anti-sortilin antibody.

51. The method of any one of claims 1-7, 26-35, 40, and 43, wherein administration to the individual of one dose of 30 mg/kg of the anti-sortilin antibody by intravenous infusion results in an increase in the level of progranulin in the cerebrospinal fluid of the individual of at least about 5%, at least about 10%, at least about 20%, at least about 30%, at least about 40%, at least about 50%, at least about 60%, or at least about 70%, compared to the level of progranulin in the cerebrospinal fluid of the individual prior to administration of the anti-sortilin antibody.

52. The method of any one of claims 1-5, 7, 26-35, 40 and 44, wherein administration to the individual of one dose of 60 mg/kg of the anti-sortilin antibody by intravenous infusion results in an increase in the level of progranulin in the cerebrospinal fluid of the individual of at least about 5%, at least about 10%, at least about 20%, at least about 30%, at least about 40%, at least about 50%, at least about 60%, or at least about 70%, compared to the level of progranulin in the cerebrospinal fluid of the individual prior to administration of the anti-sortilin antibody.

53. The method of any one of claims 49-51, wherein the increase in the level of progranulin in the cerebrospinal fluid of the individual is present at about 1 day, at about 2 days, at about 5 days, at about 7 days, at about 12 days, at about 14 days, at about 17 days, at about 21 days, at about 24 days, at about 28 days, at about 29 days, at about 35 days, at about 42 days, at about 49 days, or at about 56 days after administration of the anti-sortilin antibody.

54. The method of claim 52, wherein the increase in the level of progranulin in the cerebrospinal fluid of the individual is present at about 1 day, at about 7 days, at about 14 days, at about 21 days, at about 28 days, at about 35 days, at about 42 days, at about 49 days, at about 56 days, at about 63 days, at about 70 days, at about 77 days, or at about 84 days after administration of the anti-sortilin antibody.

55. The method of any one of claims 1-4, 18-20, 26-35, 45 and 48, wherein administration to the individual of one dose of about 150 mg of the anti-sortilin antibody by subcutaneous injection results in an increase in the level of progranulin in the cerebrospinal fluid of the individual of at least about 5%, at least about 10%, at least about 20%, at least about 30%, at least about 40%, at least about 50%, at least about 60%, or at least about 70%, compared to the level of progranulin in the cerebrospinal fluid of the individual prior to administration of the anti-sortilin antibody.

56. The method of any one of claims 1-4, 17-19, 22, 26-35, 46 and 48, wherein administration to the individual of one dose of about 300 mg of the anti-sortilin antibody by subcutaneous injection results in an increase in the level of progranulin in the cerebrospinal fluid of the individual of at least about 5%, at least about 10%, at least about 20%, at least about 30%, at least about 40%, at least about 50%, at least about 60%, or at least about 70%, compared to the level of progranulin in the cerebrospinal fluid of the individual prior to administration of the anti-sortilin antibody.

57. The method of any one of claims 1-4, 17-19, 23, 26-35, 47 and 48, wherein administration to the individual of one dose of about 600 mg of the anti-sortilin antibody by subcutaneous injection results in an increase in the level of progranulin in the cerebrospinal fluid of the individual of at least about 5%, at least about 10%, at least about 20%, at least about 30%, at least about 40%, at least about 50%, at least about 60%, or at least about 70%, compared to the level of progranulin in the cerebrospinal fluid of the individual prior to administration of the anti-sortilin antibody.

58. The method of any one of claims 55-57, wherein the increase in the level of progranulin in the cerebrospinal fluid of the individual is present at about 1 day, at about 7 days, at about 14 days, or at about 21 days after administration of the anti-sortilin antibody.

59. The method of any one of claims 1-58, wherein the disease, disorder, or injury is selected from the group consisting of frontotemporal dementia, progressive supranuclear palsy, Alzheimer's disease, vascular dementia, seizures, retinal dystrophy, amyotrophic lateral sclerosis (ALS), traumatic brain injury, a spinal cord injury, dementia, stroke, Parkinson's disease, acute disseminated encephalomyelitis, retinal degeneration, age related macular degeneration, glaucoma, multiple sclerosis, septic shock, bacterial infection, arthritis, and osteoarthritis.

60. The method of claim 59, wherein the individual is at risk for the disease, disorder, or injury.

61. The method of any one of claims 1-60, wherein the disease, disorder, or injury is Parkinson's disease.

62. The method of claim 61, wherein the disease, disorder, or injury is sporadic Parkinson's disease.

63. The method of claim 61, wherein the individual has at least one pathogenic mutation in the GBA1 gene.

64. The method of claim 63, wherein the individual is homozygous or heterozygous for the at least one pathogenic mutation in the GBA1 gene.

65. The method of claim 63 or claim 64, wherein the at least one pathogenic mutation in the GBA1 gene is selected from the group consisting of c.1226A>G, c.1448T>C, IVS2+1G>A, RecNciI, 84insGG, and any combination thereof; or wherein the at least one pathogenic mutation in the GBA1 gene is a mutation in the GBA1 gene resulting in an amino acid substitution in a GBA1 gene product selected from the group consisting of N370S, L444P, R120W, H255Q, D409H, E326K, T369M, R496H, and any combination thereof

66. The method of any one of claims 61-65 wherein the Parkinson's disease is classified as being between Stage I and Stage III, based on the Hoehn and Yahr criteria.

67. The method of any one of claims 61-66, wherein the individual is taking one or more treatments for Parkinson's disease prior to administration of the anti-sortilin antibody.

68. The method of claim 67, wherein the individual continues taking the one or more treatments for Parkinson's disease after the start of treatment with the anti-sortilin antibody.

69. The method of any one of claims 61-68, comprising administering the anti-sortilin antibody in combination with one or more treatments for Parkinson's disease.

70. The method of any one of claims 67-69, wherein the one or more treatments for Parkinson's disease are selected from the group consisting of: a glutamate antagonist, an anticholinergic, a dopamine agonist, Levodopa (L-DOPA and decarboxylase [DDC] inhibitor), a monoamine oxidase B (MAO-B) inhibitor, a catechol-O-methyltransferase (COMT) inhibitor, a beta blocker, a selective serotonin uptake inhibitor (SSRI), a tricyclic antidepressant (TCA), and indomethacin.

71. The method of any one of claims 61-70, further comprising measuring the levels of progranulin, GCase, and/or alpha-synuclein in a sample of plasma or cerebrospinal fluid obtained from the individual before and after the individual has received one or more doses of the anti-sortilin antibody.

72. The method of any one of claims 61-71, further comprising measuring the level of one or more biomarkers of lysosomal function in a sample of plasma or cerebrospinal fluid obtained from the individual before and after the individual has received one or more doses of the anti-sortilin antibody.

73. The method of claim 72, wherein the one or more biomarkers of lysosomal function are selected from the group consisting of: GCase protein, GCase activity, lyso-Gb1, one or more cathepsins, LAMP1, N-acetyl-D-glucosamine kinase (NAGK), and glucosylceramide; optionally, wherein the one or more cathepsins are selected from the group consisting of cathepsin B (CTSB) and cathepsin D (CTSD).

74. The method of any one of claims 61-73, further comprising assessing cognitive function of the individual before and after the individual has received one or more doses of the anti-sortilin antibody.

75. The method of claim 74, wherein cognitive function is assessed using the Montreal Cognitive Assessment (MoCA).

76. The method of any one of claims 61-75, further comprising assessing motor function of the individual before and after the individual has received one or more doses of the anti-sortilin antibody.

77. The method of claim 76, wherein motor function is assessed using the Movement Disorder Society (MDS)-sponsored revision of the Unified Parkinson's Disease Rating Scale (UPDRS) Part III (MDS-UPDRS Part III), or the UPDRS total scale.

78. The method of any one of claims 61-77, comprising administering the anti-sortilin antibody to the individual for at least about 6 months or at least about 24 weeks.

79. The method of any one of claims 1-60, wherein the disease, disorder, or injury is frontotemporal dementia (FTD).

80. The method of claim 79, wherein the individual:
(a) is heterozygous for one or more mutations in the GRN gene, optionally wherein the one or more mutations are loss-of-function mutations;
(b) is heterozygous for a C9orf72 hexanucleotide repeat expansion;
(c) has symptoms of FTD, does not have symptoms of FTD, or has pre-symptomatic FTD; and/or
(d) has FTD-GRN, or FTD caused by one or more mutations in the GRN gene.

81. The method of claim 80, wherein:
(a) the individual has pre-symptomatic FTD and: (i) elevated levels of one or more biomarkers selected from the group consisting of Nfl, SPP1, YWHAE, AIF1, CSF1, CHIT1, and LY86, and/or (ii) reduced levels of one or more biomarkers selected from the group consisting of NAGK and CTSB; or
(b) the individual does not have symptoms of FTD and: (i) is heterozygous for one or more mutations in the GRN gene, and/or (ii) has reduced PGRN levels or function.

82. The method of any one of claims 1-60, wherein the disease, disorder, or injury is Alzheimer's disease.

83. The method of any one of claims 1-60, wherein the disease, disorder, or injury is ALS.

84. The method of any one of claims 79-83, further comprising measuring the levels of progranulin, GCase, and/or alpha-synuclein in a sample of plasma or cerebrospinal fluid obtained from the individual before and after the individual has received one or more doses of the anti-sortilin antibody.

85. The method of any one of claims 79-83, further comprising measuring the level of one or more biomarkers of lysosomal function in a sample of plasma or cerebrospinal fluid obtained from the individual before and after the individual has received one or more doses of the anti-sortilin antibody.

86. The method of claim 85, wherein the one or more biomarkers of lysosomal function are selected from the group consisting of: GCase protein, GCase activity, lyso-Gb1, one or more cathepsins, LAMP1, N-acetyl-D-glucosamine kinase (NAGK), and glucosylceramide; optionally, wherein the one or more cathepsins are selected from the group consisting of cathepsin B (CTSB) and cathepsin D (CTSD).

87. The method of any one of claims 79-83, further comprising assessing cognitive function of the individual before and after the individual has received one or more doses of the anti-sortilin antibody.

88. The method of any one of claims 1-87, wherein the individual is a human.

89. A method of monitoring treatment of an individual being administered an anti-sortilin antibody, comprising measuring the level of one or more biomarkers, wherein the one or more biomarkers are selected from the group consisting of progranulin, GCase, neurofilament light chain (NF-L), Tau, one or more markers of neuroinflammation, one or more inflammatory biomarkers, one or more biomarkers of complement function, one or more biomarkers of microglial activity, and alpha-synuclein, wherein the level of the one or more biomarkers is measured in a sample of plasma or cerebrospinal fluid obtained from the individual before and after the individual has received one or more doses of an anti-sortilin antibody.

90. A method of monitoring treatment of an individual being administered an anti-sortilin antibody, comprising measuring the level of one or more biomarkers of lysosomal function in a sample of plasma or cerebrospinal fluid obtained from the individual before and after the individual has received one or more doses of an anti-sortilin antibody.

91. The method of claim 90, wherein the one or more biomarkers of lysosomal function are selected from the group consisting of: GCase protein, GCase activity, lyso-Gb1, one or more cathepsins, LAMP1, N-acetyl-D-glucosamine kinase (NAGK), and glucosylceramide; optionally, wherein the one or more cathepsins are selected from the group consisting of cathepsin B (CTSB) and cathepsin D (CTSD).

92. The method of any one of claims 89-91, further comprising assessing the activity of the anti-sortilin antibody in the individual based on the level of the one or more biomarkers in the sample.

93. The method of claim 89 or claim 92, wherein:
(a) the one or more markers of neuroinflammation are selected from the group consisting of IL-6, SPP1, IFI2712A, CHIT1, YKL-40, GFAP, YWHAE, CSF1, AIF1, LY86, CD86, and TOP2A;
(b) the one or more inflammatory biomarkers are selected from the group consisting of osteopontin (SPP1), YWHAE (14-3-3 protein epsilon), allograft inflammatory factor 1 (AIF1), colony stimulating factor 1 (CSF1), chitinase 1 (CHIT1), lymphocyte antigen 86 (LY86), and CD86;
(c) the one or more biomarkers of complement function are selected from the group consisting of C1qb and C1qc; and/or
(d) the one or more biomarkers of microglial activity are selected from the group consisting of YKL-40, GFAP and interleukin-6.

94. An anti-sortilin antibody at a dose of at least about 6 mg/kg for use in a method of treating or delaying the progression of a disease, disorder, or injury in an individual, wherein the antibody comprises a light chain variable domain and a heavy chain variable domain, wherein the heavy chain variable domain comprises an HVR-H1 comprising the amino acid sequence of SEQ ID NO:6, an HVR-H2 comprising the amino acid sequence of SEQ ID NO: 7, and an HVR-H3 comprising the amino acid sequence of SEQ ID NO:8, and the light chain variable domain comprises an HVR-L1 comprising the amino acid sequence of SEQ ID NO:9, an HVR-L2 comprising the amino acid sequence of SEQ ID NO:10, and an HVR-L3 comprising the amino acid sequence of SEQ ID NO: 11.

95. An anti-sortilin antibody at a dose of at least about 6 mg/kg for use in a method for increasing progranulin levels in an individual having a disease, disorder, or injury, wherein the antibody comprises a light chain variable domain and a heavy chain variable domain, wherein the heavy chain variable domain comprises an HVR-H1 comprising the amino acid sequence of SEQ ID NO:6, an HVR-H2 comprising the amino acid sequence of SEQ ID NO: 7, and an HVR-H3 comprising the amino acid sequence of SEQ ID NO:8, and the light chain variable domain comprises an HVR-L1 comprising the amino acid sequence of SEQ ID NO:9, an HVR-L2 comprising the amino acid sequence of SEQ ID NO:10, and an HVR-L3 comprising the amino acid sequence of SEQ ID NO: 11.

96. A use of an anti-sortilin antibody at a dose of at least about 6 mg/kg in the manufacture of a medicament for treating or delaying the progression of a disease, disorder, or injury in an individual, wherein the antibody comprises a light chain variable domain and a heavy chain variable domain, wherein the heavy chain variable domain comprises an HVR-H1 comprising the amino acid sequence of SEQ ID NO:6, an HVR-H2 comprising the amino acid sequence of SEQ ID NO: 7, and an HVR-H3 comprising the amino acid sequence of SEQ ID NO:8, and the light chain variable domain comprises an HVR-L1 comprising the amino acid sequence of SEQ ID NO:9, an HVR-L2 comprising the amino acid sequence of SEQ ID NO: 10, and an HVR-L3 comprising the amino acid sequence of SEQ ID NO:11.

97. A use of an anti-sortilin antibody at a dose of at least about 6 mg/kg in the manufacture of a medicament for increasing progranulin levels in an individual having a disease, disorder, or injury, wherein the antibody comprises a light chain variable domain and a heavy chain variable domain, wherein the heavy chain variable domain comprises an HVR-H1 comprising the amino acid sequence of SEQ ID NO:6, an HVR-H2 comprising the amino acid sequence of SEQ ID NO: 7, and an HVR-H3 comprising the amino acid sequence of SEQ ID NO:8, and the light chain variable domain comprises an HVR-L1 comprising the amino acid sequence of SEQ ID NO:9, an HVR-L2 comprising the amino acid sequence of SEQ ID NO: 10, and an HVR-L3 comprising the amino acid sequence of SEQ ID NO:11.


SEQUENCE LISTING (32 sequences)

SEQ ID NO: 1 - protein, 831 aa, Homo sapiens
MERPWGAADGLSRWPHGLGLLLLLQLLPPSTLSQDRLDAPPPPAAPLPRWSGPIGVSWGLRAAAAGGAFPRGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCKADLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNSVPIILAIVGLMLVTVVAGVLIVKKYVCGGRFLVHRYSVLQQHAEANGVDGVDALDTASHTNKSGYHDDSDEDLLE

SEQ ID NO: 2 - protein, 825 aa, Mus musculus
MERPRGAADGLLRWPLGLLLLLQLLPPAAVGQDRLDAPPPPAPPLLRWAGPVGVSWGLRAAAPGGPVPRAGRWRRGAPAEDQDCGRLPDFIAKLTNNTHQHVFDDLSGSVSLSWVGDSTGVILVLTTFQVPLVIVSFGQSKLYRSEDYGKNFKDITNLINNTFIRTEFGMAIGPENSGKVILTAEVSGGSRGGRVFRSSDFAKNFVQTDLPFHPLTQMMYSPQNSDYLLALSTENGLWVSKNFGEKWEEIHKAVCLAKWGPNNIIFFTTHVNGSCKADLGALELWRTSDLGKTFKTIGVKIYSFGLGGRFLFASVMADKDTTRRIHVSTDQGDTWSMAQLPSVGQEQFYSILAANEDMVFMHVDEPGDTGFGTIFTSDDRGIVYSKSLDRHLYTTTGGETDFTNVTSLRGVYITSTLSEDNSIQSMITFDQGGRWEHLRKPENSKCDATAKNKNECSLHIHASYSISQKLNVPMAPLSEPNAVGIVIAHGSVGDAISVMVPDVYISDDGGYSWAKMLEGPHYYTILDSGGIIVAIEHSNRPINVIKFSTDEGQCWQSYVFTQEPIYFTGLASEPGARSMNISIWGFTESFITRQWVSYTVDFKDILERNCEEDDYTTWLAHSTDPGDYKDGCILGYKEQFLRLRKSSVCQNGRDYVVAKQPSVCPCSLEDFLCDFGYFRPENASECVEQPELKGHELEFCLYGKEEHLTTNGYRKIPGDKCQGGMNPAREVKDLKKKCTSNFLNPTKQNSKSNSVPIILAIVGLMLVTVVAGVLIVKKYVCGGRFLVHRYSVLQQHAEADGVEALDSTSHAKSGYHDDSDEDLLE

SEQ ID NO: 3 - protein, 825 aa, Rattus norvegicus
MERPRGAADGLLRWPLGLLLLLQLLPPAAVGQDRLDAPPPPAPPLLRWAGPVGVSWGLRAAAPGGPVPRAGRWRRGAPAEDQDCGRLPDFIAKLTNNTHQHVFDDLSGSVSLSWVGDSTGVILVLTTFQVPLVIVSFGQSKLYRSEDYGKNFKDITNLINNTFIRTEFGMAIGPENSGKVILTAEVSGGSRGGRVFRSSDFAKNFVQTDLPFHPLTQMMYSPQNSDYLLALSTENGLWVSKNFGEKWEEIHKAVCLAKWGPNNIIFFTTHVNGSCKADLGALELWRTSDLGKTFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSTLSEDNSIQSMITFDQGGRWEHLQKPENSKCDATAKNKNECSLHIHASYSISQKLNVPMAPLSEPNAVGIVIAHGSVGDAISVMVPDVYISDDGGYSWAKMLEGPHYYTILDSGGIIVAIEHSNRPINVIKFSTDEGQCWQSYVFSQEPVYFTGLASEPGARSMNISIWGFTESFLTRQWVSYTIDFKDILERNCEENDYTTWLAHSTDPGDYKDGCILGYKEQFLRLRKSSVCQNGRDYVVAKQPSICPCSLEDFLCDFGYFRPENASECVEQPELKGHELEFCLYGKEEHLTTNGYRKIPGDRCQGGMNPAREVKDLKKKCTSNFLNPKKQNSKSSSVPIILAIVGLMLVTVVAGVLIVKKYVCGGRFLVHRYSVLQQHAEADGVEALDTASHAKSGYHDDSDEDLLE

SEQ ID NO: 4 - protein, 11 aa, Artificial Sequence
  Synthetic Construct
NGLWVSKNFGG

SEQ ID NO: 5 - protein, 25 aa, Artificial Sequence
  Synthetic Construct
FASVMADKDTTRRIHVSTDQGDTWS

SEQ ID NO: 6 - protein, 9 aa, Artificial Sequence
  Synthetic Construct
YTFTKYYMS

SEQ ID NO: 7 - protein, 17 aa, Artificial Sequence
  Synthetic Construct
IINPIGGSTSYAQKFQG

SEQ ID NO: 8 - protein, 22 aa, Artificial Sequence
  Synthetic Construct
ARDPSGIALAGPASRGYQGMDV

SEQ ID NO: 9 - protein, 11 aa, Artificial Sequence
  Synthetic Construct
RASQSVSSNLA

SEQ ID NO: 10 - protein, 7 aa, Artificial Sequence
  Synthetic Construct
GASTRAT

SEQ ID NO: 11 - protein, 9 aa, Artificial Sequence
  Synthetic Construct
QQARLGPWT

SEQ ID NO: 12 - protein, 26 aa, Artificial Sequence
  Synthetic Construct
QVQLVQSGAEVKKPGASVKVSCKASG

SEQ ID NO: 13 - protein, 14 aa, Artificial Sequence
  Synthetic Construct
WVRQAPGQGLEWMG

SEQ ID NO: 14 - protein, 30 aa, Artificial Sequence
  Synthetic Construct
RVTMTRDTSTSTVYMELSSLRSEDTAVYYC

SEQ ID NO: 15 - protein, 11 aa, Artificial Sequence
  Synthetic Construct
WGQGTTVTVSS

SEQ ID NO: 16 - protein, 23 aa, Artificial Sequence
  Synthetic Construct
EIVMTQSPATLSVSPGERATLSC

SEQ ID NO: 17 - protein, 15 aa, Artificial Sequence
  Synthetic Construct
WYQQKPGQAPRLLIY

SEQ ID NO: 18 - protein, 32 aa, Artificial Sequence
  Synthetic Construct
GIPARFSGSGSGTEFTLTISSLQSEDFAVYYC

SEQ ID NO: 19 - protein, 10 aa, Artificial Sequence
  Synthetic Construct
FGGGTKVEIK

SEQ ID NO: 20 - protein, 129 aa, Artificial Sequence
  Synthetic Construct
QVQLVQSGAEVKKPGASVKVSCKASGYTFTKYYMSWVRQAPGQGLEWMGIINPIGGSTSYAQKFQGRVTMTRDTSTSTVYMELSSLRSEDTAVYYCARDPSGIALAGPASRGYQGMDVWGQGTTVTVSS

SEQ ID NO: 21 - protein, 107 aa, Artificial Sequence
  Synthetic Construct
EIVMTQSPATLSVSPGERATLSCRASQSVSSNLAWYQQKPGQAPRLLIYGASTRATGIPARFSGSGSGTEFTLTISSLQSEDFAVYYCQQARLGPWTFGGGTKVEIK

SEQ ID NO: 22 - protein, 8 aa, Artificial Sequence
  Synthetic Construct
  VARIANT 1: Xaa = Ser or Thr
  VARIANT 2, 4, 5, 6, 7: Xaa = Any Amino Acid
  VARIANT 3: Xaa = Asp or Asn
  VARIANT 8: Xaa = Trp, Phe, or Tyr
XXXXXXXX

SEQ ID NO: 23 - protein, 11 aa, Artificial Sequence
  Synthetic Construct
  VARIANT 1, 2, 4, 6, 8, 11: Xaa = Any Amino Acid
  VARIANT 3: Xaa = Ser or Thr
  VARIANT 5: Xaa = Asp or Asn
  VARIANT 9: Xaa = Ser or Thr
  VARIANT 10: Xaa = Trp, Phe, or Tyr
XXXXXXGXXXX

SEQ ID NO: 24 - protein, 8 aa, Homo sapiens
SSDFAKNF

SEQ ID NO: 25 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 329 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 831 aa, Macaca fascicularis
MERPRGAADGLSRWPHGLGLLLLLQLLPPVTLGQDRLDAPPPPAAPLLRWSGPIGVSWGLRAAAAGGAFPRGGRWRRSAPGEDEECGRVRDFVAKLANNTHQHVFDDLRGSVSLSWVGDSTGVILVLTTFHVPLVIMTFGQSKLYRSEDYGKNFKDITNLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCKADLGALELWRTLDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWKHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNSVPIILAIVGLMLVTVIAGVLIVKKYVCGGRFLVHRYSVLQQHAEANGMDGVDALDTASHTNKSGYHDDSDEDLLE

SEQ ID NO: 28 - protein, 330 aa, Artificial Sequence [sequence omitted]

SEQ ID NO: 29 - protein, 329 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 214 aa, Artificial Sequence
  Synthetic Construct
EIVMTQSPATLSVSPGERATLSCRASQSVSSNLAWYQQKPGQAPRLLIYGASTRATGIPARFSGSGSGTEFTLTISSLQSEDFAVYYCQQARLGPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 459 aa, Artificial Sequence
  Synthetic Construct
QVQLVQSGAEVKKPGASVKVSCKASGYTFTKYYMSWVRQAPGQGLEWMGIINPIGGSTSYAQKFQGRVTMTRDTSTSTVYMELSSLRSEDTAVYYCARDPSGIALAGPASRGYQGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 458 aa, Artificial Sequence
  Synthetic Construct
QVQLVQSGAEVKKPGASVKVSCKASGYTFTKYYMSWVRQAPGQGLEWMGIINPIGGSTSYAQKFQGRVTMTRDTSTSTVYMELSSLRSEDTAVYYCARDPSGIALAGPASRGYQGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG